(19) United States
(12) Patent Application Publication
(10) Pub. No.: US 2019/0231795 A1
(43) Pub. Date: Aug. 1, 2019

(54) METHODS FOR TREATING CANCER AND PREDICTING DRUG RESPONSIVENESS IN CANCER PATIENTS

(71) Applicant: LiPlasome Pharma ApS, Vejle (DK)

(72) Inventors: Steen KNUDSEN, Scottsdale, AZ (US); Peter Buhl JENSEN, Farum (DK); Ulla Hald BUHL, Farum (DK); Annie RASMUSSEN, Hellerup (DK); Mogens Winkel MADSEN, Virum (DK)

(21) Appl. No.: 16/257,831

(22) Filed: Jan. 25, 2019

Related U.S. Application Data
(60) Provisional application No. 62/624,538, filed on Jan. 31, 2018.

(51) Int. Cl. A61K 31/555; A61K 9/00; C12Q 1/02; A61P 35/00; C12Q 1/6834; C12Q 1/6886

(52) U.S. Cl. CPC A61K 31/555; A61K 9/0019; C12Q 1/025; G01N 2800/52; C12Q 1/6834; C12Q 1/6886; G01N 2800/60; A61P 35/00

(57) ABSTRACT

Featured are methods of treating a patient with cancer by administering, e.g., a secretory phospholipase A₂ (sPLA₂) hydrolysable, cisplatin-containing liposome composition (e.g., LiPlaCis). The patient may be assessed for their responsiveness to the liposomal therapy prior to treatment using the methods, devices, and kits also described herein for detecting a level of one or more biomarkers in a sample from the patient with cancer.

### FIELD OF THE INVENTION

The invention pertains to methods of treating cancer in subjects in need thereof and using biomarkers to predict responsiveness of a cancer to a cancer treatment.

### BACKGROUND

Cancer remains one of the deadliest threats to human health. In 2013, the global cancer burden was estimated to be at least 14.1 million new cases and 8.2 million cancer deaths. These statistics are predicted to increase further by 2025. An effective treatment strategy is needed.

Cisplatin, an inorganic platinum-based anti-neoplastic agent, is one of the most effective and widely used anticancer drugs in the world and is commonly used for the treatment of a wide variety of cancers, such as breast, testicular, lung and ovarian cancers. A major obstacle to widespread use of cisplatin is the persistence of severe toxic side effects. Thus, there exists a need for improved cisplatin formulations and dosage regimens for treating cancer that produce fewer toxic side effects. Methods for determining whether a cancer will be responsive to a cisplatin therapy are also needed.

### SUMMARY OF THE INVENTION

Featured are methods for treating cancer using two doses of a liposomal cisplatin formulation (e.g., LiPlaCis) given on day 1 and day 8 of a three week treatment cycle. Also featured are methods for determining the responsiveness of a subject (e.g., a human) with a cancer (e.g., breast cancer) to treatment with the liposomal cisplatin formulation (e.g., LiPlaCis) by detecting a level of one or more biomarkers of sensitivity and/or resistance, such as the biomarkers set forth in one or more of Tables 2-5.

In a first aspect is a method of treating a subject (e.g., a human) with a cancer by administering to the subject at least two doses (e.g., first and second doses) of a composition that contains a secretory phospholipase A2 (sPLA₂) hydrolysable, cisplatin-containing liposome (e.g., LiPlaCis) on day 1 and day 8, respectively, of at least one three week treatment cycle, in which each of the doses of the liposomal composition contain cisplatin in an amount of about 75 mg to about 90 mg, or cisplatin in an amount of about 40 mg/m² body surface area to about 55 mg/m² body surface area of the subject.

In some embodiments of the first aspect, the first and/or second doses of the liposomal composition contain about 75 mg cisplatin. In other embodiments, the first and/or second doses of the liposomal composition contain about 90 mg cisplatin.

In other embodiments of the first aspect, the first and/or second doses of the composition contain cisplatin in an amount of about 40 mg/m² body surface area of the subject. In other embodiments, the first and/or second doses of the liposomal composition contain cisplatin in an amount of about 55 mg/m² body surface area of the subject.

The method may also involve administering the liposomal composition in an amount that provides about 150 mg to about 180 mg cisplatin to the subject in each three week treatment cycle. In some embodiments, an amount of about 150 mg cisplatin or an amount of about 180 mg cisplatin is administered to the subject in each three week treatment cycle.

In some embodiments of the first aspect, the method further includes the step of administering one or more additional therapies to the subject prior to, concurrently with, or after administration of the liposomal composition. The additional therapies may include surgery, radiation, or a therapeutic agent. The therapeutic agent may be selected from the group consisting of docetaxel, cabazitaxel, mitoxantrone, estramustine, prednisone, carboplatin, bevacizumab, paclitaxel, gemcitabine, doxorubicin, topotecan, etoposide, tamoxifen, letrozole, sorafenib, fluorouracil, capecitabine, oxaliplatin, interferon-alpha, 5-fluorouracil (5-FU), a histone deacetylase (HDAC) inhibitor, ipilimumab, bortezomib, carfilzomib, thalidomide, lenalidomide, pomalidomide, dexamethasone, cyclophosphamide, vincristine, melphalan, tegafur, irinotecan, cetuximab, leucovorin, SN-38, everolimus, temsirolimus, bleomycin, lomustine, depsipeptide, erlotinib, cisplatin, busulfan, epirubicin, arsenic trioxide, bendamustine, fulvestrant, teniposide, adriamycin, decitabine, estramustine, azaguanine, aclarubicin, mitomycin, paclitaxel, taxotere, APO010, ara-c, methylprednisolone, methotrexate, methyl-gag, belinostat, idarubicin, IL4-PR38, valproic acid, all-trans retinoic acid (ATRA), cytoxan, suberoylanilide hydroxamic acid, leukeran, fludarabine, vinblastine, dacarbazine, hydroxyurea, tegafur, daunorubicin, mechlorethamine, streptozocin, carmustine, mercaptopurine, dactinomycin, tretinoin, ifosfamide, floxuridine, thioguanine, PSC 833, herceptin, celecoxib, iressa, anastrozole, and rituximab.

In some embodiments of the first aspect, the liposomal composition is administered to the subject intravenously, intramuscularly, transdermally, intradermally, intra-arterially, intracranially, subcutaneously, intraorbitally, intraventricularly, intraspinally, intraperitoneally, or intranasally. For example, the liposomal composition is administered to the subject by intravenous infusion. In some embodiments, the liposomal composition is administered to the subject over a period of about 2-3 hours. For example, the composition is administered to the subject as a 2 or 3 hour infusion.

In some embodiments of the first aspect, the three week treatment cycle is repeated two to twenty times. For example, the three week treatment cycle can be repeated two times, three times, four times, five times, ten times, fifteen times, or twenty times. Each three week treatment cycle can begin immediately after the conclusion of the prior three week cycle or one or more of the three week cycles can be separated by a period of a day (e.g., 1-6 days), a week (e.g., 1-4 weeks), a month (e.g., 1-12 months), or a year.

In some embodiments of the first aspect, the subject has been determined to be responsive to the liposomal composition (e.g., LiPlaCis) prior to administration of the liposomal composition.

In other embodiments of the first aspect, the method of treating a subject with cancer with the liposomal composition (e.g., LiPlaCis) further includes the step of determining the responsiveness of the subject to the liposomal composition. Responsiveness of the subject to the liposomal composition can be determined, e.g., by contacting a sample from the subject (e.g., a sample containing one or more nucleic acid molecules from the subject, such as a tumor sample) with a device that contains (i) one or more single-stranded nucleic acid molecules capable of specifically hybridizing with nucleotides of one or more biomarkers of sensitivity selected from those listed in Tables 2 and/or 4, or a complement thereof; and/or (ii) one or more single-stranded nucleic acid molecules capable of specifically hybridizing with nucleotides of one or more biomarkers of resistance selected from those listed in Tables 3 and/or 5, or a complement thereof. The level of the one or more biomarkers of sensitivity or the complement thereof and/or the level of the one or more biomarkers of resistance, or a complement thereof, in the sample is detected by, e.g., detecting hybridization between the one or more single-stranded nucleic acid molecules of the device and the one or more nucleic acid molecules of the sample. In some embodiments, the one or more biomarkers of sensitivity is not C1QR1 (SEQ ID NO: 13), SLA (SEQ ID NO: 48), PTPN7 (SEQ ID NO: 77), CENTB1 (SEQ ID NO: 37), IFI16 (SEQ ID NO: 17 or 261), ARHGEF6 (SEQ ID NO: 36 or 294), CD3D (SEQ ID NO: 81), ARHGAP15 (SEQ ID NO: 30), HCLS1 (SEQ ID NO: 16 or 259), CD53 (SEQ ID NO: 282), PTPRCAP (SEQ ID NO: 8), and/or PTPRC (SEQ ID NO: 10, 18, 25, or 243).

In some embodiments of the first aspect, the subject is determined to be responsive to the liposomal composition (e.g., LiPlaCis) if: i) the level of the biomarker(s) of sensitivity, or the complement thereof, is substantially similar to the level of the biomarker(s) of sensitivity, or the complement thereof, in a cell or tissue known to be sensitive to the liposomal composition; and/or ii) the level of the biomarker(s) of resistance, or the complement thereof, is substantially dissimilar to the level of the biomarker(s) of resistance, or the complement thereof, in a cell or tissue known to be resistant to the liposomal composition.

In some embodiments, the responsiveness of the subject to the liposomal composition is determined by detecting the level of PLA2G2A (SEQ ID NO: 380), or a complement thereof, in the sample from the subject. For example, the responsiveness of the subject to the liposomal composition can be determined by detecting the level of PLA2G2A (SEQ ID NO: 380), or a complement thereof by performing microarray analysis or qRT-PCR.

In other embodiments, the method of determining the responsiveness of the subject to the liposomal composition (e.g., LiPlaCis) includes the step of detecting sPLA₂ protein in a tumor sample from the subject. The sPLA2 protein can be detected by contacting the tumor sample with an anti-sPLA₂ antibody and detecting binding between the sPLA₂ protein and the anti-sPLA₂ antibody. The method may include detecting the level of one or more biomarkers of sensitivity and/or resistance (Tables 2-5) in a sample from the subject and detecting the level of sPLA₂ protein in a tumor sample from the subject. In yet other embodiments, the method further includes the step of administering one or more cancer therapies other than the liposomal composition (e.g., LiPlaCis) to the subject when the subject is determined to be responsive to the liposomal composition.

In some embodiments of the first aspect, the cell (e.g., a cancer cell) or tissue (e.g., a tumor tissue) known to be sensitive to the liposomal composition and/or the cell or tissue known to be resistant to the liposomal composition is of the same type as a cell or tissue in the sample from the patient or from which the one or more nucleic acid molecules of the sample are derived. In particular, the cell (e.g., a cancer cell) or tissue (e.g., a tumor tissue) known to be sensitive to the liposomal composition and/or the cell or tissue known to be resistant to the liposomal composition is of the same type of cancer (e.g., breast cancer) as a cell or tissue in the sample from the subject or from which the one or more nucleic acid molecules of the sample are derived, which can provide, e.g., a control from which to assess whether the subject will be sensitive or resistant to the liposomal composition.

In some embodiments, the sample from the subject is a tumor sample. In some embodiments, the subject is resistant to one or more cancer therapies (e.g., surgery, radiation, or a therapeutic agent) other than the liposomal composition (e.g., LiPlaCis).

In some embodiments of the first aspect, the cancer is selected from a solid tumor cancer and a hematological cancer. For example, the cancer can be breast cancer, acute myelogenous leukemia (AML), acute lympho-blastic leukemia (ALL), chronic lymphocytic leukemia (CLL), myelodysplastic syndrome (MDS), chronic myelogenous leukemia—chronic phase (CMLCP), diffuse large B-cell lymphoma (DLBCL), cutaneous T-cell lymphoma (CTCL), peripheral T-cell lymphoma (PTCL), Hodgkin's lymphoma, hepatocellular carcinoma (HCC), cervical cancer, renal cell carcinoma (RCC), esophageal cancer, melanoma, glioma, pancreatic cancer, gastrointestinal stromal tumors (GIST), sarcoma, non-small cell lung carcinoma (NSCLC), prostate cancer, ovarian cancer, colon cancer, bladder cancer, and squamous cell carcinoma of the head and neck (SCCHN). In particular, the cancer can be breast cancer, such as an estrogen receptor-positive (ERpos) breast cancer and/or a metastatic form of breast cancer.

In some embodiments, the subject may exhibit cancer relapse (e.g., relapse of breast cancer), such as relapse after a first cancer treatment and prior to treatment with the liposomal composition (e.g., LiPlaCis). Alternatively, the subject may have not been administered any treatment for cancer prior to administration of the liposomal composition (e.g., LiPlaCis). Additionally, the responsiveness of the subject to the liposomal composition may not have been determined prior to treatment and/or may be determined during or after a cancer treatment (e.g., treatment with cisplatin, such as with LiPlaCis).

In some embodiments, the device for determining the responsiveness of a subject to treatment with a liposomal composition described herein (e.g., LiPlaCis) can include at least two, at least three, at least four, at least five, at least six, at least seven, at least eight, at least nine, at least ten, or more single-stranded nucleic acid molecules capable of specifically hybridizing with the nucleotides of one or more biomarkers of sensitivity selected from the biomarkers of Tables 2 and 4, or a complement thereof (e.g., COL5A2 (SEQ ID NO: 73 or 211); and/or at least two, at least three, at least four, at least five, at least six, at least seven, at least eight, at least nine, at least ten, or more single-stranded nucleic acid molecules capable of specifically hybridizing with the nucleotides of one or more biomarkers of resistance selected from the biomarkers of Tables 3 and 5, or a complement thereof (e.g., SFN (SEQ ID NO: 96 OR 324)). In particular, one or more of the single-stranded nucleic acid molecules of the device may have a length in the range of 10 to 100 nucleotides (e.g., a length in the range of 20 to 60 nucleotides). The one or more single-stranded nucleic acid molecules may also be labeled and/or immobilized on a solid substrate.

In some embodiments, the method for determining the responsiveness of a subject to treatment with a liposomal composition described herein (e.g., LiPlaCis) may include converting the level of the one or more biomarkers of sensitivity, or the complement thereof (e.g., one, two, three, four, five, ten, twenty, or all of the biomarkers shown in Tables 2 and 4, such as COL5A2 (SEQ ID NO: 73 or 211)), and/or the one or more biomarkers of resistance, or the complement thereof (e.g., one, two, three, four, five, ten, twenty, or all of the biomarkers shown in Tables 3 and 5, such as SFN (SEQ ID NO: 96 OR 324)), into a mean score, in which the mean score indicates the responsiveness of the subject to the liposomal composition (e.g., LiPlaCis). The method can further include subtracting the mean score for one or more of the biomarkers of resistance (e.g., one, two, three, four, five, ten, twenty, or all of the biomarkers shown in Tables 3 and 5, such as SFN (SEQ ID NO: 96 OR 324)) from the mean score for one or more of the biomarkers of sensitivity (e.g., one, two, three, four, five, ten, twenty, or all of the biomarkers shown in Tables 2 and 4, such as COL5A2 (SEQ ID NO: 73 or 211) to obtain a difference score, in which the difference score indicates the responsiveness of the subject to the liposomal composition. In particular, the mean score and/or the difference score above a cutoff value (e.g., a cutoff value of about 0.1, about 0.15, about 0.2, about 0.25, about 0.3, about 0.35, about 0.4, about 0.45, about 0.5, or greater) indicates that the subject is responsive to the liposomal composition.

In other embodiments, the device is a microarray, such as a deoxyribonucleic acid (DNA)-based platform. Alternatively, the device is for performing a qRT-PCR reaction (e.g., the device is used with a system for detecting the amplification product, for example, by fluorescence or by another method). The methods may also utilize both a microarray and a qRT-PCR device. Thus, the level of the biomarker(s) of sensitivity (e.g., one, two, three, four, five, ten, twenty, or all of the biomarkers shown in Tables 2 and 4, such as COL5A2 (SEQ ID NO: 73 or 211), and/or the biomarker(s) of resistance (e.g., one, two, three, four, five, ten, twenty, or all of the biomarkers shown in Tables 3 and 5, such as SFN (SEQ ID NO: 96 OR 324)), can be measured using qRT-PCR. In particular, the level of the one or more biomarkers of sensitivity, or the complement thereof (e.g., one, two, three, four, five, ten, twenty, or all of the biomarkers shown in Tables 2 and 4, such as COL5A2 (SEQ ID NO: 73 or 211)), and/or the one or more biomarkers of resistance, or the complement thereof (e.g., one, two, three, four, five, ten, twenty, or all of the biomarkers shown in Tables 3 and 5, such as SFN (SEQ ID NO: 96 OR 324)), are detected by performing microarray analysis or qRT-PCR. Additionally, the nucleic acid molecules of the sample may include mRNA or a cDNA thereof.

In still other embodiments, the biomarker of sensitivity may be selected from one or more of COL5A2 (SEQ ID NO: 73 or 211), ITGA4 (SEQ ID NO: 1), MSN (SEQ ID NO: 2), FAM46A (SEQ ID NO: 3 or 280), ITGB2 (SEQ ID NO: 4), DOCK2 (SEQ ID NO: 5 or 223), EVL (SEQ ID NO: 6), SACS (SEQ ID NO: 7), EBI2 (SEQ ID NO: 9), ANP32E (SEQ ID NO: 11), SFPQ (SEQ ID NO: 12, 38 or 272), FNBP1 (SEQ ID NO: 14 or 28), CBFB (SEQ ID NO: 15), SFRS7 (SEQ ID NO: 19 or 54), and CAP350 (SEQ ID NO: 20 or 61). The biomarker of resistance may be selected from one or more of S SFN (SEQ ID NO: 96 or 324), LISCH7 (SEQ ID NO: 97), EPB41L4B (SEQ ID NO: 98), MST1R (SEQ ID NO: 99), ITGB4 (SEQ ID NO: 100), DBNDD2 (SEQ ID NO: 102 or 365), TACSTD1 (SEQ ID NO: 104), MISP (SEQ ID NO: 105), KRT8 (SEQ ID NO: 106), JUP (SEQ ID NO: 107 or 400), KRT18 (SEQ ID NO: 108 or 306), FA2H (SEQ ID NO: 109), MGAT4B (SEQ ID NO: 110), DSG2 (SEQ ID NO:111 or 312), and LRP5 (SEQ ID NO: 112).

For example, the biomarkers of sensitivity may include COL5A2 (SEQ ID NO 73 or 211) and ITGA4 (SEQ ID NO: 1). The biomarkers of sensitivity may include COL5A2 (SEQ ID NO 73 or 211), ITGA4 (SEQ ID NO: 1), and MSN (SEQ ID NO: 2). The biomarkers of sensitivity may include COL5A2 (SEQ ID NO 73 or 211), ITGA4 (SEQ ID NO: 1), MSN (SEQ ID NO: 2), and FAM46A (SEQ ID NO: 3 OR 280). The biomarkers of sensitivity may include COL5A2 (SEQ ID NO 73 or 211), ITGA4 (SEQ ID NO: 1), MSN (SEQ ID NO: 2), FAM46A (SEQ ID NO: 3 OR 280), and ITGB2 (SEQ ID NO: 4). The biomarkers of sensitivity may include COL5A2 (SEQ ID NO 73 or 211), ITGA4 (SEQ ID NO: 1), MSN (SEQ ID NO: 2), FAM46A (SEQ ID NO: 3 OR 280), ITGB2 (SEQ ID NO: 4), and DOCK2 (SEQ ID NO: 5 OR 223). The biomarkers of sensitivity may include COL5A2 (SEQ ID NO 73 or 211), ITGA4 (SEQ ID NO: 1), MSN (SEQ ID NO: 2), FAM46A (SEQ ID NO: 3 OR 280), ITGB2 (SEQ ID NO: 4), DOCK2 (SEQ ID NO: 5 OR 223), and EVL (SEQ ID NO: 6). The biomarkers of sensitivity may include COL5A2 (SEQ ID NO 73 or 211), ITGA4 (SEQ ID NO: 1), MSN (SEQ ID NO: 2), FAM46A (SEQ ID NO: 3 OR 280), ITGB2 (SEQ ID NO: 4), DOCK2 (SEQ ID NO: 5 OR 223), EVL (SEQ ID NO: 6), and SACS (SEQ ID NO: 7). The biomarkers of sensitivity may include COL5A2 (SEQ ID NO 73 or 211), ITGA4 (SEQ ID NO: 1), MSN (SEQ ID NO: 2), FAM46A (SEQ ID NO: 3 OR 280), ITGB2 (SEQ ID NO: 4), DOCK2 (SEQ ID NO: 5 OR 223), EVL (SEQ ID NO: 6), SACS (SEQ ID NO: 7), and PTPRCAP (SEQ ID NO: 8). The biomarkers of sensitivity may include COL5A2 (SEQ ID NO 73 or 211), ITGA4 (SEQ ID NO: 1), MSN (SEQ ID NO: 2), FAM46A (SEQ ID NO: 3 OR 280), ITGB2 (SEQ ID NO: 4), DOCK2 (SEQ ID NO: 5 OR 223), EVL (SEQ ID NO: 6), SACS (SEQ ID NO: 7), PTPRCAP (SEQ ID NO: 8), and EBI2 (SEQ ID NO: 9). The biomarkers of sensitivity may include COL5A2 (SEQ ID NO 73 or 211), ITGA4 (SEQ ID NO: 1), MSN (SEQ ID NO: 2), FAM46A (SEQ ID NO: 3 OR 280), ITGB2 (SEQ ID NO: 4), DOCK2 (SEQ ID NO: 5 OR 223), EVL (SEQ ID NO: 6), SACS (SEQ ID NO: 7), PTPRCAP (SEQ ID NO: 8), EBI2 (SEQ ID NO: 9), and PTPRC (SEQ ID NO: 10, 18, 25, OR 243). The biomarkers of sensitivity may include COL5A2 (SEQ ID NO 73 or 211), ITGA4 (SEQ ID NO: 1), MSN (SEQ ID NO: 2), FAM46A (SEQ ID NO: 3 OR 280), ITGB2 (SEQ ID NO: 4), DOCK2 (SEQ ID NO: 5 OR 223), EVL (SEQ ID NO: 6), SACS (SEQ ID NO: 7), PTPRCAP (SEQ ID NO: 8), EBI2 (SEQ ID NO: 9), PTPRC (SEQ ID NO: 10, 18, 25, OR 243), and ANP32E (SEQ ID NO: 11). The biomarkers of sensitivity may include COL5A2 (SEQ ID NO 73 or 211), ITGA4 (SEQ ID NO: 1), MSN (SEQ ID NO: 2), FAM46A (SEQ ID NO: 3 OR 280), ITGB2 (SEQ ID NO: 4), DOCK2 (SEQ ID NO: 5 OR 223), EVL (SEQ ID NO: 6), SACS (SEQ ID NO: 7), PTPRCAP (SEQ ID NO: 8), EBI2 (SEQ ID NO: 9), PTPRC (SEQ ID NO: 10, 18, 25, OR 243), ANP32E (SEQ ID NO: 11), and SFPQ (SEQ ID NO: 12, 38 OR 272). The biomarkers of sensitivity may include COL5A2 (SEQ ID NO 73 or 211), ITGA4 (SEQ ID NO: 1), MSN (SEQ ID NO: 2), FAM46A (SEQ ID NO: 3 OR 280), ITGB2 (SEQ ID NO: 4), DOCK2 (SEQ ID NO: 5 OR 223), EVL (SEQ ID NO: 6), SACS (SEQ ID NO: 7), PTPRCAP (SEQ ID NO: 8), EBI2 (SEQ ID NO: 9), PTPRC (SEQ ID NO: 10, 18, 25, OR 243), ANP32E (SEQ ID NO: 11), SFPQ (SEQ ID NO: 12, 38 OR 272), and C1QR1 (SEQ ID NO: 13). The biomarkers of sensitivity may include COL5A2 (SEQ ID NO 73 or 211), ITGA4 (SEQ ID NO: 1), MSN (SEQ ID NO: 2), FAM46A (SEQ ID NO: 3 OR 280), ITGB2 (SEQ ID NO: 4), DOCK2 (SEQ ID NO: 5 OR 223), EVL (SEQ ID NO: 6), SACS (SEQ ID NO: 7), PTPRCAP (SEQ ID NO: 8), EBI2 (SEQ ID NO: 9), PTPRC (SEQ ID NO: 10, 18, 25, OR 243), ANP32E (SEQ ID NO: 11), SFPQ (SEQ ID NO: 12, 38 OR 272), C1QR1 (SEQ ID NO: 13), and FNBP1 (SEQ ID NO: 14 OR 28). The biomarkers of sensitivity may include COL5A2 (SEQ ID NO 73 or 211), ITGA4 (SEQ ID NO: 1), MSN (SEQ ID NO: 2), FAM46A (SEQ ID NO: 3 or 280), ITGB2 (SEQ ID NO: 4), DOCK2 (SEQ ID NO: 5 or 223), EVL (SEQ ID NO: 6), SACS (SEQ ID NO: 7), PTPRCAP (SEQ ID NO: 8), EBI2 (SEQ ID NO: 9), PTPRC (SEQ ID NO: 10, 18, 25, or 243), ANP32E (SEQ ID NO: 11), SFPQ (SEQ ID NO: 12, 38 or 272), C1QR1 (SEQ ID NO: 13), FNBP1 (SEQ ID NO: 14 or 28), and CBFB (SEQ ID NO: 15). The biomarkers of sensitivity may include COL5A2 (SEQ ID NO: 73 or 211), ITGA4 (SEQ ID NO: 1), MSN (SEQ ID NO: 2), FAM46A (SEQ ID NO: 3 or 280), ITGB2 (SEQ ID NO: 4), DOCK2 (SEQ ID NO: 5 or 223), EVL (SEQ ID NO: 6), SACS (SEQ ID NO: 7), EBI2 (SEQ ID NO: 9), ANP32E (SEQ ID NO: 11), SFPQ (SEQ ID NO: 12, 38 or 272), FNBP1 (SEQ ID NO: 14 or 28), CBFB (SEQ ID NO: 15), and SFRS7 (SEQ ID NO: 19 or 54). The biomarkers of sensitivity may include COL5A2 (SEQ ID NO: 73 or 211), ITGA4 (SEQ ID NO: 1), MSN (SEQ ID NO: 2), FAM46A (SEQ ID NO: 3 or 280), ITGB2 (SEQ ID NO: 4), DOCK2 (SEQ ID NO: 5 or 223), EVL (SEQ ID NO: 6), SACS (SEQ ID NO: 7), EBI2 (SEQ ID NO: 9), ANP32E (SEQ ID NO: 11), SFPQ (SEQ ID NO: 12, 38 or 272), FNBP1 (SEQ ID NO: 14 or 28), CBFB (SEQ ID NO: 15), SFRS7 (SEQ ID NO: 19 or 54), and CAP350 (SEQ ID NO: 20 or 61).

For example, the biomarkers of resistance may include SFN (SEQ ID NO: 96 or 324) and LISCH7 (SEQ ID NO: 97). The biomarkers of resistance may include SFN (SEQ ID NO: 96 or 324), LISCH7 (SEQ ID NO: 97), and EPB41L4B (SEQ ID NO: 98). The biomarkers of resistance may include SFN (SEQ ID NO: 96 or 324), LISCH7 (SEQ ID NO: 97), EPB41L4B (SEQ ID NO: 98), and MST1R (SEQ ID NO: 99). The biomarkers of resistance may include SFN (SEQ ID NO: 96 or 324), LISCH7 (SEQ ID NO: 97), EPB41L4B (SEQ ID NO: 98), MST1R (SEQ ID NO: 99), and ITGB4 (SEQ ID NO: 100). The biomarkers of resistance may include SFN (SEQ ID NO: 96 or 324), LISCH7 (SEQ ID NO: 97), EPB41L4B (SEQ ID NO: 98), MST1R (SEQ ID NO: 99), ITGB4 (SEQ ID NO: 100), and DBNDD2 (SEQ ID NO: 102 OR 365). The biomarkers of resistance may include SFN (SEQ ID NO: 96 or 324), LISCH7 (SEQ ID NO: 97), EPB41L4B (SEQ ID NO: 98), MST1R (SEQ ID NO: 99), ITGB4 (SEQ ID NO: 100), DBNDD2 (SEQ ID NO: 102 OR 365), and TACSTD1 (SEQ ID NO: 104). The biomarkers of resistance may include SFN (SEQ ID NO: 96 or 324), LISCH7 (SEQ ID NO: 97), EPB41L4B (SEQ ID NO: 98), MST1R (SEQ ID NO: 99), ITGB4 (SEQ ID NO: 100), DBNDD2 (SEQ ID NO: 102 OR 365), TACSTD1 (SEQ ID NO: 104), and MISP (SEQ ID NO: 105). The biomarkers of resistance may include SFN (SEQ ID NO: 96 or 324), LISCH7 (SEQ ID NO: 97), EPB41L4B (SEQ ID NO: 98), MST1R (SEQ ID NO: 99), ITGB4 (SEQ ID NO: 100), DBNDD2 (SEQ ID NO: 102 OR 365), TACSTD1 (SEQ ID NO: 104), MISP (SEQ ID NO: 105), and KRT8 (SEQ ID NO: 106). The biomarkers of resistance may include SFN (SEQ ID NO: 96 or 324), LISCH7 (SEQ ID NO: 97), EPB41L4B (SEQ ID NO: 98), MST1R (SEQ ID NO: 99), ITGB4 (SEQ ID NO: 100), DBNDD2 (SEQ ID NO: 102 OR 365), TACSTD1 (SEQ ID NO: 104), MISP (SEQ ID NO: 105), KRT8 (SEQ ID NO: 106), and JUP (SEQ ID NO: 107 OR 400). The biomarkers of resistance may include SFN (SEQ ID NO: 96 or 324), LISCH7 (SEQ ID NO: 97), EPB41L4B (SEQ ID NO: 98), MST1R (SEQ ID NO: 99), ITGB4 (SEQ ID NO: 100), DBNDD2 (SEQ ID NO: 102 OR 365), TACSTD1 (SEQ ID NO: 104), MISP (SEQ ID NO: 105), KRT8 (SEQ ID NO: 106), JUP (SEQ ID NO: 107 OR 400), and KRT18 (SEQ ID NO: 108 OR 306. The biomarkers of resistance may include SFN (SEQ ID NO: 96 or 324), LISCH7 (SEQ ID NO: 97), EPB41L4B (SEQ ID NO: 98), MST1R (SEQ ID NO: 99), ITGB4 (SEQ ID NO: 100), DBNDD2 (SEQ ID NO: 102 OR 365), TACSTD1 (SEQ ID NO: 104), MISP (SEQ ID NO: 105), KRT8 (SEQ ID NO: 106), JUP (SEQ ID NO: 107 OR 400), KRT18 (SEQ ID NO: 108 OR 306, and FA2H (SEQ ID NO: 109). The biomarkers of resistance may include SFN (SEQ ID NO: 96 or 324), LISCH7 (SEQ ID NO: 97), EPB41L4B (SEQ ID NO: 98), MST1R (SEQ ID NO: 99), ITGB4 (SEQ ID NO: 100), DBNDD2 (SEQ ID NO: 102 OR 365), TACSTD1 (SEQ ID NO: 104), MISP (SEQ ID NO: 105), KRT8 (SEQ ID NO: 106), JUP (SEQ ID NO: 107 OR 400), KRT18 (SEQ ID NO: 108 OR 306, FA2H (SEQ ID NO: 109), and MGAT4B (SEQ ID NO: 110). The biomarkers of resistance may include SFN (SEQ ID NO: 96 or 324), LISCH7 (SEQ ID NO: 97), EPB41L4B (SEQ ID NO: 98), MST1R (SEQ ID NO: 99), ITGB4 (SEQ ID NO: 100), DBNDD2 (SEQ ID NO: 102 OR 365), TACSTD1 (SEQ ID NO: 104), MISP (SEQ ID NO: 105), KRT8 (SEQ ID NO: 106), JUP (SEQ ID NO: 107 OR 400), KRT18 (SEQ ID NO: 108 OR 306, FA2H (SEQ ID NO: 109), MGAT4B (SEQ ID NO: 110), and DSG2 (SEQ ID NO: 111 OR 312). The biomarkers of resistance may include SFN (SEQ ID NO: 96 or 324), LISCH7 (SEQ ID NO: 97), EPB41L4B (SEQ ID NO: 98), MST1R (SEQ ID NO: 99), ITGB4 (SEQ ID NO: 100), DBNDD2 (SEQ ID NO: 102 OR 365), TACSTD1 (SEQ ID NO: 104), MISP (SEQ ID NO: 105), KRT8 (SEQ ID NO: 106), JUP (SEQ ID NO: 107 OR 400), KRT18 (SEQ ID NO: 108 OR 306, FA2H (SEQ ID NO: 109), MGAT4B (SEQ ID NO: 110), DSG2 (SEQ ID NO:111 OR 312), and LRP5 (SEQ ID NO: 112).

A second aspect features a composition containing an sPLA₂ hydrolysable, cisplatin-containing liposome (e.g., LiPlaCis) for use in treating cancer in a subject (e.g., a human, such as a human with cancer), in which the composition is formulated for administration in at least two doses (e.g., first and second doses). Each of the doses contains cisplatin in an amount of about 75 mg to about 90 mg, or cisplatin in an amount of about 40 mg/m² body surface area to about 55 mg/m² body surface area. The doses of the formulation are characterized as being prepared for administration to the subject on day 1 and day 8, respectively, of at least one three week treatment cycle.

A third aspect features a use of a composition containing an sPLA₂ hydrolysable, cisplatin-containing liposome (e.g., LiPlaCis) in the manufacture of a medicament for treating cancer in a subject in need thereof (e.g., a human, such as a human with cancer). The composition is formulated for administration in at least two doses (e.g., first and second doses). Each of the doses contain cisplatin in an amount of about 75 mg to about 90 mg or cisplatin in an amount of about 40 mg/m² body surface area to about 55 mg/m² body surface area. The doses of the formulation are characterized as being prepared for administration on day 1 and day 8, respectively, of at least one three week treatment cycle.

A fourth aspect features a kit containing: i) a composition containing an sPLA₂ hydrolysable, cisplatin-containing liposome (e.g., LiPlaCis) for use in treating cancer in a subject in need thereof (e.g., a human, such as a human with cancer), in which the composition is present in the kit in a concentrated form that can be diluted into at least two doses (e.g., first and second doses). Each of the doses contain cisplatin in an amount of about 75 mg to about 90 mg or cisplatin in an amount of about 40 mg/m² body surface area to about 55 mg/m² body surface area. The liposomal composition in the kit may also be diluted to a ready to use form that can be divided into the two doses without the need for dilution. The kit also, optionally, contains instructions for administering the composition to the subject, e.g., a first dose of the compositionon day 1 and a second dose of the composition on day 8 of at least one three week treatment cycle.

All of the embodiments discussed above in connection with the first aspect are equally applicable to each of the second, third, and fourth aspects.

### Definitions

As used herein, “a” or “an” means “at least one” or “one or more” unless otherwise indicated. In addition, the singular forms “a”, “an”, and “the” include plural referents unless the context clearly dictates otherwise.

As used herein, “about” refers to an amount that is ±10% of the recited value.

By “biomarker” is meant a nucleic acid molecule (e.g., a mRNA or its complement, for example, a cDNA) or a protein encoded by the nucleic acid molecule that is present in, or is from, a cell or tissue (e.g., a cancer cell or a tumor tissue). The expression of the biomarker correlates to the responsiveness (e.g., sensitivity or resistance) of the cell or tissue (and, thus, the patient in which the cell or tissue resides or the patient from which the cell or tissue was obtained) to a cancer treatment (e.g., LiPlaCis). In particular, a biomarker of sensitivity is a nucleic acid molecule (e.g., a mRNA or its complement) expressed from any one of the genes shown in Tables 2 and 4, or the protein encoded by the nucleic acid molecule, and a biomarker of resistance is a nucleic acid molecule (e.g., a mRNA or its complement) expressed from any one of the genes shown in Tables 3 and 5, or the protein encoded by the nucleic acid molecule.

The terms “cancer” and “cancerous” refer to or describe the physiological condition in mammals (e.g., humans) that is typically characterized by unregulated cell proliferation. Examples of cancer include, but are not limited to, prostate cancer, ovarian cancer (e.g., ovarian adenocarcinoma or embryonal carcinoma), liver cancer (e.g., hepatocellular carcinoma (HCC) or hepatoma), myeloma (e.g., multiple myeloma), colorectal cancer (e.g., colon cancer and rectal cancer), leukemia (e.g., acute myeloid leukemia, acute lymphoid leukemia, chronic myeloid leukemia, chronic lymphocytic leukemia, acute myeloblastic leukemia, acute promyelocytic leukemia, acute myelomonocytic leukemia, acute monocytic leukemia, acute erythroleukemia, and chronic leukemia), myelodysplastic syndrome, lymphoma (e.g., diffuse large B-cell lymphoma, cutaneous T-cell lymphoma, peripheral T-cell lymphoma, Hodgkin's lymphoma, non-Hodgkin's lymphoma, Waldenstrom's macroglobulinemia, and lymphocytic lymphoma), cervical cancer, esophageal cancer, melanoma, glioma (e.g., oligodendroglioma), pancreatic cancer (e.g., adenosquamous carcinoma, signet ring cell carcinoma, hepatoid carcinoma, colloid carcinoma, islet cell carcinoma, and pancreatic neuroendocrine carcinoma), gastrointestinal stromal tumor, sarcoma (e.g., fibrosarcoma, myxosarcoma, liposarcoma, chondrosarcoma, osteogenic sarcoma, angiosarcoma, endotheliosarcoma, lymphangiosarcoma, lymphangioendotheliosarcoma, leiomyosarcoma, Ewing's sarcoma, and rhabdomyosarcoma), breast cancer (e.g., medullary carcinoma), ER-positive cancer, bladder cancer, head and neck cancer (e.g., squamous cell carcinoma of the head and neck), lung cancer (e.g., non-small cell lung carcinoma, large cell carcinoma, bronchogenic carcinoma, and papillary adenocarcinoma), metastatic cancer, oral cavity cancer, uterine cancer, testicular cancer (e.g., seminoma and embryonal carcinoma), skin cancer (e.g., squamous cell carcinoma and basal cell carcinoma), thyroid cancer (e.g., papillary carcinoma and medullary carcinoma), brain cancer (e.g., astrocytoma and craniopharyngioma), stomach cancer, intra-epithelial cancer, bone cancer, biliary tract cancer, eye cancer, larynx cancer, kidney cancer (e.g., renal cell carcinoma and Wilms tumor), gastric cancer, blastoma (e.g., nephroblastoma, medulloblastoma, hemangioblastoma, neuroblastoma, and retinoblastoma), polycythemia vera, chordoma, synovioma, mesothelioma, adenocarcinoma, sweat gland carcinoma, sebaceous gland carcinoma, cystadenocarcinoma, bile duct carcinoma, choriocarcinoma, epithelial carcinoma, ependymoma, pinealoma, acoustic neuroma, schwannoma, meningioma, pituitary adenoma, nerve sheath tumor, cancer of the small intestine, cancer of the endocrine system, cancer of the penis, cancer of the urethra, cutaneous or intraocular melanoma, a gynecologic tumor, solid tumors of childhood, and neoplasms of the central nervous system. The term cancer includes solid tumors (e.g., breast cancer) and hematological cancers (e.g., cancer of the blood, such as lymphoma (e.g., cutaneous T-cell lymphoma (CTCL)).

The terms “expression level” and “level of expression,” as used herein, refer to the amount of a gene product (e.g., DNA, RNA (e.g. messenger RNA (mRNA)), or a protein encoded by a given gene) in a cell (e.g., a cancer cell), a tissue (e.g., a tumor tissue), a biological sample, or a subject (e.g., a human, such as a human with cancer).

“Gene” as used herein indicates a coding or noncoding gene whose activity can be determined by measuring the produced RNA. Examples include protein coding genes, microRNAs, small nuclear RNAs and other RNAs with catalytic, regulatory or coding properties.

As used herein, “inhibit growth” means causing a reduction in cell growth (e.g., cancer cell growth, which can be assessed using, e.g., the NCI60 cancer cell lines) in vivo or in vitro by, e.g., 10%, 20%, 30%, 40%, 50%, 60%, 70%, 80%, 90%, 95%, or 99% or more, as evident by a reduction in the proliferation of cells exposed to a treatment (e.g., an sPLA₂ hydrolysable, cisplatin-containing liposome described herein), relative to the proliferation of cells in the absence of the treatment. Growth inhibition may be the result of a treatment (e.g., treatment with the sPLA₂ hydrolysable, cisplatin-containing liposome) that induces apoptosis in a cell, induces necrosis in a cell, slows cell cycle progression, disrupts cellular metabolism, induces cell lysis, or induces some other mechanism that reduces the proliferation of cells.

As used herein, the term “microarray” refers to a device employed by any method that quantifies one or more subject oligonucleotides, e.g., RNA, DNA, cDNA, or analogues thereof, at a time. For example, many DNA microarrays, including those made by Affymetrix (e.g., an Affymetrix HG-U133A or HG-U133_Plus_2 array), use several probes for determining the level of a single biomarker. The DNA microarray may contain oligonucleotide probes that may be, e.g., full-length cDNAs complementary to an RNA or cDNA fragments that hybridize to part of an RNA. The DNA microarray may also contain modified versions of DNA or RNA, such as locked nucleic acids or LNA. Exemplary RNAs include mRNA, miRNA, and miRNA precursors.

As used herein, the term “NCI60” refers to a panel of 60 cancer cell lines from lung, colon, breast, ovarian, leukemia, renal, melanoma, prostate, and brain cancers including the following cancer cell lines: NSCLC_NCIH23, NSCLC_NCIH522, NSCLC_A549ATCC, NSCLC_EKVX, NSCLC_NCIH226, NSCLC_NCIH332M, NSCLC_H460, NSCLC_HOP62, NSCLC_HOP92, COLON_HT29, COLON_HCC-2998, COLON_HCT116, COLON_SW620, COLON_COLO205, COLON_HCT15, COLON_KM12, BREAST_MCF7, BREAST_MCF7ADRr, BREAST_MDAMB231, BREAST_HS578T, BREAST_MDAMB435, BREAST_MDN, BREAST_BT549, BREAST_T47D, OVAR_OVCAR3, OVAR_OVCAR4, OVAR_OVCAR5, OVAR_OVCAR8, OVAR_IGROV1, OVAR_SKOV3, LEUK_CCRFCEM, LEUK_K562, LEUK_MOLT4, LEUK_HL60, LEUK_RPM18266, LEUK_SR, RENAL_UO31, RENAL_SN12C, RENAL_A498, RENAL_CAKI1, RENAL_RXF393, RENAL_7860, RENAL_ACHN, RENAL_TK10, MELAN_LOXIMVI, MELAN_MALME3M, MELAN_SKMEL2, MELAN_SKMEL5, MELAN_SKMEL28, MELAN_M14, MELAN_UACC62, MELAN_UACC257, PROSTATE_PC3, PROSTATE_DU145, CNS_SNB19, CNS_SNB75, CNS_U251, CNS_SF268, CNS_SF295, and CNS_SF539.

The terms “patient” and “subject,” as used interchangeably herein, refer to any animal (e.g., a mammal, such as a human, e.g., a human with a cancer). A patient to be treated or tested for responsiveness to a treatment (e.g., treatment with an sPLA₂ hydrolysable, cisplatin-containing liposome, such as LiPlaCis) according to the methods described herein may be one who has been diagnosed with a cancer, such as those described herein, e.g., breast cancer, acute myelogenous leukemia (AML), acute lympho-blastic leukemia (ALL), chronic lymphocytic leukemia (CLL), myelodysplastic syndrome (MDS), chronic myelogenous leukemia—chronic phase (CMLCP), diffuse large B-cell lymphoma (DLBCL), cutaneous T-cell lymphoma (CTCL), peripheral T-cell lymphoma (PTCL), Hodgkin's lymphoma, hepatocellular carcinoma (HCC), cervical cancer, renal cell carcinoma (RCC), esophageal cancer, melanoma, glioma, pancreatic cancer, gastrointestinal stromal tumors (GIST), sarcoma, non-small cell lung carcinoma (NSCLC), prostate cancer, ovarian cancer, colon cancer, bladder cancer, or squamous cell carcinoma of the head and neck (SCCHN). Diagnosis may be performed by any method or technique known in the art, such as x-ray, MRI, or biopsy, and may also be confirmed by a physician. To minimize exposure of a patient to drug treatments that may not be therapeutic, the patient may be determined to be either responsive or non-responsive to a cancer treatment, such as treatment with an sPLA₂ hydrolysable, cisplatin-containing liposome, according to the methods described herein, prior to treatment.

As used herein, the term “percent (%) sequence identity” refers to the percentage of nucleic acid residues of a candidate sequence, e.g., a probe or primer of the invention, that are identical to the nucleic acid residues of a reference sequence, e.g., a biomarker sequence of the invention, after aligning the sequences and introducing gaps, if necessary, to achieve the maximum percent sequence identity (e.g., gaps can be introduced in one or both of the candidate and reference sequences for optimal alignment and non-homologous sequences can be disregarded for comparison purposes). Alignment for purposes of determining percent sequence identity can be achieved in various ways that are within the skill in the art, for instance, using computer software, such as BLAST, BLAST-2, BLAST-P, BLAST-N, BLAST-X, WU-BLAST-2, ALIGN, ALIGN-2, CLUSTAL, Megalign (DNASTAR). In addition, those skilled in the art can determine appropriate parameters for measuring alignment, including any algorithms needed to achieve optimal alignment over the length of the sequences being compared.

“Resistant” or “resistance” as used herein means that a cell (e.g., a cancer cell), a tissue containing the cell (e.g., a tumor), or the cell or tissue in a patient (e.g., a human with cancer) is non-responsive to treatment with an anti-cancer agent (e.g., an sPLA₂ hydrolysable, cisplatin-containing liposome, such as LiPlaCis). In particular, the treatment reduces the growth of a resistant cell (e.g., the cancer cell) in vitro by less than about 40%, 30%, 20%, 10%, 5%, 1%, or less, relative to the growth of a cell or tissue known to be resistant to the treatment or relative to a cell or tissue not exposed to the treatment. Resistance to treatment may be determined by a cell proliferation assay, e.g., a cell-based assay, which measures the growth of treated cells as a function of the absorbance of the cells of an incident light beam, such as the NCI60 assays described herein. In this assay, greater absorbance indicates greater cell growth, and thus, resistance to the treatment.

The terms “responsive” and “responsiveness,” as used herein, refer to the likelihood that a cancer treatment (e.g., treatment with an sPLA₂ hydrolysable, cisplatin-containing liposome) has a desired effect in a cell (e.g., a cancer cell), a tissue (e.g., a tumor), or a patient with cancer (e.g., a human with cancer). For example, the desired effect can include inhibition of the growth of a cancer cell in vitro by more than 10%, 20%, 30%, 40%, 50%, 60%, 70%, 80%, 90%, or 100% relative to the growth of a cancer cell not exposed to the treatment. The desired effect can also include reduction in tumor mass by, e.g., about 10%, 20%, 30%, 40%, 50%, 60%, 70%, 80%, 90%, or 100%. Responsiveness to treatment may be determined by a cell proliferation assay, e.g., a cell-based assay, which measures the growth of treated cells as a function of the absorbance of the cells of an incident light beam, such as the NCI60 assays described herein. In this assay, lesser absorbance indicates lesser cell growth, and thus, sensitivity to the treatment. A greater reduction in growth indicates more sensitivity to the treatment. In particular, “responsiveness” is a measure of the sensitivity or resistance of a patient (e.g., the cancer cells in a patient) to a treatment for cancer (e.g., an sPLA₂ hydrolysable, cisplatin-containing liposome, such as LiPlaCis).

The term “sample,” as used herein, refers to any specimen (such as cells, tissue (e.g., a tissue sample obtained by biopsy), blood, serum, plasma, urine, cerebrospinal fluid, or pancreatic fluid) taken from a subject (e.g., a subject with a cancer). Preferably, the sample is taken from a portion of the body affected by a cancer (e.g., a biopsy of the cancer tissue, such as breast cancer tissue). Biopsy may involve fine needle aspiration biopsy, core needle biopsy (e.g., stereotactic core needle biopsy, vacuum-assisted core biopsy, or magnetic resonance imaging (MRI) guided biopsy), or surgical biopsy (e.g., incisional biopsy or excisional biopsy). The sample may undergo additional purification and processing, for example, to remove cell debris and other unwanted molecules. Additional processing may further involve producing cDNA molecules corresponding to nucleic acid molecules (e.g., mRNA) in the sample and/or amplification of the nucleic acid molecules, e.g., using PCR, such as RT-PCR. The standard methods of sample purification, such as removal of unwanted molecules, are known in the art.

The terms “secretory phospholipase A₂ (sPLA₂) hydrolyzable, cisplatin-containing liposome,” “sPLA₂ hydrolysable, cisplatin-containing liposome,” “composition comprising liposomal formulation of cisplatin,” “liposomal cisplatin formulation,” “the liposomal composition,” “the composition,” and “the liposome,” as used herein refer to an antitumor agent that is a liposomal formulation of cisplatin. The sPLA₂ hydrolysable, cisplatin-containing liposome is formulated to release an encapsulated drug (e.g., cisplatin) from the core of a hydrophobic layer into tumor tissue. Since sPLA₂ protein is associated with tumor tissue, sPLA₂ hydrolysable liposomes may be used to preferentially deliver encapsulated drugs (e.g., cisplatin) to the tumor tissue. Exemplary sPLA2 hydrolysable, cisplatin-containing liposomes include LiPlaCis (LiPlasome Pharma ApS). An sPLA₂ hydrolysable, cisplatin-containing liposome is described in, e.g., U.S. Patent Application Publication No. 2012/0177726 and de Jonge et al. (Eur J Cancer. 46(16):3016-21, 2010), each of which is hereby incorporated by reference.

The term “LiPlaCis” as used herein refers to an antitumor agent that is a liposomal formulation of cisplatin. The liposomes—called LiPlasomes—are designed to trigger the release of an encapsulated drug (e.g., cisplatin) specifically in the tumor tissue. An enzyme especially present on tumors called secretory phospholipase A2 (sPLA2), is utilized to break down the liposomes once they have accumulated in the cancer tissue. The lipid composition of LiPlaCis is tailored to be specifically sensitive to degradation by the sPLA2 enzyme and thereby for release of the encapsulated drug. LiPlaCis is also described in de Jonge et al. (Eur J Cancer. 2010 46(16):3016-21) and U.S. Patent Application Publication No. 2012/0177726, hereby incorporated by reference. Exemplary LiPlaCis include LiPlaCis®, LiPlasome Pharma. The liposomes of LiPlaCis contain ˜70:25:5 mol % DSPC:DSPG:DSPE-PEG2000 and less than 1% cholesterol.

“Sensitive” and “sensitivity” as used herein refer to a cell (e.g., a cancer cell), a tissue containing the cell (e.g., a tumor), or a patient containing the cell or tissue having cancer (e.g., a human having cancer) that is responsive to treatment, such as an anti-cancer agent (e.g., an sPLA₂ hydrolysable, cisplatin-containing liposome) or radiation treatment. In particular, the treatment inhibits the growth of the cell (e.g., the cancer cell) in vitro by about 70%, 80%, 90%, 95%, 99% or 100% relative to the growth of a cell not exposed to the treatment. Sensitivity to treatment may be determined by a cell proliferation assay, e.g., a cell-based assay, which measures the growth of treated cells as a function of the absorbance of the cells of an incident light beam, such as the NCI60 assays described herein. In this assay, lesser absorbance indicates lesser cell growth, and thus, sensitivity to the treatment.

The term “specific hybridization” as used herein refers to when complementary nucleic acid sequences form a stable duplex under high stringency conditions, such as high hybridization temperature and low salt in hybridization buffers, which permit only hybridization between nucleic acid sequences that are highly similar. Nucleic acids are referred to as “complementary” that contain nucleotides or nucleotide homologues that can form hydrogen bonds according to Watson-Crick base-pairing rules (e.g., G with C, A with T or A with U) or other hydrogen bonding motifs such as for example diaminopurine with T, 5-methyl C with G, 2-thiothymidine with A, inosine with C, pseudoisocytosine with G, etc. Anti-sense RNA may be complementary to other oligonucleotides, e.g., mRNA.

“Treatment,” “medical treatment,” to “treat,” and “therapy,” as used interchangeably herein, refer to administering or exposing a patient with cancer (e.g., a human) to an anti-cancer agent (e.g., a drug, such as an sPLA₂ hydrolysable, cisplatin-containing liposome (e.g., LiPlaCis), a protein, an antibody, a nucleic acid, a chemotherapeutic agent, or a radioactive agent), or to some other form of medical intervention used to treat or prevent a disease, disorder, or condition (e.g., surgery, cryotherapy, radiation therapy, or combinations thereof). In particular, a medical treatment can be or can include administration of an sPLA₂ hydrolysable, cisplatin-containing liposome, as described herein. For example, the treatment may be of a cancer, such as a solid tumor or a hematological cancer. Examples of cancer include, e.g., breast cancer (e.g., medullary carcinoma or an ER-positive breast cancer), prostate cancer, ovarian cancer (e.g., ovarian adenocarcinoma or embryonal carcinoma), liver cancer (e.g., hepatocellular carcinoma (HCC) or hepatoma), myeloma (e.g., multiple myeloma), colorectal cancer (e.g., colon cancer and rectal cancer), leukemia (e.g., acute myeloid leukemia, acute lymphoid leukemia, chronic myeloid leukemia, chronic lymphocytic leukemia, acute myeloblastic leukemia, acute promyelocytic leukemia, acute myelomonocytic leukemia, acute monocytic leukemia, acute erythroleukemia, and chronic leukemia), myelodysplastic syndrome, lymphoma (e.g., diffuse large B-cell lymphoma, cutaneous T-cell lymphoma, peripheral T-cell lymphoma, Hodgkin's lymphoma, non-Hodgkin's lymphoma, Waldenstrom's macroglobulinemia, and lymphocytic lymphoma), cervical cancer, esophageal cancer, melanoma, glioma (e.g., oligodendroglioma), pancreatic cancer (e.g., adenosquamous carcinoma, signet ring cell carcinoma, hepatoid carcinoma, colloid carcinoma, islet cell carcinoma, and pancreatic neuroendocrine carcinoma), gastrointestinal stromal tumor, sarcoma (e.g., fibrosarcoma, myxosarcoma, liposarcoma, chondrosarcoma, osteogenic sarcoma, angiosarcoma, endotheliosarcoma, lymphangiosarcoma, lymphangioendotheliosarcoma, leiomyosarcoma, Ewing's sarcoma, and rhabdomyosarcoma), bladder cancer, head and neck cancer (e.g., squamous cell carcinoma of the head and neck), lung cancer (e.g., non-small cell lung carcinoma, large cell carcinoma, bronchogenic carcinoma, and papillary adenocarcinoma), metastatic cancer, oral cavity cancer, uterine cancer, testicular cancer (e.g., seminoma and embryonal carcinoma), skin cancer (e.g., squamous cell carcinoma and basal cell carcinoma), thyroid cancer (e.g., papillary carcinoma and medullary carcinoma), brain cancer (e.g., astrocytoma and craniopharyngioma), stomach cancer, intra-epithelial cancer, bone cancer, biliary tract cancer, eye cancer, larynx cancer, kidney cancer (e.g., renal cell carcinoma and Wilms tumor), gastric cancer, blastoma (e.g., nephroblastoma, medulloblastoma, hemangioblastoma, neuroblastoma, and retinoblastoma), polycythemia vera, chordoma, synovioma, mesothelioma, adenocarcinoma, sweat gland carcinoma, sebaceous gland carcinoma, cystadenocarcinoma, bile duct carcinoma, choriocarcinoma, epithelial carcinoma, ependymoma, pinealoma, acoustic neuroma, schwannoma, meningioma, pituitary adenoma, nerve sheath tumor, cancer of the small intestine, cancer of the endocrine system, cancer of the penis, cancer of the urethra, cutaneous or intraocular melanoma, a gynecologic tumor, solid tumors of childhood, and neoplasms of the central nervous system. Radiation therapy includes the administration of a radioactive agent to a patient or exposure of a patient to radiation. The radiation may be generated from sources, such as particle accelerators and related medical devices or agents that emit, e.g., X-radiation, gamma radiation, or electron (Beta radiation) beams. A treatment may be or further include surgery, e.g., to remove a tumor from a subject or living organism.

Other features and advantages of the invention will be apparent from the following Detailed Description, the drawings, and the claims.

### BRIEF DESCRIPTION OF THE DRAWINGS

FIG. 1 is a graph grouping predicted sensitivity to LiPlaCis by cancer type. Each gray circle represents the predicted LiPlaCis sensitivity of one patient calculated as the difference between the mean of the expression levels of the biomarkers of sensitivity (Table 2) and the mean of the expression levels of the biomarkers of resistance for the patient (Table 3). Patients are grouped according to cancer type. The median predicted sensitivity (black bar) for a cancer type is related to the relative response rate for that cancer type. The predictions are used for relative comparisons to compare cancer types and cannot be used for absolute predictions of response rate for a given cancer type. The predictions are normalized to a scale of 0 to 100 for all 3,522 patients.

FIG. 2 is a graph depicting the correlation between DRP score and clinical response (RECIST) in seven patients who had not received prior platinum treatment. When the response is encoded as 3,2,1 for partial response (PR), stable disease (SD), and progressive disease (PD), respectively, the one-sided Pearson correlation is 0.61 (P=0.07, below the significance level of 0.1 defined in the statistical analysis plan). Patients that received prior platinum treatment were excluded from analysis. There are no scores below 33 because patients with a score below 33 were excluded from the trial.

FIG. 3 is a graph depicting the Cox proportional hazards of seven patients who had not received prior platinum treatment, stratified by DRP score. The DRP score was used to divide the population in two: those above a cutoff of 67 (upper tertile, N=5) and those between inclusion cutoff of 33 and stratification cutoff of 67 (middle tertile, N=2). These two populations have a dramatic difference in hazard rate (ratio 4e-10, P=0.008). The median time to progression is 25 weeks and 8 weeks, respectively. Because there were no deaths before progression in the evaluable population, time to progression (TTP) and progression-free survival (PFS) are identical in this population.

FIG. 4 is a graph comparing the response to LiPlaCis with prior treatment. The hazard ratio is 0.22 (P=0.025 one sided), and median duration of treatment is 25 versus 17 weeks.

FIG. 5 is a bar graph showing the response of DRP positive advanced breast cancer patients to LiPlaCis treatment (2 doses of 75 mg each, administered on day 1 and day 8 of three week treatment cycle/s).

FIG. 6 is a bar graph showing the duration of LiPlaCis treatment in the DRP positive advanced breast cancer patients, whose response to the treatment has been depicted in the aforementioned FIG. 5.

FIG. 7 is a schematic showing protocols for a phase I/11 clinical trial of LiPlaCis.

### DETAILED DESCRIPTION OF THE INVENTION

We have discovered that a liposomal formulation of cisplatin, e.g., LiPlaCis, exhibits an improved therapeutic efficacy and an improved safety and tolerability profile compared to conventional cisplatin, in particular in subjects with cancer (e.g., advanced or refractory tumors, such as breast cancer). Subjects administered the liposomal composition containing cisplatin (e.g., in an amount of about 75-90 mg) on day 1 and day 8 of a three week treatment cycle. We observed a 5-28-fold increase in DNA platinum adducts (GG-Pt) in tumor tissue compared to normal tissue of the same patient. Administration of conventional cisplatin produces only a 4-6-fold level of DNA-platinum (GG-Pt). Our results show that LiPlaCis effectively targets and delivers cisplatin to tumor tissue.

In addition, the efficacy of treatment can be improved when the cancer subject is assessed prior to treatment using our drug response predictor (DRP) (e.g., assessing the level of one or more of the biomarkers of sensitivity of Tables 2 and 4 and/or one or more of the biomarkers of resistance of Tables 3 and 5. The DRP is an assay that, based on samples from a tumor, can predict the likelihood that a tumor will respond to a specific drug (e.g., cisplatin). The DRP method builds on a comparison of sensitive and resistant cell lines, including genomic information from the NCI (USA) NCI60 cell lines, clinical tumor biology, and clinical correlates in a systems biology network. The DRP can be performed using mRNA measurements. Biomarker signatures of the DRP can be matched to the corresponding genes in a universal microarray (which contains all genes) in order to make prediction for a specific drug (e.g., cisplatin) for a specific patient.

sPLA₂ Hydrolysable, Cisplatin-Containing Liposome (e.g., LiPlaCis)

sPLA₂ hydrolysable, cisplatin-containing liposome (e.g., LiPlaCis) used herein, is a liposomal formulation of cisplatin, designed to be specifically degraded by secretory phospholipase A2 (sPLA₂) which is over-expressed by tumor tissue. sPLA2 has been shown to be present in elevated levels in a number of different tumor tissues (e.g., prostate, lung, ovarian, and breast cancer). Thus, LiPlaCis is intended to improve the therapeutic index due to an improved therapeutic efficacy and possibly also an improved safety and tolerability profile.

LiPlaCis can be prepared by spray-drying a mixture of phospholipids:
70/25/5 mol % DSPC/DSPG/DSPE-PEG2000

The lipids are then dissolved in methanol and chloroform. The lipid intermediate is hydrated in an aqueous solution of cisplatin with agitation. At this step the liposomes are formed but they have a broad size distribution and have a mixture of single-layer and multiple-layer liposomes. In order to get a product with a narrow size distribution and mono-layer liposomes, the hydration mixture can be extruded by passing it through poly-carbonate filters of appropriate pore sizes. To remove un-encapsulated cisplatin, the mixture can be purified by a number of techniques available, for example by dialysis, gel-filtration, and ultra-filtration. For preparations ranging from a few liters and above, ultra-filtration is a preferred method. Preparations intended for parenteral administration may be sterilized, for example by sterile-filtration. Methods for formulating LiPlaCis have been described in detail in, e.g., U.S. Patent Application Publication No. 2012/0177726 and de Jonge et al. (Eur J Cancer. 46(16):3016-21, 2010), each of which is hereby incorporated by reference.

Methods of Treating Cancer Using an sPLA₂ Hydrolysable, Cisplatin-Containing Liposome (e.g., LiPlaCis)

Featured herein are methods of treating cancer using a liposomal formulation of cisplatin (e.g., LiPlaCis) administered on day 1 and day 8 of three week treatment cycle/s.

Administration of sPLA₂ Hydrolysable, Cisplatin-Containing Liposome

A cancer patient can be treated with a composition containing sPLA₂ hydrolysable, cisplatin-containing liposomes (e.g., LiPlaCis) according to the methods described herein. The sPLA₂ hydrolysable, cisplatin-containing liposome composition may be administered to the patient, for example, parenterally, enterally, or topically. Enteral routes of administration of the liposomal formulation of cisplatin include oral, buccal, sublabial, sublingual, or by inhalation. Parenteral routes of administration of the liposomal formulation of cisplatin include intravenous, transdermal, intradermal, intramuscular, intra-arterial, intracranial, subcutaneous, intraorbital, intraventricular, intraspinal, intraperitoneal, or intranasal. The preferred route for administration of the sPLA₂ hydrolysable, cisplatin-containing liposome (e.g., LiPlaCis) may be intravenous, such as intravenous infusion. The sPLA₂ hydrolysable, cisplatin-containing liposome composition may be administered as an intravenous infusion over a period of about 2-3 hours (e.g., 0.1-0.5, 0.5-1, 1-1.5, 1.5-2, 2-2.5, 2.5-3, 3-3.5, 3.5-4, 4-4.5, 4.5-5, 5-5.5, or 5.5-6 hours). The sPLA₂ hydrolysable, cisplatin-containing liposome composition can be administered as an intravenous infusion over about 2 hours (e.g., 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, or 2.9 hours), or over about 3 hours (e.g., 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or 5 hours). Particularly, the sPLA2 hydrolysable, cisplatin-containing liposome composition (e.g., LiPlaCis) can be administered as an intravenous infusion over about 2 hours.

The sPLA₂ hydrolysable, cisplatin-containing liposome composition (e.g., LiPlaCis) can be administered in one or more doses (e.g., one, two, three, four, five, six, seven, eight, nine, ten, or more doses), each dose containing about 40-225 mg of cisplatin (e.g., 40-45, 45-50, 50-55, 55-60, 60-65, 65-70, 70-75, 75-80, 80-85, 85-90, 90-95, 95-100, 100-105, 105-110, 110-115, 115-120, 120-125, 125-130, 130-135, 135-140, 140-145, 145-150, 150-155, 155-160, 160-165, 165-170, 170-175, 175-180, 180-185, 185-190, 190-195, 195-200, 200-205, 205-210, 210-2115, 215-220, or 220-225 mg cisplatin). The sPLA2 hydrolysable, cisplatin-containing liposome composition (e.g., LiPlaCis) can be administered as two doses, each dose containing an amount of about 75 mg of cisplatin (e.g., 60, 61, 62, 63, 64, 65, 66, 67, 68, 69, 70, 71, 72, 73, 74, 75, 76, 77, 78, 79, or 80 mg of cisplatin), or about 90 mg cisplatin (e.g., 81, 82, 83, 84, 85, 86, 87, 88, 89, 90, 91, 92, 93, 94, 95, 96, 97, 98, 99, or 100 mg cisplatin). Particularly, the sPLA₂ hydrolysable, cisplatin-containing liposome composition (e.g., LiPlaCis) can be administered in two doses, each dose containing an amount of about 75 mg cisplatin. Alternatively, the sPLA₂ hydrolysable, cisplatin-containing liposome (e.g., LiPlaCis) can be administered in two doses, each dose containing an amount of about 90 mg cisplatin. The two doses of the liposome composition are preferably administered on days 1 and 8 of a three week treatment cycle. The doses can also be administered according to a different schedule, if desired (e.g., a first dose on day 1 and a second dose on any one of days 5-21 of a three week treatment cycle).

As an alternative, the sPLA₂ hydrolysable, cisplatin-containing liposome composition (e.g., LiPlaCis) can be administered in two doses, the first dose containing an amount of about 75 mg cisplatin, and the second dose containing an amount of about 90 mg cisplatin. The sPLA₂ hydrolysable, cisplatin-containing liposome composition (e.g., LiPlaCis) can also be administered in two doses, the first dose containing an amount of about 90 mg cisplatin, and the second dose containing an amount of about 75 mg cisplatin. Alternatively, the sPLA₂ hydrolysable, cisplatin-containing liposome composition can be administered as one or more doses (e.g., one, two, three, four, five, six, seven, eight, nine, ten, or more doses), each dose containing an amount of cisplatin of 20-125 mg/m² body surface area of the subject (e.g., 20-25, 25-30, 30-35, 35-40, 40-45, 45-50, 50-55, 55-60, 60-65, 65-70, 70-75, 75-80, 80-85, 85-90, 90-95, 95-100, 100-105, 105-110, 110-115, or 115-120 mg/m² body surface area). For example, the sPLA₂ hydrolysable, cisplatin-containing liposome composition can be administered in one or more doses, each dose containing an amount of cisplatin of 40-55 mg/m² body surface area of the subject (e.g., 40, 41, 42, 43, 44, 45, 46, 47, 48, 49, 50, 51, 52, 53, 54, or 55 mg/m² body surface area). Particularly, the sPLA₂ hydrolysable, cisplatin-containing liposome composition (e.g., LiPlaCis) can be administered in two doses, each dose containing an amount of cisplatin of about 40 mg/m² body surface area of the subject. Alternatively, the sPLA₂ hydrolysable, cisplatin-containing liposome composition (e.g., LiPlaCis) can be administered in two doses, each dose containing an amount of cisplatin of about 55 mg/m² body surface area of the subject. The two doses of the liposome composition are preferably administered on days 1 and 8 of a three week treatment cycle. The doses can also be administered according to a different schedule, if desired (e.g., a first dose on day 1 and a second dose on any one of days 5-21 of a three week treatment cycle).

The sPLA₂ hydrolysable, cisplatin-containing liposome composition (e.g., LiPlaCis) may be administered at a frequency of, e.g., at least once hourly, once daily, twice daily, once weekly, once every two weeks, once every three weeks, once every four weeks, once monthly, once every two months, once every three months, once every six months, or once every year. For example, the sPLA₂ hydrolysable, cisplatin-containing liposome (e.g., LiPlaCis) can be administered as one or more doses once every three weeks. Particularly, the sPLA₂ hydrolysable, cisplatin-containing liposome (e.g., LiPlaCis) can be administered as two doses once every three weeks. In particular, the sPLA₂ hydrolysable, cisplatin-containing liposome (e.g., LiPlaCis) can be administered as two doses (e.g., first dose and second dose) on day 1 and day 8 of a three week cycle.

The sPLA₂ hydrolysable, cisplatin-containing liposome is administered at one or more doses such that about 80-450 mg of cisplatin (e.g., 80-90, 90-100, 100-110, 110-120, 120-130, 130-140, 140-150, 150-160, 160-170, 170-180, 180-190, 190-200, 200-210, 210-220, 220-230, 230-240, 240-250, 250-260, 260-270, 270-280, 280-290, 290-300, 300-310, 310-320, 320-330, 330-340, 340-350, 350-360, 360-370, 370-380, 380-390, 390-400, 400-410, 410-420, 420-430, 430-440, or 440-450 mg of cisplatin), or cisplatin amounting to 40-250 mg/m² body surface area (e.g., 40-50, 50-60, 60-70, 70-80, 80-90, 90-100, 100-110, 110-120, 120-130, 130-140, 140-150, 150-160, 160-170, 170-180, 180-190, 190-200, 200-210, 210-220, 220-230, 230-240, or 240-250 mg/m² body surface area) is administered in each treatment cycle. In particular, the sPLA₂ hydrolysable, cisplatin-containing liposome (e.g., LiPlaCis) can be administered as two doses in a treatment cycle such that 150 mg of cisplatin is administered in every treatment cycle. Alternatively, the sPLA₂ hydrolysable, cisplatin-containing liposome (e.g., LiPlaCis) can be administered as two doses in a treatment cycle such that 180 mg of cisplatin is administered in every treatment cycle. As yet another alternative, the sPLA₂ hydrolysable, cisplatin-containing liposome (e.g., LiPlaCis) can be administered as two doses in a treatment cycle such that cisplatin amounting to 80 mg/m² body surface area is administered in every treatment cycle. Alternatively, the sPLA₂ hydrolysable, cisplatin-containing liposome (e.g., LiPlaCis) can be administered as two doses in a treatment cycle such that cisplatin amounting to 110 mg/m² body surface area is administered in every treatment cycle.

The sPLA₂ hydrolysable, cisplatin-containing liposome composition (e.g., LiPlaCis) may be administered according to a treatment regimen of, e.g., 75 mg, 90 mg, 45 mg/m², or 55 mg/m² per dose on day 1 and day 8 (1 cycle) for up to 3 cycles or more. The treatment regimen may be repeated one to five times, one to ten times, one to fifteen times, one to twenty times, or more (e.g., 1, 2, 3, 4, 5, 6, 7, 8, 9, 10, 11, 12, 13, 14, 15, 16, 17, 18, 19, 20, or more cycles). The administration of the sPLA₂ hydrolysable, cisplatin-containing liposome composition can be repeated at such a frequency for a selected period of time, followed by a period without treatment. Such repeated administrations can occur over a course of therapy lasting a specified length of time (e.g., at least 1 week, 2 weeks, 3 weeks, 1 month, 2 months, 3 months, 6 months, 8 months, 10 months, 12 months, 18 months, 24 months, 36 months, 48 months, or 60 months). Alternatively, the administration of the sPLA₂ hydrolysable, cisplatin-containing liposome composition (e.g., LiPlaCis) can be repeated at such a frequency (e.g., a three week treatment cycle) in consecutive treatment cycles, with no time interval (e.g., no non-treatment interval) in between.

The sPLA₂ hydrolysable, cisplatin-containing liposome composition (e.g., LiPlaCis) may be administered at a treatment regimen that involves escalation of the dose in subsequent treatment cycles. For example, a liposomal cisplatin formulation (e.g., LiPlaCis) may be administered as 2 doses, each of about 75 mg of cisplatin (e.g., 60, 61, 62, 63, 64, 65, 66, 67, 68, 69, 70, 71, 72, 73, 74, 75, 76, 77, 78, 79, or 80 mg of cisplatin) on day 1 and day 8 of the first three week treatment cycle, followed by two doses, each of about 90 mg of cisplatin (e.g., 81, 82, 83, 84, 85, 86, 87, 88, 89, 90, 91, 92, 93, 94, 95, 96, 97, 98, 99, or 100 mg of cisplatin) on day 1 and day 8 of the next treatment cycle. Alternatively, liposomal cisplatin formulation (e.g., LiPlaCis) may be administered as 2 doses, each comprising cisplatin amounting to about 40 mg/mm² body surface area on day 1 and day 8 of the first three week treatment cycle, followed by two doses, each comprising cisplatin amounting to about 55 mg/mm² body surface area on day 1 and day 8 of the next treatment cycle.

The sPLA₂ hydrolysable, cisplatin-containing liposome composition (e.g., LiPlaCis) can be administered in a pharmaceutical composition that includes one or more pharmaceutically acceptable carriers, excipients, or diluents. Examples of suitable carriers, excipients, or diluents of the liposomal composition (e.g., LiPlaCis) include, e.g., saline, sterile water, polyalkylene glycols, oils of vegetable origin, hydrogenated napthalenes, suitable buffer, 1,3-butanediol, Ringer's solution and/or sodium chloride solution. Exemplary formulations for parenteral administration can include solutions prepared in water suitably mixed with a surfactant, e.g., hydroxypropylcellulose. Dispersions can also be prepared in glycerol, liquid polyethylene glycols, DMSO and mixtures thereof with or without alcohol, and in oils. Under ordinary conditions of storage and use, these preparations may contain a preservative to prevent the growth of microorganisms. Other exemplary carriers, excipients, or diluents are described in the Handbook of Pharmaceutical Excipients, 6th Edition, Rowe et al., Eds., Pharmaceutical Press (2009), hereby incorporated by reference in its entirety.

In some embodiments, administration of the sPLA₂ hydrolysable, cisplatin-containing liposome composition (e.g., LiPlaCis) can be accompanied by a hydration program as a prophylaxis against infusion reactions and as an anti-emetic regimen. An exemplary treatment scheme is outlined in Table 1.

TABLE 1
sPLA₂ hydrolysable, cisplatin-containing liposome (e.g., LiPlaCis) treatment, infusion reaction
prophylaxis, hydration schema, emesis prophylaxis.

| | |
|---|---|
| Time definition | Day = 1 at Time = 0 hour is start of LiPlaCis |
| | infusion |

Prophylaxis against infusion reaction:

| | |
|---|---|
| Prednisolone 50 mg BID PO | Day = −1 |
| Solumedrol 40 mg IV | Day = 1 at Time = −2 hour |
| Clemastine 2 mg IV | Day = 1 at Time = −2 hour |
| Paracetamol 1 g PO | Day = 1 at Time = −1 hour |
| Ibuprofen 400 mg PO | |

Pre-hydration:

| | |
|---|---|
| NaCl 0.9% 1½ L over 2 hours* | Day = 1 at Time = −2 hour to Time = 0 hour |
| Mg++ 6 mmol over 2 hours | |
| *NaCl 0.9% 1 L over 1 hour (depending on diuresis) | Day = 1 |

LiPlaCis:

| | |
|---|---|
| LiPlaCis 75 mg in 2 × 500 ml NaCl (0.9%) by 2 h | Day = 1 at Time = 0 to Time = +2 |
| infusion | |

Post-hydration:

| | |
|---|---|
| NaCl 0.9% 2½ L over 12 hours IV or equivalent PO. | Day = 1 at Time = +2 to Time = +14 |

Emesis prophylaxis (A1):

| | |
|---|---|
| Palonosetron 250 μg iv | Day = 1 at Time = −1 |
| (Solumedrol 40 mg iv) | (Day = 1 at Time = −2) Also listed in |
| Prednisolone 25 mg BID PO days 2-3 | prophylaxis section |
| Prednisolone 25 mg OD days 4-5 | Day 2 and day 3 |
| | Day 4 and day 5 |
| Aprepitant p.n. 125 mg PO + 80 mg PO days 1 + 2 | Day 1 and day 2 |

Preparation of sPLA₂ Hydrolysable, Cisplatin-Containing Liposome Composition for Administration

The sPLA₂ hydrolysable, cisplatin-containing liposome composition (e.g., LiPlaCis) can be supplied as a concentrate for infusion (e.g., LiPlaCis Concentrate for Infusion), which can be aseptically diluted in sterile 0.9% NaCl (aq) in an infusion bag before administration. For example, the infusion bag (e.g., LiPlaCis infusion bag system) can be Fresenius FREEFLEX® Sodium Chloride 0.9%, 500 ml. Two such infusion bags can be used for each dose, each containing 50% of the dose.

The liposomal concentrate for infusion (e.g., LiPlaCis Concentrate for Infusion) can be supplied as a white to off-white opalescent dispersion in 30 ml vials, each containing 20 ml. The product can be stored at −80° C. and the concentration (in mg/ml) can be marked on the label. The volume of liposomal concentrate for infusion (e.g., LiPlaCis Concentrate for Infusion) that is to be diluted in order to prepare the final liquid for infusion may vary from patient to patient depending on the desired dose.

The liposomal concentrate for infusion (e.g., LiPlaCis Concentrate for Infusion) may be diluted by the following procedure:

(i) For each dose, the total volume (Vₜₒₜ) of the liposomal concentrate for infusion (e.g., LiPlaCis Concentrate for Infusion) to be used can be calculated according to the following formula:

Vₜₒₜ=D/C

The volume (V_{bag}) to be added to each of the two infusion bags can be calculated according to the following formula:

V_{bag}=Vₜₒₜ/2

Where, Vₜₒₜ is the volume of the liposomal concentrate for infusion (e.g., LiPlaCis Concentrate for Infusion) to be used, in ml; V_{bag} is the volume of liposomal concentrate for infusion (e.g., LiPlaCis Concentrate for Infusion) to be added to each of the two infusions bags, in ml; D is the dose, in mg; and C is the concentration of cisplatin in the liposomal concentrate for infusion (e.g., LiPlaCis Concentrate for Infusion), in mg/ml, stated on the label.

(ii) An appropriate amount of the liposomal concentrate for infusion (e.g., LiPlaCis Concentrate for Infusion) (according to the calculation above) can be thawed prior to use. The thawing can be done in a water bath at 10-25° C. Once thawed, the liposomal concentrate for infusion (e.g., LiPlaCis Concentrate for Infusion) should not be refrozen.

(iii) The calculated total volume Vₜₒₜ is withdrawn, and the volume V_{bag} is added to each of the two infusion bags via a medication valve.

(iv) The infusion liquid should be mixed thoroughly, kept protected from light, and used within about 8 hours.

Cancer Patients that can be Treated with the sPLA₂ Hydrolysable, Cisplatin-Containing Liposome Composition (e.g., LiPlaCis)

A patient who can be treated with the dosage regimen of sPLA₂ hydrolysable, cisplatin-containing liposome (e.g., LiPlaCis) according to the methods described herein, may include, e.g., a patient that has been diagnosed with cancer, a patient that has not received a cancer treatment (e.g., the liposomal formulation of cisplatin, an anti-cancer agent other than the liposomal formulation of cisplatin, or radiation), a patient that has received a cancer treatment (e.g., an anti-cancer agent other than the liposomal formulation of cisplatin or radiation), or a patient during treatment with the liposomal formulation of cisplatin.

For example, the patient may have a solid tumor or a hematological cancer, such as a cancer type selected from prostate cancer, ovarian cancer (e.g., ovarian adenocarcinoma or embryonal carcinoma), liver cancer (e.g., hepatocellular carcinoma (HCC) or hepatoma), myeloma (e.g., multiple myeloma), colorectal cancer (e.g., colon cancer and rectal cancer), leukemia (e.g., acute myeloid leukemia, acute lymphoid leukemia, chronic myeloid leukemia, chronic lymphocytic leukemia, acute myeloblastic leukemia, acute promyelocytic leukemia, acute myelomonocytic leukemia, acute monocytic leukemia, acute erythroleukemia, and chronic leukemia), myelodysplastic syndrome, lymphoma (e.g., diffuse large B-cell lymphoma, cutaneous T-cell lymphoma, peripheral T-cell lymphoma, Hodgkin's lymphoma, non-Hodgkin's lymphoma, Waldenstrom's macroglobulinemia, and lymphocytic lymphoma), cervical cancer, esophageal cancer, melanoma, glioma (e.g., oligodendroglioma), pancreatic cancer (e.g., adenosquamous carcinoma, signet ring cell carcinoma, hepatoid carcinoma, colloid carcinoma, islet cell carcinoma, and pancreatic neuroendocrine carcinoma), gastrointestinal stromal tumor, sarcoma (e.g., fibrosarcoma, myxosarcoma, liposarcoma, chondrosarcoma, osteogenic sarcoma, angiosarcoma, endotheliosarcoma, lymphangiosarcoma, lymphangioendotheliosarcoma, leiomyosarcoma, Ewing's sarcoma, and rhabdomyosarcoma), breast cancer (e.g., medullary carcinoma), ER-positive cancer, bladder cancer, head and neck cancer (e.g., squamous cell carcinoma of the head and neck), lung cancer (e.g., non-small cell lung carcinoma, large cell carcinoma, bronchogenic carcinoma, and papillary adenocarcinoma), metastatic cancer, oral cavity cancer, uterine cancer, testicular cancer (e.g., seminoma and embryonal carcinoma), skin cancer (e.g., squamous cell carcinoma and basal cell carcinoma), thyroid cancer (e.g., papillary carcinoma and medullary carcinoma), brain cancer (e.g., astrocytoma and craniopharyngioma), stomach cancer, intra-epithelial cancer, bone cancer, biliary tract cancer, eye cancer, larynx cancer, kidney cancer (e.g., renal cell carcinoma and Wilms tumor), gastric cancer, blastoma (e.g., nephroblastoma, medulloblastoma, hemangioblastoma, neuroblastoma, and retinoblastoma), polycythemia vera, chordoma, synovioma, mesothelioma, adenocarcinoma, sweat gland carcinoma, sebaceous gland carcinoma, cystadenocarcinoma, bile duct carcinoma, choriocarcinoma, epithelial carcinoma, ependymoma, pinealoma, acoustic neuroma, schwannoma, meningioma, pituitary adenoma, nerve sheath tumor, cancer of the small intestine, cancer of the endocrine system, cancer of the penis, cancer of the urethra, cutaneous or intraocular melanoma, a gynecologic tumor, solid tumors of childhood, and neoplasms of the central nervous system. In particular, the cancer of the patient is, e.g., prostate cancer, ovarian cancer, hepatocellular carcinoma (HCC), multiple myeloma, breast cancer, acute myelogenous leukemia (AML), acute lympho-blastic leukemia (ALL), chronic lymphocytic leukemia (CLL), myelodysplastic syndrome (MDS), chronic myelogenous leukemia—chronic phase (CMLCP), diffuse large B-cell lymphoma (DLBCL), cutaneous T-cell lymphoma (CTCL), peripheral T-cell lymphoma (PTCL), Hodgkin's lymphoma, cervical cancer, renal cell carcinoma (RCC), esophageal cancer, melanoma, glioma, pancreatic cancer, gastrointestinal stromal tumors (GIST), sarcoma, estrogen receptor-positive (ERpos) breast cancer, non-small cell lung carcinoma (NSCLC), colon cancer, bladder cancer, or squamous cell carcinoma of the head and neck (SCCHN).

The patient may have a cancer (e.g., breast cancer) that is resistant to one or more cancer therapies other than the liposomal formulation of cisplatin (e.g., docetaxel, cabazitaxel, mitoxantrone, estramustine, prednisone, carboplatin, bevacizumab, paclitaxel, gemcitabine, doxorubicin, topotecan, etoposide, tamoxifen, letrozole, sorafenib, fluorouracil, capecitabine, oxaliplatin, interferon-alpha, 5-fluorouracil (5-FU), a histone deacetylase (HDAC) inhibitor, ipilimumab, bortezomib, carfilzomib, thalidomide, lenalidomide, pomalidomide, dexamethasone, cyclophosphamide, vincristine, melphalan, tegafur, irinotecan, cetuximab, leucovorin, SN-38, everolimus, temsirolimus, bleomycin, lomustine, depsipeptide, erlotinib, conventional cisplatin, busulfan, epirubicin, arsenic trioxide, bendamustine, fulvestrant, teniposide, adriamycin, decitabine, estramustine, azaguanine, aclarubicin, mitomycin, paclitaxel, taxotere, APO010, ara-c, methylprednisolone, methotrexate, methyl-gag, belinostat, idarubicin, IL4-PR38, valproic acid, all-trans retinoic acid (ATRA), cytoxan, suberoylanilide hydroxamic acid, leukeran, fludarabine, vinblastine, dacarbazine, hydroxyurea, tegafur, daunorubicin, mechlorethamine, streptozocin, carmustine, mercaptopurine, dactinomycin, tretinoin, ifosfamide, floxuridine, thioguanine, PSC 833, herceptin, celecoxib, iressa, anastrozole, and rituximab), surgery, or radiation. The patient may also have experienced a recurrence following surgery, radiation, or treatment with a cancer therapy other than the liposomal formulation of cisplatin.

### Methods of Predicting Responsiveness of Patients Prior to Treatment

Also featured herein are methods of determining responsiveness of a patient to the liposomal formulation of cisplatin (e.g., LiPlaCis), e.g., prior to treatment with the same. For example, a patient can be identified as responsive to the liposomal formulation of cisplatin by determining the expression level of one or more biomarkers (e.g., one or more of the biomarkers shown in Tables 2-5, such as COL5A2 (SEQ ID NO: 73 OR 211) in a biological sample (e.g., a tumor sample) obtained from the patient, and subsequently administered the liposomal formulation of cisplatin (e.g., LiPlaCis). Alternatively, a patient can be identified as less likely to be responsive to the liposomal formulation of cisplatin by determining the expression level of one or more biomarkers (e.g., one or more of the biomarkers shown in Tables 2-5, such as COL5A2 (SEQ ID NO: 73 OR 211) in a biological sample obtained from the patient. If the patient exhibits expression levels of one or more biomarkers indicative of non-responsiveness to the liposomal formulation of cisplatin, the patient may be treated with or offered a treatment with an agent other than the liposomal formulation of cisplatin. In particular, the patient may be treated with, e.g., radiation and/or administration of a therapeutic agent, such as docetaxel, cabazitaxel, mitoxantrone, estramustine, prednisone, carboplatin, bevacizumab, paclitaxel, gemcitabine, doxorubicin, topotecan, etoposide, tamoxifen, letrozole, sorafenib, fluorouracil, capecitabine, oxaliplatin, interferon-alpha, 5-fluorouracil (5-FU), a histone deacetylase (HDAC) inhibitor, ipilimumab, bortezomib, carfilzomib, thalidomide, lenalidomide, pomalidomide, dexamethasone, cyclophosphamide, vincristine, melphalan, tegafur, irinotecan, cetuximab, leucovorin, SN-38, everolimus, temsirolimus, bleomycin, lomustine, depsipeptide, erlotinib, cisplatin, busulfan, epirubicin, arsenic trioxide, bendamustine, fulvestrant, teniposide, adriamycin, decitabine, estramustine, azaguanine, aclarubicin, mitomycin, paclitaxel, taxotere, APO010, ara-c, methylprednisolone, methotrexate, methyl-gag, belinostat, idarubicin, IL4-PR38, valproic acid, all-trans retinoic acid (ATRA), cytoxan, suberoylanilide hydroxamic acid, leukeran, fludarabine, vinblastine, dacarbazine, hydroxyurea, tegafur, daunorubicin, mechlorethamine, streptozocin, carmustine, mercaptopurine, dactinomycin, tretinoin, ifosfamide, floxuridine, thioguanine, PSC 833, herceptin, celecoxib, iressa, anastrozole, or rituximab.

Expression levels of the biomarkers shown in Tables 2-5 may be detected in a subject/patient having cancer and are useful for predicting the responsiveness of the patient to sPLA₂ hydrolysable, cisplatin-containing liposome (e.g., LiPlaCis). These patients may already be determined to be resistant to a therapy other than the sPLA₂ hydrolysable, cisplatin-containing liposome, such as docetaxel, cabazitaxel, mitoxantrone, estramustine, prednisone, carboplatin, bevacizumab, paclitaxel, gemcitabine, doxorubicin, topotecan, etoposide, tamoxifen, letrozole, sorafenib, fluorouracil, capecitabine, oxaliplatin, interferon-alpha, 5-fluorouracil (5-FU), a histone deacetylase (HDAC) inhibitor, ipilimumab, bortezomib, carfilzomib, thalidomide, lenalidomide, pomalidomide, dexamethasone, cyclophosphamide, vincristine, melphalan, tegafur, irinotecan, cetuximab, leucovorin, SN-38, everolimus, temsirolimus, bleomycin, lomustine, depsipeptide, erlotinib, conventional cisplatin, busulfan, epirubicin, arsenic trioxide, bendamustine, fulvestrant, teniposide, adriamycin, decitabine, estramustine, azaguanine, aclarubicin, mitomycin, paclitaxel, taxotere, APO010, ara-c, methylprednisolone, methotrexate, methyl-gag, belinostat, idarubicin, IL4-PR38, valproic acid, all-trans retinoic acid (ATRA), cytoxan, suberoylanilide hydroxamic acid, leukeran, fludarabine, vinblastine, dacarbazine, hydroxyurea, tegafur, daunorubicin, mechlorethamine, streptozocin, carmustine, mercaptopurine, dactinomycin, tretinoin, ifosfamide, floxuridine, thioguanine, PSC 833, herceptin, celecoxib, iressa, anastrozole, or rituximab.

A device, such as a microarray, with one or more single-stranded oligonucleotide probes that have substantial identity (e.g., at least 85%, 90%, 95%, 99%, or 100% sequence identity) to a sequence that is complementary or identical to the nucleic acid sequence of one or more biomarkers shown in Tables 2-5 can be used according to the methods described herein to assess the responsiveness of a cancer patient to treatment with the sPLA₂ hydrolysable, cisplatin-containing liposome (e.g., LiPlaCis). For example, the probes can be used to detect one or more (e.g., two, three, four, five, ten, twenty, or all) of the biomarkers of sensitivity listed in Tables 2 and 4, such as COL5A2 (SEQ ID NO 73 or 211), in a sample (e.g., a tumor sample) from a patient having cancer (e.g., breast cancer). Additionally, the probes can be used to detect one or more (e.g., two, three, four, five, ten, twenty, or all) of the biomarkers of resistance listed in Tables 3 and 5, such as SFN (SEQ ID NO: 96 or 324), in a sample (e.g., a tumor sample) from a patient having cancer (e.g., breast cancer).

Individual biomarkers (e.g., COL5A2 (SEQ ID NO 73 or 211) or SFN (SEQ ID NO: 96 or 324)) and sets of biomarkers shown in Tables 2-5 that can be used to determine the responsiveness of a cancer patient to sPLA₂ hydrolysable, cisplatin-containing liposome (e.g., LiPlaCis) at various stages of disease progression (e.g., patients diagnosed with cancer or patients after cancer recurrence) and at different times during the treatment process (e.g., prior to administration of any cancer treatment, after administration of one or more cancer treatments other than the sPLA₂ hydrolysable, cisplatin-containing liposome, prior to administration of the sPLA₂ hydrolysable, cisplatin-containing liposome, or during administration of the sPLA₂ hydrolysable, cisplatin-containing liposome). Additionally, the methods can be used to determine the responsiveness to sPLA₂ hydrolysable, cisplatin-containing liposome (e.g., LiPlaCis) in a patient with cancer that is resistant to one or more cancer therapies other than LiPlaCis, such as docetaxel, cabazitaxel, mitoxantrone, estramustine, prednisone, carboplatin, bevacizumab, paclitaxel, gemcitabine, doxorubicin, topotecan, etoposide, tamoxifen, letrozole, sorafenib, fluorouracil, capecitabine, oxaliplatin, interferon-alpha, 5-fluorouracil (5-FU), a histone deacetylase (HDAC) inhibitor, ipilimumab, bortezomib, carfilzomib, thalidomide, lenalidomide, pomalidomide, dexamethasone, cyclophosphamide, vincristine, melphalan, tegafur, irinotecan, cetuximab, leucovorin, SN-38, everolimus, temsirolimus, bleomycin, lomustine, depsipeptide, erlotinib, conventional (e.g., free) cisplatin, busulfan, epirubicin, arsenic trioxide, bendamustine, fulvestrant, teniposide, adriamycin, decitabine, estramustine, azaguanine, aclarubicin, mitomycin, paclitaxel, taxotere, APO010, ara-c, methylprednisolone, methotrexate, methyl-gag, belinostat, idarubicin, IL4-PR38, valproic acid, all-trans retinoic acid (ATRA), cytoxan, suberoylanilide hydroxamic acid, leukeran, fludarabine, vinblastine, dacarbazine, hydroxyurea, tegafur, daunorubicin, mechlorethamine, streptozocin, carmustine, mercaptopurine, dactinomycin, tretinoin, ifosfamide, floxuridine, thioguanine, PSC 833, herceptin, celecoxib, iressa, anastrozole, or rituximab.

In particular, featured are methods for determining whether a patient may be responsive to sPLA₂ hydrolysable, cisplatin-containing liposome composition (e.g., LiPlaCis) by, e.g., detecting the expression level (e.g., mRNA or protein produced therefrom) of one or more of the biomarkers shown in Tables 2-5 (e.g., COL5A2 (SEQ ID NO 73 or 211)) in a biological sample (e.g., a tumor biopsy) obtained from the subject using a device (e.g., a microarray or a protein array). The expression level of one or more of the biomarkers of sensitivity may then be compared to the expression level of the biomarkers in a cell or tissue known to be sensitive or resistant to the sPLA₂ hydrolysable, cisplatin-containing liposome (e.g., LiPlaCis) to determine the patient's responsiveness to the sPLA₂ hydrolysable, cisplatin-containing liposome. The patient may be responsive to the sPLA₂ hydrolysable, cisplatin-containing liposome if the expression level of the one or more of the biomarkers of sensitivity (e.g., one or more of COL5A2 (SEQ ID NO 73 or 211), ITGA4 (SEQ ID NO: 1), MSN (SEQ ID NO: 2), FAM46A (SEQ ID NO: 3 or 280), ITGB2 (SEQ ID NO: 4), DOCK2 (SEQ ID NO: 5 or 223), EVL (SEQ ID NO: 6), SACS (SEQ ID NO: 7), PTPRCAP (SEQ ID NO: 8), EBI2 (SEQ ID NO: 9), PTPRC (SEQ ID NO: 10, 18, 25, or 243), ANP32E (SEQ ID NO: 11), SFPQ (SEQ ID NO: 12, 38 or 272), C1QR1 (SEQ ID NO: 13), FNBP1 (SEQ ID NO: 14 or 28), CBFB (SEQ ID NO: 15)) is substantially similar to the expression level of the biomarkers of sensitivity in a cell or tissue known to be sensitive to sPLA₂ hydrolysable, cisplatin-containing liposome (e.g., from a patient sensitive to LiPlaCis). The patient may also be responsive to sPLA₂ hydrolysable, cisplatin-containing liposome if the level of expression of one or more of the biomarkers of resistance (e.g., one or more of SFN (SEQ ID NO: 96 or 324), LISCH7 (SEQ ID NO: 97), EPB41L4B (SEQ ID NO: 98), MST1R (SEQ ID NO: 99), ITGB4 (SEQ ID NO: 100), DBNDD2 (SEQ ID NO: 102 or 365), TACSTD1 (SEQ ID NO: 104), MISP (SEQ ID NO: 105), KRT8 (SEQ ID NO: 106), JUP (SEQ ID NO: 107 or 400), KRT18 (SEQ ID NO: 108 or 306), FA2H (SEQ ID NO: 109), MGAT4B (SEQ ID NO: 110), DSG2 (SEQ ID NO:111 or 312), LRP5 (SEQ ID NO: 112)) is substantially dissimilar to the expression level of the biomarkers of resistance in a cell or tissue known to be resistant to sPLA₂ hydrolysable, cisplatin-containing liposome (e.g., from a patient resistant to LiPlaCis).

Also featured are methods of treating a patient having cancer, such as a patient having a cancer that is resistant to one or more cancer therapies other than the sPLA₂ hydrolysable, cisplatin-containing liposome (e.g., LiPlaCis), by detecting the expression levels of one or more of the biomarkers shown in Tables 2-5 (e.g., COL5A2 (SEQ ID NO: 73 OR 211) in a sample (e.g., a tumor sample) from the patient, and then administering the sPLA₂ hydrolysable, cisplatin-containing liposome (e.g., LiPlaCis) based on the expression levels of the biomarkers. In particular, a patient having cancer may be administered sPLA₂ hydrolysable, cisplatin-containing liposome if the expression level of one or more biomarkers of sensitivity is substantially similar to the expression level of the biomarkers of sensitivity in a cell or tissue known to be sensitive to the same. Additionally, a patient having cancer may be administered sPLA₂ hydrolysable, cisplatin-containing liposome (e.g., LiPlaCis) if the expression level of one or more biomarkers of resistance is substantially dissimilar to the expression level of the biomarkers of resistance in a cell or tissue known to be resistant to the same. Thus, the methods can be used to treat cancer patients predicted to be responsive to the sPLA₂ hydrolysable, cisplatin-containing liposome (e.g., LiPlaCis), such as patients having, e.g., breast cancer, prostate cancer, ovarian cancer, hepatocellular carcinoma (HCC), cervical cancer, renal cell carcinoma (RCC), esophageal cancer, melanoma, glioma, pancreatic cancer, gastrointestinal stromal tumors (GIST), sarcoma, estrogen receptor-positive (ERpos) breast cancer, non-small cell lung carcinoma (NSCLC), colon cancer, bladder cancer, squamous cell carcinoma of the head and neck (SCCHN), acute myelogenous leukemia (AML), acute lympho-blastic leukemia (ALL), chronic lymphocytic leukemia (CLL), myelodysplastic syndrome (MDS), chronic myelogenous leukemia—chronic phase (CMLCP), diffuse large B-cell lymphoma (DLBCL), cutaneous T-cell lymphoma (CTCL), peripheral T-cell lymphoma (PTCL), and Hodgkin's lymphoma. Alternatively, a patient having cancer may not be administered sPLA₂ hydrolysable, cisplatin-containing liposome (e.g. LiPlaCis) if the expression level of one or more biomarkers of sensitivity is substantially dissimilar to the expression level of the biomarkers of sensitivity in a cell or tissue known to be sensitive to the sPLA₂ hydrolysable, cisplatin-containing liposome. Likewise, a patient having cancer may not be administered sPLA₂ hydrolysable, cisplatin-containing liposome (e.g., LiPlaCis) if the expression level of one or more biomarkers of resistance is substantially similar to the expression level of the biomarkers of resistance in a cell or tissue known to be resistant to the sPLA₂ hydrolysable, cisplatin-containing liposome.

Methods are described herein for identifying biomarkers of drug responsiveness, detecting biomarker gene expression in cancer patients, determining the responsiveness of a cancer patient to sPLA₂ hydrolysable, cisplatin-containing liposome (e.g., LiPlaCis), and treating cancer patients with sPLA₂ hydrolysable, cisplatin-containing liposome (e.g., LiPlaCis). Also described are devices and kits for use in these methods.

### Methods for Identifying Biomarkers of Drug Responsiveness

Featured herein are methods for identifying biomarkers (e.g., one or more of the biomarkers of Tables 2-5) for determining the responsiveness of a cancer patient to a cancer treatment, such as sPLA₂ hydrolysable, cisplatin-containing liposome (e.g., LiPlaCis). Such methods can involve, for example, an algorithm based on growth inhibition values (GI50) of cell lines (e.g., NCI60 cell lines) subjected to treatment with sPLA₂ hydrolysable, cisplatin-containing liposome (e.g., LiPlaCis), followed by measurement of gene expression (e.g., using a microarray (e.g., an Affymetrix HG-U133A or HG-U133_Plus_2 array)).

### Methodology of the In Vitro Cancer Growth Inhibition Screen

The human tumor cell lines of the cancer screening panel may be grown in RPMI 1640 medium containing 5% fetal bovine serum and 2 mM L-glutamine. Cells may be inoculated into 96 well microtiter plates in 100 μL at plating densities ranging from 5,000 to 40,000 cells/well depending on the doubling time of individual cell lines. After cell inoculation, the microtiter plates may be incubated at 37° C., 5% CO2, 95% air and 100% relative humidity for 24 hours prior to addition of experimental compounds.

After 24 hours, two plates of each cell line may be fixed in situ with TCA, to represent a measurement of the cell population for each cell line at the time of compound addition (Tz). Experimental compounds may be solubilized in dimethyl sulfoxide at 400-fold the desired final maximum test concentration and stored frozen prior to use. At the time of compound (e.g., sPLA₂ hydrolysable, cisplatin-containing liposome, such as LiPlaCis) addition, an aliquot of frozen concentrate may be thawed and diluted to twice the desired final maximum test concentration with complete medium containing 50 μg/ml Gentamicin. A total of four additional 10-fold or ½ log serial dilutions are made to provide a total of five concentrations plus control. Aliquots of 100 μl of these different compound dilutions are added to the appropriate microtiter wells already containing 100 μl of medium, resulting in the required final compound concentrations.

Following compound (e.g., sPLA₂ hydrolysable, cisplatin-containing liposome, such as LiPlaCis) addition, the plates may be incubated for an additional 48 h at 37° C., 5% CO2, 95% air, and 100% relative humidity. For adherent cells, the assay may be terminated by the addition of cold TCA. Cells may be fixed in situ by the gentle addition of 50 μl of cold 50% (w/v) TCA (final concentration, 10% TCA) and incubated for 60 minutes at 4° C. The supernatant may be discarded, and the plates may be washed five times with tap water and air-dried. Sulforhodamine B (SRB) solution (100 μl) at 0.4% (w/v) in 1% acetic acid may be added to each well, and the plates may be incubated for 10 minutes at room temperature. After staining, unbound dye may be removed by washing five times with 1% acetic acid and the plates may be air-dried. Bound stain may be subsequently solubilized with 10 mM trizma base, and the absorbance may be read on an automated plate reader at a wavelength of 515 nm. For suspension cells, the methodology may be the same, except that the assay may be terminated by fixing settled cells at the bottom of the wells by gently adding 50 μl of 80% TCA (final concentration, 16% TCA). Using the seven absorbance measurements [time zero, (Tz), control growth, (C), and test growth in the presence of compound (e.g., sPLA₂ hydrolysable, cisplatin-containing liposome, such as LiPlaCis) at the five concentration levels (Ti)], the percentage growth may be calculated at each of the compound concentrations levels. Percentage growth inhibition may be calculated as:

[(Ti−Tz)/(C−Tz)]×100 for concentrations for which Ti>/=Tz

[(Ti−Tz)/Tz]×100 for concentrations for which Ti<Tz

Three dose response parameters may be calculated for each experimental agent (e.g., sPLA₂ hydrolysable, cisplatin-containing liposome, such as LiPlaCis). Growth inhibition of 50% (GI50) is calculated from [(Ti−Tz)/(C−Tz)]×100=50, which is the agent (e.g., sPLA₂ hydrolysable, cisplatin-containing liposome, such as LiPlaCis) concentration resulting in a 50% reduction in the net protein increase (as measured by SRB staining) in control cells during the compound incubation. The compound concentration resulting in total growth inhibition (TGI) is calculated from Ti=Tz. The LC50 (concentration of compound resulting in a 50% reduction in the measured protein at the end of the compound treatment as compared to that at the beginning) indicating a net loss of cells following treatment is calculated from [(Ti−Tz)/Tz]×100=−50. Values are calculated for each of these three parameters if the level of activity is reached; however, if the effect is not reached or is exceeded, the value for that parameter is expressed as greater or less than the maximum or minimum concentration tested.

### Gene Expression and Growth Inhibition Analysis

The gene expression measurements of NCI60 cancer cell lines can be obtained from a publically available database (e.g., the National Cancer Institute and the Massachusetts Institute of Technology). Each dataset can be normalized so that sample expression measured by different chips can be compared. The preferred method of normalization is the logit transformation, which may be performed for each gene y on each chip, as follows:

logit(y)=log[(y-background)/(saturation−y)],

where background is calculated as the minimum intensity measured on the chip minus 0.1% of the signal intensity range: min-0.001*(max-min), and saturation is calculated as the maximum intensity measured on the chip plus 0.1% of the signal intensity range: max+0.001*(max-min). The resulting logit transformed data may then be z-transformed to mean zero and standard deviation 1.

Next, gene expression can be correlated to cancer cell growth inhibition. Growth inhibition data (G150) of the NCI60 cell lines in the presence of a cancer treatment, such as LiPlaCis, can be obtained from the NCI. The correlation between the logit-transformed expression level of each gene in each cell line and the logarithm of G150 (the concentration of a given compound that results in a 50% inhibition of growth) can be calculated, e.g., using the Pearson correlation coefficient or the Spearman Rank-Order correlation coefficient. Instead of using GI50s, any other measure of patient sensitivity to a given treatment (e.g., sPLA₂ hydrolysable, cisplatin-containing liposome, such as LiPlaCis) may be correlated to gene expression levels of the patient. Since a plurality of measurements may be available for a single gene, the most accurate determination of correlation coefficient can be, e.g., the median of the correlation coefficients calculated for all probes measuring expression of the same gene.

For example, the median correlation coefficient of gene expression measured on a probe to growth inhibition or patient sensitivity to sPLA₂ hydrolysable, cisplatin-containing liposome (e.g., LiPlaCis) can be calculated for all genes of interest. Genes that have a median correlation above, e.g., 0.20, 0.21 0.22. 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, or higher (e.g., 0.2 or higher), can be used as biomarkers of sensitivity for assessing responsiveness of a cancer patient (e.g., a patient having cancer that is resistant to one or more cancer therapies other than sPLA₂ hydrolysable, cisplatin-containing liposome, such as LiPlaCis) to sPLA₂ hydrolysable, cisplatin-containing liposome (e.g., LiPlaCis). Likewise, genes that have a median correlation below, e.g., −0.20, −0.21, −0.22. −0.23, −0.24, −0.25, −0.26, −0.27, −0.28, −0.29, −0.30, −0.31, −0.32, −0.33, −0.34, −0.35, −0.36, −0.37, −0.38, −0.39, −0.40, or lower (e.g., −0.2 or lower), can be used as biomarkers of resistance for assessing responsiveness of a cancer patient (e.g., a patient having cancer that is resistant to one or more cancer therapies other than sPLA₂ hydrolysable, cisplatin-containing liposome, such as LiPlaCis) to sPLA₂ hydrolysable, cisplatin-containing liposome (e.g., LiPlaCis). Preferably, the correlation coefficient of a biomarker of sensitivity will exceed 0.2, while the correlation coefficient of a biomarker of resistance will be less than −0.2. The result is a list of biomarker genes that correlate to sensitivity or resistance to sPLA₂ hydrolysable, cisplatin-containing liposome (e.g., LiPlaCis), as shown in Tables 2 and 4 and Tables 3 and 5, respectively.

### Cancer Types

The methods, devices, and kits of the invention can be used for diagnosing, prognosing, monitoring, treating, and/or reducing cancer in a subject suffering from, diagnosed with, or susceptible to cancer. Non-limiting examples of cancers that can be diagnosed, prognosed, monitored, treated, or reduced using the methods include hematological and solid tumors. In particular, cancers include, e.g., breast cancer, prostate cancer, ovarian cancer (e.g., ovarian adenocarcinoma or embryonal carcinoma), liver cancer (e.g., hepatocellular carcinoma (HCC) or hepatoma), myeloma (e.g., multiple myeloma), colorectal cancer (e.g., colon cancer and rectal cancer), leukemia (e.g., acute myeloid leukemia, acute lymphoid leukemia, chronic myeloid leukemia, chronic lymphocytic leukemia, acute myeloblastic leukemia, acute promyelocytic leukemia, acute myelomonocytic leukemia, acute monocytic leukemia, acute erythroleukemia, and chronic leukemia), myelodysplastic syndrome, lymphoma (e.g., diffuse large B-cell lymphoma, cutaneous T-cell lymphoma, peripheral T-cell lymphoma, Hodgkin's lymphoma, non-Hodgkin's lymphoma, Waldenstrom's macroglobulinemia, and lymphocytic lymphoma), cervical cancer, esophageal cancer, melanoma, glioma (e.g., oligodendroglioma), pancreatic cancer (e.g., adenosquamous carcinoma, signet ring cell carcinoma, hepatoid carcinoma, colloid carcinoma, islet cell carcinoma, and pancreatic neuroendocrine carcinoma), gastrointestinal stromal tumor, sarcoma (e.g., fibrosarcoma, myxosarcoma, liposarcoma, chondrosarcoma, osteogenic sarcoma, angiosarcoma, endotheliosarcoma, lymphangiosarcoma, lymphangioendotheliosarcoma, leiomyosarcoma, Ewing's sarcoma, and rhabdomyosarcoma), breast cancer (e.g., medullary carcinoma), ER-positive cancer, bladder cancer, head and neck cancer (e.g., squamous cell carcinoma of the head and neck), lung cancer (e.g., non-small cell lung carcinoma, large cell carcinoma, bronchogenic carcinoma, and papillary adenocarcinoma), metastatic cancer, oral cavity cancer, uterine cancer, testicular cancer (e.g., seminoma and embryonal carcinoma), skin cancer (e.g., squamous cell carcinoma and basal cell carcinoma), thyroid cancer (e.g., papillary carcinoma and medullary carcinoma), brain cancer (e.g., astrocytoma and craniopharyngioma), stomach cancer, intra-epithelial cancer, bone cancer, biliary tract cancer, eye cancer, larynx cancer, kidney cancer (e.g., renal cell carcinoma and Wilms tumor), gastric cancer, blastoma (e.g., nephroblastoma, medulloblastoma, hemangioblastoma, neuroblastoma, and retinoblastoma), polycythemia vera, chordoma, synovioma, mesothelioma, adenocarcinoma, sweat gland carcinoma, sebaceous gland carcinoma, cystadenocarcinoma, bile duct carcinoma, choriocarcinoma, epithelial carcinoma, ependymoma, pinealoma, acoustic neuroma, schwannoma, meningioma, pituitary adenoma, nerve sheath tumor, cancer of the small intestine, cancer of the endocrine system, cancer of the penis, cancer of the urethra, cutaneous or intraocular melanoma, a gynecologic tumor, solid tumors of childhood, and neoplasms of the central nervous system.

In particular, the methods are useful for diagnosing, prognosing, monitoring, treating, or preventing, e.g., breast cancer, prostate cancer, ovarian cancer, hepatocellular carcinoma (HCC), cervical cancer, renal cell carcinoma (RCC), esophageal cancer, melanoma, glioma, pancreatic cancer, gastrointestinal stromal tumors (GIST), sarcoma, estrogen receptor-positive (ERpos) breast cancer, non-small cell lung carcinoma (NSCLC), colon cancer, bladder cancer, squamous cell carcinoma of the head and neck (SCCHN), acute myelogenous leukemia (AML), acute lympho-blastic leukemia (ALL), chronic lymphocytic leukemia (CLL), myelodysplastic syndrome (MDS), chronic myelogenous leukemia—chronic phase (CMLCP), diffuse large B-cell lymphoma (DLBCL), cutaneous T-cell lymphoma (CTCL), peripheral T-cell lymphoma (PTCL), and Hodgkin's lymphoma.

For example, the cancer can be prostate cancer, such as Stage I, II (e.g., IIA or IIB), III, or IV prostate cancer. In particular, the cancer may be prostate cancer that is resistant to one or more cancer therapies, such as docetaxel, cabazitaxel, mitoxantrone, estramustine, prednisone, and/or surgery. Alternatively, the cancer is an ovarian cancer. The ovarian cancer can be, for example, a Stage I (e.g., Stage IA, IB, or IC), Stage II (e.g., Stage IIA or IIB), Stage III (e.g., Stage IIIA1, IIIA2, IIIB, or IIIC), or Stage IV (e.g., Stage IVA or IVB) ovarian cancer. In particular, the cancer can be ovarian cancer that is resistant to one or more cancer therapies, such as docetaxel, carboplatin, bevacizumab, paclitaxel, gemcitabine, doxorubicin, topotecan, etoposide, tamoxifen, and/or letrozole. Additionally, the cancer can be HCC, such as Stage I, Stage II, Stage III (e.g., Stage IlIA, IIIB, or IIIC), or Stage IV (e.g., Stage IVA or IVB) HCC. In particular, the cancer can be HCC that is resistant to one or more cancer therapies, such as sorafenib, doxorubicin, cisplatin, gemcitabine, capecitabine, oxaliplatin, interferon-alpha, and/or 5-fluorouracil (5-FU).

### Methods for Detecting Biomarker Gene Expression in Cancer Patients

A cancer patient can be assessed for sensitivity or resistance to sPLA₂ hydrolysable, cisplatin-containing liposome (e.g., LiPlaCis) by detecting gene expression of a biomarker (e.g., one or more of the biomarkers of Tables 2-5) in a biological sample obtained from the cancer patient (e.g., a patient having cancer that is resistant to one or more cancer therapies other than the sPLA₂ hydrolysable, cisplatin-containing liposome such as LiPlaCis). The biological sample can include, for example, cells, tissue (e.g., a tissue sample obtained by biopsy), blood, serum, plasma, urine, sputum, cerebrospinal fluid, lymph tissue or fluid, or pancreatic fluid. For example, the biological sample can be fresh frozen or formalin-fixed paraffin embedded (FFPE) tissue obtained from the subject, such as a tumor sample (e.g., a biopsy) from the tissue of interest (e.g., prostate, ovarian, lung, lymph nodes, thymus, spleen, bone marrow, breast, colorectal, pancreatic, cervical, bladder, gastrointestinal, head, or neck tissue).

### RNA Extraction and Biomarker Expression Measurement

Cell samples or tissue samples may be snap frozen in liquid nitrogen until processing. RNA may be extracted using, e.g., Trizol Reagent from Invitrogen following manufacturer's instructions, and detected directly or converted to cDNA for detection. RNA may be amplified using, e.g., MessageAmp kit from Ambion following manufacturer's instructions. Amplified RNA may be quantified using, e.g., HG-U133A or HG-U133_Plus2 GeneChip from Affymetrix Inc. or a compatible apparatus, e.g., the GCS3000Dx GENECHIP® System from Affymetrix Inc., using the manufacturer's instructions. The resulting biomarker expression measurements may be further analyzed as described herein. The procedures described can be implemented using, e.g., R software available from R-Project and supplemented with packages available from Bioconductor.

One or more of the biomarkers shown in Tables 2-5 (e.g., COL5A2 (SEQ ID NO: 73 OR 211) may be measured in a biological sample (e.g., a tumor sample) obtained from the cancer patient (e.g., a patient with any of the cancer types described herein, such as a patient having cancer that is resistant to one or more cancer therapies other than sPLA₂ hydrolysable, cisplatin-containing liposome, such as LiPlaCis) using, e.g., polymerase chain reaction (PCR), reverse transcriptase PCR (RT-PCR), quantitative real-time PCR (qRT-PCR), an array (e.g., a microarray), a genechip, pyrosequencing, nanopore sequencing, sequencing by synthesis, sequencing by expansion, single molecule real time technology, sequencing by ligation, microfluidics, infrared fluorescence, next generation sequencing (e.g., RNA-Seq techniques), Northern blots, Western blots, Southern blots, NanoString nCounter technologies (e.g., those described in U.S. Patent Application Nos. US 2011/0201515, US 2011/0229888, and US 2013/0017971, each of which is incorporated by reference in its entirety), proteomic techniques (e.g., mass spectrometry or protein arrays), and combinations thereof.

### Devices

Devices of the invention can be used for detecting the level of expression of one or more biomarkers shown in Tables 2-5. The device may include at least one single-stranded nucleic acid (e.g., a probe) having at least 85% sequence identity (e.g., 85%, 90%, 95%, 97%, 98%, 99%, or 100% sequence identity) to a nucleic acid sequence that is complementary or identical to at least 5 (e.g., at least 10, at least 15, at least 20, or more) consecutive nucleotides of one or more biomarkers shown in Tables 2-5 (e.g., COL5A2 (SEQ ID NO 73 or 211) or SFN (SEQ ID NO: 96 or 324)), in which the at least one single-stranded nucleic acid is sufficient for the detection of the expression level of the one or more biomarkers. The device may be used to detect the expression level of a given biomarker by specific hybridization between the single-stranded nucleic acid and the biomarker (e.g., an mRNA, genomic DNA, or non-coding RNA), a nucleic acid encoding the biomarker (e.g., an mRNA), or a complementary nucleic acid thereof. The device may be or include a microarray. The device may also include or be used with reagents and materials for next generation sequence (e.g., sequencing by synthesis). The device may also include or be used with NanoString reagents and at least one nCounter cartridge. The device may be or include a protein array, which contains one or more protein binding moieties (e.g., proteins, antibodies, nucleic acids, aptamers, affibodies, lipids, phospholipids, small molecules, labeled variants of any of the above, and any other moieties useful for protein detection as well known in the art) capable of detectably binding to the polypeptide product(s) of one or more biomarkers shown in Tables 2-5. The device may also be a cartridge for measuring an amplification product resulting from hybridization between one or more nucleic acid molecules from the patient and at least one single-stranded nucleic acid single-stranded nucleic acid molecules of the device, such as a device for performing qRT-PCR.

### Microarrays

The expression levels of the biomarkers (e.g., the biomarkers listed in Tables 2-5 (e.g., COL5A2 (SEQ ID NO: 73 OR 211) may be determined using high-throughput expression profiling platforms, such as microarrays. In particular, a microarray for use in the methods for assessing the responsiveness of a cancer patient (e.g., a patient having cancer that is resistant to one or more cancer therapies other than sPLA₂ hydrolysable, cisplatin-containing liposome, such as LiPlaCis) to sPLA₂ hydrolysable, cisplatin-containing liposome (e.g., LiPlaCis) contains or is produced by generating oligonucleotide probes (e.g., DNA, cDNA, or RNA probes) capable of hybridizing to one or more biomarkers of interest (e.g., one or more of the biomarkers of Tables 2-5) or the complement sequences thereof. Each probe can have, e.g., at least 10, 15, 20, 25, 30, or more contiguous nucleic acid residues (e.g., at least 15) that are complementary or identical to a nucleic acid sequence of a selected biomarker. The probe nucleic sequence can also have at least 85% (e.g., 90%, 95%, 99%, or 100%) sequence identity to the nucleic acid sequence of the gene coding the biomarker (e.g., COL5A2 (SEQ ID NO 73 or 211)) or the complement sequence thereof. In particular, the probe sequences can be complementary to all or a portion of the nucleic acid sequence of the biomarker(s).

For example, microarrays of the invention for determining responsiveness to sPLA₂ hydrolysable, cisplatin-containing liposome (e.g., LiPlaCis) can include probes for one or more (e.g., at least 5, 10, 15, or 20 or more (e.g., all)) biomarkers of sensitivity shown in Tables 2 and 4, such as COL5A2 (SEQ ID NO 73 or 211), ITGA4 (SEQ ID NO: 1), MSN (SEQ ID NO: 2), FAM46A (SEQ ID NO: 3 or 280), ITGB2 (SEQ ID NO: 4), DOCK2 (SEQ ID NO: 5 or 223), EVL (SEQ ID NO: 6), SACS (SEQ ID NO: 7), PTPRCAP (SEQ ID NO: 8), EBI2 (SEQ ID NO: 9), PTPRC (SEQ ID NO: 10, 18, 25, or 243), ANP32E (SEQ ID NO: 11), SFPQ (SEQ ID NO: 12, 38 or 272), C1QR1 (SEQ ID NO: 13), FNBP1 (SEQ ID NO: 14 or 28), CBFB (SEQ ID NO: 15)

Microarrays for determining responsiveness to sPLA₂ hydrolysable, cisplatin-containing liposome (e.g., LiPlaCis) can also include probes for one or more (e.g., at least 5, 10, 15, or 20 or more (e.g., all)) biomarkers of resistance listed in Tables 3 and 5, such as SFN (SEQ ID NO: 96 or 324), LISCH7 (SEQ ID NO: 97), EPB41L4B (SEQ ID NO: 98), MST1R (SEQ ID NO: 99), ITGB4 (SEQ ID NO: 100), DBNDD2 (SEQ ID NO: 102 or 365), TACSTD1 (SEQ ID NO: 104), MISP (SEQ ID NO: 105), KRT8 (SEQ ID NO: 106), JUP (SEQ ID NO: 107 or 400), KRT18 (SEQ ID NO: 108 or 306), FA2H (SEQ ID NO: 109), MGAT4B (SEQ ID NO: 110), DSG2 (SEQ ID NO:111 or 312), LRP5 (SEQ ID NO: 112).

Microarrays for determining responsiveness to sPLA₂ hydrolysable, cisplatin-containing liposome (e.g., LiPlaCis) can also include probes for one or more (e.g., at least 5, 10, 15, or 20 or more (e.g., all)) biomarkers of sensitivity and biomarkers of resistance shown in Tables 2-5, such as COL5A2 (SEQ ID NO 73 or 211), ITGA4 (SEQ ID NO: 1), MSN (SEQ ID NO: 2), FAM46A (SEQ ID NO: 3 or 280), ITGB2 (SEQ ID NO: 4), DOCK2 (SEQ ID NO: 5 or 223), EVL (SEQ ID NO: 6), SACS (SEQ ID NO: 7), PTPRCAP (SEQ ID NO: 8), EBI2 (SEQ ID NO: 9), PTPRC (SEQ ID NO: 10, 18, 25, or 243), ANP32E (SEQ ID NO: 11), SFPQ (SEQ ID NO: 12, 38 or 272), C1QR1 (SEQ ID NO: 13), FNBP1 (SEQ ID NO: 14 or 28), CBFB (SEQ ID NO: 15), SFN (SEQ ID NO: 96 or 324), LISCH7 (SEQ ID NO: 97), EPB41L4B (SEQ ID NO: 98), MST1R (SEQ ID NO: 99), ITGB4 (SEQ ID NO: 100), DBNDD2 (SEQ ID NO: 102 or 365), TACSTD1 (SEQ ID NO: 104), MISP (SEQ ID NO: 105), KRT8 (SEQ ID NO: 106), JUP (SEQ ID NO: 107 or 400), KRT18 (SEQ ID NO: 108 or 306), FA2H (SEQ ID NO: 109), MGAT4B (SEQ ID NO: 110), DSG2 (SEQ ID NO:111 or 312), LRP5 (SEQ ID NO: 112).

A microarray probe may be single-stranded or double-stranded. The probe may be labeled (e.g., detectably labeled with a fluorescent molecule, dye molecule, small molecule, epitope tag, barcode sequence, polypeptide, or any other detectable molecule). Probes can be detectably labeled and immobilized on a solid support to form the microarray. For example, probes can be either prefabricated and spotted to the surface or directly synthesized on to the surface (in situ) of the microarray. The microarray can also be configured such that the sequence and position of each member (e.g., probe) of the array is known. For example, a selection of biomarkers whose expression correlates with an increased likelihood of responsiveness to sPLA₂ hydrolysable, cisplatin-containing liposome (e.g., LiPlaCis) can be arrayed on a solid support. Hybridization of a labeled probe with a particular target nucleic acid (e.g., an mRNA corresponding to one or more biomarkers of Tables 2-5) indicates that the sample from which the mRNA was derived expresses that biomarker (e.g., the biomarker of sensitivity or resistance to sPLA₂ hydrolysable, cisplatin-containing liposome, such as LiPlaCis).

### PCR-Based Techniques

As few as one to thirty (e.g., 5 to 30 or 10 to 30, or at least the first 15 of the biomarkers listed in Tables 2-5) biomarkers may be used to determine patient responsiveness to sPLA₂ hydrolysable, cisplatin-containing liposome (e.g., LiPlaCis) using the methods described herein. Tissue or cell samples from a cancer patient (e.g., a patient having cancer that is resistant to one or more cancer therapies other than sPLA₂ hydrolysable, cisplatin-containing liposome (e.g., LiPlaCis) can be conveniently assayed for gene expression levels using polymerase chain reaction (PCR) analysis, such as quantitative real-time PCR (qPCR), or quantitative loop-mediated isothermal amplification (q-LAMP). For example, an mRNA corresponding to a biomarker of Tables 2-5 can be detected in a biological sample by (a) producing cDNA from the sample by reverse transcription using at least one primer; (b) amplifying the cDNA so produced using a target polynucleotide as sense and antisense primers to amplify target cDNAs therein; and (c) detecting the presence of the amplified target cDNA using polynucleotide probes. The primers and probes including the target sequences shown in Tables 2-5, such as COL5A2 (SEQ ID NO 73 or 211) and/or SFN (SEQ ID NO: 96 or 324), may be used to detect expression of one or more of the indicated biomarkers using PCR. The methods can include one or more steps that allow determination of the levels of target mRNA in a biological sample (e.g., by simultaneously examining the levels of a comparative control mRNA sequence or “housekeeping” gene, such as an actin family member or GAPDH). The primers for these PCR-based assays may be labeled for detection according to methods known in the art.

### Sequencing

The expression levels of the biomarkers shown in Tables 2-5, such as COL5A2 (SEQ ID NO 73 or 211) and/or SFN (SEQ ID NO: 96 or 324), may be determined using sequencing technologies, such as next generation sequencing platforms (e.g., RNA-Seq), as described in Mortazavi et al., Nat. Methods 5: 621-628, 2008, hereby incorporated by reference. RNA-Seq is a robust technology for monitoring expression by direct sequencing of the RNA molecules in a sample. This methodology may include fragmentation of RNA to an average length of, e.g., 200 nucleotides, conversion to cDNA by random priming, and synthesis of double-stranded cDNA (e.g., using the PROTOSCRIPT® First Strand cDNA Synthesis Kit from New England Biosciences). The cDNA may then be converted into a molecular library for sequencing by addition of sequence adapters for each library (e.g., from ILLUMINA®/Solexa), and the resulting 50 to 100 nucleotide reads are mapped onto the genome. Exemplary sequencing platforms suitable for use according to the methods include, e.g., pyrosequencing, ILLUMINA® sequencing by synthesis, SOLID® sequencing, ION TORRENT® sequencing, and SMRT® sequencing.

Methods of Determining the Responsiveness of a Patient to sPLA₂ Hydrolysable, Cisplatin-Containing Liposome

Featured are methods for determining the responsiveness of a cancer patient to treatment with one or more cancer therapies, in particular, a liposomal cisplatin composition, such as LiPlaCis. The patient may also be resistant to one or more cancer therapies other than sPLA₂ hydrolysable, cisplatin-containing liposome, such as LiPlaCis). The diagnostic methods include assaying the level of expression of one or more of the biomarkers shown in Tables 2-5 (e.g., COL5A2 (SEQ ID NO 73 or 211) or SFN (SEQ ID NO: 96 or 324)). The methods of the invention may be used for predicting a patient's responsiveness to sPLA₂ hydrolysable, cisplatin-containing liposome (e.g., LiPlaCis), and optionally, treating the cancer patient throughout the progression of cancer and/or in cases of recurrence (e.g., after a first line treatment, a second line treatment, and/or a third line treatment).

The invention provides individual biomarkers (e.g., COL5A2 (SEQ ID NO: 73 OR 211) and sets of biomarkers (e.g., two or more of the biomarkers listed in Tables 2-5), the expression levels of which, as detected in a biological sample (e.g., a tumor sample, such as a biopsy) obtained from a cancer patient (e.g., a human with cancer), are indicative of responsiveness to sPLA₂ hydrolysable, cisplatin-containing liposome (e.g., LiPlaCis). The biomarkers were identified using methods similar to those previously described in, e.g., Chen et al. (Mol. Cancer Ther. 11:34-33, 2012), Wang et al. (J. Nat. Cancer Inst. 105: 1284-1291, 2013), and Knudsen et al. (PLoS One, 9: e87415, 2014), each of which are incorporated by reference herein in their entirety. In particular, an algorithm based on growth inhibition values (GI50) of a cell line (e.g., NCI60 cells) is subjected to treatment with sPLA₂ hydrolysable, cisplatin-containing liposome (e.g., LiPlaCis) and gene expression is determined (e.g., by microarray analysis, reverse transcriptase polymerase chain reaction (RT-PCR), quantitative real-time PCR (qPCR), or next generation sequencing). After normalization, genes with, e.g., a Pearson correlation coefficient greater than about 0.2 or below about −0.2 can be classified as biomarkers of sensitivity or resistance, respectively. In particular, a correlation coefficient of about 0.2 or greater is a statistically significant cut-off known in the art for establishing whether the expression level of A GENE, e.g., the genes shown in Tables 2-5, correlate with the likelihood of cancer treatment sensitivity, such as sensitivity to sPLA₂ hydrolysable, cisplatin-containing liposome (e.g., LiPlaCis). Thus, a correlation coefficient of about 0.2 or greater or about −0.2 or lower can be used to estimate the statistical significance of the expression level of the genes of Tables 2-5 for predicting patient responsiveness to treatment with sPLA₂ hydrolysable, cisplatin-containing liposome (e.g., LiPlaCis) according to the methods described herein.

### Comparison of Biomarker Expression Levels

One or more biomarkers of sensitivity and/or resistance, identified as described herein, can be used to predict responsiveness to sPLA₂ hydrolysable, cisplatin-containing liposome (e.g., LiPlaCis) by measuring the expression level of the biomarkers in a biological sample obtained from the cancer patient. A single biomarker (e.g., any of the biomarkers of Tables 2-5, such as COL5A2 (SEQ ID NO: 73 OR 211) may be used to determine the responsiveness of a cancer patient (e.g., a patient having cancer that is resistant to one or more cancer therapies other than LiPlaCis) to sPLA₂ hydrolysable, cisplatin-containing liposome (e.g., LiPlaCis). After determining the expression level of the biomarker(s) in a sample (e.g., a tumor sample) from the cancer patient, the expression level of the biomarker(s) in the sample may be compared to the expression level of the biomarker(s) in a cell (e.g., a cancer cell) or tissue (e.g., a tumor tissue) known to be sensitive to treatment with sPLA₂ hydrolysable, cisplatin-containing liposome (e.g., LiPlaCis). If the expression level of the biomarker(s) in the sample from the cancer patient is substantially similar (e.g., identical to or has the same trend of expression level) to the expression level of the biomarker(s) in the cell or tissue known to be sensitive to sPLA₂ hydrolysable, cisplatin-containing liposome (e.g., LiPlaCis), then the cancer patient is predicted to be responsive to treatment with the sPLA₂ hydrolysable, cisplatin-containing liposome. Alternatively, if the expression level of the biomarker(s) in the sample from the cancer patient is substantially dissimilar to the expression level of the biomarker(s) in the cell or tissue known to be sensitive to sPLA₂ hydrolysable, cisplatin-containing liposome (e.g., LiPlaCis), then the cancer patient is predicted to be non-responsive to treatment with the sPLA₂ hydrolysable, cisplatin-containing liposome.

The expression level of the biomarker (e.g., COL5A2 (SEQ ID NO: 73 OR 211) in a sample from the cancer patient may also be compared to the expression level of the biomarker in a cell (e.g., a cancer cell) or tissue (e.g., a tumor tissue) known to be resistant to treatment with sPLA₂ hydrolysable, cisplatin-containing liposome (e.g., LiPlaCis). If the expression level of the biomarker in the sample from the cancer patient is substantially similar to the expression level of the biomarker in the cell or tissue known to be resistant to sPLA₂ hydrolysable, cisplatin-containing liposome (e.g., LiPlaCis), then the cancer patient is predicted to be non-responsive to treatment with the sPLA₂ hydrolysable, cisplatin-containing liposome. Alternatively, if the expression level of the biomarker in the sample from the cancer patient is substantially dissimilar to the expression level of the biomarker in the cell or tissue known to be sensitive to sPLA₂ hydrolysable, cisplatin-containing liposome (e.g., LiPlaCis), then the cancer patient is predicted to be responsive to treatment with the sPLA₂ hydrolysable, cisplatin-containing liposome.

The responsiveness of a cancer patient (e.g., a patient having cancer that is resistant to one or more cancer therapies other than sPLA₂ hydrolysable, cisplatin-containing liposome) to sPLA₂ hydrolysable, cisplatin-containing liposome (e.g., LiPlaCis) can also be predicted by comparing the expression level of a biomarker (e.g., COL5A2 (SEQ ID NO: 73 OR 211) to the expression level of the biomarker in one or more cells or tissues (e.g., from a cancer patient population) known to be sensitive to treatment with sPLA₂ hydrolysable, cisplatin-containing liposome and one or more cells or tissues (e.g., from a cancer patient population) known to be resistant to treatment with sPLA₂ hydrolysable, cisplatin-containing liposome. In particular, the patient may be responsive to treatment with sPLA₂ hydrolysable, cisplatin-containing liposome (e.g., LiPlaCis) if the expression level of the biomarker is more similar to the expression level of the biomarker in a cell or tissue known to be sensitive to treatment with the sPLA₂ hydrolysable, cisplatin-containing liposome than to a cell or tissue known to be resistant to treatment with the same. Alternatively, the patient may be non-responsive to treatment with sPLA₂ hydrolysable, cisplatin-containing liposome (e.g., LiPlaCis) if the expression level of the biomarker is more similar to the expression level of the biomarker in a cell or tissue known to be resistant to treatment with the sPLA₂ hydrolysable, cisplatin-containing liposome than to a cell or tissue known to be sensitive to treatment with the same.

Additionally, one or more biomarkers of sensitivity (e.g., one or more of COL5A2 (SEQ ID NO 73 or 211), ITGA4 (SEQ ID NO: 1), MSN (SEQ ID NO: 2), FAM46A (SEQ ID NO: 3 or 280), ITGB2 (SEQ ID NO: 4), DOCK2 (SEQ ID NO: 5 or 223), EVL (SEQ ID NO: 6), SACS (SEQ ID NO: 7), PTPRCAP (SEQ ID NO: 8), EBI2 (SEQ ID NO: 9), PTPRC (SEQ ID NO: 10, 18, 25, or 243), ANP32E (SEQ ID NO: 11), SFPQ (SEQ ID NO: 12, 38 or 272), C1QR1 (SEQ ID NO: 13), FNBP1 (SEQ ID NO: 14 or 28), CBFB (SEQ ID NO: 15)) and one or more biomarkers of resistance (e.g., one or more of SFN (SEQ ID NO: 96 or 324), LISCH7 (SEQ ID NO: 97), EPB41L4B (SEQ ID NO: 98), MST1R (SEQ ID NO: 99), ITGB4 (SEQ ID NO: 100), DBNDD2 (SEQ ID NO: 102 or 365), TACSTD1 (SEQ ID NO: 104), MISP (SEQ ID NO: 105), KRT8 (SEQ ID NO: 106), JUP (SEQ ID NO: 107 or 400), KRT18 (SEQ ID NO: 108 or 306), FA2H (SEQ ID NO: 109), MGAT4B (SEQ ID NO: 110), DSG2 (SEQ ID NO:111 or 312), LRP5 (SEQ ID NO: 112)) may be used in combination to determine the responsiveness of a cancer patient (e.g., a patient having cancer that is resistant to one or more cancer therapies other than sPLA₂ hydrolysable, cisplatin-containing liposome) to treatment with sPLA₂ hydrolysable, cisplatin-containing liposome (e.g., LiPlaCis). For example, the predicted responsiveness of a cancer patient may be determined from, e.g., the difference score, which may be defined as the difference between the mean of the expression level of the one or more biomarkers of sensitivity of Tables 2 and 4 and the mean of the expression level of the one or more biomarkers of resistance of Tables 3 and 5.

The difference score of the cancer patient can then be compared to the difference score based on the expression level of the biomarkers in a cell (e.g., a cancer cell) or tissue (e.g., a tumor tissue) known to be sensitive or resistant to treatment with sPLA₂ hydrolysable, cisplatin-containing liposome. In particular, the patient may be responsive to treatment with sPLA₂ hydrolysable, cisplatin-containing liposome (e.g., LiPlaCis) if the difference score is substantially similar to the expression level of the biomarkers in a cell or tissue known to be sensitive to treatment with the sPLA₂ hydrolysable, cisplatin-containing liposome. Alternatively, the patient may be non-responsive to treatment with sPLA₂ hydrolysable, cisplatin-containing liposome (e.g., LiPlaCis) if the difference score is substantially similar to the expression level of the biomarkers in a cell or tissue known to be resistant to treatment with the sPLA₂ hydrolysable, cisplatin-containing liposome. Additionally, the patient may be responsive to treatment with sPLA₂ hydrolysable, cisplatin-containing liposome (e.g., LiPlaCis) if the difference score is substantially similar to the expression level of the biomarkers in a cell or tissue known to be sensitive to treatment with the sPLA₂ hydrolysable, cisplatin-containing liposome than a cell or tissue known to be resistant to treatment with the same. Alternatively, the patient may be non-responsive to treatment with sPLA₂ hydrolysable, cisplatin-containing liposome (e.g., LiPlaCis) if the difference score is substantially similar to the expression level of the biomarkers in a cell or tissue known to be resistant to treatment with the sPLA₂ hydrolysable, cisplatin-containing liposome than a cell or tissue known to be sensitive to treatment with the same.

One or more biomarkers of sensitivity and/or resistance, identified as described herein, can be used to predict responsiveness to sPLA₂ hydrolysable, cisplatin-containing liposome (e.g., LiPlaCis) by measuring the expression level of the biomarkers in a biological sample obtained from the cancer patient. A single biomarker (e.g., any of the biomarkers of Tables 2-5, such as COL5A2 (SEQ ID NO: 73 OR 211) may be used to determine the responsiveness of a cancer patient (e.g., a patient having cancer that is resistant to one or more cancer therapies other than sPLA₂ hydrolysable, cisplatin-containing liposome) to sPLA₂ hydrolysable, cisplatin-containing liposome (e.g., LiPlaCis). After determining the expression level of the biomarker(s) in a sample (e.g., a tumor sample) from the cancer patient, the expression level of the biomarker(s) in the sample may be compared to the expression level of the biomarker(s) in a cell (e.g., a cancer cell) or tissue (e.g., a tumor tissue) known to be sensitive to treatment with sPLA₂ hydrolysable, cisplatin-containing liposome. If the expression level of the biomarker(s) in the sample from the cancer patient corresponds to (e.g., is identical to or has the same trend of expression level as) the expression level of the biomarker(s) in the cell or tissue known to be sensitive to sPLA₂ hydrolysable, cisplatin-containing liposome, then the cancer patient is predicted to be responsive to treatment with the sPLA₂ hydrolysable, cisplatin-containing liposome. Alternatively, if the expression level of the biomarker(s) in the sample from the cancer patient is substantially dissimilar to the expression level of the biomarker(s) in the cell or tissue known to be sensitive to sPLA₂ hydrolysable, cisplatin-containing liposome, then the cancer patient is predicted to be non-responsive to treatment with the sPLA₂ hydrolysable, cisplatin-containing liposome.

The expression level of the biomarker (e.g., COL5A2 (SEQ ID NO: 73 OR 211) in a sample from the cancer patient may also be compared to the expression level of the biomarker in a cell (e.g., a cancer cell) or tissue (e.g., a tumor tissue) known to be resistant to treatment with sPLA₂ hydrolysable, cisplatin-containing liposome (e.g., LiPlaCis). If the expression level of the biomarker in the sample from the cancer patient corresponds to the expression level of the biomarker in the cell or tissue known to be resistant to sPLA₂ hydrolysable, cisplatin-containing liposome, then the cancer patient is predicted to be non-responsive to treatment with the sPLA₂ hydrolysable, cisplatin-containing liposome. Alternatively, if the expression level of the biomarker in the sample from the cancer patient is substantially dissimilar to the expression level of the biomarker in the cell or tissue known to be resistant to sPLA₂ hydrolysable, cisplatin-containing liposome, then the cancer patient is predicted to be responsive to treatment with the sPLA₂ hydrolysable, cisplatin-containing liposome.

The responsiveness of a cancer patient (e.g., a patient having cancer that is resistant to one or more cancer therapies other than sPLA₂ hydrolysable, cisplatin-containing liposome) to sPLA₂ hydrolysable, cisplatin-containing liposome (e.g., LiPlaCis) can also be predicted by comparing the expression level of a biomarker (e.g., COL5A2 (SEQ ID NO: 73 OR 211) to the expression level of the biomarker in one or more cells or tissues (e.g., from a cancer patient population) known to be sensitive to treatment with sPLA₂ hydrolysable, cisplatin-containing liposome and one or more cells or tissues (e.g., from a cancer patient population) known to be resistant to treatment with the same. In particular, the patient may be responsive to treatment with sPLA₂ hydrolysable, cisplatin-containing liposome (e.g., LiPlaCis) if the expression level of the biomarker(s) corresponds to the expression level of the biomarker(s) in a cell or tissue known to be sensitive to treatment with the sPLA₂ hydrolysable, cisplatin-containing liposome relative to the expression level of the biomarkers in a cell or tissue known to be resistant to treatment with the same. Alternatively, the patient may be non-responsive to treatment with sPLA₂ hydrolysable, cisplatin-containing liposome (e.g., LiPlaCis) if the expression level of the biomarker(s) corresponds to the expression level of the biomarker(s) in a cell or tissue known to be resistant to treatment with the sPLA₂ hydrolysable, cisplatin-containing liposome relative to the expression level of the biomarkers in a cell or tissue known to be resistant to treatment with the same.

Additionally, one or more biomarkers of sensitivity (e.g., one or more of COL5A2 (SEQ ID NO 73 or 211), ITGA4 (SEQ ID NO: 1), MSN (SEQ ID NO: 2), FAM46A (SEQ ID NO: 3 or 280), ITGB2 (SEQ ID NO: 4), DOCK2 (SEQ ID NO: 5 or 223), EVL (SEQ ID NO: 6), SACS (SEQ ID NO: 7), PTPRCAP (SEQ ID NO: 8), EBI2 (SEQ ID NO: 9), PTPRC (SEQ ID NO: 10, 18, 25, or 243), ANP32E (SEQ ID NO: 11), SFPQ (SEQ ID NO: 12, 38 or 272), C1QR1 (SEQ ID NO: 13), FNBP1 (SEQ ID NO: 14 or 28), CBFB (SEQ ID NO: 15)) and one or more biomarkers of resistance (e.g., one or more of SFN (SEQ ID NO: 96 or 324), LISCH7 (SEQ ID NO: 97), EPB41L4B (SEQ ID NO: 98), MST1R (SEQ ID NO: 99), ITGB4 (SEQ ID NO: 100), DBNDD2 (SEQ ID NO: 102 or 365), TACSTD1 (SEQ ID NO: 104), MISP (SEQ ID NO: 105), KRT8 (SEQ ID NO: 106), JUP (SEQ ID NO: 107 or 400), KRT18 (SEQ ID NO: 108 or 306), FA2H (SEQ ID NO: 109), MGAT4B (SEQ ID NO: 110), DSG2 (SEQ ID NO:111 or 312), LRP5 (SEQ ID NO: 112)) may be used in combination to determine the responsiveness of a cancer patient (e.g., a patient having cancer that is resistant to one or more cancer therapies other than sPLA₂ hydrolysable, cisplatin-containing liposome) to treatment with sPLA₂ hydrolysable, cisplatin-containing liposome (e.g., LiPlaCis). For example, the predicted responsiveness of a cancer patient may be determined from, e.g., the difference score, which may be defined as the difference between the mean of the expression level of the one or more biomarkers of sensitivity of Tables 2 and 4 and the mean of the expression level of the one or more biomarkers of resistance of Tables 3 and 5.

The difference score of the cancer patient can then be compared to the difference score based on the expression level of the biomarkers in a cell (e.g., a cancer cell) or tissue (e.g., a tumor tissue) known to be sensitive or resistant to treatment with sPLA₂ hydrolysable, cisplatin-containing liposome. In particular, the patient may be responsive to treatment with sPLA₂ hydrolysable, cisplatin-containing liposome (e.g., LiPlaCis) if the difference score corresponds to the expression level of the biomarkers in a cell or tissue known to be sensitive to treatment with the sPLA₂ hydrolysable, cisplatin-containing liposome. Alternatively, the patient may be non-responsive to treatment with sPLA₂ hydrolysable, cisplatin-containing liposome (e.g., LiPlaCis) if the difference score corresponds to the expression level of the biomarkers in a cell or tissue known to be resistant to treatment with the sPLA₂ hydrolysable, cisplatin-containing liposome. Additionally, the patient may be responsive to treatment with sPLA₂ hydrolysable, cisplatin-containing liposome (e.g., LiPlaCis) if the difference score corresponds to the expression level of the biomarkers in a cell or tissue known to be sensitive to treatment with the sPLA₂ hydrolysable, cisplatin-containing liposome relative to the expression level of the biomarkers in a cell or tissue known to be resistant to treatment with the same. Alternatively, the patient may be non-responsive to treatment with sPLA₂ hydrolysable, cisplatin-containing liposome (e.g., LiPlaCis) if the difference score corresponds to the expression level of the biomarkers in a cell or tissue known to be resistant to treatment with the sPLA₂ hydrolysable, cisplatin-containing liposome relative to the expression level of the biomarkers in a cell or tissue known to be sensitive to treatment with the same.

Preferably, the cell or tissue known to be either sensitive or resistant to sPLA₂ hydrolysable, cisplatin-containing liposome (e.g., LiPlaCis) is of the same cancer type as the cancer patient with an unknown responsiveness to sPLA₂ hydrolysable, cisplatin-containing liposome. For example, the cancer patient and the cell or tissue known to be either sensitive or resistant to sPLA₂ hydrolysable, cisplatin-containing liposome (e.g., LiPlaCis) may both have a cancer type selected from a solid tumor or a hematological cancer, e.g., prostate cancer, ovarian cancer (e.g., ovarian adenocarcinoma or embryonal carcinoma), liver cancer (e.g., hepatocellular carcinoma (HCC) or hepatoma), myeloma (e.g., multiple myeloma), colorectal cancer (e.g., colon cancer and rectal cancer), leukemia (e.g., acute myeloid leukemia, acute lymphoid leukemia, chronic myeloid leukemia, chronic lymphocytic leukemia, acute myeloblastic leukemia, acute promyelocytic leukemia, acute myelomonocytic leukemia, acute monocytic leukemia, acute erythroleukemia, and chronic leukemia), myelodysplastic syndrome, lymphoma (e.g., diffuse large B-cell lymphoma, cutaneous T-cell lymphoma, peripheral T-cell lymphoma, Hodgkin's lymphoma, non-Hodgkin's lymphoma, Waldenstrom's macroglobulinemia, and lymphocytic lymphoma), cervical cancer, esophageal cancer, melanoma, glioma (e.g., oligodendroglioma), pancreatic cancer (e.g., adenosquamous carcinoma, signet ring cell carcinoma, hepatoid carcinoma, colloid carcinoma, islet cell carcinoma, and pancreatic neuroendocrine carcinoma), gastrointestinal stromal tumor, sarcoma (e.g., fibrosarcoma, myxosarcoma, liposarcoma, chondrosarcoma, osteogenic sarcoma, angiosarcoma, endotheliosarcoma, lymphangiosarcoma, lymphangioendotheliosarcoma, leiomyosarcoma, Ewing's sarcoma, and rhabdomyosarcoma), breast cancer (e.g., medullary carcinoma), ER-positive cancer, bladder cancer, head and neck cancer (e.g., squamous cell carcinoma of the head and neck), lung cancer (e.g., non-small cell lung carcinoma, large cell carcinoma, bronchogenic carcinoma, and papillary adenocarcinoma), metastatic cancer, oral cavity cancer, uterine cancer, testicular cancer (e.g., seminoma and embryonal carcinoma), skin cancer (e.g., squamous cell carcinoma and basal cell carcinoma), thyroid cancer (e.g., papillary carcinoma and medullary carcinoma), brain cancer (e.g., astrocytoma and craniopharyngioma), stomach cancer, intra-epithelial cancer, bone cancer, biliary tract cancer, eye cancer, larynx cancer, kidney cancer (e.g., renal cell carcinoma and Wilms tumor), gastric cancer, blastoma (e.g., nephroblastoma, medulloblastoma, hemangioblastoma, neuroblastoma, and retinoblastoma), polycythemia vera, chordoma, synovioma, mesothelioma, adenocarcinoma, sweat gland carcinoma, sebaceous gland carcinoma, cystadenocarcinoma, bile duct carcinoma, choriocarcinoma, epithelial carcinoma, ependymoma, pinealoma, acoustic neuroma, schwannoma, meningioma, pituitary adenoma, nerve sheath tumor, cancer of the small intestine, cancer of the endocrine system, cancer of the penis, cancer of the urethra, cutaneous or intraocular melanoma, a gynecologic tumor, solid tumors of childhood, and neoplasms of the central nervous system. In particular, the cancer of the patient and the cell or tissue with known resistance or sensitivity to LiPlaCis is, e.g., prostate cancer, ovarian cancer, hepatocellular carcinoma (HCC), multiple myeloma, breast cancer, acute myelogenous leukemia (AML), acute lympho-blastic leukemia (ALL), chronic lymphocytic leukemia (CLL), myelodysplastic syndrome (MDS), chronic myelogenous leukemia—chronic phase (CMLCP), diffuse large B-cell lymphoma (DLBCL), cutaneous T-cell lymphoma (CTCL), peripheral T-cell lymphoma (PTCL), Hodgkin's lymphoma, cervical cancer, renal cell carcinoma (RCC), esophageal cancer, melanoma, glioma, pancreatic cancer, gastrointestinal stromal tumors (GIST), sarcoma, estrogen receptor-positive (ERpos) breast cancer, non-small cell lung carcinoma (NSCLC), colon cancer, bladder cancer, or squamous cell carcinoma of the head and neck (SCCHN).

Machine learning techniques such as Neural Networks, Support Vector Machines, K Nearest Neighbor, and Nearest Centroids may also be employed to develop models that discriminate patients sensitive to treatment with sPLA₂ hydrolysable, cisplatin-containing liposome from those resistant to treatment with sPLA₂ hydrolysable, cisplatin-containing liposome using biomarker expression as model variables which assign each patient a classification as sensitive or resistant to treatment with the same. Machine learning techniques used to classify patients using various measurements are described in U.S. Pat. No. 5,822,715; U.S. Patent Application Publication Nos. 2003/0073083, 2005/0227266, 2005/0208512, 2005/0123945, 2003/0129629, and 2002/0006613; and in Vapnik V N. Statistical Learning Theory, John Wiley & Sons, New York, 1998; Hastie et al., 2001, The Elements of Statistical Learning: Data Mining, Inference, and Prediction, Springer, N.Y.; Agresti, 1996, An Introduction to Categorical Data Analysis, John Wiley & Sons, New York; V. Tresp et al., “Neural Network Modeling of Physiological Processes,” in Hanson S. J. et al. (Eds.), Computational Learning Theory and Natural Learning Systems 2, MIT Press, 1994, each of which are hereby incorporated by reference in their entirety.

### Biomarkers of Sensitivity and Resistance

The expression levels of one or more biomarkers of Tables 2-5 can be used to determine cancer patient responsiveness to treatment with sPLA₂ hydrolysable, cisplatin-containing liposome (e.g., LiPlaCis). Once determined to be responsive, the patient can be treated with sPLA₂ hydrolysable, cisplatin-containing liposome (e.g., LiPlaCis). In particular, the biomarker COL5A2 (SEQ ID NO 73 or 211) may be used to assess a cancer patient's (e.g., a patient with cancer that is resistant to one or more cancer therapies other than sPLA₂ hydrolysable, cisplatin-containing liposome) responsiveness to sPLA₂ hydrolysable, cisplatin-containing liposome. The expression level of the biomarker COL5A2 (SEQ ID NO 73 or 211) may be assessed using nucleic acid amplification methods (e.g., PCR) or a device (e.g., a microarray). As is described above, the expression level of COL5A2 (SEQ ID NO 73 or 211) in the patient sample may then be compared, e.g., to the expression level of COL5A2 (SEQ ID NO 73 or 211) in a cell (e.g., a cancer cell) or tissue (e.g., a tumor tissue) known to be sensitive or resistant to treatment with sPLA₂ hydrolysable, cisplatin-containing liposome and used to determine the cancer patient's responsiveness to the same. The biomarker COL5A2 (SEQ ID NO 73 or 211) may be used alone to predict cancer patient responsiveness to treatment with sPLA₂ hydrolysable, cisplatin-containing liposome or in combination with one or more additional biomarkers (e.g., one, two, three, four, five, ten, or all of the biomarkers shown in Tables 2-5), such as ITGA4 (SEQ ID NO: 1), MSN (SEQ ID NO: 2), FAM46A (SEQ ID NO: 3 or 280), ITGB2 (SEQ ID NO: 4), DOCK2 (SEQ ID NO: 5 or 223), EVL (SEQ ID NO: 6), SACS (SEQ ID NO: 7), PTPRCAP (SEQ ID NO: 8), EBI2 (SEQ ID NO: 9), PTPRC (SEQ ID NO: 10, 18, 25, or 243), ANP32E (SEQ ID NO: 11), SFPQ (SEQ ID NO: 12, 38 or 272), C1QR1 (SEQ ID NO: 13), FNBP1 (SEQ ID NO: 14 or 28), CBFB (SEQ ID NO: 15), SFN (SEQ ID NO: 96 or 324), LISCH7 (SEQ ID NO: 97), EPB41L4B (SEQ ID NO: 98), MST1R (SEQ ID NO: 99), ITGB4 (SEQ ID NO: 100), DBNDD2 (SEQ ID NO: 102 or 365), TACSTD1 (SEQ ID NO: 104), MISP (SEQ ID NO: 105), KRT8 (SEQ ID NO: 106), JUP (SEQ ID NO: 107 or 400), KRT18 (SEQ ID NO: 108 or 306), FA2H (SEQ ID NO: 109), MGAT4B (SEQ ID NO: 110), DSG2 (SEQ ID NO:111 or 312), LRP5 (SEQ ID NO: 112). The expression level of the biomarker(s) may be determined using, e.g., a microarray, PCR, or other techniques described herein, for example, using a nucleic acid probe sequence based on the target sequences shown in Tables 2-5.

The expression level of the biomarker ITGA4 (SEQ ID NO: 1) may be assessed using nucleic acid amplification methods (e.g., PCR) or a device (e.g., a microarray). As is described above, the expression level of ITGA4 (SEQ ID NO: 1) in the patient sample may then be compared, e.g., to the expression level of ITGA4 (SEQ ID NO: 1) in a cell (e.g., a cancer cell) or tissue (e.g., a tumor tissue) known to be sensitive or resistant to treatment with sPLA₂ hydrolysable, cisplatin-containing liposome and used to determine the cancer patient's responsiveness to the same. The biomarker ITGA4 (SEQ ID NO: 1) may be used alone to predict cancer patient responsiveness to treatment with sPLA₂ hydrolysable, cisplatin-containing liposome or in combination with one or more additional biomarkers (e.g., one, two, three, four, five, ten, or all of the biomarkers shown in Tables 2-5), such as COL5A2 (SEQ ID NO 73 or 211), MSN (SEQ ID NO: 2), FAM46A (SEQ ID NO: 3 or 280), ITGB2 (SEQ ID NO: 4), DOCK2 (SEQ ID NO: 5 or 223), EVL (SEQ ID NO: 6), SACS (SEQ ID NO: 7), PTPRCAP (SEQ ID NO: 8), EBI2 (SEQ ID NO: 9), PTPRC (SEQ ID NO: 10, 18, 25, or 243), ANP32E (SEQ ID NO: 11), SFPQ (SEQ ID NO: 12, 38 or 272), C1QR1 (SEQ ID NO: 13), FNBP1 (SEQ ID NO: 14 or 28), CBFB (SEQ ID NO: 15), SFN (SEQ ID NO: 96 or 324), LISCH7 (SEQ ID NO: 97), EPB41L4B (SEQ ID NO: 98), MST1R (SEQ ID NO: 99), ITGB4 (SEQ ID NO: 100), DBNDD2 (SEQ ID NO: 102 or 365), TACSTD1 (SEQ ID NO: 104), MISP (SEQ ID NO: 105), KRT8 (SEQ ID NO: 106), JUP (SEQ ID NO: 107 or 400), KRT18 (SEQ ID NO: 108 or 306), FA2H (SEQ ID NO: 109), MGAT4B (SEQ ID NO: 110), DSG2 (SEQ ID NO:111 or 312), LRP5 (SEQ ID NO: 112). The expression level of the biomarker(s) may be determined using, e.g., a microarray, PCR, or other techniques described herein, for example, using a nucleic acid probe sequence based on the target sequences shown in Tables 2-5.

The biomarker MSN (SEQ ID NO: 2) may be used to assess a cancer patient's (e.g., a patient having cancer that is resistant to one or more cancer therapies other than sPLA₂ hydrolysable, cisplatin-containing liposome) responsiveness to sPLA₂ hydrolysable, cisplatin-containing liposome (e.g., LiPlaCis). The expression level of the biomarker MSN (SEQ ID NO: 2) may be assessed using nucleic acid amplification methods (e.g., PCR) or a device (e.g., a microarray). As is described above, the expression level of MSN (SEQ ID NO: 2) in the patient sample may then be compared, e.g., to the expression level of MSN (SEQ ID NO: 2) in a cell (e.g., a cancer cell) or tissue (e.g., a tumor tissue) known to be sensitive or resistant to treatment with sPLA₂ hydrolysable, cisplatin-containing liposome and used to determine the cancer patient's responsiveness to the same. The biomarker MSN (SEQ ID NO: 2) may be used alone to predict cancer patient responsiveness to treatment with sPLA₂ hydrolysable, cisplatin-containing liposome or in combination with one or more additional biomarkers (e.g., one, two, three, four, five, ten, or all of the biomarkers shown in Tables 2-5), such as COL5A2 (SEQ ID NO 73 or 211), ITGA4 (SEQ ID NO: 1), FAM46A (SEQ ID NO: 3 or 280), ITGB2 (SEQ ID NO: 4), DOCK2 (SEQ ID NO: 5 or 223), EVL (SEQ ID NO: 6), SACS (SEQ ID NO: 7), PTPRCAP (SEQ ID NO: 8), EBI2 (SEQ ID NO: 9), PTPRC (SEQ ID NO: 10, 18, 25, or 243), ANP32E (SEQ ID NO: 11), SFPQ (SEQ ID NO: 12, 38 or 272), C1QR1 (SEQ ID NO: 13), FNBP1 (SEQ ID NO: 14 or 28), CBFB (SEQ ID NO: 15), SFN (SEQ ID NO: 96 or 324), LISCH7 (SEQ ID NO: 97), EPB41L4B (SEQ ID NO: 98), MST1R (SEQ ID NO: 99), ITGB4 (SEQ ID NO: 100), DBNDD2 (SEQ ID NO: 102 or 365), TACSTD1 (SEQ ID NO: 104), MISP (SEQ ID NO: 105), KRT8 (SEQ ID NO: 106), JUP (SEQ ID NO: 107 or 400), KRT18 (SEQ ID NO: 108 or 306), FA2H (SEQ ID NO: 109), MGAT4B (SEQ ID NO: 110), DSG2 (SEQ ID NO:111 or 312), LRP5 (SEQ ID NO: 112). The expression level of the biomarker(s) may be determined using, e.g., a microarray, PCR, or other techniques described herein, for example, using a nucleic acid probe sequence based on the target sequences shown in Tables 2-5.

The biomarker FAM46A (SEQ ID NO: 3 or 280) may be used to assess a cancer patient's (e.g., a patient having cancer that is resistant to one or more cancer therapies other than sPLA₂ hydrolysable, cisplatin-containing liposome) responsiveness to sPLA₂ hydrolysable, cisplatin-containing liposome (e.g., LiPlaCis). The expression level of the biomarker FAM46A (SEQ ID NO: 3 or 280) may be assessed using nucleic acid amplification methods (e.g., PCR) or a device (e.g., a microarray). As is described above, the expression level of FAM46A (SEQ ID NO: 3 or 280) in the patient sample may then be compared, e.g., to the expression level of FAM46A (SEQ ID NO: 3 or 280) in a cell (e.g., a cancer cell) or tissue (e.g., a tumor tissue) known to be sensitive or resistant to treatment with sPLA₂ hydrolysable, cisplatin-containing liposome and used to determine the cancer patient's responsiveness to the same. The biomarker FAM46A (SEQ ID NO: 3 or 280) may be used alone to predict cancer patient responsiveness to treatment with sPLA₂ hydrolysable, cisplatin-containing liposome or in combination with one or more additional biomarkers (e.g., one, two, three, four, five, ten, or all of the biomarkers shown in Tables 2-5), such as COL5A2 (SEQ ID NO 73 or 211), ITGA4 (SEQ ID NO: 1), MSN (SEQ ID NO: 2), ITGB2 (SEQ ID NO: 4), DOCK2 (SEQ ID NO: 5 or 223), EVL (SEQ ID NO: 6), SACS (SEQ ID NO: 7), PTPRCAP (SEQ ID NO: 8), EBI2 (SEQ ID NO: 9), PTPRC (SEQ ID NO: 10, 18, 25, or 243), ANP32E (SEQ ID NO: 11), SFPQ (SEQ ID NO: 12, 38 or 272), C1QR1 (SEQ ID NO: 13), FNBP1 (SEQ ID NO: 14 or 28), CBFB (SEQ ID NO: 15), SFN (SEQ ID NO: 96 or 324), LISCH7 (SEQ ID NO: 97), EPB41L4B (SEQ ID NO: 98), MST1R (SEQ ID NO: 99), ITGB4 (SEQ ID NO: 100), DBNDD2 (SEQ ID NO: 102 or 365), TACSTD1 (SEQ ID NO: 104), MISP (SEQ ID NO: 105), KRT8 (SEQ ID NO: 106), JUP (SEQ ID NO: 107 or 400), KRT18 (SEQ ID NO: 108 or 306), FA2H (SEQ ID NO: 109), MGAT4B (SEQ ID NO: 110), DSG2 (SEQ ID NO:111 or 312), LRP5 (SEQ ID NO: 112). The expression level of the biomarker(s) may be determined using, e.g., a microarray, PCR, or other techniques described herein, for example, using a nucleic acid probe sequence based on the target sequences shown in Tables 2-5.

The biomarker ITGB2 (SEQ ID NO: 4) may be used to assess a cancer patient's (e.g., a patient having cancer that is resistant to one or more cancer therapies other than sPLA₂ hydrolysable, cisplatin-containing liposome) responsiveness to sPLA₂ hydrolysable, cisplatin-containing liposome (e.g., LiPlaCis). The expression level of the biomarker ITGB2 (SEQ ID NO: 4) may be assessed using nucleic acid amplification methods (e.g., PCR) or a device (e.g., a microarray). As is described above, the expression level of ITGB2 (SEQ ID NO: 4) in the patient sample may then be compared, e.g., to the expression level of ITGB2 (SEQ ID NO: 4) in a cell (e.g., a cancer cell) or tissue (e.g., a tumor tissue) known to be sensitive or resistant to treatment with sPLA₂ hydrolysable, cisplatin-containing liposome and used to determine the cancer patient's responsiveness to the same. The biomarker ITGB2 (SEQ ID NO: 4) may be used alone to predict cancer patient responsiveness to treatment with sPLA₂ hydrolysable, cisplatin-containing liposome or in combination with one or more additional biomarkers (e.g., one, two, three, four, five, ten, or all of the biomarkers shown in Tables 2-5), such as COL5A2 (SEQ ID NO 73 or 211), ITGA4 (SEQ ID NO: 1), MSN (SEQ ID NO: 2), FAM46A (SEQ ID NO: 3 or 280), DOCK2 (SEQ ID NO: 5 or 223), EVL (SEQ ID NO: 6), SACS (SEQ ID NO: 7), PTPRCAP (SEQ ID NO: 8), EBI2 (SEQ ID NO: 9), PTPRC (SEQ ID NO: 10, 18, 25, or 243), ANP32E (SEQ ID NO: 11), SFPQ (SEQ ID NO: 12, 38 or 272), C1QR1 (SEQ ID NO: 13), FNBP1 (SEQ ID NO: 14 or 28), CBFB (SEQ ID NO: 15), SFN (SEQ ID NO: 96 or 324), LISCH7 (SEQ ID NO: 97), EPB41L4B (SEQ ID NO: 98), MST1R (SEQ ID NO: 99), ITGB4 (SEQ ID NO: 100), DBNDD2 (SEQ ID NO: 102 or 365), TACSTD1 (SEQ ID NO: 104), MISP (SEQ ID NO: 105), KRT8 (SEQ ID NO: 106), JUP (SEQ ID NO: 107 or 400), KRT18 (SEQ ID NO: 108 or 306), FA2H (SEQ ID NO: 109), MGAT4B (SEQ ID NO: 110), DSG2 (SEQ ID NO:111 or 312), LRP5 (SEQ ID NO: 112). The expression level of the biomarker(s) may be determined using, e.g., a microarray, PCR, or other techniques described herein, for example, using a nucleic acid probe sequence based on the target sequences shown in Tables 2-5.

The biomarker DOCK2 (SEQ ID NO: 5 or 223) may be used to assess a cancer patient's (e.g., a patient having cancer that is resistant to one or more cancer therapies other than sPLA₂ hydrolysable, cisplatin-containing liposome) responsiveness to sPLA₂ hydrolysable, cisplatin-containing liposome (e.g., LiPlaCis). The expression level of the biomarker DOCK2 (SEQ ID NO: 5 or 223) may be assessed using nucleic acid amplification methods (e.g., PCR) or a device (e.g., a microarray). As is described above, the expression level of DOCK2 (SEQ ID NO: 5 or 223) in the patient sample may then be compared, e.g., to the expression level of DOCK2 (SEQ ID NO: 5 or 223) in a cell (e.g., a cancer cell) or tissue (e.g., a tumor tissue) known to be sensitive or resistant to treatment with sPLA₂ hydrolysable, cisplatin-containing liposome and used to determine the cancer patient's responsiveness to the same. The biomarker DOCK2 (SEQ ID NO: 5 or 223) may be used alone to predict cancer patient responsiveness to treatment with sPLA₂ hydrolysable, cisplatin-containing liposome or in combination with one or more additional biomarkers (e.g., one, two, three, four, five, ten, or all of the biomarkers shown in Tables 2-5), such as COL5A2 (SEQ ID NO 73 or 211), ITGA4 (SEQ ID NO: 1), MSN (SEQ ID NO: 2), FAM46A (SEQ ID NO: 3 or 280), ITGB2 (SEQ ID NO: 4), EVL (SEQ ID NO: 6), SACS (SEQ ID NO: 7), PTPRCAP (SEQ ID NO: 8), EBI2 (SEQ ID NO: 9), PTPRC (SEQ ID NO: 10, 18, 25, or 243), ANP32E (SEQ ID NO: 11), SFPQ (SEQ ID NO: 12, 38 or 272), C1QR1 (SEQ ID NO: 13), FNBP1 (SEQ ID NO: 14 or 28), CBFB (SEQ ID NO: 15), SFN (SEQ ID NO: 96 or 324), LISCH7 (SEQ ID NO: 97), EPB41L4B (SEQ ID NO: 98), MST1R (SEQ ID NO: 99), ITGB4 (SEQ ID NO: 100), DBNDD2 (SEQ ID NO: 102 or 365), TACSTD1 (SEQ ID NO: 104), MISP (SEQ ID NO: 105), KRT8 (SEQ ID NO: 106), JUP (SEQ ID NO: 107 or 400), KRT18 (SEQ ID NO: 108 or 306), FA2H (SEQ ID NO: 109), MGAT4B (SEQ ID NO: 110), DSG2 (SEQ ID NO:111 or 312), LRP5 (SEQ ID NO: 112). The expression level of the biomarker(s) may be determined using, e.g., a microarray, PCR, or other techniques described herein, for example, using a nucleic acid probe sequence based on the target sequences shown in Tables 2-5.

The biomarker EVL (SEQ ID NO: 6) may be used to assess a cancer patient's (e.g., a patient having cancer that is resistant to one or more cancer therapies other than sPLA₂ hydrolysable, cisplatin-containing liposome) responsiveness to sPLA₂ hydrolysable, cisplatin-containing liposome (e.g., LiPlaCis). The expression level of the biomarker EVL (SEQ ID NO: 6) may be assessed using nucleic acid amplification methods (e.g., PCR) or a device (e.g., a microarray). As is described above, the expression level of EVL (SEQ ID NO: 6) in the patient sample may then be compared, e.g., to the expression level of EVL (SEQ ID NO: 6) in a cell (e.g., a cancer cell) or tissue (e.g., a tumor tissue) known to be sensitive or resistant to treatment with sPLA₂ hydrolysable, cisplatin-containing liposome and used to determine the cancer patient's responsiveness to the same. The biomarker EVL (SEQ ID NO: 6) may be used alone to predict cancer patient responsiveness to treatment with sPLA₂ hydrolysable, cisplatin-containing liposome or in combination with one or more additional biomarkers (e.g., one, two, three, four, five, ten, or all of the biomarkers shown in Tables 2-5), such as COL5A2 (SEQ ID NO 73 or 211), ITGA4 (SEQ ID NO: 1), MSN (SEQ ID NO: 2), FAM46A (SEQ ID NO: 3 or 280), ITGB2 (SEQ ID NO: 4), DOCK2 (SEQ ID NO: 5 or 223), SACS (SEQ ID NO: 7), PTPRCAP (SEQ ID NO: 8), EBI2 (SEQ ID NO: 9), PTPRC (SEQ ID NO: 10, 18, 25, or 243), ANP32E (SEQ ID NO: 11), SFPQ (SEQ ID NO: 12, 38 or 272), C1QR1 (SEQ ID NO: 13), FNBP1 (SEQ ID NO: 14 or 28), CBFB (SEQ ID NO: 15), SFN (SEQ ID NO: 96 or 324), LISCH7 (SEQ ID NO: 97), EPB41L4B (SEQ ID NO: 98), MST1R (SEQ ID NO: 99), ITGB4 (SEQ ID NO: 100), DBNDD2 (SEQ ID NO: 102 or 365), TACSTD1 (SEQ ID NO: 104), MISP (SEQ ID NO: 105), KRT8 (SEQ ID NO: 106), JUP (SEQ ID NO: 107 or 400), KRT18 (SEQ ID NO: 108 or 306), FA2H (SEQ ID NO: 109), MGAT4B (SEQ ID NO: 110), DSG2 (SEQ ID NO:111 or 312), LRP5 (SEQ ID NO: 112). The expression level of the biomarker(s) may be determined using, e.g., a microarray, PCR, or other techniques described herein, for example, using a nucleic acid probe sequence based on the target sequences shown in Tables 2-5.

The biomarker SACS (SEQ ID NO: 7) may be used to assess a cancer patient's (e.g., a patient having cancer that is resistant to one or more cancer therapies other than sPLA₂ hydrolysable, cisplatin-containing liposome) responsiveness to sPLA₂ hydrolysable, cisplatin-containing liposome (e.g., LiPlaCis). The expression level of the biomarker SACS (SEQ ID NO: 7) may be assessed using nucleic acid amplification methods (e.g., PCR) or a device (e.g., a microarray). As is described above, the expression level of SACS (SEQ ID NO: 7) in the patient sample may then be compared, e.g., to the expression level of SACS (SEQ ID NO: 7) in a cell (e.g., a cancer cell) or tissue (e.g., a tumor tissue) known to be sensitive or resistant to treatment with sPLA₂ hydrolysable, cisplatin-containing liposome and used to determine the cancer patient's responsiveness to the same. The biomarker SACS (SEQ ID NO: 7) may be used alone to predict cancer patient responsiveness to treatment with sPLA₂ hydrolysable, cisplatin-containing liposome or in combination with one or more additional biomarkers (e.g., one, two, three, four, five, ten, or all of the biomarkers shown in Tables 2-5), such as COL5A2 (SEQ ID NO 73 or 211), ITGA4 (SEQ ID NO: 1), MSN (SEQ ID NO: 2), FAM46A (SEQ ID NO: 3 or 280), ITGB2 (SEQ ID NO: 4), DOCK2 (SEQ ID NO: 5 or 223), EVL (SEQ ID NO: 6), PTPRCAP (SEQ ID NO: 8), EBI2 (SEQ ID NO: 9), PTPRC (SEQ ID NO: 10, 18, 25, or 243), ANP32E (SEQ ID NO: 11), SFPQ (SEQ ID NO: 12, 38 or 272), C1QR1 (SEQ ID NO: 13), FNBP1 (SEQ ID NO: 14 or 28), CBFB (SEQ ID NO: 15), SFN (SEQ ID NO: 96 or 324), LISCH7 (SEQ ID NO: 97), EPB41L4B (SEQ ID NO: 98), MST1R (SEQ ID NO: 99), ITGB4 (SEQ ID NO: 100), DBNDD2 (SEQ ID NO: 102 or 365), TACSTD1 (SEQ ID NO: 104), MISP (SEQ ID NO: 105), KRT8 (SEQ ID NO: 106), JUP (SEQ ID NO: 107 or 400), KRT18 (SEQ ID NO: 108 or 306), FA2H (SEQ ID NO: 109), MGAT4B (SEQ ID NO: 110), DSG2 (SEQ ID NO:111 or 312), LRP5 (SEQ ID NO: 112). The expression level of the biomarker(s) may be determined using, e.g., a microarray, PCR, or other techniques described herein, for example, using a nucleic acid probe sequence based on the target sequences shown in Tables 2-5.

The expression levels of one or more biomarkers of Tables 2-5 can be used to determine cancer patient responsiveness to treatment with sPLA₂ hydrolysable, cisplatin-containing liposome. Once determined to be responsive, the patient can be treated with sPLA₂ hydrolysable, cisplatin-containing liposome (e.g., LiPlaCis). In particular, the biomarker PTPRCAP (SEQ ID NO: 8) may be used to assess a cancer patient's (e.g., a patient having cancer that is resistant to one or more cancer therapies other than sPLA₂ hydrolysable, cisplatin-containing liposome) responsiveness to sPLA₂ hydrolysable, cisplatin-containing liposome. The expression level of the biomarker PTPRCAP (SEQ ID NO: 8) may be assessed using nucleic acid amplification methods (e.g., PCR) or a device (e.g., a microarray). As is described above, the expression level of PTPRCAP (SEQ ID NO: 8) in the patient sample may then be compared, e.g., to the expression level of PTPRCAP (SEQ ID NO: 8) in a cell (e.g., a cancer cell) or tissue (e.g., a tumor tissue) known to be sensitive or resistant to treatment with sPLA₂ hydrolysable, cisplatin-containing liposome and used to determine the cancer patient's responsiveness to the same. The biomarker PTPRCAP (SEQ ID NO: 8) may be used alone to predict cancer patient responsiveness to treatment with sPLA₂ hydrolysable, cisplatin-containing liposome or in combination with one or more additional biomarkers (e.g., one, two, three, four, five, ten, or all of the biomarkers shown in Tables 2-5), such as COL5A2 (SEQ ID NO 73 or 211), ITGA4 (SEQ ID NO: 1), MSN (SEQ ID NO: 2), FAM46A (SEQ ID NO: 3 or 280), ITGB2 (SEQ ID NO: 4), DOCK2 (SEQ ID NO: 5 or 223), EVL (SEQ ID NO: 6), SACS (SEQ ID NO: 7), EBI2 (SEQ ID NO: 9), PTPRC (SEQ ID NO: 10, 18, 25, or 243), ANP32E (SEQ ID NO: 11), SFPQ (SEQ ID NO: 12, 38 or 272), C1QR1 (SEQ ID NO: 13), FNBP1 (SEQ ID NO: 14 or 28), CBFB (SEQ ID NO: 15), SFN (SEQ ID NO: 96 or 324), LISCH7 (SEQ ID NO: 97), EPB41L4B (SEQ ID NO: 98), MST1R (SEQ ID NO: 99), ITGB4 (SEQ ID NO: 100), DBNDD2 (SEQ ID NO: 102 or 365), TACSTD1 (SEQ ID NO: 104), MISP (SEQ ID NO: 105), KRT8 (SEQ ID NO: 106), JUP (SEQ ID NO: 107 or 400), KRT18 (SEQ ID NO: 108 or 306), FA2H (SEQ ID NO: 109), MGAT4B (SEQ ID NO: 110), DSG2 (SEQ ID NO:111 or 312), LRP5 (SEQ ID NO: 112). The expression level of the biomarker(s) may be determined using, e.g., a microarray, PCR, or other techniques described herein, for example, using a nucleic acid probe sequence based on the target sequences shown in Tables 2-5.

The biomarker EBI2 (SEQ ID NO: 9) may be used to assess a cancer patient's (e.g., a patient having cancer that is resistant to one or more cancer therapies other than sPLA₂ hydrolysable, cisplatin-containing liposome) responsiveness to sPLA₂ hydrolysable, cisplatin-containing liposome (e.g., LiPlaCis). The expression level of the biomarker EBI2 (SEQ ID NO: 9) may be assessed using nucleic acid amplification methods (e.g., PCR) or a device (e.g., a microarray). As is described above, the expression level of EBI2 (SEQ ID NO: 9) in the patient sample may then be compared, e.g., to the expression level of EBI2 (SEQ ID NO: 9) in a cell (e.g., a cancer cell) or tissue (e.g., a tumor tissue) known to be sensitive or resistant to treatment with sPLA₂ hydrolysable, cisplatin-containing liposome and used to determine the cancer patient's responsiveness to the same. The biomarker EBI2 (SEQ ID NO: 9) may be used alone to predict cancer patient responsiveness to treatment with sPLA₂ hydrolysable, cisplatin-containing liposome or in combination with one or more additional biomarkers (e.g., one, two, three, four, five, ten, or all of the biomarkers shown in Tables 2-5), such as COL5A2 (SEQ ID NO 73 or 211), ITGA4 (SEQ ID NO: 1), MSN (SEQ ID NO: 2), FAM46A (SEQ ID NO: 3 or 280), ITGB2 (SEQ ID NO: 4), DOCK2 (SEQ ID NO: 5 or 223), EVL (SEQ ID NO: 6), SACS (SEQ ID NO: 7), PTPRCAP (SEQ ID NO: 8), PTPRC (SEQ ID NO: 10, 18, 25, or 243), ANP32E (SEQ ID NO: 11), SFPQ (SEQ ID NO: 12, 38 or 272), C1QR1 (SEQ ID NO: 13), FNBP1 (SEQ ID NO: 14 or 28), CBFB (SEQ ID NO: 15), SFN (SEQ ID NO: 96 or 324), LISCH7 (SEQ ID NO: 97), EPB41L4B (SEQ ID NO: 98), MST1R (SEQ ID NO: 99), ITGB4 (SEQ ID NO: 100), DBNDD2 (SEQ ID NO: 102 or 365), TACSTD1 (SEQ ID NO: 104), MISP (SEQ ID NO: 105), KRT8 (SEQ ID NO: 106), JUP (SEQ ID NO: 107 or 400), KRT18 (SEQ ID NO: 108 or 306), FA2H (SEQ ID NO: 109), MGAT4B (SEQ ID NO: 110), DSG2 (SEQ ID NO:111 or 312), LRP5 (SEQ ID NO: 112). The expression level of the biomarker(s) may be determined using, e.g., a microarray, PCR, or other techniques described herein, for example, using a nucleic acid probe sequence based on the target sequences shown in Tables 2-5.

The biomarker PTPRC (SEQ ID NO: 10, 18, 25, or 243) may be used to assess a cancer patient's (e.g., a patient having cancer that is resistant to one or more cancer therapies other than sPLA₂ hydrolysable, cisplatin-containing liposome) responsiveness to sPLA₂ hydrolysable, cisplatin-containing liposome (e.g., LiPlaCis). The expression level of the biomarker PTPRC (SEQ ID NO: 10, 18, 25, or 243) may be assessed using nucleic acid amplification methods (e.g., PCR) or a device (e.g., a microarray). As is described above, the expression level of PTPRC (SEQ ID NO: 10, 18, 25, or 243) in the patient sample may then be compared, e.g., to the expression level of PTPRC (SEQ ID NO: 10, 18, 25, or 243) in a cell (e.g., a cancer cell) or tissue (e.g., a tumor tissue) known to be sensitive or resistant to treatment with sPLA₂ hydrolysable, cisplatin-containing liposome and used to determine the cancer patient's responsiveness to the same. The biomarker PTPRC (SEQ ID NO: 10, 18, 25, or 243) may be used alone to predict cancer patient responsiveness to treatment with sPLA₂ hydrolysable, cisplatin-containing liposome or in combination with one or more additional biomarkers (e.g., one, two, three, four, five, ten, or all of the biomarkers shown in Tables 2-5), such as COL5A2 (SEQ ID NO 73 or 211), ITGA4 (SEQ ID NO: 1), MSN (SEQ ID NO: 2), FAM46A (SEQ ID NO: 3 or 280), ITGB2 (SEQ ID NO: 4), DOCK2 (SEQ ID NO: 5 or 223), EVL (SEQ ID NO: 6), SACS (SEQ ID NO: 7), PTPRCAP (SEQ ID NO: 8), EBI2 (SEQ ID NO: 9), ANP32E (SEQ ID NO: 11), SFPQ (SEQ ID NO: 12, 38 or 272), C1QR1 (SEQ ID NO: 13), FNBP1 (SEQ ID NO: 14 or 28), CBFB (SEQ ID NO: 15), SFN (SEQ ID NO: 96 or 324), LISCH7 (SEQ ID NO: 97), EPB41L4B (SEQ ID NO: 98), MST1R (SEQ ID NO: 99), ITGB4 (SEQ ID NO: 100), DBNDD2 (SEQ ID NO: 102 or 365), TACSTD1 (SEQ ID NO: 104), MISP (SEQ ID NO: 105), KRT8 (SEQ ID NO: 106), JUP (SEQ ID NO: 107 or 400), KRT18 (SEQ ID NO: 108 or 306), FA2H (SEQ ID NO: 109), MGAT4B (SEQ ID NO: 110), DSG2 (SEQ ID NO:111 or 312), LRP5 (SEQ ID NO: 112). The expression level of the biomarker(s) may be determined using, e.g., a microarray, PCR, or other techniques described herein, for example, using a nucleic acid probe sequence based on the target sequences shown in Tables 2-5.

The biomarker ANP32E (SEQ ID NO: 11) may be used to assess a cancer patient's (e.g., a patient having cancer that is resistant to one or more cancer therapies other than sPLA₂ hydrolysable, cisplatin-containing liposome) responsiveness to sPLA₂ hydrolysable, cisplatin-containing liposome (e.g., LiPlaCis). The expression level of the biomarker ANP32E (SEQ ID NO: 11) may be assessed using nucleic acid amplification methods (e.g., PCR) or a device (e.g., a microarray). As is described above, the expression level of ANP32E (SEQ ID NO: 11) in the patient sample may then be compared, e.g., to the expression level of ANP32E (SEQ ID NO: 11) in a cell (e.g., a cancer cell) or tissue (e.g., a tumor tissue) known to be sensitive or resistant to treatment with sPLA₂ hydrolysable, cisplatin-containing liposome and used to determine the cancer patient's responsiveness to the same. The biomarker ANP32E (SEQ ID NO: 11) may be used alone to predict cancer patient responsiveness to treatment with LiPlaCis or in combination with one or more additional biomarkers (e.g., one, two, three, four, five, ten, or all of the biomarkers shown in Tables 2-5), such as COL5A2 (SEQ ID NO 73 or 211), ITGA4 (SEQ ID NO: 1), MSN (SEQ ID NO: 2), FAM46A (SEQ ID NO: 3 or 280), ITGB2 (SEQ ID NO: 4), DOCK2 (SEQ ID NO: 5 or 223), EVL (SEQ ID NO: 6), SACS (SEQ ID NO: 7), PTPRCAP (SEQ ID NO: 8), EBI2 (SEQ ID NO: 9), PTPRC (SEQ ID NO: 10, 18, 25, or 243), SFPQ (SEQ ID NO: 12, 38 or 272), C1QR1 (SEQ ID NO: 13), FNBP1 (SEQ ID NO: 14 or 28), CBFB (SEQ ID NO: 15), SFN (SEQ ID NO: 96 or 324), LISCH7 (SEQ ID NO: 97), EPB41L4B (SEQ ID NO: 98), MST1R (SEQ ID NO: 99), ITGB4 (SEQ ID NO: 100), DBNDD2 (SEQ ID NO: 102 or 365), TACSTD1 (SEQ ID NO: 104), MISP (SEQ ID NO: 105), KRT8 (SEQ ID NO: 106), JUP (SEQ ID NO: 107 or 400), KRT18 (SEQ ID NO: 108 or 306), FA2H (SEQ ID NO: 109), MGAT4B (SEQ ID NO: 110), DSG2 (SEQ ID NO:111 or 312), LRP5 (SEQ ID NO: 112). The expression level of the biomarker(s) may be determined using, e.g., a microarray, PCR, or other techniques described herein, for example, using a nucleic acid probe sequence based on the target sequences shown in Tables 2-5.

The biomarker SFPQ (SEQ ID NO: 12, 38 or 272) may be used to assess a cancer patient's (e.g., a patient having cancer that is resistant to one or more cancer therapies other than sPLA₂ hydrolysable, cisplatin-containing liposome) responsiveness to sPLA₂ hydrolysable, cisplatin-containing liposome (e.g., LiPlaCis). The expression level of the biomarker SFPQ (SEQ ID NO: 12, 38 or 272) may be assessed using nucleic acid amplification methods (e.g., PCR) or a device (e.g., a microarray). As is described above, the expression level of SFPQ (SEQ ID NO: 12, 38 or 272) in the patient sample may then be compared, e.g., to the expression level of SFPQ (SEQ ID NO: 12, 38 or 272) in a cell (e.g., a cancer cell) or tissue (e.g., a tumor tissue) known to be sensitive or resistant to treatment with sPLA₂ hydrolysable, cisplatin-containing liposome and used to determine the cancer patient's responsiveness to the same. The biomarker SFPQ (SEQ ID NO: 12, 38 or 272) may be used alone to predict cancer patient responsiveness to treatment with sPLA₂ hydrolysable, cisplatin-containing liposome or in combination with one or more additional biomarkers (e.g., one, two, three, four, five, ten, or all of the biomarkers shown in Tables 2-5), such as COL5A2 (SEQ ID NO 73 or 211), ITGA4 (SEQ ID NO: 1), MSN (SEQ ID NO: 2), FAM46A (SEQ ID NO: 3 or 280), ITGB2 (SEQ ID NO: 4), DOCK2 (SEQ ID NO: 5 or 223), EVL (SEQ ID NO: 6), SACS (SEQ ID NO: 7), PTPRCAP (SEQ ID NO: 8), EBI2 (SEQ ID NO: 9), PTPRC (SEQ ID NO: 10, 18, 25, or 243), ANP32E (SEQ ID NO: 11), C1QR1 (SEQ ID NO: 13), FNBP1 (SEQ ID NO: 14 or 28), CBFB (SEQ ID NO: 15), SFN (SEQ ID NO: 96 or 324), LISCH7 (SEQ ID NO: 97), EPB41L4B (SEQ ID NO: 98), MST1R (SEQ ID NO: 99), ITGB4 (SEQ ID NO: 100), DBNDD2 (SEQ ID NO: 102 or 365), TACSTD1 (SEQ ID NO: 104), MISP (SEQ ID NO: 105), KRT8 (SEQ ID NO: 106), JUP (SEQ ID NO: 107 or 400), KRT18 (SEQ ID NO: 108 or 306), FA2H (SEQ ID NO: 109), MGAT4B (SEQ ID NO: 110), DSG2 (SEQ ID NO: 11 or 312), LRP5 (SEQ ID NO: 112). The expression level of the biomarker(s) may be determined using, e.g., a microarray, PCR, or other techniques described herein, for example, using a nucleic acid probe sequence based on the target sequences shown in Tables 2-5.

The biomarker C1QR1 (SEQ ID NO: 13) may be used to assess a cancer patient's (e.g., a patient having cancer that is resistant to one or more cancer therapies other than sPLA₂ hydrolysable, cisplatin-containing liposome) responsiveness to sPLA₂ hydrolysable, cisplatin-containing liposome (e.g., LiPlaCis). The expression level of the biomarker C1QR1 (SEQ ID NO: 13) may be assessed using nucleic acid amplification methods (e.g., PCR) or a device (e.g., a microarray). As is described above, the expression level of C1QR1 (SEQ ID NO: 13) in the patient sample may then be compared, e.g., to the expression level of C1QR1 (SEQ ID NO: 13) in a cell (e.g., a cancer cell) or tissue (e.g., a tumor tissue) known to be sensitive or resistant to treatment with sPLA₂ hydrolysable, cisplatin-containing liposome and used to determine the cancer patient's responsiveness to the same. The biomarker C1QR1 (SEQ ID NO: 13) may be used alone to predict cancer patient responsiveness to treatment with sPLA₂ hydrolysable, cisplatin-containing liposome or in combination with one or more additional biomarkers (e.g., one, two, three, four, five, ten, or all of the biomarkers shown in Tables 2-5), such as COL5A2 (SEQ ID NO 73 or 211), ITGA4 (SEQ ID NO: 1), MSN (SEQ ID NO: 2), FAM46A (SEQ ID NO: 3 or 280), ITGB2 (SEQ ID NO: 4), DOCK2 (SEQ ID NO: 5 or 223), EVL (SEQ ID NO: 6), SACS (SEQ ID NO: 7), PTPRCAP (SEQ ID NO: 8), EBI2 (SEQ ID NO: 9), PTPRC (SEQ ID NO: 10, 18, 25, or 243), ANP32E (SEQ ID NO: 11), SFPQ (SEQ ID NO: 12, 38 or 272), FNBP1 (SEQ ID NO: 14 or 28), CBFB (SEQ ID NO: 15), SFN (SEQ ID NO: 96 or 324), LISCH7 (SEQ ID NO: 97), EPB41L4B (SEQ ID NO: 98), MST1R (SEQ ID NO: 99), ITGB4 (SEQ ID NO: 100), DBNDD2 (SEQ ID NO: 102 or 365), TACSTD1 (SEQ ID NO: 104), MISP (SEQ ID NO: 105), KRT8 (SEQ ID NO: 106), JUP (SEQ ID NO: 107 or 400), KRT18 (SEQ ID NO: 108 or 306), FA2H (SEQ ID NO: 109), MGAT4B (SEQ ID NO: 110), DSG2 (SEQ ID NO:111 or 312), LRP5 (SEQ ID NO: 112). The expression level of the biomarker(s) may be determined using, e.g., a microarray, PCR, or other techniques described herein, for example, using a nucleic acid probe sequence based on the target sequences shown in Tables 2-5.

The biomarker FNBP1 (SEQ ID NO: 14 or 28) may be used to assess a cancer patient's (e.g., a patient having cancer that is resistant to one or more cancer therapies other than sPLA₂ hydrolysable, cisplatin-containing liposome) responsiveness to sPLA₂ hydrolysable, cisplatin-containing liposome (e.g., LiPlaCis). The expression level of the biomarker FNBP1 (SEQ ID NO: 14 or 28) may be assessed using nucleic acid amplification methods (e.g., PCR) or a device (e.g., a microarray). As is described above, the expression level of FNBP1 (SEQ ID NO: 14 or 28) in the patient sample may then be compared, e.g., to the expression level of FNBP1 (SEQ ID NO: 14 or 28) in a cell (e.g., a cancer cell) or tissue (e.g., a tumor tissue) known to be sensitive or resistant to treatment with sPLA₂ hydrolysable, cisplatin-containing liposome and used to determine the cancer patient's responsiveness to the same. The biomarker FNBP1 (SEQ ID NO: 14 or 28) may be used alone to predict cancer patient responsiveness to treatment with sPLA₂ hydrolysable, cisplatin-containing liposome or in combination with one or more additional biomarkers (e.g., one, two, three, four, five, ten, or all of the biomarkers shown in Tables 2-5), such as COL5A2 (SEQ ID NO 73 or 211), ITGA4 (SEQ ID NO: 1), MSN (SEQ ID NO: 2), FAM46A (SEQ ID NO: 3 or 280), ITGB2 (SEQ ID NO: 4), DOCK2 (SEQ ID NO: 5 or 223), EVL (SEQ ID NO: 6), SACS (SEQ ID NO: 7), PTPRCAP (SEQ ID NO: 8), EBI2 (SEQ ID NO: 9), PTPRC (SEQ ID NO: 10, 18, 25, or 243), ANP32E (SEQ ID NO: 11), SFPQ (SEQ ID NO: 12, 38 or 272), C1QR1 (SEQ ID NO: 13), CBFB (SEQ ID NO: 15), SFN (SEQ ID NO: 96 or 324), LISCH7 (SEQ ID NO: 97), EPB41L4B (SEQ ID NO: 98), MST1R (SEQ ID NO: 99), ITGB4 (SEQ ID NO: 100), DBNDD2 (SEQ ID NO: 102 or 365), TACSTD1 (SEQ ID NO: 104), MISP (SEQ ID NO: 105), KRT8 (SEQ ID NO: 106), JUP (SEQ ID NO: 107 or 400), KRT18 (SEQ ID NO: 108 or 306), FA2H (SEQ ID NO: 109), MGAT4B (SEQ ID NO: 110), DSG2 (SEQ ID NO:111 or 312), LRP5 (SEQ ID NO: 112). The expression level of the biomarker(s) may be determined using, e.g., a microarray, PCR, or other techniques described herein, for example, using a nucleic acid probe sequence based on the target sequences shown in Tables 2-5.

The biomarker SFN (SEQ ID NO: 96 or 324) may be used to assess a cancer patient's (e.g., a patient having cancer that is resistant to one or more cancer therapies other than sPLA₂ hydrolysable, cisplatin-containing liposome) responsiveness to sPLA₂ hydrolysable, cisplatin-containing liposome (e.g., LiPlaCis). The expression level of the biomarker SFN (SEQ ID NO: 96 or 324) may be assessed using nucleic acid amplification methods (e.g., PCR) or a device (e.g., a microarray). As is described above, the expression level of SFN (SEQ ID NO: 96 or 324) in the patient sample may then be compared, e.g., to the expression level of SFN (SEQ ID NO: 96 or 324) in a cell (e.g., a cancer cell) or tissue (e.g., a tumor tissue) known to be sensitive or resistant to treatment with sPLA₂ hydrolysable, cisplatin-containing liposome and used to determine the cancer patient's responsiveness to the same. The biomarker SFN (SEQ ID NO: 96 or 324) may be used alone to predict cancer patient responsiveness to treatment with sPLA₂ hydrolysable, cisplatin-containing liposome or in combination with one or more additional biomarkers (e.g., one, two, three, four, five, ten, or all of the biomarkers shown in Tables 2-5), such as COL5A2 (SEQ ID NO 73 or 211), ITGA4 (SEQ ID NO: 1), MSN (SEQ ID NO: 2), FAM46A (SEQ ID NO: 3 or 280), ITGB2 (SEQ ID NO: 4), DOCK2 (SEQ ID NO: 5 or 223), EVL (SEQ ID NO: 6), SACS (SEQ ID NO: 7), PTPRCAP (SEQ ID NO: 8), EBI2 (SEQ ID NO: 9), PTPRC (SEQ ID NO: 10, 18, 25, or 243), ANP32E (SEQ ID NO: 11), SFPQ (SEQ ID NO: 12, 38 or 272), C1QR1 (SEQ ID NO: 13), FNBP1 (SEQ ID NO: 14 or 28), CBFB (SEQ ID NO: 15), LISCH7 (SEQ ID NO: 97), EPB41L4B (SEQ ID NO: 98), MST1R (SEQ ID NO: 99), ITGB4 (SEQ ID NO: 100), DBNDD2 (SEQ ID NO: 102 or 365), TACSTD1 (SEQ ID NO: 104), MISP (SEQ ID NO: 105), KRT8 (SEQ ID NO: 106), JUP (SEQ ID NO: 107 or 400), KRT18 (SEQ ID NO: 108 or 306), FA2H (SEQ ID NO: 109), MGAT4B (SEQ ID NO: 110), DSG2 (SEQ ID NO:111 or 312), LRP5 (SEQ ID NO: 112). The expression level of the biomarker(s) may be determined using, e.g., a microarray, PCR, or other techniques described herein, for example, using a nucleic acid probe sequence based on the target sequences shown in Tables 2-5.

The biomarker LISCH7 (SEQ ID NO: 97) may be used to assess a cancer patient's (e.g., a patient having cancer that is resistant to one or more cancer therapies other than sPLA₂ hydrolysable, cisplatin-containing liposome) responsiveness to sPLA₂ hydrolysable, cisplatin-containing liposome (e.g., LiPlaCis). The expression level of the biomarker LISCH7 (SEQ ID NO: 97) may be assessed using nucleic acid amplification methods (e.g., PCR) or a device (e.g., a microarray). As is described above, the expression level of LISCH7 (SEQ ID NO: 97) in the patient sample may then be compared, e.g., to the expression level of LISCH7 (SEQ ID NO: 97) in a cell (e.g., a cancer cell) or tissue (e.g., a tumor tissue) known to be sensitive or resistant to treatment with sPLA₂ hydrolysable, cisplatin-containing liposome and used to determine the cancer patient's responsiveness to the same. The biomarker LISCH7 (SEQ ID NO: 97) may be used alone to predict cancer patient responsiveness to treatment with sPLA₂ hydrolysable, cisplatin-containing liposome or in combination with one or more additional biomarkers (e.g., one, two, three, four, five, ten, or all of the biomarkers shown in Tables 2-5), such as COL5A2 (SEQ ID NO 73 or 211), ITGA4 (SEQ ID NO: 1), MSN (SEQ ID NO: 2), FAM46A (SEQ ID NO: 3 or 280), ITGB2 (SEQ ID NO: 4), DOCK2 (SEQ ID NO: 5 or 223), EVL (SEQ ID NO: 6), SACS (SEQ ID NO: 7), PTPRCAP (SEQ ID NO: 8), EBI2 (SEQ ID NO: 9), PTPRC (SEQ ID NO: 10, 18, 25, or 243), ANP32E (SEQ ID NO: 11), SFPQ (SEQ ID NO: 12, 38 or 272), C1QR1 (SEQ ID NO: 13), FNBP1 (SEQ ID NO: 14 or 28), CBFB (SEQ ID NO: 15), SFN (SEQ ID NO: 96 or 324), EPB41L4B (SEQ ID NO: 98), MST1R (SEQ ID NO: 99), ITGB4 (SEQ ID NO: 100), DBNDD2 (SEQ ID NO: 102 or 365), TACSTD1 (SEQ ID NO: 104), MISP (SEQ ID NO: 105), KRT8 (SEQ ID NO: 106), JUP (SEQ ID NO: 107 or 400), KRT18 (SEQ ID NO: 108 or 306), FA2H (SEQ ID NO: 109), MGAT4B (SEQ ID NO: 110), DSG2 (SEQ ID NO:111 or 312), LRP5 (SEQ ID NO: 112). The expression level of the biomarker(s) may be determined using, e.g., a microarray, PCR, or other techniques described herein, for example, using a nucleic acid probe sequence based on the target sequences shown in Tables 2-5.

The biomarker EPB41L4B (SEQ ID NO: 98) may be used to assess a cancer patient's (e.g., a patient having cancer that is resistant to one or more cancer therapies other than sPLA₂ hydrolysable, cisplatin-containing liposome) responsiveness to sPLA₂ hydrolysable, cisplatin-containing liposome (e.g., LiPlaCis). The expression level of the biomarker EPB41L4B (SEQ ID NO: 98) may be assessed using nucleic acid amplification methods (e.g., PCR) or a device (e.g., a microarray). As is described above, the expression level of EPB41L4B (SEQ ID NO: 98) in the patient sample may then be compared, e.g., to the expression level of EPB41L4B (SEQ ID NO: 98) in a cell (e.g., a cancer cell) or tissue (e.g., a tumor tissue) known to be sensitive or resistant to treatment with sPLA₂ hydrolysable, cisplatin-containing liposome and used to determine the cancer patient's responsiveness to the same. The biomarker EPB41L4B (SEQ ID NO: 98) may be used alone to predict cancer patient responsiveness to treatment with sPLA₂ hydrolysable, cisplatin-containing liposome or in combination with one or more additional biomarkers (e.g., one, two, three, four, five, ten, or all of the biomarkers shown in Tables 2-5), such as COL5A2 (SEQ ID NO 73 or 211), ITGA4 (SEQ ID NO: 1), MSN (SEQ ID NO: 2), FAM46A (SEQ ID NO: 3 or 280), ITGB2 (SEQ ID NO: 4), DOCK2 (SEQ ID NO: 5 or 223), EVL (SEQ ID NO: 6), SACS (SEQ ID NO: 7), PTPRCAP (SEQ ID NO: 8), EBI2 (SEQ ID NO: 9), PTPRC (SEQ ID NO: 10, 18, 25, or 243), ANP32E (SEQ ID NO: 11), SFPQ (SEQ ID NO: 12, 38 or 272), C1QR1 (SEQ ID NO: 13), FNBP1 (SEQ ID NO: 14 or 28), CBFB (SEQ ID NO: 15), SFN (SEQ ID NO: 96 or 324), LISCH7 (SEQ ID NO: 97), MST1R (SEQ ID NO: 99), ITGB4 (SEQ ID NO: 100), DBNDD2 (SEQ ID NO: 102 or 365), TACSTD1 (SEQ ID NO: 104), MISP (SEQ ID NO: 105), KRT8 (SEQ ID NO: 106), JUP (SEQ ID NO: 107 or 400), KRT18 (SEQ ID NO: 108 or 306), FA2H (SEQ ID NO: 109), MGAT4B (SEQ ID NO: 110), DSG2 (SEQ ID NO:111 or 312), LRP5 (SEQ ID NO: 112). The expression level of the biomarker(s) may be determined using, e.g., a microarray, PCR, or other techniques described herein, for example, using a nucleic acid probe sequence based on the target sequences shown in Tables 2-5.

The biomarker MST1R (SEQ ID NO: 99) may be used to assess a cancer patient's (e.g., a patient having cancer that is resistant to one or more cancer therapies other than sPLA₂ hydrolysable, cisplatin-containing liposome) responsiveness to sPLA₂ hydrolysable, cisplatin-containing liposome (e.g., LiPlaCis). The expression level of the biomarker MST1R (SEQ ID NO: 99) may be assessed using nucleic acid amplification methods (e.g., PCR) or a device (e.g., a microarray). As is described above, the expression level of MST1R (SEQ ID NO: 99) in the patient sample may then be compared, e.g., to the expression level of MST1R (SEQ ID NO: 99) in a cell (e.g., a cancer cell) or tissue (e.g., a tumor tissue) known to be sensitive or resistant to treatment with sPLA₂ hydrolysable, cisplatin-containing liposome and used to determine the cancer patient's responsiveness to the same. The biomarker MST1R (SEQ ID NO: 99) may be used alone to predict cancer patient responsiveness to treatment with sPLA₂ hydrolysable, cisplatin-containing liposome or in combination with one or more additional biomarkers (e.g., one, two, three, four, five, ten, or all of the biomarkers shown in Tables 2-5), such as COL5A2 (SEQ ID NO 73 or 211), ITGA4 (SEQ ID NO: 1), MSN (SEQ ID NO: 2), FAM46A (SEQ ID NO: 3 or 280), ITGB2 (SEQ ID NO: 4), DOCK2 (SEQ ID NO: 5 or 223), EVL (SEQ ID NO: 6), SACS (SEQ ID NO: 7), PTPRCAP (SEQ ID NO: 8), EBI2 (SEQ ID NO: 9), PTPRC (SEQ ID NO: 10, 18, 25, or 243), ANP32E (SEQ ID NO: 11), SFPQ (SEQ ID NO: 12, 38 or 272), C1QR1 (SEQ ID NO: 13), FNBP1 (SEQ ID NO: 14 or 28), CBFB (SEQ ID NO: 15), SFN (SEQ ID NO: 96 or 324), LISCH7 (SEQ ID NO: 97), EPB41L4B (SEQ ID NO: 98), ITGB4 (SEQ ID NO: 100), DBNDD2 (SEQ ID NO: 102 or 365), TACSTD1 (SEQ ID NO: 104), MISP (SEQ ID NO: 105), KRT8 (SEQ ID NO: 106), JUP (SEQ ID NO: 107 or 400), KRT18 (SEQ ID NO: 108 or 306), FA2H (SEQ ID NO: 109), MGAT4B (SEQ ID NO: 110), DSG2 (SEQ ID NO:111 or 312), LRP5 (SEQ ID NO: 112). The expression level of the biomarker(s) may be determined using, e.g., a microarray, PCR, or other techniques described herein, for example, using a nucleic acid probe sequence based on the target sequences shown in Tables 2-5.

The biomarker ITGB4 (SEQ ID NO: 100) may be used to assess a cancer patient's (e.g., a patient having cancer that is resistant to one or more cancer therapies other than sPLA₂ hydrolysable, cisplatin-containing liposome) responsiveness to sPLA₂ hydrolysable, cisplatin-containing liposome (e.g., LiPlaCis). The expression level of the biomarker ITGB4 (SEQ ID NO: 100) may be assessed using nucleic acid amplification methods (e.g., PCR) or a device (e.g., a microarray). As is described above, the expression level of ITGB4 (SEQ ID NO: 100) in the patient sample may then be compared, e.g., to the expression level of ITGB4 (SEQ ID NO: 100) in a cell (e.g., a cancer cell) or tissue (e.g., a tumor tissue) known to be sensitive or resistant to treatment with sPLA₂ hydrolysable, cisplatin-containing liposome and used to determine the cancer patient's responsiveness to the same. The biomarker ITGB4 (SEQ ID NO: 100) may be used alone to predict cancer patient responsiveness to treatment with sPLA₂ hydrolysable, cisplatin-containing liposome or in combination with one or more additional biomarkers (e.g., one, two, three, four, five, ten, or all of the biomarkers shown in Tables 2-5), such as COL5A2 (SEQ ID NO 73 or 211), ITGA4 (SEQ ID NO: 1), MSN (SEQ ID NO: 2), FAM46A (SEQ ID NO: 3 or 280), ITGB2 (SEQ ID NO: 4), DOCK2 (SEQ ID NO: 5 or 223), EVL (SEQ ID NO: 6), SACS (SEQ ID NO: 7), PTPRCAP (SEQ ID NO: 8), EBI2 (SEQ ID NO: 9), PTPRC (SEQ ID NO: 10, 18, 25, or 243), ANP32E (SEQ ID NO: 11), SFPQ (SEQ ID NO: 12, 38 or 272), C1QR1 (SEQ ID NO: 13), FNBP1 (SEQ ID NO: 14 or 28), CBFB (SEQ ID NO: 15), SFN (SEQ ID NO: 96 or 324), LISCH7 (SEQ ID NO: 97), EPB41L4B (SEQ ID NO: 98), MST1R (SEQ ID NO: 99), DBNDD2 (SEQ ID NO: 102 or 365), TACSTD1 (SEQ ID NO: 104), MISP (SEQ ID NO: 105), KRT8 (SEQ ID NO: 106), JUP (SEQ ID NO: 107 or 400), KRT18 (SEQ ID NO: 108 or 306), FA2H (SEQ ID NO: 109), MGAT4B (SEQ ID NO: 110), DSG2 (SEQ ID NO:111 or 312), LRP5 (SEQ ID NO: 112). The expression level of the biomarker(s) may be determined using, e.g., a microarray, PCR, or other techniques described herein, for example, using a nucleic acid probe sequence based on the target sequences shown in Tables 2-5.

The biomarker DBNDD2 (SEQ ID NO: 102 or 365) may be used to assess a cancer patient's (e.g., a patient having cancer that is resistant to one or more cancer therapies other than sPLA₂ hydrolysable, cisplatin-containing liposome) responsiveness to sPLA₂ hydrolysable, cisplatin-containing liposome (e.g., LiPlaCis). The expression level of the biomarker DBNDD2 (SEQ ID NO: 102 or 365) may be assessed using nucleic acid amplification methods (e.g., PCR) or a device (e.g., a microarray). As is described above, the expression level of DBNDD2 (SEQ ID NO: 102 or 365) in the patient sample may then be compared, e.g., to the expression level of DBNDD2 (SEQ ID NO: 102 or 365) in a cell (e.g., a cancer cell) or tissue (e.g., a tumor tissue) known to be sensitive or resistant to treatment with sPLA₂ hydrolysable, cisplatin-containing liposome and used to determine the cancer patient's responsiveness to the same. The biomarker DBNDD2 (SEQ ID NO: 102 or 365) may be used alone to predict cancer patient responsiveness to treatment with sPLA₂ hydrolysable, cisplatin-containing liposome or in combination with one or more additional biomarkers (e.g., one, two, three, four, five, ten, or all of the biomarkers shown in Tables 2-5), such as COL5A2 (SEQ ID NO 73 or 211), ITGA4 (SEQ ID NO: 1), MSN (SEQ ID NO: 2), FAM46A (SEQ ID NO: 3 or 280), ITGB2 (SEQ ID NO: 4), DOCK2 (SEQ ID NO: 5 or 223), EVL (SEQ ID NO: 6), SACS (SEQ ID NO: 7), PTPRCAP (SEQ ID NO: 8), EBI2 (SEQ ID NO: 9), PTPRC (SEQ ID NO: 10, 18, 25, or 243), ANP32E (SEQ ID NO: 11), SFPQ (SEQ ID NO: 12, 38 or 272), C1QR1 (SEQ ID NO: 13), FNBP1 (SEQ ID NO: 14 or 28), CBFB (SEQ ID NO: 15), SFN (SEQ ID NO: 96 or 324), LISCH7 (SEQ ID NO: 97), EPB41L4B (SEQ ID NO: 98), MST1R (SEQ ID NO: 99), ITGB4 (SEQ ID NO: 100), TACSTD1 (SEQ ID NO: 104), MISP (SEQ ID NO: 105), KRT8 (SEQ ID NO: 106), JUP (SEQ ID NO: 107 or 400), KRT18 (SEQ ID NO: 108 or 306), FA2H (SEQ ID NO: 109), MGAT4B (SEQ ID NO: 110), DSG2 (SEQ ID NO:111 or 312), LRP5 (SEQ ID NO: 112). The expression level of the biomarker(s) may be determined using, e.g., a microarray, PCR, or other techniques described herein, for example, using a nucleic acid probe sequence based on the target sequences shown in Tables 2-5.

The biomarker TACSTD1 (SEQ ID NO: 104) may be used to assess a cancer patient's (e.g., a patient having cancer that is resistant to one or more cancer therapies other than sPLA₂ hydrolysable, cisplatin-containing liposome) responsiveness to sPLA₂ hydrolysable, cisplatin-containing liposome (e.g., LiPlaCis). The expression level of the biomarker TACSTD1 (SEQ ID NO: 104) may be assessed using nucleic acid amplification methods (e.g., PCR) or a device (e.g., a microarray). As is described above, the expression level of TACSTD1 (SEQ ID NO: 104) in the patient sample may then be compared, e.g., to the expression level of TACSTD1 (SEQ ID NO: 104) in a cell (e.g., a cancer cell) or tissue (e.g., a tumor tissue) known to be sensitive or resistant to treatment with sPLA₂ hydrolysable, cisplatin-containing liposome and used to determine the cancer patient's responsiveness to the same. The biomarker TACSTD1 (SEQ ID NO: 104) may be used alone to predict cancer patient responsiveness to treatment with sPLA₂ hydrolysable, cisplatin-containing liposome or in combination with one or more additional biomarkers (e.g., one, two, three, four, five, ten, or all of the biomarkers shown in Tables 2-5), such as COL5A2 (SEQ ID NO 73 or 211), ITGA4 (SEQ ID NO: 1), MSN (SEQ ID NO: 2), FAM46A (SEQ ID NO: 3 or 280), ITGB2 (SEQ ID NO: 4), DOCK2 (SEQ ID NO: 5 or 223), EVL (SEQ ID NO: 6), SACS (SEQ ID NO: 7), PTPRCAP (SEQ ID NO: 8), EBI2 (SEQ ID NO: 9), PTPRC (SEQ ID NO: 10, 18, 25, or 243), ANP32E (SEQ ID NO: 11), SFPQ (SEQ ID NO: 12, 38 or 272), C1QR1 (SEQ ID NO: 13), FNBP1 (SEQ ID NO: 14 or 28), CBFB (SEQ ID NO: 15), SFN (SEQ ID NO: 96 or 324), LISCH7 (SEQ ID NO: 97), EPB41L4B (SEQ ID NO: 98), MST1R (SEQ ID NO: 99), ITGB4 (SEQ ID NO: 100), DBNDD2 (SEQ ID NO: 102 or 365), MISP (SEQ ID NO: 105), KRT8 (SEQ ID NO: 106), JUP (SEQ ID NO: 107 or 400), KRT18 (SEQ ID NO: 108 or 306), FA2H (SEQ ID NO: 109), MGAT4B (SEQ ID NO: 110), DSG2 (SEQ ID NO:111 or 312), LRP5 (SEQ ID NO: 112). The expression level of the biomarker(s) may be determined using, e.g., a microarray, PCR, or other techniques described herein, for example, using a nucleic acid probe sequence based on the target sequences shown in Tables 2-5.

The biomarker MISP (SEQ ID NO: 105) may be used to assess a cancer patient's (e.g., a patient having cancer that is resistant to one or more cancer therapies other than sPLA₂ hydrolysable, cisplatin-containing liposome) responsiveness to sPLA₂ hydrolysable, cisplatin-containing liposome (e.g., LiPlaCis). The expression level of the biomarker MISP (SEQ ID NO: 105) may be assessed using nucleic acid amplification methods (e.g., PCR) or a device (e.g., a microarray). As is described above, the expression level of MISP (SEQ ID NO: 105) in the patient sample may then be compared, e.g., to the expression level of MISP (SEQ ID NO: 105) in a cell (e.g., a cancer cell) or tissue (e.g., a tumor tissue) known to be sensitive or resistant to treatment with sPLA₂ hydrolysable, cisplatin-containing liposome and used to determine the cancer patient's responsiveness to the same. The biomarker MISP (SEQ ID NO: 105) may be used alone to predict cancer patient responsiveness to treatment with sPLA₂ hydrolysable, cisplatin-containing liposome or in combination with one or more additional biomarkers (e.g., one, two, three, four, five, ten, or all of the biomarkers shown in Tables 2-5), such as COL5A2 (SEQ ID NO 73 or 211), ITGA4 (SEQ ID NO: 1), MSN (SEQ ID NO: 2), FAM46A (SEQ ID NO: 3 or 280), ITGB2 (SEQ ID NO: 4), DOCK2 (SEQ ID NO: 5 or 223), EVL (SEQ ID NO: 6), SACS (SEQ ID NO: 7), PTPRCAP (SEQ ID NO: 8), EBI2 (SEQ ID NO: 9), PTPRC (SEQ ID NO: 10, 18, 25, or 243), ANP32E (SEQ ID NO: 11), SFPQ (SEQ ID NO: 12, 38 or 272), C1QR1 (SEQ ID NO: 13), FNBP1 (SEQ ID NO: 14 or 28), CBFB (SEQ ID NO: 15), SFN (SEQ ID NO: 96 or 324), LISCH7 (SEQ ID NO: 97), EPB41L4B (SEQ ID NO: 98), MST1R (SEQ ID NO: 99), ITGB4 (SEQ ID NO: 100), DBNDD2 (SEQ ID NO: 102 or 365), TACSTD1 (SEQ ID NO: 104), KRT8 (SEQ ID NO: 106), JUP (SEQ ID NO: 107 or 400), KRT18 (SEQ ID NO: 108 or 306), FA2H (SEQ ID NO: 109), MGAT4B (SEQ ID NO: 110), DSG2 (SEQ ID NO:111 or 312), LRP5 (SEQ ID NO: 112). The expression level of the biomarker(s) may be determined using, e.g., a microarray, PCR, or other techniques described herein, for example, using a nucleic acid probe sequence based on the target sequences shown in Tables 2-5.

The biomarker KRT8 (SEQ ID NO: 106) may be used to assess a cancer patient's (e.g., a patient having cancer that is resistant to one or more cancer therapies other than sPLA₂ hydrolysable, cisplatin-containing liposome) responsiveness to sPLA₂ hydrolysable, cisplatin-containing liposome (e.g., LiPlaCis) using nucleic acid amplification methods (e.g., PCR) or a device (e.g., a microarray). As is described above, the expression level of KRT8 (SEQ ID NO: 106) in the patient sample may then be compared, e.g., to the expression level of KRT8 (SEQ ID NO: 106) in a cell (e.g., a cancer cell) or tissue (e.g., a tumor tissue) known to be sensitive or resistant to treatment with sPLA₂ hydrolysable, cisplatin-containing liposome and used to determine the cancer patient's responsiveness to the same. The biomarker KRT8 (SEQ ID NO: 106) may be used alone to predict cancer patient responsiveness to treatment with sPLA₂ hydrolysable, cisplatin-containing liposome or in combination with one or more additional biomarkers (e.g., one, two, three, four, five, ten, or all of the biomarkers shown in Tables 2-5), such as COL5A2 (SEQ ID NO 73 or 211), ITGA4 (SEQ ID NO: 1), MSN (SEQ ID NO: 2), FAM46A (SEQ ID NO: 3 or 280), ITGB2 (SEQ ID NO: 4), DOCK2 (SEQ ID NO: 5 or 223), EVL (SEQ ID NO: 6), SACS (SEQ ID NO: 7), PTPRCAP (SEQ ID NO: 8), EBI2 (SEQ ID NO: 9), PTPRC (SEQ ID NO: 10, 18, 25, or 243), ANP32E (SEQ ID NO: 11), SFPQ (SEQ ID NO: 12, 38 or 272), C1QR1 (SEQ ID NO: 13), FNBP1 (SEQ ID NO: 14 or 28), CBFB (SEQ ID NO: 15), SFN (SEQ ID NO: 96 or 324), LISCH7 (SEQ ID NO: 97), EPB41L4B (SEQ ID NO: 98), MST1R (SEQ ID NO: 99), ITGB4 (SEQ ID NO: 100), DBNDD2 (SEQ ID NO: 102 or 365), TACSTD1 (SEQ ID NO: 104), MISP (SEQ ID NO: 105), JUP (SEQ ID NO: 107 or 400), KRT18 (SEQ ID NO: 108 or 306), FA2H (SEQ ID NO: 109), MGAT4B (SEQ ID NO: 110), DSG2 (SEQ ID NO:111 or 312), LRP5 (SEQ ID NO: 112). The expression level of the biomarker(s) may be determined using, e.g., a microarray, PCR, or other techniques described herein, for example, using a nucleic acid probe sequence based on the target sequences shown in Tables 2-5.

The biomarker JUP (SEQ ID NO: 107 or 400) may be used to assess a cancer patient's (e.g., a patient having cancer that is resistant to one or more cancer therapies other than sPLA₂ hydrolysable, cisplatin-containing liposome) responsiveness to sPLA₂ hydrolysable, cisplatin-containing liposome (e.g., LiPlaCis). The expression level of the biomarker JUP (SEQ ID NO: 107 or 400) may be assessed using nucleic acid amplification methods (e.g., PCR) or a device (e.g., a microarray). As is described above, the expression level of JUP (SEQ ID NO: 107 or 400) in the patient sample may then be compared, e.g., to the expression level of JUP (SEQ ID NO: 107 or 400) in a cell (e.g., a cancer cell) or tissue (e.g., a tumor tissue) known to be sensitive or resistant to treatment with sPLA₂ hydrolysable, cisplatin-containing liposome and used to determine the cancer patient's responsiveness to the same. The biomarker JUP (SEQ ID NO: 107 or 400) may be used alone to predict cancer patient responsiveness to treatment with sPLA₂ hydrolysable, cisplatin-containing liposome or in combination with one or more additional biomarkers (e.g., one, two, three, four, five, ten, or all of the biomarkers shown in Tables 2-5), such as COL5A2 (SEQ ID NO 73 or 211), ITGA4 (SEQ ID NO: 1), MSN (SEQ ID NO: 2), FAM46A (SEQ ID NO: 3 or 280), ITGB2 (SEQ ID NO: 4), DOCK2 (SEQ ID NO: 5 or 223), EVL (SEQ ID NO: 6), SACS (SEQ ID NO: 7), PTPRCAP (SEQ ID NO: 8), EBI2 (SEQ ID NO: 9), PTPRC (SEQ ID NO: 10, 18, 25, or 243), ANP32E (SEQ ID NO: 11), SFPQ (SEQ ID NO: 12, 38 or 272), C1QR1 (SEQ ID NO: 13), FNBP1 (SEQ ID NO: 14 or 28), CBFB (SEQ ID NO: 15), SFN (SEQ ID NO: 96 or 324), LISCH7 (SEQ ID NO: 97), EPB41L4B (SEQ ID NO: 98), MST1R (SEQ ID NO: 99), ITGB4 (SEQ ID NO: 100), DBNDD2 (SEQ ID NO: 102 or 365), TACSTD1 (SEQ ID NO: 104), MISP (SEQ ID NO: 105), KRT8 (SEQ ID NO: 106), KRT18 (SEQ ID NO: 108 or 306), FA2H (SEQ ID NO: 109), MGAT4B (SEQ ID NO: 110), DSG2 (SEQ ID NO:111 or 312), LRP5 (SEQ ID NO: 112). The expression level of the biomarker(s) may be determined using, e.g., a microarray, PCR, or other techniques described herein, for example, using a nucleic acid probe sequence based on the target sequences shown in Tables 2-5.

The biomarker KRT18 (SEQ ID NO: 108 or 306) may be used to assess a cancer patient's (e.g., a patient having cancer that is resistant to one or more cancer therapies other than sPLA₂ hydrolysable, cisplatin-containing liposome) responsiveness to sPLA₂ hydrolysable, cisplatin-containing liposome (e.g., LiPlaCis). The expression level of the biomarker KRT18 (SEQ ID NO: 108 or 306) may be assessed using nucleic acid amplification methods (e.g., PCR) or a device (e.g., a microarray). As is described above, the expression level of KRT18 (SEQ ID NO: 108 or 306) in the patient sample may then be compared, e.g., to the expression level of KRT18 (SEQ ID NO: 108 or 306) in a cell (e.g., a cancer cell) or tissue (e.g., a tumor tissue) known to be sensitive or resistant to treatment with sPLA₂ hydrolysable, cisplatin-containing liposome and used to determine the cancer patient's responsiveness to the same. The biomarker KRT18 (SEQ ID NO: 108 or 306) may be used alone to predict cancer patient responsiveness to treatment with sPLA₂ hydrolysable, cisplatin-containing liposome or in combination with one or more additional biomarkers (e.g., one, two, three, four, five, ten, or all of the biomarkers shown in Tables 2-5), such as COL5A2 (SEQ ID NO 73 or 211), ITGA4 (SEQ ID NO: 1), MSN (SEQ ID NO: 2), FAM46A (SEQ ID NO: 3 or 280), ITGB2 (SEQ ID NO: 4), DOCK2 (SEQ ID NO: 5 or 223), EVL (SEQ ID NO: 6), SACS (SEQ ID NO: 7), PTPRCAP (SEQ ID NO: 8), EBI2 (SEQ ID NO: 9), PTPRC (SEQ ID NO: 10, 18, 25, or 243), ANP32E (SEQ ID NO: 11), SFPQ (SEQ ID NO: 12, 38 or 272), C1QR1 (SEQ ID NO: 13), FNBP1 (SEQ ID NO: 14 or 28), CBFB (SEQ ID NO: 15), SFN (SEQ ID NO: 96 or 324), LISCH7 (SEQ ID NO: 97), EPB41L4B (SEQ ID NO: 98), MST1R (SEQ ID NO: 99), ITGB4 (SEQ ID NO: 100), DBNDD2 (SEQ ID NO: 102 or 365), TACSTD1 (SEQ ID NO: 104), MISP (SEQ ID NO: 105), KRT8 (SEQ ID NO: 106), JUP (SEQ ID NO: 107 or 400), FA2H (SEQ ID NO: 109), MGAT4B (SEQ ID NO: 110), DSG2 (SEQ ID NO: 11 or 312), LRP5 (SEQ ID NO: 112). The expression level of the biomarker(s) may be determined using, e.g., a microarray, PCR, or other techniques described herein, for example, using a nucleic acid probe sequence based on the target sequences shown in Tables 2-5.

The biomarker FA2H (SEQ ID NO: 109) may be used to assess a cancer patient's (e.g., a patient having cancer that is resistant to one or more cancer therapies other than sPLA₂ hydrolysable, cisplatin-containing liposome) responsiveness to sPLA₂ hydrolysable, cisplatin-containing liposome (e.g., LiPlaCis). The expression level of the biomarker FA2H (SEQ ID NO: 109) may be assessed using nucleic acid amplification methods (e.g., PCR) or a device (e.g., a microarray). As is described above, the expression level of FA2H (SEQ ID NO: 109) in the patient sample may then be compared, e.g., to the expression level of FA2H (SEQ ID NO: 109) in a cell (e.g., a cancer cell) or tissue (e.g., a tumor tissue) known to be sensitive or resistant to treatment with sPLA₂ hydrolysable, cisplatin-containing liposome s and used to determine the cancer patient's responsiveness to the same. The biomarker FA2H (SEQ ID NO: 109) may be used alone to predict cancer patient responsiveness to treatment with sPLA₂ hydrolysable, cisplatin-containing liposome or in combination with one or more additional biomarkers (e.g., one, two, three, four, five, ten, or all of the biomarkers shown in Tables 2-5), such as COL5A2 (SEQ ID NO 73 or 211), ITGA4 (SEQ ID NO: 1), MSN (SEQ ID NO: 2), FAM46A (SEQ ID NO: 3 or 280), ITGB2 (SEQ ID NO: 4), DOCK2 (SEQ ID NO: 5 or 223), EVL (SEQ ID NO: 6), SACS (SEQ ID NO: 7), PTPRCAP (SEQ ID NO: 8), EBI2 (SEQ ID NO: 9), PTPRC (SEQ ID NO: 10, 18, 25, or 243), ANP32E (SEQ ID NO: 11), SFPQ (SEQ ID NO: 12, 38 or 272), C1QR1 (SEQ ID NO: 13), FNBP1 (SEQ ID NO: 14 or 28), CBFB (SEQ ID NO: 15), SFN (SEQ ID NO: 96 or 324), LISCH7 (SEQ ID NO: 97), EPB41L4B (SEQ ID NO: 98), MST1R (SEQ ID NO: 99), ITGB4 (SEQ ID NO: 100), DBNDD2 (SEQ ID NO: 102 or 365), TACSTD1 (SEQ ID NO: 104), MISP (SEQ ID NO: 105), KRT8 (SEQ ID NO: 106), JUP (SEQ ID NO: 107 or 400), KRT18 (SEQ ID NO: 108 or 306), MGAT4B (SEQ ID NO: 110), DSG2 (SEQ ID NO:111 or 312), LRP5 (SEQ ID NO: 112). The expression level of the biomarker(s) may be determined using, e.g., a microarray, PCR, or other techniques described herein, for example, using a nucleic acid probe sequence based on the target sequences shown in Tables 2-5.

The biomarker MGAT4B (SEQ ID NO: 110) may be used to assess a cancer patient's (e.g., a patient having cancer that is resistant to one or more cancer therapies other than sPLA₂ hydrolysable, cisplatin-containing liposome) responsiveness to sPLA₂ hydrolysable, cisplatin-containing liposome (e.g., LiPlaCis). The expression level of the biomarker MGAT4B (SEQ ID NO: 110) may be assessed using nucleic acid amplification methods (e.g., PCR) or a device (e.g., a microarray). As is described above, the expression level of MGAT4B (SEQ ID NO: 110) in the patient sample may then be compared, e.g., to the expression level of MGAT4B (SEQ ID NO: 110) in a cell (e.g., a cancer cell) or tissue (e.g., a tumor tissue) known to be sensitive or resistant to treatment with sPLA₂ hydrolysable, cisplatin-containing liposome and used to determine the cancer patient's responsiveness to the same. The biomarker MGAT4B (SEQ ID NO: 110) may be used alone to predict cancer patient responsiveness to treatment with sPLA₂ hydrolysable, cisplatin-containing liposome or in combination with one or more additional biomarkers (e.g., one, two, three, four, five, ten, or all of the biomarkers shown in Tables 2-5), such as COL5A2 (SEQ ID NO 73 or 211), ITGA4 (SEQ ID NO: 1), MSN (SEQ ID NO: 2), FAM46A (SEQ ID NO: 3 or 280), ITGB2 (SEQ ID NO: 4), DOCK2 (SEQ ID NO: 5 or 223), EVL (SEQ ID NO: 6), SACS (SEQ ID NO: 7), PTPRCAP (SEQ ID NO: 8), EBI2 (SEQ ID NO: 9), PTPRC (SEQ ID NO: 10, 18, 25, or 243), ANP32E (SEQ ID NO: 11), SFPQ (SEQ ID NO: 12, 38 or 272), C1QR1 (SEQ ID NO: 13), FNBP1 (SEQ ID NO: 14 or 28), CBFB (SEQ ID NO: 15), SFN (SEQ ID NO: 96 or 324), LISCH7 (SEQ ID NO: 97), EPB41L4B (SEQ ID NO: 98), MST1R (SEQ ID NO: 99), ITGB4 (SEQ ID NO: 100), DBNDD2 (SEQ ID NO: 102 or 365), TACSTD1 (SEQ ID NO: 104), MISP (SEQ ID NO: 105), KRT8 (SEQ ID NO: 106), JUP (SEQ ID NO: 107 or 400), KRT18 (SEQ ID NO: 108 or 306), FA2H (SEQ ID NO: 109), DSG2 (SEQ ID NO:111 or 312), LRP5 (SEQ ID NO: 112). The expression level of the biomarker(s) may be determined using, e.g., a microarray, PCR, or other techniques described herein, for example, using a nucleic acid probe sequence based on the target sequences shown in Tables 2-5.

The biomarker DSG2 (SEQ ID NO:111 or 312) may be used to assess a cancer patient's (e.g., a patient having cancer that is resistant to one or more cancer therapies other than sPLA₂ hydrolysable, cisplatin-containing liposome) responsiveness to sPLA₂ hydrolysable, cisplatin-containing liposome (e.g., LiPlaCis). The expression level of the biomarker DSG2 (SEQ ID NO: 11 or 312) may be assessed using nucleic acid amplification methods (e.g., PCR) or a device (e.g., a microarray). As is described above, the expression level of DSG2 (SEQ ID NO:111 or 312) in the patient sample may then be compared, e.g., to the expression level of DSG2 (SEQ ID NO:111 or 312) in a cell (e.g., a cancer cell) or tissue (e.g., a tumor tissue) known to be sensitive or resistant to treatment with sPLA₂ hydrolysable, cisplatin-containing liposome and used to determine the cancer patient's responsiveness to the same. The biomarker DSG2 (SEQ ID NO:111 or 312) may be used alone to predict cancer patient responsiveness to treatment with sPLA₂ hydrolysable, cisplatin-containing liposome or in combination with one or more additional biomarkers (e.g., one, two, three, four, five, ten, or all of the biomarkers shown in Tables 2-5), such as COL5A2 (SEQ ID NO 73 or 211), ITGA4 (SEQ ID NO: 1), MSN (SEQ ID NO: 2), FAM46A (SEQ ID NO: 3 or 280), ITGB2 (SEQ ID NO: 4), DOCK2 (SEQ ID NO: 5 or 223), EVL (SEQ ID NO: 6), SACS (SEQ ID NO: 7), PTPRCAP (SEQ ID NO: 8), EBI2 (SEQ ID NO: 9), PTPRC (SEQ ID NO: 10, 18, 25, or 243), ANP32E (SEQ ID NO: 11), SFPQ (SEQ ID NO: 12, 38 or 272), C1QR1 (SEQ ID NO: 13), FNBP1 (SEQ ID NO: 14 or 28), CBFB (SEQ ID NO: 15), SFN (SEQ ID NO: 96 or 324), LISCH7 (SEQ ID NO: 97), EPB41L4B (SEQ ID NO: 98), MST1R (SEQ ID NO: 99), ITGB4 (SEQ ID NO: 100), DBNDD2 (SEQ ID NO: 102 or 365), TACSTD1 (SEQ ID NO: 104), MISP (SEQ ID NO: 105), KRT8 (SEQ ID NO: 106), JUP (SEQ ID NO: 107 or 400), KRT18 (SEQ ID NO: 108 or 306), FA2H (SEQ ID NO: 109), MGAT4B (SEQ ID NO: 110), LRP5 (SEQ ID NO: 112). The expression level of the biomarker(s) may be determined using, e.g., a microarray, PCR, or other techniques described herein, for example, using a nucleic acid probe sequence based on the target sequences shown in Tables 2-5.

The biomarker LRP5 (SEQ ID NO: 112) may be used to assess a cancer patient's (e.g., a patient having cancer that is resistant to one or more cancer therapies other than sPLA₂ hydrolysable, cisplatin-containing liposome) responsiveness to sPLA₂ hydrolysable, cisplatin-containing liposome (e.g., LiPlaCis). The expression level of the biomarker LRP5 (SEQ ID NO: 112) may be assessed using nucleic acid amplification methods (e.g., PCR) or a device (e.g., a microarray). As is described above, the expression level of LRP5 (SEQ ID NO: 112) in the patient sample may then be compared, e.g., to the expression level of LRP5 (SEQ ID NO: 112) in a cell (e.g., a cancer cell) or tissue (e.g., a tumor tissue) known to be sensitive or resistant to treatment with sPLA₂ hydrolysable, cisplatin-containing liposome and used to determine the cancer patient's responsiveness to the same. The biomarker LRP5 (SEQ ID NO: 112) may be used alone to predict cancer patient responsiveness to treatment with sPLA₂ hydrolysable, cisplatin-containing liposome or in combination with one or more additional biomarkers (e.g., one, two, three, four, five, ten, or all of the biomarkers shown in Tables 2-5), such as COL5A2 (SEQ ID NO 73 or 211), ITGA4 (SEQ ID NO: 1), MSN (SEQ ID NO: 2), FAM46A (SEQ ID NO: 3 or 280), ITGB2 (SEQ ID NO: 4), DOCK2 (SEQ ID NO: 5 or 223), EVL (SEQ ID NO: 6), SACS (SEQ ID NO: 7), PTPRCAP (SEQ ID NO: 8), EBI2 (SEQ ID NO: 9), PTPRC (SEQ ID NO: 10, 18, 25, or 243), ANP32E (SEQ ID NO: 11), SFPQ (SEQ ID NO: 12, 38 or 272), C1QR1 (SEQ ID NO: 13), FNBP1 (SEQ ID NO: 14 or 28), CBFB (SEQ ID NO: 15), SFN (SEQ ID NO: 96 or 324), LISCH7 (SEQ ID NO: 97), EPB41L4B (SEQ ID NO: 98), MST1R (SEQ ID NO: 99), ITGB4 (SEQ ID NO: 100), DBNDD2 (SEQ ID NO: 102 or 365), TACSTD1 (SEQ ID NO: 104), MISP (SEQ ID NO: 105), KRT8 (SEQ ID NO: 106), JUP (SEQ ID NO: 107 or 400), KRT18 (SEQ ID NO: 108 or 306), FA2H (SEQ ID NO: 109), MGAT4B (SEQ ID NO: 110), DSG2 (SEQ ID NO:111 or 312). The expression level of the biomarker(s) may be determined using, e.g., a microarray, PCR, or other techniques described herein, for example, using a nucleic acid probe sequence based on the target sequences shown in Tables 2-5.

TABLE 2
mRNA biomarkers of sensitivity to cisplatin. Dashes mean that the Affymetrix
probeset has not been mapped to a specific gene. Affymetrix IDs refer to the
array type HG-U133A.

| | | | | SEQ ID |
|---|---|---|---|---|
| Gene | Affymetrix ID | Correlation | Affymetrix Probe Sequence | NO: |
| ITGA4 | 213416_at | 0.46 | CAGGCCTCTCAGATACAAGGGGAAC | 1 |
| MSN | 200600_at | 0.45 | ATAGCTGCCTTAAAGTCAGTAACTT | 2 |
| FAM46A | 221766_s_at | 0.41 | CACCATGCTGGCTATCCGGGTGTTA | 3 |
| ITGB2 | 202803_s_at | 0.39 | CTCCACTCTGACTGGCACAGTCTTT | 4 |
| DOCK2 | 213160_at | 0.39 | GATTCCTGAACTCAAGGTACCAGCA | 5 |
| EVL | 217838_s_at | 0.39 | GATCATCGACGCCATCAGGCAGGAG | 6 |
| SACS | 213262_at | 0.38 | GTGTGGTTGAACAGGATGCAATCTT | 7 |
| PTPRCAP | 204960_at | 0.37 | GCTTCCCAAGATGCCATGGCTGGAC | 8 |
| EBI2 | 205419_at | 0.37 | GCAGGACTTCCCTTATAAAGCAAAA | 9 |
| PTPRC | 212587_s_at | 0.37 | GATTATAACCGTGTTGAACTCTCTG | 10 |
| ANP32E | 221505_at | 0.37 | GTTTTCGGTCCTATTTTAATGCTCT | 11 |
| SFPQ | 201586_s_at | 0.36 | AAAGACCAACAAATCTCAAGCCCTA | 12 |
| C1QR1 | 202878_s_at | 0.36 | GGTCTGTTCTTGTAGATAATGCCCT | 13 |
| FNBP1 | 213940_s_at | 0.36 | TGCTGGCCACGGATTTTGACGACGA | 14 |
| CBFB | 202370_s_at | 0.35 | GGTGTTGTACAGCTCACATGTTTAC | 15 |
| HCLS1 | 202957_at | 0.35 | GGTTTGCCTCATTGTGCTATTTGCC | 16 |
| IFI16 | 208965_s_at | 0.35 | ATAAGCATTGATTCCTGCATTTCTG | 17 |
| PTPRC | 212588_at | 0.35 | GCATTTAGTCCAATGTCTTTTTAAG | 18 |
| SFRS7 | 213649_at | 0.35 | ATCATGCTGAGGCGCCTTGCAAATC | 19 |
| CAP350 | 204373_s_at | 0.34 | ATGACTGGTATGATAGCTCTTGACA | 20 |
| IGLL1 | 206660_at | 0.34 | CAATCCAAGCATAACTCAGTGACGC | 21 |
| DOCK10 | 219279_at | 0.34 | GAATGTGTAGCTCAAATGCAAACCA | 22 |
| WASPIP | 202664_at | 0.33 | TTCCCTCCTTATAGTCAAGGACCGT | 23 |
| FLI1 | 204236_at | 0.33 | TGACCTCGGTCACAAAAGCAGTTTT | 24 |
| PTPRC | 207238_s_at | 0.33 | GAACAGTTTGTACAGACGTATGCTT | 25 |
| IFI16 | 208966_x_at | 0.33 | TACAACACTATACATACACACCACC | 26 |
| HDGFRP3 | 209524_at | 0.33 | TTATGCCAGCTTATATTGTGAGAAC | 27 |
| FNBP1 | 212288_at | 0.33 | GAGTTGCCTGTTTGTCTCTGGAGAT | 28 |
| SEPT6 | 212414_s_at | 0.33 | GCTGCAGTGTAGATGGCTCTTGTTT | 29 |
| ARHGAP15 | 218870_at | 0.33 | ACGTTGTCACCGGAGCACTGAAGAT | 30 |
| RASSF2 | 203185_at | 0.32 | ATAGCAGCACACATTTTCACGTTTC | 31 |
| GMFG | 204220_at | 0.32 | AAGACCGGCAGATGGTGGTGCTGGA | 32 |
| SYNCRIP | 209025_s_at | 0.32 | ATTTGGCTCAAGTCCATTTGGCTGT | 33 |
| HDGFRP3 | 209526_s_at | 0.32 | GCATGAAGTTGCCCTTAACCACTAA | 34 |
| ARHGEF6 | 209539_at | 0.32 | TAACCATGCTTACACACTAAACTAT | 35 |
| TMEM5 | 204808_s_at | 0.31 | TGCCCGGTCGGAGTAAACACAGAAT | 36 |
| CENTB1 | 205213_at | 0.31 | GATGTCAACTGGGTCAATGGGGGCC | 37 |
| SFPQ | 214016_s_at | 0.31 | GTTGGCTGATATTGGAGTGCTCATT | 38 |
| BCAT1 | 214452_at | 0.31 | CCTTTTGTACTTCACTCAGATACTA | 39 |
| LCP1 | 208885_at | 0.3 | TAAGCATCCTTAGGGTTCTGCCTCT | 40 |
| CORO1A | 209083_at | 0.3 | CTCATCTCCCTCAAGGATGGCTACG | 41 |
| SLC4A7 | 209884_s_at | 0.3 | TGTGAATCATCCTGCCTTTCAAATT | 42 |
| RAFTLIN | 212646_at | 0.3 | TACAAACCACATTACTTCTGTCACT | 43 |
| CKIP-1 | 218223_s_at | 0.3 | GTCCCGGATCCAGGACCTGGTAGCA | 44 |
| SNRP70 | 201221_s_at | 0.29 | AGTGAAGAGGTCGTCCTCTCCATCT | 45 |
| BNIP3 | 201849_at | 0.29 | GCTGAAGGCACCTACTCAGTATCTT | 46 |
| SLA | 203761_at | 0.29 | TAAGCATTCCGTCCATCTAAGCTCA | 47 |
| MFNG | 204153_s_at | 0.29 | TGATGGAGCATAACGGGTCCCAGCC | 48 |
| LOC57821 | 206721_at | 0.29 | ATGATTTCTTAGGGTCTGTGTACTT | 49 |
| CBLB | 209682_at | 0.29 | GTTCCATTTCTCTCATTCACAAGAT | 50 |
| QKI | 212636_at | 0.29 | GAGGCCAAGAAATTCCATGTTGTTT | 51 |
| ZRF1 | 213097_s_at | 0.29 | AAAGCTGTGAATCTGTTCCCTGCTG | 52 |
| FTL | 213187_x_at | 0.29 | ATGAGCTCCCAGATTCGTCAGAATT | 53 |
| SFRS7 | 214141_x_at | 0.29 | TCCCCATCAGGAAGTCCTCGCAGAA | 54 |
| VIM | 201426_s_at | 0.28 | TGAGTCCCTGGAACGCCAGATGCGT | 55 |
| PWP1 | 201606_s_at | 0.28 | TTAGAGCCAGTCTTCACACTCGGAA | 56 |
| AKAP7 | 205771_s_at | 0.28 | AAAACTTCCCCGGTATGATGATTGT | 57 |
| AF1Q | 211071_s_at | 0.28 | TCAGTGGGCACAGTTCTTCAGCTAC | 58 |
| DICER1 | 213229_at | 0.28 | ACTAGCTCATTATTTCCATCTTTGG | 59 |
| PDE4DIP | 213388_at | 0.28 | AATTATGAGTTTCTATCTGTGTCCA | 60 |
| CAP350 | 213956_at | 0.28 | GGGAAGTCCACATAGCGTCATTAAT | 61 |
| AIF1 | 215051_x_at | 0.28 | TTCAGCTACCCTGACTTTCTCAGGA | 62 |
| TRAF3 | 221571_at | 0.28 | GGCATGATGTCCGGTGATTTCTGTA | 63 |
| MBNL1 | 201152_s_at | 0.27 | ACTCTTGAGGGTTGATTATGCTGCA | 64 |
| FMNL1 | 204789_at | 0.27 | GGACCTCATCTCTGAGCTGAAACGG | 65 |
| TMEFF1 | 205122_at | 0.27 | GTTGGTGTTTAAAGATCTGAAGTGT | 66 |
| IL6R | 205945_at | 0.27 | GAAGCACCATAACTTTGTTTAGCCC | 67 |
| SIVA | 210792_x_at | 0.27 | ACAGCATGAGGCGGCCGGGGAGCTG | 68 |
| MCAM | 211340_s_at | 0.27 | GCTATGGTTATATTAGCACCAAACT | 69 |
| POLR2I | 212955_s_at | 0.27 | GGCCGACAACAGCTGCATCTATGTC | 70 |
| T3JAM | 213888_s_at | 0.27 | TGAAAAAGGGTTTCTATTCTCTCTG | 71 |
| C1orf24 | 217967_s_at | 0.27 | AGTATCAGTCGGTGCAACAGTTGGC | 72 |
| COL5A2 | 221730_at | 0.27 | TGAAGTTGATCCTGAGACTCTTGAA | 73 |
| LAPTM5 | 201720_s_at | 0.26 | TACTCAGAGGTGTGACCCTCGCCAG | 74 |
| JARID1A | 202040_s_at | 0.26 | GTCGTACTATCTTACTGAGCCACAG | 75 |
| CUGBP2 | 202156_s_at | 0.26 | AAGGCGTAACGAGTTCATCTTTCTT | 76 |
| PTPN7 | 204852_s_at | 0.26 | CCTTGATACCAGCTCTCTGTGGAAA | 77 |
| LCP2 | 205269_at | 0.26 | AAATCACTAAACCTCGTTTTCTCAG | 78 |
| RASA4 | 212706_at | 0.26 | AGCGTCCTTATCTTTCAGAGCTACA | 79 |
| FTL | 212788_x_at | 0.26 | AAACCCCAGACGCCATGAAAGCTGC | 80 |
| CD3D | 213539_at | 0.26 | GGGAACACTGCTCTCAGACATTACA | 81 |
| EIF4A1 | 214805_at | 0.26 | CTTTTTCCTGGGTCATGCTGCAACA | 82 |
| NKTR | 215338_s_at | 0.26 | GATGGGGTGCATGTAGTCTTTGGAC | 83 |
| C1orf24 | 217966_s_at | 0.26 | GAAGGTGTGATCTGTGGGACTGTCT | 84 |
| C2orf33 | 219137_s_at | 0.26 | GTACGTTTTTACTCAGTTCATGCGT | 85 |
| TMEM22 | 219569_s_at | 0.26 | GCTTCTCGTGCTGCACATATTTCCT | 86 |
| GIMAP6 | 219777_at | 0.26 | GTGAACAGACTTGAAACTCCAGAGC | 87 |
| RAP1B | 200833_s_at | 0.25 | ATCATTTTCAGGCTTCTGCAGCTGT | 88 |
| SRRM1 | 201225_s_at | 0.25 | GCATGTTGTTTGCCAGGACACTGTG | 89 |
| PWP1 | 201608_s_at | 0.25 | TTGTGCTTGCTCTTCAGATGGATGG | 90 |
| EDG1 | 204642_at | 0.25 | TAGCCAGGATCCTTGGTGTCCTAGG | 91 |
| CD47 | 211075_s_at | 0.25 | GCGGCGTGTATACCAATGCATGGCC | 92 |
| CG018 | 213375_s_at | 0.25 | GAATAACTTTTGGCTGTTGTGCTAA | 93 |
| TPK1 | 221218_s_at | 0.25 | TGGCCCGCGTGATTGTGGCATTTAA | 94 |
| COL5A2 | 221729_at | 0.25 | CATAACTGTTAGACTTCCCGTTTCT | 95 |

TABLE 3
mRNA biomarkers of resistance to cisplatin. Dashes mean that the Affymetrix
probeset has not been mapped to a specific gene. Affymetrix IDs refer to
the array type HG-U133A.

| | | | | SEQ ID |
|---|---|---|---|---|
| Gene | Affymetrix ID | Correlation | Affymetrix Probe Sequence | NO: |
| SFN | 33323_r_at | -0.48 | TCAATAAAGTTCCCCTGTGACACTC | 96 |
| LISCH7 | 208190_s_at | -0.47 | CTCCCCTATGATGGGCGGCTACTGG | 97 |
| EPB41L4B | 220161_s_at | -0.47 | ATCAGTTGATTCTTGTGCCATTTTT | 98 |
| MST1R | 205455_at | -0.46 | TGAGCCAGTGAGGGCAGTCCTGCAA | 99 |
| ITGB4 | 204990_s_at | -0.45 | GCATCATCACCATAGAGTCCCAGGA | 100 |
| SFN | 209260_at | -0.45 | TCTTGCTCCAAAGGGCTCCGTGGAG | 101 |
| C200rf35 | 218094_s_at | -0.45 | ATACGCCCTTGGCACAGTCGGATGA | 102 |
| SFN | 33322_i_at | -0.45 | GTCTGCTGGGTGTGACCATGTTTCC | 103 |
| TACSTD1 | 20183_s_at | -0.43 | GTGCGTGGGACGAAGACATCTTTGA | 104 |
| C19orf21 | 212925_at | -0.42 | TGGTCCCCTTCACCTGGGAGAAAAG | 105 |
| KRT8 | 209008_x_at | -0.41 | GGGCCAAGCAGGACATGGCGCGGCA | 106 |
| JUP | 201015_s_at | -0.4 | AGCTTCAGACTCAAGTACCCATTCT | 107 |
| KRT18 | 201596_x_at | -0.4 | GAGCTGCTGAGACGACGCTCACAGA | 108 |
| FA2H | 219429_at | -0.39 | GAGAAGCAGTTTGACGGACCTTGTG | 109 |
| MGAT4B | 220189_s_at | -0.38 | GGTGATTCTGAGCGAGATCTTCCTG | 110 |
| DSG2 | 217901_at | -0.37 | GCAGCCTTGGAAACCTAACCTGCCT | 111 |
| LRP5 | 209468_at | -0.36 | CCTGCAGCACCGACGTGTGTGACAG | 112 |
| GJB3 | 215243_s_at | -0.36 | ACTTGGCTCAGTGGAAGCCCTCTTT | 113 |
| TACSTD2 | 202286_s_at | -0.35 | ACATTGCCCGGAAACTCAGTCTATT | 114 |
| LAD1 | 203287_at | -0.35 | GCTGTGGATCTGTTTGGCCAGGGTC | 115 |
| AGR2 | 209173_at | -0.35 | GTTAGAGCCGATATCACTGGAAGAT | 116 |
| HTATIP2 | 209448_at | -0.35 | AGATTTGTCAGCCCTATCTCAAACT | 117 |
| LOC57228 | 209679_s_at | -0.35 | AGGTCTTCCCAGAGGCTGGATACCA | 118 |
| BCL2L1 | 212312_at | -0.35 | GTCTTCCCTACCTCAGGCAGGAAGG | 119 |
| GPX2 | 202831_at | -0.34 | CTACCCTTATGATGACCCATTTTCC | 120 |
| SOX9 | 202935_s_at | -0.34 | AAATGCTCTTATTTTTCCAACAGCT | 121 |
| TPBG | 203476_at | -0.34 | GTGTATAGTGTTTTACCCTCTTCTT | 122 |
| LGALS4 | 204272_at | -0.34 | TCATCAAGGGCTATGTGCCTCCCAC | 123 |
| PHLDA1 | 217996_at | -0.34 | CCCCGCACCAGATCAAGTAGTTTGG | 124 |
| PLEK2 | 218644_at | -0.34 | CCCTCCTACCAGATGACACAGACAA | 125 |
| TNFRSF21 | 218856_at | -0.34 | TGTATGGTTTTCACCTGGACACCGT | 126 |
| IER3 | 201631_s_at | -0.33 | AACTCCGTCTGTCTACTGTGTGAGA | 127 |
| RAI3 | 203108_at | -0.33 | CCCACTGGCCTGAATCTACACTGGA | 128 |
| BENE | 209373_at | -0.33 | ACATTACATCCGTGGATTCTCCTGC | 129 |
| MGC50853 | 212400_at | -0.33 | GGCCCTGGGCCAGGGTGATTGGACT | 130 |
| RAI3 | 212444_at | -0.33 | TTTAGCCCTCATGACTGTATTTTCT | 131 |
| CLIC3 | 219529_at | -0.33 | ACACGCTGCAGATCGAGGACTTTCT | 132 |
| CLDN3 | 203954_x_at | -0.32 | ACCGGCAGCCCTGGAAGGGGCACTT | 133 |
| FGFR4 | 204579_at | -0.32 | TACCAGCAGGAGGTTCTGGGCCTCT | 134 |
| PPARG | 208510_s_at | -0.32 | CATCTTTCAGGGCTGCCAGTTTCGC | 135 |
| FBP1 | 209696_at | -0.32 | GGGCTACGCCAAGGACTTTGACCCT | 136 |
| CPNE3 | 202119_s_at | -0.31 | AATCTAGTCACCTAACCTTGTGGTT | 137 |
| AREG | 205239_at | -0.31 | ATTTCAAAATTTCTGCATTCACGGA | 138 |
| VIL1 | 205506_at | -0.31 | AACACCTGTCCATTGAAGATTTCAC | 139 |
| GATA6 | 210002_at | -0.31 | GACATTCTTATGCTTCTTTTACAAC | 140 |
| TCF7L2 | 212761_at | -0.31 | AATGTTTCCTAACAGTTGTGATGTT | 141 |
| PP1201 | 217730_at | -0.31 | GGGTGAAGAGAGACTCGGTGCGGGC | 142 |
| FLJ20847 | 219053_s_at | -0.31 | CGACCGCCTGTATGTTTGTGTAATT | 143 |
| GPR172A | 222155_s_at | -0.31 | AAGGCCTATCAGCTTCTATCAGCCC | 144 |
| ITGA6 | 201656_at | -0.3 | GTCACTGGTCTGTTTGCATTTGATA | 145 |
| ZNF165 | 206683_at | -0.3 | AGCTCAAAACTTGCTAGGCATCAGA | 146 |
| FLNB | 208613_s_at | -0.3 | GCAGCAAAGCTGGCTCCAACATGCT | 147 |
| MCCC2 | 209623_at | -0.3 | AAACACTATCTACTTCCTTTGTCAT | 148 |
| FLJ20273 | 218035_s_at | -0.3 | GAGGATCATGCCCTTAGCAAGTACT | 149 |
| TMEM16A | 218804_at | -0.3 | AACATCATTTTAGCAAAGGCCAGGA | 150 |
| RAB11FIP1 | 219681_s_at | -0.3 | TGTCCTTGTTACATTGAGGTTAAGA | 151 |
| SLC3A2 | 200924_s_at | -0.29 | TCCCTACTGCATGGGGACTTCCACG | 152 |
| EFNA1 | 202023_at | -0.29 | CCACCTTCACCTCGGAGGGACGGAG | 153 |
| SORL1 | 203509_at | -0.29 | TAATTACACGTTCACCGTCCAAGCA | 154 |
| PLS1 | 205190_at | -0.29 | TTCCCTTTCTACCATTGATTTAAAT | 155 |
| GALIG | 208949_s_at | -0.29 | AGTACTGGTTGAACCTGACCACTTC | 156 |
| EHD1 | 209038_s_at | -0.29 | AAATACATAAGCTAGTTTCTGTTCT | 157 |
| NR2F2 | 209120_at | -0.29 | GTAACGTGATTGATTCAGTATCTTA | 158 |
| SERPINB1 | 213572_s_at | -0.29 | AATACATCCGATGCGTAGATTCTTG | 159 |
| PCK2 | 202847_at | -0.28 | AGAATGCTCGGGTGCTAGACTGGAT | 160 |
| ARF6 | 203311_s_at | -0.28 | GGACGGACTCTATGAGGGGCTCACA | 161 |
| TGFA | 205016_at | -0.28 | GGAATGACTCAAATGCCCAAAACCA | 162 |
| CST6 | 206595_at | -0.28 | TCCTCTCAGCTCCTAAAGCACAACT | 163 |
| PXN | 211823_s_at | -0.28 | ACATGTTCGCACCCAAGTGTGGCGG | 164 |
| SORL1 | 212560_at | -0.28 | TTTCAGATGGAGTACCAGCACCGAA | 165 |
| SLC39A4 | 219215_s_at | -0.28 | TGGCACTCGCGGTTGGAGTCAGCGA | 166 |
| GCNT3 | 219508_at | -0.28 | GGCCATCTATGGGACTGAACTTTGA | 167 |
| S100A11 | 200660_at | -0.27 | GAAGAAACTGGACACCAACAGTGAT | 168 |
| ITPR3 | 201189_s_at | -0.27 | GCTGTAGCCAGTGCAGACCTCACTG | 169 |
| DHCR7 | 201790_s_at | -0.27 | AGGTGTCCAGTACCTAATCACGCTC | 170 |
| TCIRG1 | 204158_s_at | -0.27 | TTGCCGTGATGACCGTGGCTATCCT | 171 |
| NR2F2 | 209121_x_at | -0.27 | GAATACGTTAGGAGCCAGTACCCCA | 172 |
| SLC25A1 | 210010_s_at | -0.27 | GAAGCTGCTCAACAAAGTGTGGAAG | 173 |
| SERPINB6 | 211474_s_at | -0.27 | GGAATGTCCCAGACAGACCTGTCTC | 174 |
| ARTN | 216052_x_at | -0.27 | CCTTCATGGACGTCAACAGCACCTG | 175 |
| LOC51123 | 218059_at | -0.27 | GGCCCGGATATGGCTCGTGGACAGC | 176 |
| S100A14 | 218677_at | -0.27 | AGGAGTCTCCACCAGAGGGAGGCTC | 177 |
| FCGRT | 218831_s_at | -0.27 | GAGCACCACTACTGCTGCATTGTGC | 178 |
| RAB20 | 219622_at | -0.27 | ACTCTGACATTTCTTGTTCTCAAGC | 179 |
| SPDEF | 220192_x_at | -0.27 | CCAGCATTTCCAGAGCAGAGCCTAC | 180 |
| PNAS-4 | 221648_s_at | -0.27 | GCGTGTCTTGAGTTCCATGCAAATT | 181 |
| PXN | 201087_at | -0.26 | AATGGTGACAGTCCAAACCACTCCA | 182 |
| TPD52L2 | 201379_s_at | -0.26 | GGCCCTGCATGTCAGATGGCGTGGT | 183 |
| ALDH3A2 | 202054_s_at | -0.26 | TGATCATAAATTCTCCCCAACTATA | 184 |
| ARF6 | 203312_x_at | -0.26 | AAAGTTGCCAAGATGCTCCTTGTTG | 185 |
| GPA33 | 205929_at | -0.26 | GTCTCACCCAACTGCAGTTTACTAT | 186 |
| — | 208540_x_at | -0.26 | GACGGAGTTCCTAAGCTTCATGAAT | 187 |
| FLNB | 208614_s_at | -0.26 | TCAGCCTGGGCAGTCTTACCAAAAT | 188 |
| TSPAN-1 | 209114_at | -0.26 | TGCTGTGGCTTCACCAACTATACGG | 189 |
| CDH17 | 209847_at | -0.26 | CCTTGACTCCTTTGGTATTTCACTG | 190 |
| SERPINB1 | 212268_at | -0.26 | ACAGCAGGCATCGCAACTTTCTGCA | 191 |
| LCN2 | 212531_at | -0.26 | CAAGAGCTACAATGTCACCTCCGTC | 192 |
| KIAA0984 | 213913_s_at | -0.26 | GTTTGTCTCTTGTTGTTCTGAAGGA | 193 |
| ACSL5 | 218322_s_at | -0.26 | CTCTCTAGTTAGATATCTGACTTGG | 194 |
| MUC13 | 218687_s_at | -0.26 | TCCAGCCTCGGGGTGTAGGTTTCTG | 195 |
| FAM11B | 219253_at | -0.26 | ACTCGTCTCACGCCGTGTTTGAGAT | 196 |
| SH2D3A | 219513_s_at | -0.26 | GCCAGAGTTCAAATGTGACTCCACC | 197 |
| ANXA2 | 201590_x_at | -0.25 | CAAGCCCCTGTATTTTGCTGATCGG | 198 |
| TM4SF3 | 203824_at | -0.25 | AGACCACAGATATCTTCTAGACATA | 199 |
| NT5E | 203939_at | -0.25 | GTCACTGTAAATCATTCTTAAGCCC | 200 |
| TETRAN | 209215_at | -0.25 | AAGGCTGTCAGGGCTTCTGTTTGTT | 201 |
| CTBP2 | 210835_s_at | -0.25 | GTAGACACCTGCACGCATAGGATTG | 202 |
| SCD | 211708_s_at | -0.25 | TTGCCACTTTCTTGCGATATGCTGT | 203 |
| DNMBP | 212838_at | -0.25 | GCCATTCCAGAAGTAGCTTATCCTA | 204 |
| TMC5 | 219580_s_at | -0.25 | CCAATACCCCACCGTGATGACTTGA | 205 |

TABLE 4
mRNA biomarkers of sensitivity to LiPlaCis. Dashes mean that the Affymetrix
probeset has not been mapped to a specific gene. Affymetrix IDs refer to
the array type HG-U133A.

| | | | | SEQ ID |
|---|---|---|---|---|
| Gene | Affymetrix ID | Covariance | Affymetrix Probe Sequence | NO: |
| CALD1 | 212077_at | 10861321835689.1 | AATTCTCTGTTATCTTTACGAGGTA | 206 |
| COL6A2 | 209156_s_at | 8535698909744.43 | CACGAGAAGGACTATGACAGCCTGG | 207 |
| FERMT2 | 209210_s_at | 5291552917682.63 | TGATTTGCCACAATGTCCTTAACTC | 208 |
| BNIP3 | 201849_at | 5145685657339.48 | GCTGAAGGCACCTACTCAGTATCTT | 209 |
| RAB31 | 217762_s_at | 4734688539598.5 | AGACCTGGCACTTCAGTAACTCAGC | 210 |
| COL5A2 | 221730_at | 4647176466910.36 | GACTCTTGAAGTAATGGCTGATCCT | 211 |
| MPO | 203948_s_at | 4518211644157.6 | GGGACTTTGTCAACTGCAGTACACT | 212 |
| SRPX | 204955_at | 4340511505629.07 | CCTTTCTTTACTCCATCATGGCTGG | 213 |
| ARHGDIB | 201288_at | 4263392661779.67 | ATCACTAACAGGTCTTTGACTCAGG | 214 |
| TMEM47 | 209656_s_at | 4156685173988.01 | GAATTCATGGTATCCTGGTTATTTT | 215 |
| CSRP2 | 207030_s_at | 3960151282910.27 | AACTACTGTGAAATTCTACCAGCAT | 216 |
| DPYSL3 | 201431_s_at | 3876388962016.02 | GACACCTGAGCCTGGATTTTCACTC | 217 |
| HTRA1 | 201185_at | 3845854940391.73 | TCAAACGGCCGAAGTTGCCTCTTTT | 218 |
| SLC39A6 | 202088_at | 3547148987590.88 | ATACTAGGCCTGTCTGTGGCATTCT | 219 |
| LAT2 | 221581_s_at | 3545380986375.43 | GGATTTAGGATAAGCTGTCACCCAG | 220 |
| ENAH | 217820_s_at | 3385939870513.75 | GGTCAGCAACCTCTTTTGATTTTGT | 221 |
| RPS4Y1 | 201909_at | 3384951327956.31 | GACAGGTGAACATTTCCGCCTGGTC | 222 |
| DOCK2 | 213160_at | 3367491706976.35 | GATTCCTGAACTCAAGGTACCAGCA | 223 |
| COL1A1 | 202311_s_at | 3222960671378.67 | TGTTCCTTTTTGTTCAAAGTCTATT | 224 |
| GMFG | 204220_at | 3013566458581.29 | AGGTGTTCGAAATCCGCACCACTGA | 225 |
| CYR61 | 201289_at | 2999506373414.97 | GTGGAGTTGATGACTTTCTGTTTTC | 226 |
| RHOB | 212099_at | 2978300392812.93 | TGCAGGTCATGCACACAGTTTTGAT | 227 |
| CORO1A | 209083_at | 2968352455386.15 | GCTCCAGAAGCGCTTGGACAGGCTG | 228 |
| ID4 | 209291_at | 2948241975028.96 | GGCATAATGGCAAATCCTTCAAGCA | 229 |
| RARRES2 | 209496_at | 2907180844659.6 | CCCCATAGAGACCCAAGTTCTGCGG | 230 |
| SOX4 | 201417_at | 2862450307972.36 | GTAAACCACATCTTTTTTGCACTTT | 231 |
| NID1 | 202007_at | 2798544570884.12 | CACTTTTTGTATTTATCGTTTCATA | 232 |
| CALD1 | 201616_s_at | 2776573094080.12 | GACGCAGGACGAGCTCAGTTGTAGA | 233 |
| SERPINE2 | 212190_at | 2767126943194.04 | TGTTGTGCAGTGTGCCTGTCACTAC | 234 |
| CTSL1 | 202087_s_at | 2681524741399.96 | CACTTACTGACTTTGCATTTTCGTT | 235 |
| C3orf14 | 219288_at | 2679480387909.32 | GGTGGTTTCTCTTGAGACTCGTTAC | 236 |
| DKK3 | 202196_s_at | 2608335983440.84 | TTGGCAGTTGCATTAGTAACTTTGA | 237 |
| SCRN1 | 201462_at | 2582074623391.62 | TCATGTGCACATGCCGTTGCAGCAC | 238 |
| MT1M | 217546_at | 2555792977629.17 | CGTTGGAGAACTGCAGCTGCTGTGC | 239 |
| PLAU | 205479_s_at | 2529115320523.6 | AGCAGCTGAGGTCTCTTGAGGGAGC | 240 |
| NREP | 201310_s_at | 2514590941976.06 | CATTGGCCTGAGTTTCTTGTGCATT | 241 |
| HLA-B | 208729_x_at | 2501423496784.03 | GAGCCTACCTGGAGGGCGAGTGCGT | 242 |
| PTPRC | 212588_at | 2494855639496.51 | GTTTTCAATTTTGCATGCTCGATTA | 243 |
| HDGFRP3 | 209524_at | 2438222715080.89 | TTATGTGTACATTATTGTTGCTATT | 244 |
| CELF2 | 202157_s_at | 2427790438608.2 | CTTCCCGGTCACTGGTAACAATAGC | 245 |
| SFRP1 | 202037_s_at | 2413217767593.8 | GTACCTGTGGGTTAGCATCAAGTTC | 246 |
| HLA-B | 211911_x_at | 2358346288074.42 | CTGAGAGCCTACCTGGAGGGCCTGT | 247 |
| LOX | 215446_s_at | 2354236167712.24 | TTGGGCCTTTTATCTGTCTATCCAT | 248 |
| CLU | 208791_at | 2341547177698.15 | CAGTGTGACAAGTGCCGGGAGATCT | 249 |
| SH3BGRL | 201312_s_at | 2249866543302.91 | AGAATCTTTTCTATGCCTCTATTCC | 250 |
| INHBA | 210511_s_at | 2238550007854.02 | GCCATATAGCAGGCACGTCCGGGTC | 251 |
| MMP1 | 204475_at | 2203074303300.14 | GGCAAGGGATAACTCTTCTAACACA | 252 |
| WIPF1 | 202664_at | 2194537285288.12 | TTCCCTCCTTATAGTCAAGGACCGT | 253 |
| ADAMTS1 | 222162_s_at | 2144423953975.08 | AATAACGCAAATGGCTTCCTCTTTC | 254 |
| THY1 | 208850_s_at | 2141423198789.74 | GGCCTAGCACGGACATGGTCTGTCC | 255 |
| UCHL1 | 201387_s_at | 2140899985376.98 | TGATGGACGAATGCCTTTTCCGGTG | 256 |
| MYH10 | 212372_at | 2139390916542.17 | GATCCTCTGCAATGTGCTTGAAAAC | 257 |
| TYMS | 202589_at | 2131876162229.91 | TCACAAGCTATTCCCTCAAATCTGA | 258 |
| HCLS1 | 202957_at | 2089924252642.24 | TGATGAGCTTTCCTTTGATCCGGAC | 259 |
| HLA-B | 209140_x_at | 2085546519988.6 | GAGACAGCTGTCTTGTGAGGGACTG | 260 |
| IFI16 | 208966_x_at | 2061722348570.95 | TACACACCACCATATATACTAGCTG | 261 |
| PRKCB | 207957_s_at | 2037662863122.06 | GTGTAGGTGAATGCAAACTCCATCG | 262 |
| BNIP3 | 201848_s_at | 2008580245730.46 | TTCCTCTTTAAACACCCGAAGCGCA | 263 |
| TUSC3 | 213423_x_at | 1987545095813.27 | AACTGTTCCTGACTTTATACTATTT | 264 |
| WNT5A | 205990_s_at | 1982235386738.35 | GCATAATGATATTCACATCCCCTCA | 265 |
| CALD1 | 201617_x_at | 1981280027254.5 | TGTTGTTTCTGCACTTTATAATAAA | 266 |
| HLA-C | 216526_x_at | 1955999731784.71 | AGAGGTGGGGCTGGATGTCTCCATC | 267 |
| IL1R1 | 202948_at | 1955342562611.76 | AAGTGCAAAGTTATTCCCCATCTTC | 268 |
| AUTS2 | 212599_at | 1927738178390.84 | TACTTACACCCAAACAGATCCTGAA | 269 |
| THBS2 | 203083_at | 1912997768879.9 | TTGCGTGTGGAGCTGTATTCCCGAG | 270 |
| CHRDL1 | 209763_at | 1895325557387.3 | CCCTTTCACTGTTCTCACAGGACAT | 271 |
| SFPQ | 214016_s_at | 1886539698542.15 | GTTGGCTGATATTGGAGTGCTCATT | 272 |
| CXCL12 | 209687_at | 1857308403453.12 | CAGCAGGGTTTCAGGTTCCAATCAG | 273 |
| HOXC6 | 206858_s_at | 1831591158444.48 | CTGTATTTGTGGTCTCTGTATTTAT | 274 |
| PLAGL1 | 209318_x_at | 1827870818957.99 | ACATCCAAAATGACGGCTGCTATAT | 275 |
| RDX | 212397_at | 1815278384492.07 | GTGGACCCTACTATTCATGTTTTGA | 276 |
| HNRNPH1 | 213619_at | 1813815711802.08 | GCTTAAACTTACGTGCCTTACAGGT | 277 |
| KRAS | 214352_s_at | 1802923545775.42 | CATGCAGACTGTTAGCTTTTACCTT | 278 |
| IL8 | 211506_s_at | 1788698391848.43 | GTCAGTGCATAAAGACATACTCCAA | 279 |
| FAM46A | 221766_s_at | 1787987145165.06 | GGAGTCCTATTTGCAGAACCACTTT | 280 |
| QKI | 212265_at | 1787672566876.18 | ATAACCAACCTATTGCCTATGAGAA | 281 |
| CD53 | 203416_at | 1777870731216.97 | CGAATTAGTCTCCAGCCTCTAAATA | 282 |
| LAPTM5 | 201720_s_at | 1763708973603.65 | TCGGGTCTCTCCATAATTCAGCCCA | 283 |
| FOXG1 | 206018_at | 1752375753099.1 | ACGATTGCCTTCAGTTTGTGTTGTG | 284 |
| MST4 | 218499_at | 1732353014841.79 | AATTCTTTTTATTGGTGCCTATATT | 285 |
| GAPDH | AFFX- | 1692594771893.01 | AAGCTCACTGGCATGGCCTTCCGTG | 286 |
| GAPDH | HUMGAPDH/ | | | |
| | M33197_M_at | | | |
| TUBB2B | 214023_x_at | 1672014039622.35 | GAGATATTTCTGAATTACTGTTGTA | 287 |
| GAPDH | 212581_x_at | 1649610188507.54 | TTTGACGCTGGGGCTGGCATTGCCC | 288 |
| CEBPD | 203973_s_at | 1623762464226.23 | GGACAGCAGACTGCCGGTAACGCGC | 289 |
| PLAU | 211668_s_at | 1604895332856.59 | GCTCTGAAGTCACCACCAAAATGCT | 290 |
| CAV1 | 203065_s_at | 1604187716818.41 | GGTGCCAATTTCAAGTTCCAAGTTG | 291 |
| GAPDH | AFFX- | 1601834913853.31 | TAGGGAGCCGCACCTTGTCATGTAC | 292 |
| GAPDH | HUMGAPDH/ | | | |
| | M33197_3_at | | | |
| — | 213158_at | 1597303398144.17 | ACGTATATTTACCTGTGACTTGTAT | 293 |
| ARHGEF6 | 209539_at | 1586970619512.16 | TAAACTGCTGCCCGTAGAGGCCTTT | 294 |
| PRKCB | 209685_s_at | 1580850725622.13 | TGGATGTTAGCGGTACTCTTCCACT | 295 |
| SRGN | 201859_at | 1549790579490.15 | TTTTCCTGGATATCTGTGTATTTTC | 296 |
| TLE4 | 204872_at | 1549011037374.17 | ACTGTGCGTTGTACATAGTTCTAAT | 297 |
| LOC | 202350_s_at | 1544181853329.71 | GAACACTGGCCATAGGAAATGCTGT | 298 |
| 100506558 | | | | |
| MATN2 | | | | |
| BHLHE40 | 201170_s_at | 1537151135133.25 | GATCCTTTCTGTAGGCTAATTCCTC | 299 |
| SGCE | 204688_at | 1519398433064.38 | AACGCAGCAGAACTTGCCACATCAG | 300 |
| — | 222288_at | 1511518722955.02 | GAAGCTTGGCTTTAGTGGTAGAATG | 301 |
| PCBP2 | 204031_s_at | 1507948521040.68 | AGCCTGGCTCAATATCTAATCAATG | 302 |
| TFAP2A | 204653_at | 1493277682055.65 | GAACTTCAAACATTTGGGACCACCT | 303 |
| SPON1 | 209436_at | 1472949317341.51 | CCACCCTAGTGTCTCATGTTTGTAT | 304 |
| COL4A2 | 211966_at | 1468135692764.19 | TGGTGATGTCTGCTACTATGCCAGC | 305 |

TABLE 5
mRNA biomarkers of resistance to LiPlaCis. Dashes mean that the Affymetrix
probeset has not been mapped to a specific gene. Affymetrix IDs refer to the
array type HG-U133A.

| | | | | SEQ ID |
|---|---|---|---|---|
| Gene | Affymetrix ID | Covariance | Affymetrix Probe Sequence | NO: |
| KRT18 | 201596_x_at | -22426211704708.5 | AAGCTGGAGGCTGAGATCGCCACCT | 306 |
| LGALS3 | 208949_s_at | -11456296973610.8 | CACTTTAACCCACGCTTCAATGAGA | 307 |
| DSP | 200606_at | -10269594517738.5 | TGGAATGAGTCTCCTTTAGTTTCAG | 308 |
| IGFBP4 | 201508_at | -8435796702432.14 | AGAGACATGTACCTTGACCATCGTC | 309 |
| SPINT2 | 210715_s_at | -8294729535462.05 | TGGAAATCCTCTAGGAGGCTCCTCC | 310 |
| CDH1 | 201131_s_at | -7786548077136.61 | TGTGTGGGTGCTGATAATTGTGTAT | 311 |
| DSG2 | 217901_at | -7061991934030.4 | TACTCTTCCATCATCTAGAATTGTT | 312 |
| RAB25 | 218186_at | -6195270978776.59 | GCACCCTCAGGGTCTTAAGGTCTTC | 313 |
| PTPRF | 200636_s_at | -6131832886305.69 | GTACACAGTCTGTTTTCTATTTGTT | 314 |
| SOX9 | 202936_s_at | -5835576205162.92 | TGGGCTGCCTTATATTGTGTGTGTG | 315 |
| LYZ | 213975_s_at | -5458342909996.32 | TAACCCAGACTTAATCTTGAATGAT | 316 |
| IER3 | 201631_s_at | -5365171123958.73 | GAGACTTCGGCGGACCATTAGGAAT | 317 |
| PERP | 217744_s_at | -5097068499548.16 | ATGCACGTGAAACTTAACACTTTAT | 318 |
| SOX9 | 202935_s_at | -5050052756141.07 | AGTTGAACAGTGTGCCCTAGCTTTT | 319 |
| ATP1B1 | 201243_s_at | -4753436553865.35 | GATCTTGTATTCAGTCAGGTTAAAA | 320 |
| IFI27 | 202411_at | -4636709898452.9 | CCAAAGTGGTCAGGGTGGCCTCTGG | 321 |
| PHLDA2 | 209803_s_at | -4623467982538.76 | GGACGAGTCGGACCGAGGCTAGGA | 322 |
| | | | C | |
| CTTN | 201059_at | -4563342040423.69 | ATTTGTGGCCACTCACTTTGTAGGA | 323 |
| SFN | 209260_at | -4455761701170.73 | TCTTGCTCCAAAGGGCTCCGTGGAG | 324 |
| MALL | 209373_at | -4327230558082.54 | CTCCTCCATGAGTCTGACATCTCGG | 325 |
| S100A11 | 200660_at | -4322815561525.15 | GGTTGAGGAGAGGCTCCAGACCCGC | 326 |
| TSPAN13 | 217979_at | -4261036366041.2 | ACAGCAACTTGTCAAACCTAAGCAT | 327 |
| AKR1C3 | 209160_at | -4207721689216.25 | ACGCAGAGGACGTCTCTATGCCGGT | 328 |
| FAT1 | 201579_at | -4082641838983.11 | GTAGTCATTCATTTCTAGCTGTACA | 329 |
| DSTN | 201021_s_at | -4020978397283.39 | GTAGCTGATGAAGTATGTCGCATTT | 330 |
| EFEMP1 | 201842_s_at | -3992766849062.55 | GATGATCTTCTGTGGTGCTTAAGGA | 331 |
| TFF3 | 204623_at | -3853023482644 | CTGTGATTGCTGCCAGGCACTGTTC | 332 |
| HSPB1 | 201841_s_at | -3835026328384.26 | TTCACGCGGAAATACACGCTGCCCC | 333 |
| SDC1 | 201286_at | -3731984524505.92 | TCATCTGCTGGTCCGTGGGACGGTG | 334 |
| PLAC8 | 219014_at | -3720610591317.68 | GAAGGAGAGCCATGCGTACTTTCTA | 335 |
| TPBG | 203476_at | -3655713541808.07 | GTGTATAGTGTTTTACCCTCTTCTT | 336 |
| LCN2 | 212531_at | -3340240709988.96 | CAGGACTTTTGTTCCAGGTTGCCAG | 337 |
| CEACAM6 | 203757_s_at | -3279054777343.26 | GTGCAGTTTCTGACACTTGTTGTTG | 338 |
| ELF3 | 210827_s_at | -3241469160886.13 | GGGAGCACCGTGATGGAGAGGACA | 339 |
| | | | G | |
| CLDN3 | 203953_s_at | -3192796314939.69 | AAGGCCAAGATCACCATCGTGGCAG | 340 |
| TPD52L1 | 203786_s_at | -3049121447681.89 | TATTCAAATGGCCCCTCCAGAAAGT | 341 |
| VAMP8 | 202546_at | -2969692217517 | AAGCCACATCTGAGCACTTCAAGAC | 342 |
| C1orf106 | 219010_at | -2931724791122.81 | GTTCCAAGAACTCTGGTGTCTGACC | 343 |
| RBM47 | 218035_s_at | -2891974033193.95 | GAGGATCATGCCCTTAGCAAGTACT | 344 |
| C3 | 217767_at | -2846605120573.62 | GGTCTACGCCTATTACAACCTGGAG | 345 |
| CAPN2 | 208683_at | -2829130992700.86 | AATCGTTCTCCTTACAATCAAGTTC | 346 |
| ERBB3 | 202454_s_at | -2788407249074.31 | GGAACTAGGCTCTTATGTGTGCCTT | 347 |
| SLPI | 203021_at | -2755718313124.09 | TCTGTCCTCCTAAGAAATCTGCCCA | 348 |
| SPATS2L | 222154_s_at | -2729322838596.83 | GAGGCTCAGTTAGCAACCTGTGTTG | 349 |
| ERBB2 | 216836_s_at | -2698032874395.93 | AGACTGTCCCTGAAACCTAGTACTG | 350 |
| SERPINB1 | 212268_at | -2694341115802.62 | ACTTTCTGCATGTTGATGCCCGAAG | 351 |
| CEACAM6 | 211657_at | -2643169692661.57 | GTTCTTGTATTGTATTGCCCAGGGG | 352 |
| AKR1B10 | 206561_s_at | -2617913243059.4 | AAAAACCGCAGCCCAGGTTCTGATC | 353 |
| ID1 | 208937_s_at | -2607302720347.48 | GACATGAACGGCTGTTACTCACGCC | 354 |
| PPAP2C | 209529_at | -2576535604785.95 | TGTTCTTGGCGCTGTATGTGCAGGC | 355 |
| AQP3 | 39248_at | -2561344001860.94 | CTTCTACAGGCTTTTGGGAAGTAGG | 356 |
| PODXL | 201578_at | -2559443301040.98 | TGGAGGACACAGATGACTCTTTGGT | 357 |
| PRR15L | 219127_at | -2483388299723.69 | GAGTGGGTGGGGAATTTTCTCCTCT | 358 |
| EMP2 | 204975_at | -2470436470609.79 | CTGCACCTTCATCAGCGGCATGATG | 359 |
| MYO10 | 201976_s_at | -2463058577194.03 | TATAAACCACTCTTCAACAGCTGGC | 360 |
| SERPINB1 | 213572_s_at | -2374385129062.88 | AATACATCCGATGCGTAGATTCTTG | 361 |
| SDC4 | 202071_at | -2371552687950.61 | TGGCTTAGCCTGGGCAGGTCGTGTC | 362 |
| CRABP2 | 202575_at | -2354608471952.81 | GAGCAGGGTCTCTCTAAAGGGGACT | 363 |
| HTATIP2 | 209448_at | -2354028532889.45 | GTCTCTGAGTTACAAAAGTGCTAAT | 364 |
| DBNDD2 | 218094_s_at | -2352744142308.53 | ACCAGTTTTTGGCTTACTCCTGAGA | 365 |
| SYS1 | | | | |
| SYS1- | | | | |
| DBNDD2 | | | | |
| ESRP1 | 219121_s_at | -2312028194710.22 | TTGTCTACACTCAGGCTGCAGTATT | 366 |
| HSD17611 | 217989_at | -2304068718020.79 | TCCTGAGAGATACCTCACATTCCAA | 367 |
| GFPT1 | 202722_s_at | -2272343431090.56 | GGTTAGCCTTAGTTTCTCAGACTTG | 368 |
| S100A14 | 218677_at | -2240432231078.46 | TGTCCTCATCTCTGCAAAGTTCAGC | 369 |
| IGFBP7 | 201162_at | -2225724813680 | TTCCCAAGGACAGGCTTCAGCATCA | 370 |
| PTPRF | 200637_s_at | -2190473907894.45 | CTCCTACGCAGATGCTGTCACTGGC | 371 |
| HMGA1 | 206074_s_at | -2178312788057.87 | TGAGCAAGGGGGCCCGAATCGACCA | 372 |
| YWHAZ | 200641_s_at | -2145016988259.93 | AAGCCTGCTCTCTTGCAAAGACAGC | 373 |
| SCD | 200832_s_at | -2143962895648.8 | TAACTATAAGGTGCCTCAGTTTTCC | 374 |
| SH3YL1 | 204019_s_at | -2139236372988.65 | CATATGGCATCTCTCAACTTTTCTT | 375 |
| UCP2 | 208998_at | -2139031352031.13 | GAAAGTTCAGCCAGAATCTTCGTCC | 376 |
| F3 | 204363_at | -2113802654784.93 | GGGCAGCTTCCTAATATGCTTTACA | 377 |
| AZGP1 | 209309_at | -2089576575474.55 | GCCTGTCTTGAGTAGACTTGGACCC | 378 |
| LIMCH1 | 212327_at | -2089195209441.08 | GATCCACCTCATATGTGAGTCCGTC | 379 |
| PLA2G2A | 203649_s_at | -2069037053701.26 | CGCTGCTGTGTCACTCATGACTGTT | 380 |
| ITGB5 | 201125_s_at | -2028321449243.62 | GCCTGTTGAAGGTACATCGTTTGCA | 381 |
| ABCC3 | 208161_s_at | -2007168680009.07 | TCTCCCGATTCCCAACTGAGTGTTA | 382 |
| DDR1 | 207169_x_at | -2000582844983.07 | AGGCAATTTTAATCCCCTGCACTAG | 383 |
| MIR4640 | | | | |
| GATA3 | 209604_s_at | -1995114130212.84 | GGACAAACTGCCAGTTTTGTTTCCT | 384 |
| CYB561 | 209163_at | -1981172434786.63 | GTTCTTCAATCAGCTGGCACACACT | 385 |
| C10orf116 | 203571_s_at | -1962923571527.29 | ACCACCCAGGAAACCATCGACAAGA | 386 |
| PTPRF | 200635_s_at | -1924144465806.05 | AAGGACAGAACATTGCCTTCCTCGT | 387 |
| DKK1 | 204602_at | -1893211415469.31 | GGATATACAAGTTCTGTGGTTTCAG | 388 |
| SERPINB5 | 204855_at | -1863934443254.52 | GTGGTTGGCACTAGACTGGTGGCAG | 389 |
| ARHGAP29 | 203910_at | -1818117319379.63 | ATGTACTTGTTCTACCTGGATTGTC | 390 |
| GAS6 | 202177_at | -1817533234900.07 | CGCGGCTGCATGACACTGGAGGTCA | 391 |
| LAMB3 | 209270_at | -1817170377879.96 | GGTGCCCGGATCCAGAGTGTGAAGA | 392 |
| KLF5 | 209212_s_at | -1814910338390.4 | CTCCATCCTATGCTGCTACAATTGC | 393 |
| MAOA | 212741_at | -1811716715860.48 | TGAATGCCAGTCCAGATGTGCCTAG | 394 |
| NET1 | 201830_s_at | -1789348130490.25 | TTACATTCATTTAACCTGCCGATTA | 395 |
| CYBA | 203028_s_at | -1775049034494.02 | CACCCAGTGGTACTTTGGTGCCTAC | 396 |
| TGM2 | 201042_at | -1772139742186.19 | AGTGCTGGTCACTAACCAACAAGGT | 397 |
| ALDH2 | 201425_at | -1757839520621.92 | CTCTCTGAAACGCTTCCTATAACTC | 398 |
| HSPA1A | 200799_at | -1730673434053.48 | TTGTCAGTTCTCAATTTCCTGTGTT | 399 |
| HSPA1B | | | | |
| JUP | 201015_s_at | -1729139912998.84 | ATTATCGCTTTATGTTTTTGGTTAT | 400 |
| HSPA1A | 200800_s_at | -1722098969341.57 | GGGGCTCAAGGGCAAGATCAGCGA | 401 |
| HSPA1B | | | G | |
| F11R | 221664_s_at | -1642391094616.93 | GAATAGGTATCTTGAGCTTGGTTCT | 402 |
| HBG1 | 204419_x_at | -1595966820539.76 | ACACTCGCTTCTGGAACGTCTGAGG | 403 |
| HBG2 | | | | |
| LOC | | | | |
| 100653006 | | | | |
| LOC | | | | |
| 100653319 | | | | |
| KLF4 | 221841_s_at | -1553919884310.19 | AATCTATATTTGTCTTCCGATCAAC | 404 |
| CA12 | 214164_x_at | -1551710888005.42 | ACAAGGCCCAGGCTGGGGCCAGGG | 405 |
| | | | C | |

### Kits

Kits of the invention can be used for determining the responsiveness of a cancer patient (e.g., a patient having a solid tumor cancer, such as breast cancer, or a hematological cancer, such as lymphoma (e.g., CTCL) to liposomal formulation of cisplatin (e.g., LiPlaCis). Kits of the invention can include reagents and/or materials for, e.g., collecting and/or purifying nucleic acids from biological samples (such as those obtained from a patient to be treated with a target drug(s) of the invention), reagents for amplifying such nucleic acids to produce an amplified sample, and/or at least one device of the invention. Reagents for amplifying nucleic acids may include, e.g., PCR reagents, including but not limited to DNA polymerase, RNA polymerase, PCR buffer, magnesium chloride solutions, nucleic acid primers (e.g., primers designed to target particular biomarkers of responsiveness to a target drug(s) of interest), and/or any other PCR reagents as are well known in the art. In particular, kits useful in the method may include one or more of the following: a kit for RNA extraction from tumors (e.g., Trizol for mRNA, mirVana miRNA isolation kit from Ambion Inc), a kit for RNA labeling (e.g., MessageAmp from Ambion Inc., FlashTag from Genisphere Inc), a microarray for measuring biomarker expression (e.g., HG-U133A, HG-U133_Plus2 or miRNA-1.0 from Affymetrix Inc), a microarray hybridization station and scanner (e.g., GeneChip System 3000Dx from Affymetrix Inc), and/or software for analyzing the expression of biomarker genes or RNAs (e.g., miRNAs) as described in herein (e.g., implemented in R from R-Project or S-Plus from Insightful Corp.).

For example, a kit of the invention can include one or more probes capable of detecting one or more biomarkers of Tables 2-5 (e.g., the kit may include probes for the biomarkers of Tables 2-5). Such probes can, for example, include nucleic acids capable of hybridizing to the biomarker based on nucleic acid sequence complementarity. In particular, a probe has at least 85% sequence identity (e.g., 85%, 90%, 95%, 97%, 98%, 99%, or 100% sequence identity) to a nucleic acid sequence that is complementary or identical to at least 5 (e.g., at least 15) consecutive nucleotides of one or more biomarkers. The probes can be attached to a solid surface, such as a microarray. The kit may include NanoString capture probes, NanoString reporter probes, and/or one or more nCounter cartridges. The kit may include reagents for next generation sequencing, including but not limited to poly(T) oligonucleotides, dye terminators, sequencing adapters, adapter ligation reagents, reverse transcriptase, primers (e.g., random primers), DNA-cleaving enzymes, polymerases, and/or any combination thereof. The kit may also be one that includes a protein array and/or reagents for detection of the polypeptide product(s) of one or more biomarkers of Tables 2-5.

The following examples are intended to illustrate, rather than limit, the invention.

### EXAMPLES

### Example 1. Identification of Biomarkers of Sensitivity and Resistance to Cisplatin Using Affymetrix HG-U133A Arrays

A key component of LiPlaCis is cisplatin, a common cancer drug that is encapsulated in a liposomal formulation. It is obvious that LiPlaCis will not work on a tumor if cisplatin does not work. Thus is it possible to predict part of the response to LiPlaCis as the response to cisplatin. The liposomal delivery to the tumor cell is a separate part of the requirement for LiPlaCis to work, and can be modeled separately, e.g. by measuring sPLA₂ on the surface of tumor cells.

DNA chip measurements of the 60 cancer cell lines of the NCI60 data set were performed using Affymetrix HG-U133A arrays and logit normalized. For each array, the logit transformation was performed followed by a Z-transformation to mean zero and SD 1, and correlated to growth inhibition (log(G150)). Growth inhibition data of LiPlaCis against the same cell lines were downloaded from the National Cancer Institute. Each gene's expression in each cell line was correlated to the growth of those cell lines (log(G150)) in the presence of LiPlaCis. The Pearson correlation coefficient was then determined to identify genes positively and negatively correlated to sensitivity to LiPlaCis. Tables 2 and 3 show the top positively correlated genes (the biomarkers of sensitivity) and negatively correlated genes (the biomarkers of resistance), respectively, using the Affymetrix HG-U133A arrays.

### Example 2. Identification of Biomarkers of Sensitivity and Resistance to LiPlaCis Using Affymetrix Hg-U133A Arrays

DNA chip measurements of the 60 cancer cell lines of the NCI60 data set were also performed using HG-U133_Plus_2 arrays and logit normalized. For each array, the logit transformation was performed followed by a Z-transformation to mean zero and SD 1, and correlated to growth inhibition (log(G150)). Growth inhibition data of LiPlaCis against the same cell lines were downloaded from the National Cancer Institute. Each gene's expression in each cell line was correlated to the growth of those cell lines (log(G150)) in the presence of LiPlaCis. The covariance (Pearson correlation coefficient multiplied by standard deviation) was then determined to identify genes positively and negatively correlated to sensitivity to LiPlaCis. Tables 4 and 5 show the top positively correlated genes (the biomarkers of sensitivity) and negatively correlated genes (the biomarkers of resistance), respectively, using the Affymetrix HG-U133A arrays.

### Example 3. Predicting Responsiveness to LiPlaCis in Various Cancer Patient Populations

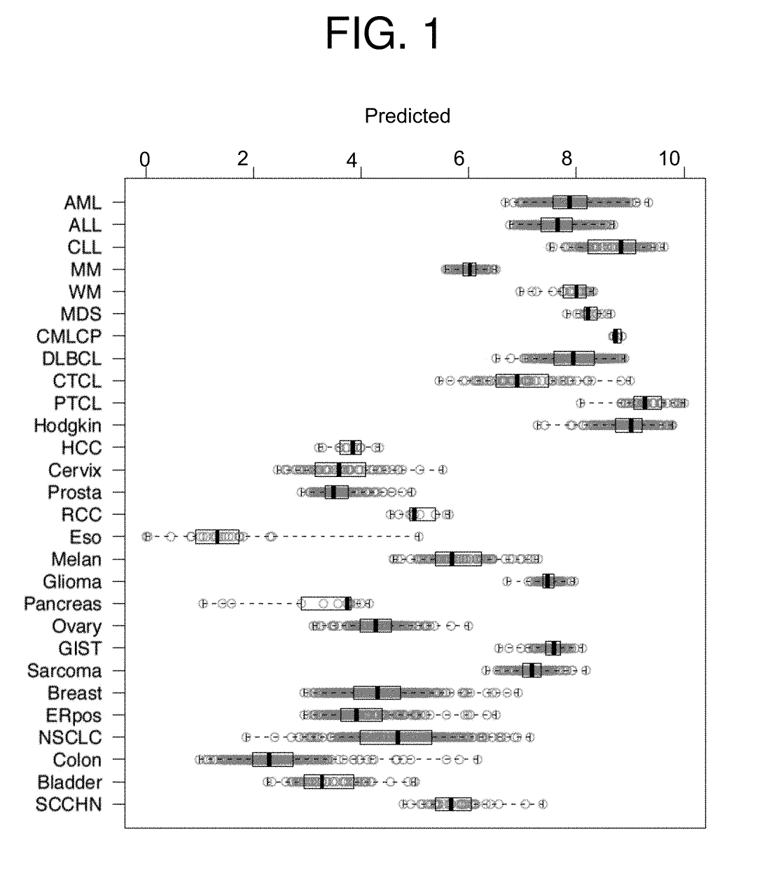

An mRNA-based predictor of responsiveness to LiPlaCis developed according to the methods of the invention was applied to 3,522 patients having a variety of cancers. Each patient had a pre-treatment measurement of gene expression with an Affymetrix array. The predicted LiPlaCis sensitivity of each patient was calculated as the difference between the mean of the expression levels of the biomarkers of sensitivity (Table 2) and the mean of the expression levels of the biomarkers of resistance (Table 3) for the patient. When the patients were grouped by cancer types, and cancer types predicted to be more responsive to LiPlaCis were identified (FIG. 1).

Of 27 different cancer types, solid tumor cancers were predicted to be more responsive to LiPlaCis treatment than hematological cancers. In particular, patients with hematological cancer types were predicted to be responsive to LiPlaCis treatment.

The median of the boxplots shown in FIG. 1 is a cutoff that may be used to separate patients predicted to be responsive to LiPlaCis treatment from patients predicted to be non-responsive to LiPlaCis treatment for a given cancer type. Values above the median indicate patients predicted to be responsive to LiPlaCis, while values below the median indicate patients predicted to be non-responsive to LiPlaCis. For a test sample from an individual patient, it is useful to compare the test sample to the reference population for the same cancer type. If the test sample is above the median for the reference population of the same cancer type, then the patient is predicted to be responsive to LiPlaCis treatment. If the test sample is below the median for the reference population of the same cancer type, then the patient is predicted to be non-responsive to LiPlaCis treatment. This method for predicting patient responsiveness can also be used when the reference cancer population consists of only two patients: a patient responsive to LiPlaCis treatment and a patient non-responsive to LiPlaCis treatment.

### Example 4. Determining the Expression of Secreted Phospholipase A2

In addition to determining the responsiveness to cisplatin or LiPlaCis using the genes in Tables 2-5, it is also possible to test for the presence of secreted phospholipase A2 (sPLA2-IIA) in the tumor tissue. sPLA2 is required for degradation of the liposomes that deliver the cisplatin to the tumor cell, and can be measured using standard immunocytochemistry techniques with a monoclonal antibody against sPLA2-IIA, e.g. Clone SCACC353 from Cayman Chemical. Any staining in this assay indicates the presence of sPLA2 and suggests susceptibility to LiPlaCis. Alternatively, the expression of sPLA2-IIA can be detected on the microarray as PLA2G2A (SEQ ID NO: 380). While in cancer cell lines growing in vitro there is a negative covariance between PLA2G2A expression and LiPlaCis response, in tissue there is a positive correlation between sPLA2A mRNA and immunohistochemistry (Mirtti et al APMIS 2009, 117: 151-161).

### Example 5. Predicting Responsiveness of Breast Cancer Patients to LiPlaCis

The diagnostic methods of the present invention can be used to predict the responsiveness of a breast cancer patient to treatment with LiPlaCis. In particular, the breast cancer patient may be one that has not previously received any cancer treatment or one that has received a cancer treatment other than LiPlaCis. Moreover, the patient may be one diagnosed with breast cancer or one with recurrence of prostate cancer.

A biological sample (e.g., a breast cancer tissue sample) may be obtained from the patient through methods well known in the art. The sample may be frozen and/or prepared, e.g., by formalin fixation and paraffin embedding. In particular, mRNA can be isolated from the sample and a gene expression profile can be determined, e.g., using a microarray platform, such as the Affymetrix HG-U133A or HG-U133_Plus_2 array, for one or more of the biomarkers shown in Tables 2-5. One or more of the biomarkers shown in Tables 2-5 can also be measured, e.g., by sequencing or PCR-based techniques, such as those described herein.

For example, the expression level of one or more biomarkers of sensitivity to LiPlaCis can be determined in the sample from the patient, such as one or more of COL5A2 (SEQ ID NO 73 or 211), ITGA4 (SEQ ID NO: 1), MSN (SEQ ID NO: 2), FAM46A (SEQ ID NO: 3 or 280), ITGB2 (SEQ ID NO: 4), DOCK2 (SEQ ID NO: 5 or 223), EVL (SEQ ID NO: 6), SACS (SEQ ID NO: 7), PTPRCAP (SEQ ID NO: 8), EBI2 (SEQ ID NO: 9), PTPRC (SEQ ID NO: 10, 18, 25, or 243), ANP32E (SEQ ID NO: 11), SFPQ (SEQ ID NO: 12, 38 or 272), C1QR1 (SEQ ID NO: 13), FNBP1 (SEQ ID NO: 14 or 28), CBFB (SEQ ID NO: 15). In particular, the biomarker is COL5A2 (SEQ ID NO 73 or 211). The expression level of one or more biomarkers of resistance to LiPlaCis can also be determined in the sample from the patient, such as one or more of SFN (SEQ ID NO: 96 or 324), LISCH7 (SEQ ID NO: 97), EPB41L4B (SEQ ID NO: 98), MST1R (SEQ ID NO: 99), ITGB4 (SEQ ID NO: 100), DBNDD2 (SEQ ID NO: 102 or 365), TACSTD1 (SEQ ID NO: 104), MISP (SEQ ID NO: 105), KRT8 (SEQ ID NO: 106), JUP (SEQ ID NO: 107 or 400), KRT18 (SEQ ID NO: 108 or 306), FA2H (SEQ ID NO: 109), MGAT4B (SEQ ID NO: 110), DSG2 (SEQ ID NO:111 or 312), LRP5 (SEQ ID NO: 112). In particular, the biomarker is SFN (SEQ ID NO: 96 or 324).

The breast cancer patient may be responsive to LiPlaCis if the expression level of one or more of the biomarkers of sensitivity is substantially similar to the expression level of the biomarkers of sensitivity in a cell or tissue known to be sensitive to LiPlaCis. The breast cancer patient may also be responsive to LiPlaCis if the expression level of one or more of the biomarkers of resistance is substantially dissimilar to the expression level of the biomarkers of resistance in a cell or tissue known to be resistant to LiPlaCis.

In addition to determining the responsiveness to cisplatin or LiPlaCis using the genes in Tables 2-5, it is also possible to test for the presence of secreted phospholipase A2 (sPLA2-IIA) in the tumor tissue. sPLA2 is required for degradation of the liposomes that deliver the cisplatin to the tumor cell, and can be measured using standard immunocytochemistry techniques with a monoclonal antibody against sPLA-IIA, e.g. Clone SCACC353 from Cayman Chemical. Any staining in this assay indicates the presence of sPLA2 and suggests susceptibility to LiPlaCis. Alternatively, the expression of sPLA2-IIA can be detected on the microarray as PLA2G2A (SEQ ID NO: 380). While in cancer cell lines growing in vitro there is a negative covariance between PLA2G2A expression and LiPlaCis response, in tissue there is a positive correlation between sPLA2A-IIA mRNA and immunohistochemistry (Mirtti et al APMIS 2009, 117: 151-161)

If the patient is predicted to be responsive, then the patient can be administered LiPlaCis, such as LiPlaCis administered intravenously at a dose of about 75 mg, or about 90 mg, or about 40 mg/mm² body surface area, or about 55 mg/mm² body surface area on day 1 and day 8 of a three week regimen. Conversely, if the patient is predicted to be non-responsive to LiPlaCis treatment, then the patient can be administered one or more therapies other than LiPlaCis.

### Example 6. Correlation Between DRP Score and Clinical Response (RECIST) in Advanced Breast Cancer Patients

The cisplatin response profile described in Example 1 and using the biomarkers of Tables 2-3 was validated in a Phase I/II clinical study. The purpose of the study was to correlate the DRP score to the response of the patients to LiPlaCis. The study population consisted of advanced breast cancer patients who provided informed consent to be included in a clinical trial of LiPlaCis and its companion diagnostic DRP (clinicaltrials.gov number NCT01861496). Ten hospitals in Denmark collected diagnostic biopsies from advanced breast cancer patients diagnosed between 1997 and 2016 with a mixture of receptor status. Twelve patients were above the cutoff 33 used for inclusion in the trial and were initiated on LiPlaCis treatment. Ten patients were evaluable for response. The overall outcome of the ten patients are described in Table 6.

TABLE 6
Overall outcome of first 10 patients in LiPlaCis

| | | | | | | | Duration |
|---|---|---|---|---|---|---|---|
| | | | | | | | of latest |
| | | | | | | | treatment |
| | | | Best | | Unique | | before |
| | DRP | Weeks in | response in | Line | anticancer | Platins | LiPlaCis |
| All | score | study | LiPlaCis | treatments | treatments | (line) | in weeks |
| Patient #1 | 99 | 32 | PR | 6 | 9 | 0 | 17 |
| Patient #2 | 35 | 13 | SD | 12 | 13 | Carboplatin | 19 |
| | | | | | | (12) | |
| Patient #3 | 66 | 5 | PD | 10 | 13 | Carboplatin | 9 |
| | | | | | | (12) | |
| Patient #4 | 94.9 | 6 | PD | 8 | 16 | (4) | 8 |
| | | | | | | Oxaliplatin inter | |
| | | | | | | Hepar | |
| Patient #5 | 94.3 | 36 | SD+24 weeks | 6 | 8 | 0 | 20 |
| Patient #6 | 80.1 | 25 | PR | 8 | 10 | 0 | 5 |
| Patient #7 | 39.4 | 9 | PD | 8 | 9 | 0 | 7 |
| Patient #8 | 94.9 | 12 | SD | 5 | 8 | 0 | 12 |
| Patient #9 | 80.6 | 18 | SD | 4 | 4 | 0 | 17 |
| Patient #10 | 39.4 | 6 | PD | 9 | 11 | 0 | 12 |
| Mean | 72.36 | 16.19 | | 7.60 | | | 12.60 |
| Median | 80.35 | 12.43 | | 8.00 | | | 12.00 |

| | | Gain in | | | Gain in | |
|---|---|---|---|---|---|---|
| | | relation | | Mean | relation | |
| | | to latest | Best | duration of | to mean | |
| | | previous | response | treatments | previous | |
| | | treatment | before | before | treatment | comments + |
| All | Outcome | in weeks | LiPlaCis | LiPlaCis | in weeks | best response |
| Patient #1 | SD | 15 | SD^{+24 weeks} | 33.71 | −1.57 | BR previous: |
| | | | | | | SD 46 weeks |
| Patient #2 | SD | −6 | PR | 16.26 | −3.40 | Carboplatin, (in |
| | | | | | | combination with |
| | | | | | | trastuzumab) (SD) |
| Patient #3 | SD | −4 | CR | 38.10 | −33.24 | Carboplatin, |
| | | | | | | (i kombimed |
| | | | | | | Gemcitabin + |
| | | | | | | trastuzumab) (SD) |
| Patient #4 | PD | −2 | SD^{+24 weeks} | 26.33 | −19.90 | Oxaliplatin, (in |
| | | | | | | combination with |
| | | | | | | capecitabine/5FU + |
| | | | | | | trastuzumab). BR: |
| | | | | | | SD 78 weeks |
| Patient #5 | SD | 16 | SD^{+24 weeks} | 27.93 | 8.21 | BR: SD 60 weeks |
| Patient #6 | PD | 20 | SD^{+24 weeks} | 19.24 | 5.90 | BR previous: SD 52 |
| | | | | | | weeks |
| Patient #7 | PD | 2 | SD^{+24 weeks} | 29.43 | −20.43 | BR: SD 25 weeks |
| Patient #8 | PD | 0 | SD | 11.07 | 0.93 | BR: SD 16 weeks |
| Patient #9 | SD | 1 | SD | 8.32 | 9.25 | BR previous: |
| | | | | | | SD 17 weeks |
| Patient #10 | PD | −6 | SD^{+24 weeks} | 13.64 | −7.93 | BR: SD 36 weeks |
| Mean | | 3.59 | SUM | 224.04 | −62.18 | |
| Median | | 0.29 | Mean | 22.40 | −6.22 | |

Of these, three patients had received prior treatment with a platinum based compound, which could interfere with the ability of the DRP to predict response to subsequent platinum based treatment unless a new biopsy is obtained.

The overall outcome of the top third DRP, excluding patients who had previously been treated with platins is outlined in Table 7.

TABLE 7
Top third DRP excluding patients treated with platins before LiPlaCis treatment

| | | | | | Duration of | Gain in | | Gain in |
|---|---|---|---|---|---|---|---|---|
| DRP > 66 | | | | | latest | relation to | Mean | relation to |
| and no | | | | | treatment | latest | duration of | mean of all |
| previous | | | | | before | previous | treatments | previous |
| platin- | DRP | Weeks in | Best | Line | LiPlaCis/ | treatment | before | treatment |
| treatment | score | study | response | treatments | Outcome | in weeks | LiPlaCis | in weeks |
| Patient #1 | 99 | 32 | PR | 8 | 17 | 15 | 33.71 | −1.57 |
| Patient #8 | 94.9 | 12 | SD | 7 | 12 | 0 | 11.07 | 0.93 |
| Patient #5 | 94.3 | 36 | SD^{+24 weeks} | 7 | 20 | 16 | 27.93 | 8.21 |
| Patient #9 | 80.6 | 18 | SD | 3 | 17 | 1 | 8.32 | 9.25 |
| Patient #6 | 80.1 | 25 | PR | 9 | 5 | 20 | 19.24 | 5.90 |
| Mean | 89.78 | 24.60 | | 6.80 | 14.20 | 10.40 | 20.06 | 4.54 |
| Median | 94.30 | 25.14 | | 7.00 | 17.00 | 15.14 | 19.24 | 5.90 |

The statistical analysis was pre-planned in a statistical analysis plan (v2.0, Jun. 21, 2017) before initiation of analysis. The primary analysis was a one-sided Pearson correlation between the DRP score and the tumor response (RECIST criteria encoded as 4,3,2,1 for CR (complete remission), PR (partial response), SD (stable disease), PD (progressive disease), respectively). A secondary analysis was a cox proportional hazards analysis of time to progression or death using the median DRP score as a cutoff. A logrank test with a p-value of 0.05 or less was considered significant. Patients were stratified according to prior platinum treatment.

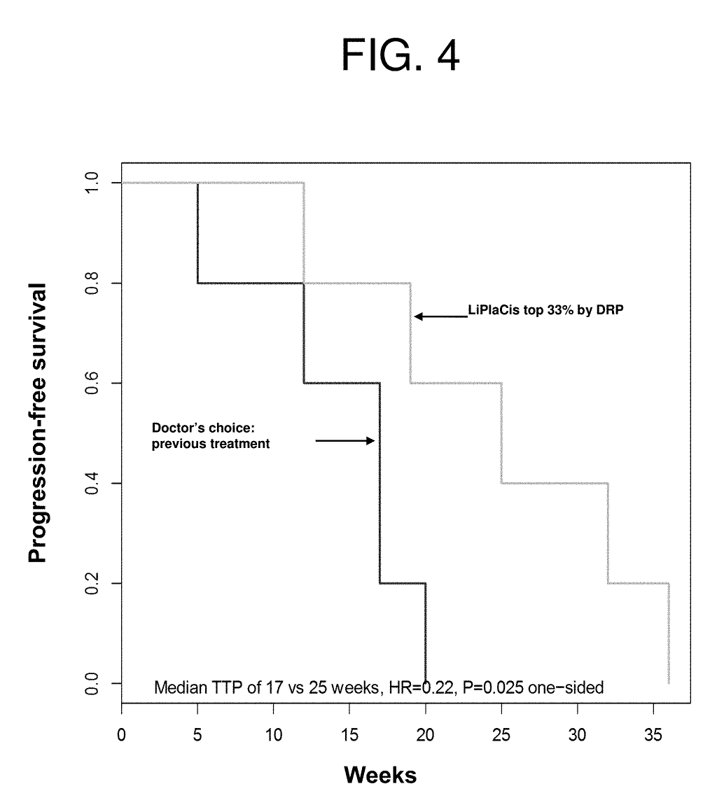

Patients treated with LiPlaCis on average had 8 prior treatments. Patients in the upper tertile defined by the DRP on average had a longer duration of treatment than on all prior treatments (median 25 weeks versus 20 weeks). FIG. 4 shows the duration of treatment on LiPlaCis compared to the most recent prior treatment. Comparing the LiPlaCis treatment to the most recent prior treatment is a surrogate for “doctor's choice” often used in randomized trials. The most recent prior treatment is by doctor's choice, but patients were not treated while on study, thus the frequency of response evaluation and the depth of data monitoring might not be the same as when patients entered the study.

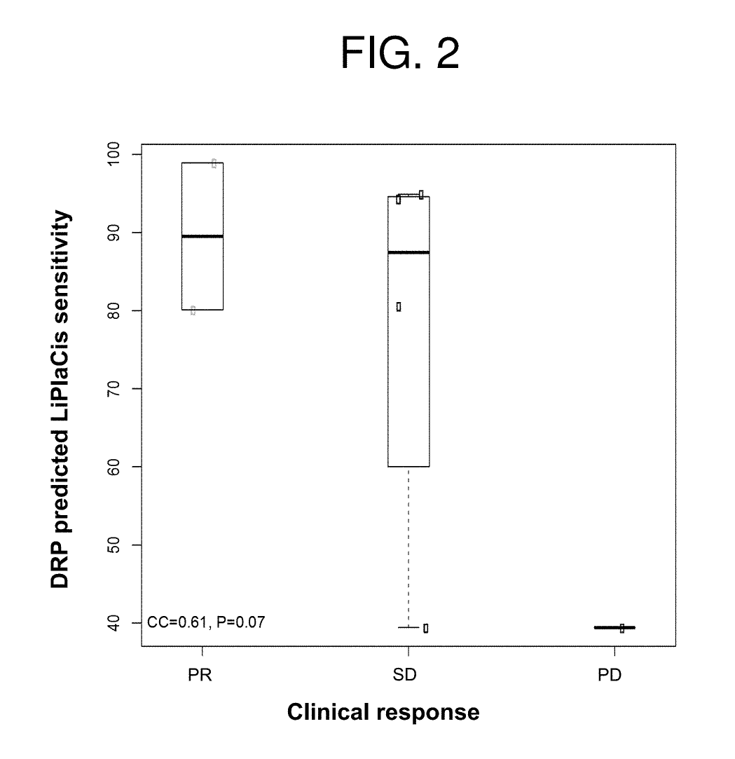

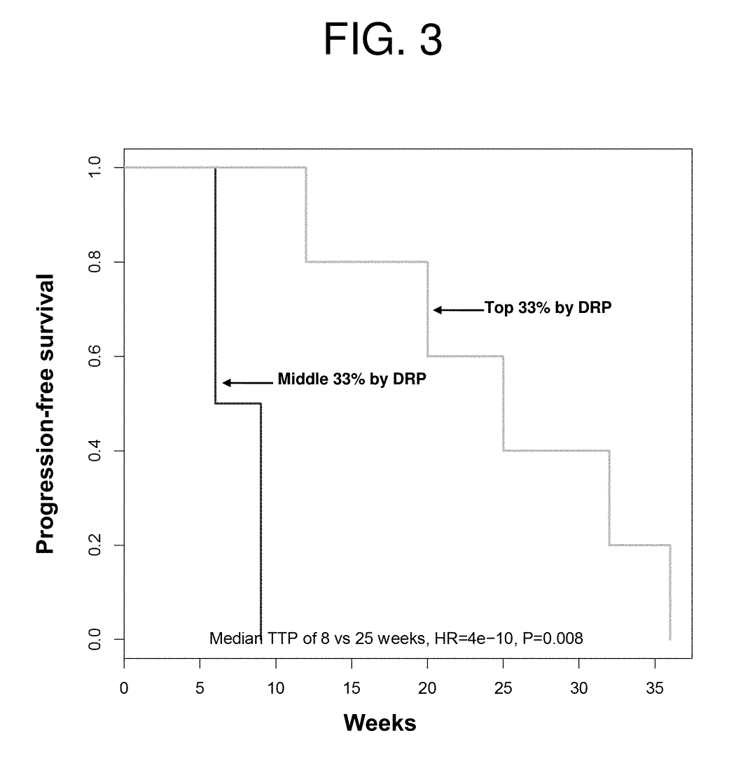

The DRP was very precise in predicting who will benefit from LiPlaCis in this study population. Patients in the upper tertile performed much better than patients in the middle tertile as defined by the DRP score (FIGS. 2 and 3). Patients in the upper tertile as defined by DRP score also, on average, benefited more from LiPlaCis treatment than from all other previous treatments. The improvement was most dramatic when comparing to the most recent treatment, where patients' risk per time unit of terminating drug was four times lower on LiPlaCis (HR=0.22, FIG. 4) than on the previous treatment.

In other words, for all patients in the upper tertile defined by DRP, LiPlaCis was, on average, a clear improvement over the previous treatment.

Further trials should as soon as possible confirm this result so breast cancer patients in the future can gain the obvious and clear clinical benefit from treatment with LiPlaCis

### Example 8. Response of DRP Positive Patients to LiPlaCis Treatment

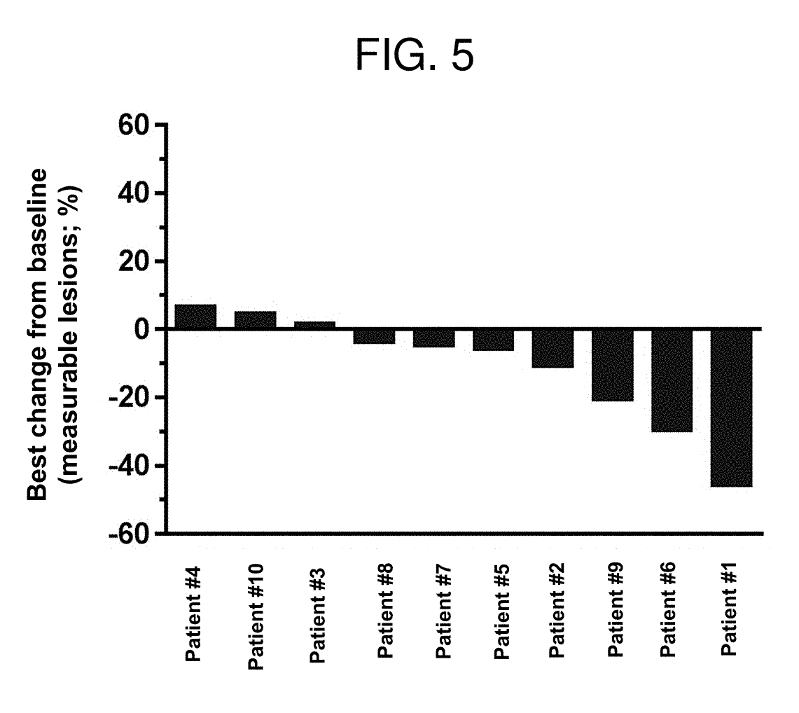

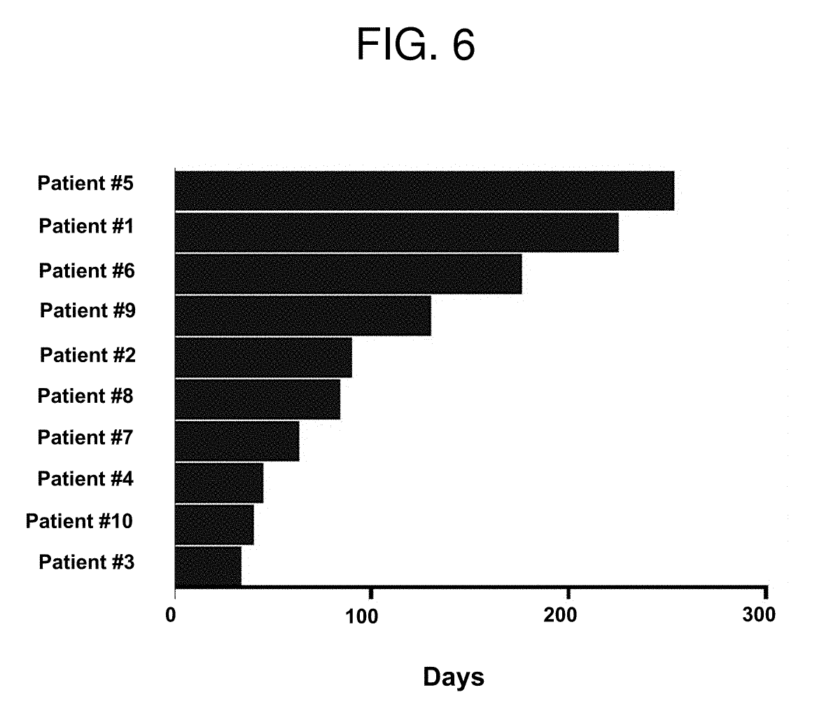

The effectiveness of LiPlaCis administered as per the dosage regimen described herein was validated in a PhaseI/II clinical study. The study population consisted of advanced breast cancer patients who had been identified as DRP positive as per the methods described herein. The patients received LiPlaCis in the following dosage regimen: 2 doses of 75 mg each, administered on day 1 and day 8 of three week treatment cycle/s. Table 8 and FIG. 5 elucidate the promising response of the DRP positive patients to this LiPlaCis treatment regimen. The duration of treatment in these patients is illustrated in FIG. 6.

TABLE 8
Status and response of DRP positive patients to LiPlaCis treatment.

| Subject | | | | | Best | DRP |
|---|---|---|---|---|---|---|
| No. | Age | First dose | Cycles | Status | response | score |
| Patient #1 | 51 | 30 May 2016 | 8 full cycles →4 doses | Off Study | PR | 99 |
| | | | (every 2nd week) | | | |
| Patient #2 | 55 | 17 Jan. 2017 | 4 (1 treatment cancelled) | Off Study | SD | 35 |
| Patient #3 | 71 | 31 Jan. 2017 | 2 (1 treatment cancelled) | Off Study | PD | 66 |
| Patient #5 | 47 | 27 Mar. 2017 | 10 | Off Study | PR | 94.3 |
| Patient #6 | 51 | 23-maj-17 | 8.5 | Off Study | PR | 80.1 |
| Patient #7 | 59 | 22 Aug. 2017 | 3 | Off Study | SD | 39.4 |
| Patient #9 | 73 | 14 Sep. 2017 | 6 | Ongoing | SD | 80.6 |
| Patient #8 | 52 | 18 Sep. 2017 | 4 | Off Study | SD | 94.9 |
| Patient #10 | 61 | 04-okt-17 | 2 | Off Study | PD | 39.4 |
| Patient #11 | 60 | 04-okt-17 | 1 treatment | Off Study | NA - erstattes | 46.5 |
| | | | | Dead/Cioms | | |
| Patient #12 | 60 | 05-okt-17 | 1 treatment | Off Study | NA - erstattes | — |
| | | | | Dead/PD | | |
| Patient #4 | 51 | 14 Nov. 2017 | 2 | Off Study | PD | 94.9 |

### Example 9. Analysis of Adverse Effects of LiPlaCis

Twelve patients treated with LiPlaCis in a Phase II clinical study, were analyzed for adverse effects of LiPlaCis, if any. The interim data shows that LiPlaCis is well tolerated, with mainly mild and only few moderate side effects; only four grade 3 events and two grade 4 events being recorded as related to study drug in the treated patients. While ototoxicity and nephrotoxicity are well known and frequent related adverse events to conventional cisplatin, no clinically relevant ototoxicity and nephrotoxicity was observed with LiPlaCis. Both ototoxicity and nephrotoxicity occurred at a much lower and milder grades than known with cisplatin. Fever, cytopenia, or clinically relevant platelet toxicity was also not observed. Hand-foot syndrome, a possible adverse effect due to liposomal drug delivery was expected, but not found in the study cohort. Conventional cisplatin treatment of metastatic breast cancer has a 10% response rate.

### Example 10. Preparation of LiPlaCis for Administration

According to the methods described herein, LiPlaCis infusion liquid can be prepared by withdrawing the required amount of concentrate from vials of LiPlaCis Concentrate for Infusion, and diluting it in two infusion bags, each bag containing 50% of the dose. The amount of concentrate to be withdrawn from the vials of LiPlaCis Concentrate for Infusion can be calculated according to the dose that is to be administered, and the concentration of cisplatin in the LiPlaCis Concentrate for Infusion, as stated in the label. For example, if a patient is to receive a dose of 75 mg of LiPlaCis, which is to be prepared from vials of LiPlaCis Concentrate for Infusion, where the concentration of cisplatin is labeled as 1.1 mg/ml, the amount of LiPlaCis that is to be withdrawn from the vial can be calculated as follows:

Vₜₒₜ=75 mg/1.1 mg/ml=68.2 ml

The required number of vials of LiPlaCis Concentrate for Infusion can be thawed, 68.2 ml can be withdrawn, and 34.2 ml can be added to each of two infusion bags (each bag containing 0.9% sodium chloride, 500 ml) via the medication valve. The infusion liquid can be mixed thoroughly, kept protected from light, and used within 8 hours.

### Example 11. Treating a Breast Cancer Patient with LiPlaCis

A physician of skill in the art can treat a patient, such as a human patient with cancer (e.g., breast cancer) by administering LiPlaCis as per the dosage regimens described herein. For example, a patient can be administered two doses of cisplatin, each of about 75 mg of cisplatin, or 90 mg of cisplatin, or each dose comprising cisplatin amounting to about 40 mg/mm² body surface area, or about 55 mg/mm² body surface area, on day 1 and day 8 of a three week treatment cycle. The regimen can be repeated for 3 cycles or more. Alternatively, the patient can also be treated by administering escalated doses of cisplatin in subsequent treatment cycles. For example, a patient can be administered two doses of cisplatin, each of about 75 mg of cisplatin, or comprising cisplatin amounting to about 40 mg/mm² body surface area on day 1 and day 8 of the first three week treatment cycle, followed by two doses of cisplatin, each of about 90 mg of cisplatin, or comprising cisplatin amounting to about 55 mg/mm² body surface area on day 1 and day 8 of the next three week treatment cycle. Alternatively, a patient can also be administered two doses of cisplatin, each of about 75 mg of cisplatin, or comprising cisplatin amounting to about 40 mg/mm² body surface area on day 1 and day 8 of the first and second three week treatment cycles, followed by two doses of cisplatin, each of about 90 mg of cisplatin, or comprising cisplatin amounting to about 55 mg/mm² body surface area on day 1 and day 8 of the third three week treatment cycle. Alternatively, a patient can also be administered two doses of cisplatin, each of about 75 mg of cisplatin, or comprising cisplatin amounting to about 40 mg/mm² body surface area on day 1 and day 8 of the first three week treatment cycle, followed by two doses of cisplatin, each of about 90 mg of cisplatin, or comprising cisplatin amounting to about 55 mg/mm² body surface area on day 1 and day 8 of the second and third three week treatment cycles.

### Example 12. Evaluating Safety and Tolerability, and Determining Maximum Tolerable Dose (MTD) of LiPlaCis

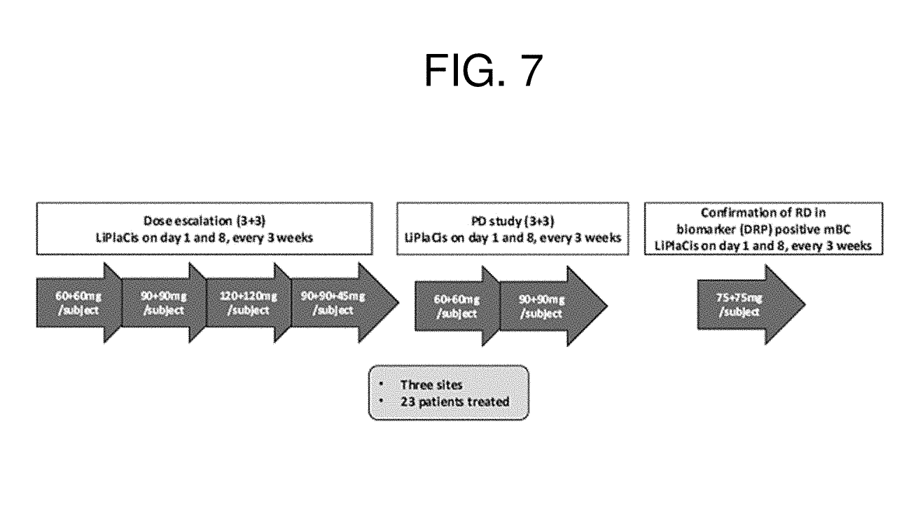

A Phase I/II study was conducted to evaluate safety and tolerability and to determine the maximum tolerated dose (MTD) of LiPlaCis (Liposomal Cisplatin formulation) in patients with advanced or refractory tumors (see FIG. 7). In cohort B and in dose step 5 (after 20 patients; see below), the patient population was limited to skin cancer (not screened for sensitivity) and metastatic breast cancer patients screened by the LiPlaCis DRP (described herein) to be sensitive to LiPlaCis. A Pharmacodynamic (PD) Proof of Concept study was performed in a cohort of 6 patients to investigate the targeted delivery of cisplatin (the active drug in LiPlaCis) in the tumor. Data from this study showed a 5-28-fold increase in DNA platinum adducts (GG-Pt) in tumor tissue over normal tissue of the same patient, compared to a 4-6-fold increase of DNA-platinum (GG-Pt) that is seen with conventional cisplatin, indicating targeted delivery of cisplatin to tumor with LiPlaCis.

### Primary Objectives of the Study:

To evaluate the safety and tolerability of LiPlaCis given on day 1 and day 8 (and possible day 15) every 3 weeks.
To determine the MTD and the recommended dose (RD) of LiPlaCis given on day 1 and day 8 (and possible day 15) every 3 weeks.

### Secondary Objectives of the Study:

To evaluate pharmacokinetics (PK) of LiPlaCis given on day 1 and day 8 (and possible day 15) every 3 weeks.
To evaluate the therapeutic efficacy of LiPlaCis given on day 1 and day 8 (and possible day 15) every 3 weeks.
To evaluate the pharmacodynamics (PD) of LiPlaCis in selected patients.
Progression-free survival (PFS) for patients from dose step 5.

### Disposition of Subjects and Exposure:

Thirty patients were included in the phase I/II study. Four patients were included in dose step 1 (60+60 mg), one patient (Patient #14) was not properly screened and was replaced. Four patients were included in dose step 2 (90+90 mg), 3 patients were included in dose step 3 (120+120 mg) and two patients were included in the dose step 4 (90+90+45 mg). At dose step 4, both patients were withdrawn from the study, one due to infusion reaction and the other due to rapid progression of disease. Three patients were included in cohort A, and four patients in cohort B as one patient (Patient #30) was replaced. Seven patients were included in dose step 5. Table 9 outlines demographics, exposure, response and prior treatment in 25 patients.

TABLE 9
Demographics, exposure, response and prior treatment on 25 patients from Phase I/II study

| | | | | Dose, | | | Previous lines | Best |
|---|---|---|---|---|---|---|---|---|
| Subject No. | Gender | Age | Diagnosis | mg/subject | Cycles adm. | | of treatment | response |
| Patient #13 | M | 64 | Hepatocellular | 60 + 60 mg | 2 | Cycles | 1 | PD |
| Patient #14 | F | 55 | Colorectal | 60 + 60 mg | ½cycle | | 5 Incl. | PD |
| | | | Cancer - | | | | Oxaliplatin | |
| | | | Adenocarcinoma | | | | | |
| Patient #15 | M | 66 | Colorectal | 60 + 60 mg | 2 | Cycles | 4 Incl. | PD |
| | | | Cancer - | | | | Oxaliplatin | |
| | | | Adenocarcinoma | | | | | |
| Patient #16+ | F | 57 | NSCLC, | 60 + 60 mg | 2 | Cycles | 5 Incl. | PD |
| | | | Adenocarcinoma | | | | Carboplatin | |
| Patient #17*+ | F | 71 | Colon Cancer - | 90 + 90 mg | 6 | Cycles | 3 Incl. | SD-18 weeks |
| | | | Adenocarcinoma | | | | Oxaliplatin | |
| Patient #18′ | M | 52 | Esophagus | 90 + 90 mg | 8 | Cycles | 4 Incl. | PR23 weeks |
| | | | Cancer - | | | | Carboplatin | |
| | | | Adenocarcinoma | | | | | |
| Patient #19 | F | 60 | Colorectal | 90 + 90 mg | 2 | Cycles | 4 Incl. | PD |
| | | | Cancer - | | | | Oxaliplatin + | |
| | | | Adenocarcinoma | | | | Cis | |
| Patient #20+ | F | 60 | Colorectal | 120 + 120 mg | 1 | Cycle | 8 Incl. | PD |
| | | | Cancer - | | | | Oxaliplatin | |
| | | | Adenocarcinoma | | | | Carboplatin + | |
| Patient #21*+ | M | 65 | Cancer cutis - | 120 + 120 mg | 3 | Cycles | 2 Incl. | PR |
| | | | Squamous cell | | | | Carboplatin | CR after |
| | | | carcinoma | | | | | Operation |
| Patient #22+ | F | 50 | Colon Cancer - | 120 + 120 mg | 1½ | Cycles | 3 Incl. | PD |
| | | | Adenocarcinoma | | | | Oxaliplatin | |
| Patient #23 | M | 44 | NSCLC - | 90 + 90 mg | 2 | Cycles | 5 Incl. | PD |
| | | | Adenocarcinoma | | | | Carboplatin | |
| Patient #24 | M | 59 | NSCLC - | 90 + 90 + 45 mg | 2 | Cycles | 2 Incl. | PD |
| | | | Squamous cell | | | | Carboplatin | |
| | | | carcinoma | | | | | |
| Patient #25 | M | 60 | Pancreatic | 90 + 90 + 45 mg | ⅓ of | Cycle | 2 Incl. | PD |
| | | | Cancer - | | | | Oxaliplatin | |
| | | | Adenocarcinoma | | | | | |
| Patient #26* | M | 59 | Larynx cancer - | 60 + 60 mg | 6 | Cycles | 6 Incl. | SD-23 weeks |
| | | | Planocellulaer | | | | Carboplatin + | |
| | | | carcinoma | | | | Cis | |
| Patient #27 | M | 48 | Gastric - | 60 + 60 mg | 3 | Cycles | 4 Incl. | SD-8 weeks |
| | | | Mixed Adeno- | | | | Carboplatin + | |
| | | | neuroendocrine | | | | Cis | |
| | | | Carcinoma | | | | | |
| Patient #28* | F | 47 | Breast cancer - | 60 + 60 mg | 4 | Cycles | 8 Incl. | SD-14 weeks |
| | | | Adenocarcinoma | | | | Carboplatin | |
| Patient #29*+ | F | 38 | Breast cancer | 90 + 90 mg | 6 | Cycles | 8 | SD-18 weeks |
| Patient #30+ | F | 62 | Pancreatic | 90 + 90 mg | ½ | cycle | 3 Incl. | PD |
| | | | cancer - | | | | Oxaliplatin | |
| | | | Adenocarcinoma | | | | | |
| Patient #31*+ | M | 72 | Liver cancer - | 90 + 90 mg | 6 | Cycles | 3 | SD-18 weeks |
| | | | hepatocellular | | | | | |
| | | | carcinoma | | | | | |
| Patient #32+ | M | 64 | Colon cancer - | 90 + 90 mg | 1 | Cycle | 3 | PD |
| | | | Adenocarcinoma | | | | | |
| Patient #33*+ | F | 50 | Breast cancer - | 75 + 75 mg | 10 | Cycles | 8 | PR-32 weeks |
| | | | Ductal | | | | | |
| | | | carcinoma | | | | | |
| Patient #34+ | F | 55 | Breast cancer - | 75 + 75 mg | 4 | Cycles | 12 Incl. | SD-13 weeks |
| | | | Carcinoma in | | | | Carboplatin | |
| | | | situ | | | | | |
| Patient #35+ | F | 72 | Breast cancer - | 75 + 75 mg | 2 | Cycles | 12 Incl. | PD |
| | | | Carcinoma | | | | Carboplatin | |

| | | | | | | | |
|---|---|---|---|---|---|---|---|
| Patient #36*+ | F | 46 | Breast cancer | 75 + 75 mg | 9 Cycles - | 7 | SD-27 weeks |
| | | | | | ongoing | | |
| Patient #37*+ | F | 50 | Breast cancer - | 75 + 75 mg | 6 Cycles - | 9 | SD 19 weeks |
| | | | Carcinoma | | ongoing | | |

*Narratives describing individual cases of patients responding to LiPlaCis therapy.
+DRP evaluated patients

### Dose Step 1 (60+60 mg):

No dose-limiting toxicity (DLT) was reported for this dose level. Three severe adverse events (SAEs) were reported. Two hospitalizations due to drug related reversible fever and one hospitalization due to hypomagnesaemia to administer IV magnesium were reported. It was decided to escalate the dose to 90+90 mg.

### Dose Step 2 (90+90 mg):

No DLTs were reported for this dose level. Patient 01-006 had creatinine and Cr-EDTA values that corresponds to grade 2. Patient 01-005 and 01-007 experienced a rise in temperature corresponding to a grade 1 and grade 2, respectively. According to protocol, the next dose step should have been 135+135 mg, however it was decided that a dose increase from a total dose of 180 mg to 270 mg was a too large dose step to take and the dose should be increased to only 120+120 mg.

### Dose Step 3 (120+120 mg):

Two DLTs were reported at this dose step after inclusion of 3 patients. Kidney toxicity and 2 SAEs were reported for patient 01-008 and 01-010.

### Dose Step 4 (90+90+45 mg):

The next three patients that were included received 90+90 mg (01-011), 90+90+45 mg (01-012) and 90+90+45 mg (01-013). The day 15 treatment of 45 mg (Cohort 4) was added to investigate if a three-weekly schedule was feasible. At the same time, paracetamol was given prophylactic as pre-medication and the infusion time was increased to 3 hours for dosages above 90 mg to prevent infusion related reactions. Furthermore Cr-EDTA on day 8 prior to treatment was implemented.

### Measurement of PD Markers

Cohorts A (60+60 mg) and B (90+90 mg) were used to measure PD before the dose for the extension phase was decided. Total platinum, DNA-platinum and sPLA₂-IIA protein levels was measured in these. The dose for the extension phase was decided to be 75+75 mg due to fatigue at dose step 90 +90 mg. The regimen of pre-medication was extended with prednisolone and ibuprofen. Post hydration was prolonged at the same time to protect the kidneys.

### Dose Step 5 (75+75 mg) Chosen to be the RD

In this phase II part of the study only DRP screened advanced breast cancer patients and a few not screened skin cancer patients were enrolled. Recommended Dose (RD) was chosen at 75+75 mg, though there was no Dose Limiting Toxicity (DLT) at the 90+90 mg level.

### Adverse Events in 25 Patients

In the ongoing study, all patients experienced one or more treatment emergent adverse events (TEAEs), and in all except 1 patient, one or more of the TEAEs were considered LiPlaCis related. A total of 485 AEs were reported for 25 patients, of whom 2 patients were ongoing, of these 62% were deemed possibly related to the study drug. Most of the LiPlaCis-related AEs were of mild to moderate severity, i.e., in 40% (10/25) of the patients. Severe TEAEs were reported for 12 patients (48%); in 8 patients (32%), one or more of the severe TEAEs were considered LiPlaCis-related, 3 patients in the 60+60 mg, 3 patients in the 90+90 mg, and 2 patients in the 120+120 mg dose groups.

The most frequently reported LiPlaCis-related AEs were nausea (16/25; 64%) and fatigue (14/25; 56%), followed by hypomagnesaemia (12/25; 48%), vomiting (11/25; 44%), anorexia (8/25; 32%), fever (7/25; 28%), nephrotoxicity (6/25 24%), infusion-related reaction (IRR) (10/25; 40%), Chills (5/25; 20%), hypokalaemia (4/25; 17%). The other LiPlaCis related AEs were reported for 1, 2 or 3 patients each.

In total, 12 patients (48%) experienced one or more SAEs, and in 8 patients (32%) one or more of these SAEs were considered LiPlaCis-related. For 8 patients (32%), study treatment was discontinued because of an AE, 5 patients in the 90+90 mg dose group, and 3 patients in the 120+120 mg dose group. In all 8 patients, the AE leading to study treatment discontinuation was LiPlaCis-related. There were no deaths on the study.

### Adverse Events in 12 Patients (Patient 14 to 25)

The regimen of pre-medication was extended with prednisolone and ibuprofen to prevent IRR. AEs on infusion-related reaction was subsequently reduced from 40% to 17% (2/12).

### SAE and Study Discontinuations

Twenty SAE's were reported. In total twelve patients (48%) experienced one or more SAE and in 8 patients (22%) one or more of these SAEs were considered LiPlaCis-related. Fever (Grade 2) was the most frequently reported SAE, namely for 3/25 (12%) patients, these SAE's were not considered LiPlaCis-related, all three at 90+90 mg. The LiPlaCis-related SAEs seen were acute kidney injury in two patients, one Grade 3, and one Grade 1 (at 90+90 mg (CTC 2) and 120+120 mg (CTC 1)), respectively; infusion related fever in 2 patients (at 60+60 mg), hypomagnesaemia in 2 patients, one Grade 2 and one Grade 3 (both at 60+60 mg); Grade 3 thromboembolic event in one patient (90+90 mg), Grade 2 nausea in one patient (90+90 mg), and Grade 2 elevated kidney counts in one patient (120+120 mg), where elevated kidney counts (120+120 mg) and acute kidney injury (120+120 mg) led to DLT. No treatment related deaths were reported in the study.

### Drug Response Prediction (DRP)

DRP is an assay that based on samples from a tumor can predict the likelihood for a tumor to respond to a specific drug. The DRP method builds on the comparison of sensitive and resistant cell lines including genomic information from the NCI60 cell lines, clinical tumor biology and clinical correlates in a systems biology network. mRNA measurements are used to make such drug prediction. Pre-clinical and clinical validation of response predictors have been developed for a number of drugs, with a unique signature of genes for each drug. This signature is matched to the corresponding genes in the universal microarray (which contains all genes) in order to make prediction for a specific drug for a specific patient. All breast cancer patients included in the phase II part of this study were predicted to be sensitive to LiPlaCis.

DRP in 11 Patients Treated in this Study:

Data from this Phase I/II study shows that tumor response to LiPlaCis can be predicted by DRP independent of tumor type and including breast cancer. Of the 11 patients analysed (8 from the phase I part and 3 from the phase II part) with mixed solid tumors, 2 patients had a Partial Response (PR) (one of these was operated and in Complete Remission (CR) 1 year after) and 4 patients had Stable Disease (SD). The correlation between prediction and response to treatment was 0.5 with a one-sided p-value of 0.06. Due to the small number of patients and mixed tumor types, this is a successful validation of the DRP's ability to predict response. These early data suggest that patients predicted sensitive by DRP to LiPlaCis (top third) have a 67% probability of response, and a median of 18 weeks to progression.

### Conclusion:

In this study (30 of approximately 40 patients were included), 2 DLTs were reported. This was a Grade 1 acute kidney injury in the first treatment cycle and a Grade 3 elevated kidney counts in the first treatment cycle both in the 120 mg dose group. It was decided to lower the dosage hereafter, and no further DLTs have been observed.

The toxicity observed in this study seems similar to what has been experienced with common cis-platinum containing regimen. Nephrotoxic effects have been observed with cisplatin therapies, although LiPlaCis appears to be well-tolerated. No ototoxicity or neurotoxicity was observed. These types of toxicities should be carefully looked for as these toxicities may depend on, e.g., the individual cumulated dose of LiPlaCis, numbers of prior treatment regimens and the type of anticancer drugs the patients have been exposed to. It should be mentioned that no Hand and Foot Syndrome, as well as no indication of bone marrow depletion and alopecia were observed.

Response and clinical benefit is notable as 3 PR are observed as well as 11 SD lasting from 8 to 32 weeks median time to progression 18 weeks. One of the PR patients was curative operated on and after one year still in CR. At present the study is including patients in the phase II part of the trial. The recommended dosage is two weekly doses of 75 mg in a 3 weekly cycle. Ten patients have been included on this dosage and further up to 20, mainly breast cancer patients are planned to be included.

### Example 13. Narratives Describing Individual Cases of Patients Responding to LiPlaCis Therapy

Outlined below are narratives describing the individual patients treated with LiPlaCis.

### Patient #17:

This patient is a 68-year-old woman diagnosed with colon cancer in April 2010. The patient underwent surgery in April 2010 and was subsequently treated with oxaliplatin+5-FU/irinotecan+5-FU/bevacizumab/Regorafinib (four lines of treatment were given). PR was observed in all cases as the best response. The patient met the entrance requirements for the LiPlaCis trial (Liver and lymph nodes, 0.54 mm, PS 0, normal Cr-EDTA).

In October 2013, the patient entered the LiPlaCis protocol in a dose-escalation part at 90+90 mg day 1+8 every 3 weeks and received 6 cycles (Cumulative dose: 1080 mg).

The patient exhibited a best response of SD of 18 weeks, as determined in November 2013 (verified December 2013). The patient exhibited AE Grade 1: Fever, Vomiting, Nausea, Chills, and AE Grade 2: Hypomagnesemia, Fatigue, Bronchospasm. No grades 3 or 4 AE were observed.

The patient exited the LiPlaCis protocol in February 2014 after PD (new lesions) with status: PS 1, normal Cr-EDTA.

### Patient #18:

This patient is a 47-year-old man diagnosed with esophagus cancer in September 2008. The patient had radiation therapy in February 2010, and underwent surgery in August 2012. From 2008, the patient was treated with Carboplatin+docetaxel+capecitabine/Cisplatin+5-FU/Carboplatin+docetaxel+capecitabine/Irinotecan (four lines of treatment were given). PR was observed in all cases as the best response. The patient met the entrance requirements for the LiPlaCis trial (Lymph node 53 mm, PS 1, normal (lower end) Cr-EDTA).

In November 2013, the patient entered the LiPlaCis protocol in dose-escalation part at 90+90 mg day 1+8 every 3 weeks and received 8 cycles (Cumulative dose: 1170 mg).

The patient exhibited a best response of PR of 23 weeks, as determined in January 2014 (verified in February 2014). The patient exhibited AE Grade 1: Nausea, Vomiting, Diarrhea, Nutrition disorder, Chills, Hypomagnesemia, and AE Grade 2: Fatigue, Hypomagnesemia, Nausea. No grades 3 or 4 AE were observed.

The patient exited the LiPlaCis protocol in April 2014 after PD (new lesions) with status: PS 1, below normal Cr-EDTA (40 ml/min).

### Patient #21:

This patient is a 65-year-old man diagnosed with cancer cutis, squamous cell carcinoma (well diff.) in May 2007. The patient underwent surgery in 2007, 2009 and 2010, had radiotherapy in 2011, and was treated with capecitabine+paclitaxel/vinorelbine+carboplatin (2 lines of treatment were given). PR was observed in all cases as the best response. The patient met the entrance requirements for the LiPlaCis trial (Tumor scalp wound 60 mm, PS 1, normal Cr-EDTA).

In January 2014, the patient entered the LiPlaCis protocol in dose-escalation part at 120+120 mg day 1+8 every 3 weeks and received 2% cycles (Cumulative dose: 540 mg).

The patient exhibited a best response PR enabling CR after surgery and remained disease free after 12 months. Latest measurement were not evaluable by RECIST criteria. The patient exhibited AE Grade 1: Vomiting, Anorexia, Headache, Flu like symptoms, Hypomagnesemia, Nausea. AE Grade 2: Infusion Related Reaction, Fatigue, Dyspnea, Renal disorders. No grades 3 or 4 AE were observed.

The patient exited the LiPlaCis protocol in April 2014 for renal disorders (Cr-EDTA 42 ml/min).

### Patient #26:

This patient is a 54-year-old male diagnosed with larynx cancer (Poorly diff.) in October 2009. The patient received radiation and underwent surgery in 2009, and was subsequently treated with Zalutumumab+Cisplatin/Taxol+Xeloda/Carboplatin+Vinorelbine/Bleomycin/Cetuximab+R05479599/Bleomycin (six lines of treatment were given). SD was observed in all cases as best response. The patient met the entrance requirements for the LiPlaCis trial (Right side neck 145 mm, PS 1, normal Cr-EDTA).

In June 2015, the patient entered the LiPlaCis protocol in dose-escalation part at 60+60 mg day 1+8 every 3 weeks and received 6% cycles (Cumulative dose: 780 mg).

The patient exhibited a best response of PR of 23 weeks, as determined in July 2015 (verified August 2015). Significant clinical response was observed on neck tumor and food intake. The patient exhibited AE Grade 1: Nausea, Flu like symptoms, Edema, Fatigue, Vomiting, Palmar Plantar Erythrodysethesia, Anemia, Hypokalemia, Weight loss, Headache, Diarrhea, Skin infection, AE Grade 2: Fatigue, Constipation, Weight loss, Anemia, Nausea, AE Grade 3: Hypomagnesemia, Hypermagnesemia, and SAE: Hypomagnesemia grade 3, Tracheal hemorrhage grade 3 (not related). The patient exited the LiPlaCis protocol in November 2015 after PD (new lesions) with status: PS 1, normal Cr-EDTA.

### Patient #28

This patient is a 41-year-old woman diagnosed with breast cancer (Poorly diff.) in March 2009. The patient received radiation and underwent surgery to the left axil in 2009, and was subsequently treated with Taxotere+Herceptin/Vinorelbine+Herceptin/Xeloda+Lapatinib/Trastuzumab/Trastuzumab+Perstuzumab+Gemcitabin+Carboplatin/Epirubicin/Trastuzumab+Eribulin/R06895882 (eight lines of treatment were given). PR was observed in all cases as best response. The patient met the entrance requirements for the LiPlaCis trial (Lymph Nodes 52 mm, PS 0, normal Cr-EDTA).

In November 2015, the patient entered the LiPlaCis protocol in dose-escalation part at 60+60 mg day 1+8 every 3 weeks and received 4 cycles (Cumulative dose: 480 mg).

The patient exhibited a best response of SD of 14 weeks, as determined in July 2015 (verified August 2015). The patient exhibited AE Grade 1: Vomiting, Edema, Diarrhea, Nausea, Peripheral sensory neuropathy, Dyspnea, Pain groin, Cramps in hands, and AE Grade 2: Fever, Nausea, Anemia, Hypomagnesemia, Infection in port-a-cath, Thromboembolic event, Weight loss, Infection, Creatinine increased, Edema both legs. No Grade 3 and Grade 4 AE were observed. The patient exhibited SAE: Fever (Not related) on 23 Nov. 2015, Infection (Not related) as determined on 18 Jan. 2016.

The patient exited the LiPlaCis protocol in February 2016 at Principal Investigator's decision (PS 1, normal Cr-EDTA).

### Patient #29:

This patient is a 38-year-old woman diagnosed with breast cancer in August 2008. The patient underwent Mastectomy (left side) and was treated with Cyclophosphamid+Epirubicin+5-FU and tamoxifen. I n 2009, the patient underwent prophylactic removal of right side breast and ovaries. The patient exhibited relapse in brain and liver in 2011 (ER neg, HER2 pos). The patient was treated with Herceptin/Herceptin+vinorelbine/docetaxel+Herceptin/capecitabine+lapatinib/Trastuzumab+Emtanzine/Herceptin+Lapatinib and whole-brain radiation (eight lines of treatment were given). CR was observed in one of the treatments as the best response. The patient met the entrance requirements for the LiPlaCis trial (PS 1, normal Cr-EDTA. Index tumors in liver, 37 mm).

In December 2015, the patient entered the LiPlaCis protocol at 90+90 mg day 1+8 every 3 weeks and received 6 cycles (Cumulative dose: 1080 mg).

The patient exhibited a best response of SD of 22 weeks, as determined in February 2016 (verified March 2016). The patient exhibited AE Grade 1: Mucositis, Pain drainage tube, Weight loss, Hypokalemia, Edema ankles, Cushingoid, Hypomagnesemia, and AE Grade 2: Constipation, Urinary tract infection, Pain Back, Anemia, Stomach Pain, Fatigue, Biloma, Infection drainage cavity, Ulcus, Acute kidney injury, Ataxia. No Grade 3 and Grade 4 AE were observed. The patient exhibited SAE: Constipation (Not related) in January 2016, Infection of insertion of former drainage cavity (Not related) in March 2016.

The patient exited the LiPlaCis protocol in February 2016 (PS 2, Cr-EDTA 54 ml/min).

### Patient #31:

This patient is a 71-year-old male diagnosed with liver cancer in August 2015. The patient did not undergo radiotherapy or surgery, and was treated with Doxorubicin/Naxavar/Ly3039478 (three lines of treatment were given). SD was observed in all cases as best response. The patient met the entrance requirements for the LiPlaCis trial (Liver 166 mm, PS 1, normal Cr-EDTA).

In February 2016, the patient entered the LiPlaCis protocol in dose-escalation part at 90+90 mg day 1+8 every 3 weeks and received 6 cycles (Cumulative dose: 990 mg).

The patient exhibited best response of SD of 18 weeks, as determined in April 2016 (verified May 2016). The patient exhibited AE Grade 1: Infusion related reaction, Nausea, Vomiting, Anorexia, Fever, Creatinine increased, and AE Grade 2: Fatigue, Dry skin, Cronic kidney disease. No Grade 3 and Grade 4 AE were observed. The patient exited the LiPlaCis protocol in February 2016 due to increased kidney toxicity (PS 1, below normal Cr-EDTA 51 ml/min).

### Patient #33:

This patient is a 51-year-old woman diagnosed with breast cancer in October 2008. The patient underwent mastectomy (right side), and was treated with Adjuvant Epirubicin+Cyclophosphamid/Docetaxel, radiation and Tamoxifen. The patient exhibited relapse in bone and liver in December 2012, and was treated with docetaxell letrozole/vinorelbine-capecitabinel eribulin/paclitaxel (eight lines of treatment were given). SD was observed as best response. The patient met the entrance requirements for the LiPlaCis trial (Multiple liver met 78 mm. PS 0, normal Cr-EDTA).

In May 2016, the patient entered the LiPlaCis protocol at 75+75 mg Phase II part day 1+8 every 3 weeks at Rigshospitalet and received 12 cycles (Cumulative dose: 1500 mg).

The patient exhibited a best response of PR of 32 weeks, as determined in July 2016 (verified August 2017). The patient exhibited AE Grade 1: Nausea, PSN in ankles. Edema, Fatigue, Neuropathy intermittent, Hypomagnesemia, Tinnitus, Vomiting, Anorexia, Constipation, Dyspepsia, Hyponatremia, Neuropathy in fingers, Pain right femur, AE Grade 2: Pain in epigastrium, Headache (infusion related), and AE Grade 3: Neutrophil Count Decreased. The patient exited the LiPlaCis protocol in January 2017 after PD (PS 1, normal Cr-EDTA).

### Patient #34:

This patient is a 55-year-old woman diagnosed with breast cancer in August 2008. The patient underwent mastectomy left side, and was treated with Adjuvant Epirubicin+Herceptin+Tamoxifen+Docetaxel+radiation/Vinorelbine+Herceptin/Docetaxel+Herceptin/Lapatinib+Capecitabine/TDM-1/Eribulin+Trastuzumab/Paclitaxel+Trastuzumab/Letrozol+Trastuzumab/Epirubicin/Exemestan/Capecitabine+Trastuzumab/Carboplatin+Trastuzumab (twelve lines of treatment were given). SD was observed as the best response. The patient met the entrance requirements for the LiPlaCis trial (Multiple liver met. 147 mm, PS 1, normal CrEDTA).

In January 2017, the patient entered the LiPlaCis protocol at 75+75 mg Phase II part day 1 +8 every 3 weeks at Herlev and received 4 cycles (Cumulative dose: 525 mg).

The patient exhibited a best response of SD of 12 weeks, as determined in April 2017 ((not verified as patient went out of study due to new lesion). The patient exhibited AE Grade 1: Tremor, Stomach pain, Palpitation, Nausea, Hypomagnesemia, Edema extremities, Malaise, Vomiting, Dyspnea, Vertigo, Bloating, AE Grade 2: Anemia, Fatigue, Malaise, Nausea, AE Grade 3: Insomnia, High cholesterol, and SAE: Grade 3 Bilirubinemia. The patient exited the LiPlaCis protocol in April 2017 due to SAE and new lesions (PS 1, normal Cr-EDTA).

### Patient #36 (Ongoing):

This patient is a 39-year-old woman diagnosed with breast cancer in August 2009. The patient underwent mastectomy (right side), and was treated with Neo adjuvant docetaxel/Adjuvant letrozole/Radiation/capecitabine+vinorelbine/tamoxifen/epirubicin/fulvestrant/pactitaxel (seven lines of treatment were given). SD was observed as the best response. The patient met the entrance requirements for the LiPlaCis trial (Lung left side met. mm, PS 1, normal Cr-EDTA).

In March 2017, the patient entered the LiPlaCis protocol at 75+75 mg Phase II part, day 1+8 every 3 weeks at Vejle and have received 9 cycles (Cumulative dose: 1350 mg). The patient exhibited a best response of SD of 28 weeks, as determined in June 2017 (verified August 2017). A SD of more than 24 weeks changes response status to PR. The patient exhibited AE Grade 1: Constipation, Nausea, Closed auditory canal, Prickly sensation tongue. No Grade 2, 3 and 4 AE were observed. The patient is still in the LiPlaCis protocol (October 2017 values: PS 0, below normal Cr-EDTA).

### Patient #37 (Ongoing):

This patient is a 40-year-old woman diagnosed with breast cancer in May 2006. The patient underwent mastectomy (right side), and was treated with Adjuvant epirubicin+cyclophosphamide+5-FU/tamoxifen/Radiation/Docetaxel/Letrozol/Fulvestrant/Docetaxel/Capecitabine/Eribulin/Paclitaxel (nine lines of treatment were given). SD was observed as the best response. The patient met the entrance requirements for the LiPlaCis trial (Liver met. PS 0, normal Cr-EDTA, ALT/AST/Alkaline Phosphatase above 5×ULN).

In March 2017, the patient entered the LiPlaCis protocol at 75+75 mg Phase II part, day 1 +8 every 3 weeks at Vejle and have received 7 cycles (Cumulative dose: 1050 mg). The patient best response of SD of 20 weeks, as determined in June 2017 (verified August 2017). The patient exhibited AE Grade 1: Dyspnea, Fatigue, Anorexia. The patient is still in the LiPlaCis protocol (October 2017 values: PS 0, normal Cr-EDTA, normal ALT/AST/Alkaline Phosphatase).

### Other Embodiments

All publications, patents, and patent applications mentioned in the above specification are hereby incorporated by reference. Various modifications and variations of the described device and methods of use of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention. For example, it is anticipated that measuring the level of proteins, metabolites, identifying genetic mutations and DNA copy number variations, all will be useful in determining patient responsiveness.

1. A method of treating a subject with cancer comprising administering to the subject at least first and second doses of a composition comprising a secretory phospholipase A2 (sPLA₂) hydrolysable, cisplatin-containing liposome on day 1 and day 8, respectively, of at least one three week treatment cycle, wherein each of the doses of the composition comprises cisplatin in an amount of about 75 mg to about 90 mg or cisplatin in an amount of about 40 mg/m² body surface area to about 55 mg/m² body surface area of the subject.

2.-5. (canceled)

6. The method of claim 1, wherein:
(i) an amount of about 150 mg to about 180 mg cisplatin is administered to the subject in each three week treatment cycle;
(ii) the three week treatment cycle is repeated two to twenty times; and/or
(iii) one or more additional therapies is administered to the subject prior to, concurrently with, or after administration of the composition.

7.-8. (canceled)

9. The method of claim 6, wherein the one or more additional therapies comprise surgery, radiation, or a therapeutic agent, and wherein optionally the therapeutic agent is selected from the group consisting of docetaxel, cabazitaxel, mitoxantrone, estramustine, prednisone, carboplatin, bevacizumab, paclitaxel, gemcitabine, doxorubicin, topotecan, etoposide, tamoxifen, letrozole, sorafenib, fluorouracil, capecitabine, oxaliplatin, interferon-alpha, 5-fluorouracil (5-FU), a histone deacetylase (HDAC) inhibitor, ipilimumab, bortezomib, carfilzomib, thalidomide, lenalidomide, pomalidomide, dexamethasone, cyclophosphamide, vincristine, melphalan, tegafur, irinotecan, cetuximab, leucovorin, SN-38, everolimus, temsirolimus, bleomycin, lomustine, depsipeptide, erlotinib, cisplatin, busulfan, epirubicin, arsenic trioxide, bendamustine, fulvestrant, teniposide, adriamycin, decitabine, estramustine, azaguanine, aclarubicin, mitomycin, paclitaxel, taxotere, APO010, ara-c, methylprednisolone, methotrexate, methyl-gag, belinostat, idarubicin, IL4-PR38, valproic acid, all-trans retinoic acid (ATRA), cytoxan, suberoylanilide hydroxamic acid, leukeran, fludarabine, vinblastine, dacarbazine, hydroxyurea, tegafur, daunorubicin, mechlorethamine, streptozocin, carmustine, mercaptopurine, dactinomycin, tretinoin, ifosfamide, floxuridine, thioguanine, PSC 833, herceptin, celecoxib, iressa, anastrozole, and rituximab.

10. The method of claim 1, wherein the composition is administered to the subject intravenously, intramuscularly, transdermally, intradermally, intra-arterially, intracranially, subcutaneously, intraorbitally, intraventricularly, intraspinally, intraperitoneally, or intranasally.

11. The method of claim 10, wherein:
(i) the composition is administered to the subject by intravenous infusion;
(ii) the composition is administered to the subject over a period of 2-3 hours; and/or
(iii) the subject has been determined to be responsive to the composition prior to administration of the composition.

12.-16. (canceled)

17. The method of claim 1 further comprising determining the responsiveness of the subject to the composition, wherein the method comprises:
(a) contacting a sample comprising one or more nucleic acid molecules from the subject with a device comprising:
i) one or more single-stranded nucleic acid molecules capable of specifically hybridizing with nucleotides of one or more biomarkers of sensitivity selected from those listed in Tables 2 and/or 4, or a complement thereof; and/or
ii) one or more single-stranded nucleic acid molecules capable of specifically hybridizing with nucleotides of one or more biomarkers of resistance selected from those listed in Tables 3 and/or 5, or a complement thereof; and
(b) detecting a level of the one or more biomarkers of sensitivity or the complement thereof and/or the one or more biomarkers of resistance or the complement thereof in the sample by detecting hybridization between the one or more single-stranded nucleic acid molecules of the device and the one or more nucleic acid molecules of the sample.

18. (canceled)

19. The method of claim 17, wherein:
(a) the subject is determined to be responsive to the composition comprising sPLA₂ hydrolysable, cisplatin-containing liposome if:
i) the level of the biomarkers of sensitivity or the complement thereof is substantially similar to the level of the biomarkers of sensitivity or the complement thereof in a cell or tissue known to be sensitive to the composition; and/or
ii) the level of the biomarkers of resistance or the complement thereof is substantially dissimilar to the level of the biomarkers of resistance or the complement thereof in a cell or tissue known to be resistant to the composition; and/or
(b) the method further comprises detecting a level of PLA2G2A (SEQ ID NO: 380), or a complement thereof, in the sample from the subject.

20. (canceled)

21. The method of claim 19, wherein:
(i) the method comprises determining the level of PLA2G2A, or a complement thereof, by performing microarray analysis or qRT-PCR; and/or
(ii) the method further comprises detecting sPLA₂ protein in a tumor sample from the subject, comprising contacting the tumor sample with an anti-sPLA₂ antibody and detecting binding between the sPLA₂ protein and the anti-sPLA₂ antibody.

22.-24. (canceled)

25. The method of claim 17, wherein:
(i) the one or more biomarkers of sensitivity is not C1QR1 (SEQ ID NO: 13), SLA (SEQ ID NO: 48), PTPN7 (SEQ ID NO: 77), CENTB1 (SEQ ID NO: 37), IFI16 (SEQ ID NO: 17 or 261), ARHGEF6 (SEQ ID NO: 36 or 294), CD3D (SEQ ID NO: 81), ARHGAP15 (SEQ ID NO: 30), HCLS1 (SEQ ID NO: 16 or 259), CD53 (SEQ ID NO: 282), PTPRCAP (SEQ ID NO: 8), or PTPRC (SEQ ID NO: 10, 18, 25, or 243);
(ii) the cell or tissue known to be sensitive to the composition comprising sPLA₂ hydrolysable, cisplatin-containing liposome and/or the cell or tissue known to be resistant to the composition is of the same type as a cell or tissue in the sample from the patient or from which the one or more nucleic acid molecules of the sample are derived; and/or
(iii) the sample from the subject is a tumor sample.

26. (canceled)

27. The method of claim 1, wherein:
(i) the subject is resistant to one or more cancer therapies other than the composition; and/or
(ii) the subject exhibits cancer relapse after treatment with the one or more cancer therapies,
wherein optionally the one or more cancer therapies comprise surgery, radiation, or a therapeutic agent.

28. (canceled)

29. The method of claim 1, wherein:
(i) the cancer is selected from a solid tumor cancer and a haematological cancer; and/or
(ii) the cancer is selected from the group consisting of breast cancer, acute myelogenous leukemia (AML), acute lympho-blastic leukemia (ALL), chronic lymphocytic leukemia (CLL), myelodysplastic syndrome (MDS), chronic myelogenous leukemia—chronic phase (CMLCP), diffuse large B-cell lymphoma (DLBCL), cutaneous T-cell lymphoma (CTCL), peripheral T-cell lymphoma (PTCL), Hodgkin's lymphoma, hepatocellular carcinoma (HCC), cervical cancer, renal cell carcinoma (RCC), esophageal cancer, melanoma, glioma, pancreatic cancer, gastrointestinal stromal tumors (GIST), sarcoma, non-small cell lung carcinoma (NSCLC), prostate cancer, ovarian cancer, colon cancer, bladder cancer, and squamous cell carcinoma of the head and neck (SCCHN), wherein optionally the breast cancer is an estrogen receptor-positive (Erpos) breast cancer and/or a metastatic form of breast cancer.

30.-31. (canceled)

32. The method of claim 1, wherein:
(i) the subject has not been administered a treatment for cancer; or
(ii) the subject exhibits cancer relapse after a first cancer treatment and prior to treatment with the composition,
wherein optionally the responsiveness of the subject to the composition is not determined prior to administration of the compound to the subject.

33.-34. (canceled)

35. The method of claim 17, wherein:
(i) the device comprises at least two, at least three, at least four, at least five, at least six, at least seven, at least eight, at least nine, at least ten, or more single-stranded nucleic acid molecules of i) and/or ii);
(ii) the one or more single-stranded nucleic acid molecules of the device have a length in the range of 10 to 100 nucleotides in length, wherein optionally, the one or more of the single-stranded nucleic acid molecules have a length in the range of 20 to 60 nucleotides; and/or
(iii) the one or more single-stranded nucleic acid molecules are labeled or immobilized on a solid substrate.

36.-37. (canceled)

38. The method of claim 17, comprising:
(i) converting the level of the one or more biomarkers of sensitivity or the complement thereof and/or the one or more biomarkers of resistance or the complement thereof into a mean score, wherein the mean score indicates the responsiveness of the subject to the composition; and, optionally,
(ii) subtracting the mean score for the one or more of the biomarkers of resistance from the mean score for the one or more of the biomarkers of sensitivity to obtain a difference score, wherein the difference score indicates the responsiveness of the subject to the composition.

39. (canceled)

40. The method of claim 38, wherein the mean score and/or the difference score above a cutoff value indicates that the subject is responsive to the composition, wherein optionally the cutoff value is about 0.1, about 0.15, about 0.2, about 0.25, about 0.3, about 0.35, about 0.4, about 0.45, about 0.5, or greater.

41. (canceled)

42. The method of claim 17, wherein:
(i) the device is a microarray or is for performing a quantitative reverse transcriptase polymerase chain reaction (qRT-PCR) reaction;
(ii) the level of the one or more biomarkers of sensitivity or the complement thereof and/or the one or more biomarkers of resistance or the complement thereof are detected by performing microarray analysis or qRT-PCR; and/or
(iii) the nucleic acid molecules of the sample comprise mRNA or a cDNA thereof.

43.-44. (canceled)

45. The method of claim 17, wherein:
(a) the biomarker of sensitivity is selected from one or more of COL5A2 (SEQ ID NO: 73 or 211), ITGA4 (SEQ ID NO: 1), MSN (SEQ ID NO: 2), FAM46A (SEQ ID NO: 3 or 280), ITGB2 (SEQ ID NO: 4), DOCK2 (SEQ ID NO: 5 or 223), EVL (SEQ ID NO: 6), SACS (SEQ ID NO: 7), EBI2 (SEQ ID NO: 9), ANP32E (SEQ ID NO: 11), SFPQ (SEQ ID NO: 12, 38 or 272), FNBP1 (SEQ ID NO: 14 or 28), CBFB (SEQ ID NO: 15), SFRS7 (SEQ ID NO: 19 or 54), and CAP350 (SEQ ID NO: 20 or 61), wherein optionally the biomarkers of sensitivity comprise:
(i) COL5A2 (SEQ ID NO 73 or 211) and ITGA4 (SEQ ID NO: 1);
(ii) COL5A2 (SEQ ID NO 73 or 211), ITGA4 (SEQ ID NO: 1), and MSN (SEQ ID NO: 2);
(iii) COL5A2 (SEQ ID NO 73 or 211), ITGA4 (SEQ ID NO: 1), MSN (SEQ ID NO: 2), and FAM46A (SEQ ID NO: 3 or 280);
(iv) COL5A2 (SEQ ID NO 73 or 211), ITGA4 (SEQ ID NO: 1), MSN (SEQ ID NO: 2), FAM46A (SEQ ID NO: 3 or 280), ITGB2 (SEQ ID NO: 4), and DOCK2 (SEQ ID NO: 5 or 223);
(v) COL5A2 (SEQ ID NO 73 or 211), ITGA4 (SEQ ID NO: 1), MSN (SEQ ID NO: 2), FAM46A (SEQ ID NO: 3 or 280), ITGB2 (SEQ ID NO: 4), DOCK2 (SEQ ID NO: 5 or 223), and EVL (SEQ ID NO: 6);
(vi) COL5A2 (SEQ ID NO 73 or 211), ITGA4 (SEQ ID NO: 1), MSN (SEQ ID NO: 2), FAM46A (SEQ ID NO: 3 or 280), ITGB2 (SEQ ID NO: 4), DOCK2 (SEQ ID NO: 5 or 223), EVL (SEQ ID NO: 6), and SACS (SEQ ID NO: 7);
(vii) COL5A2 (SEQ ID NO 73 or 211), ITGA4 (SEQ ID NO: 1), MSN (SEQ ID NO: 2), FAM46A (SEQ ID NO: 3 or 280), ITGB2 (SEQ ID NO: 4), DOCK2 (SEQ ID NO: 5 or 223), EVL (SEQ ID NO: 6), SACS (SEQ ID NO: 7), and PTPRCAP (SEQ ID NO: 8);
(viii) COL5A2 (SEQ ID NO 73 or 211), ITGA4 (SEQ ID NO: 1), MSN (SEQ ID NO: 2), FAM46A (SEQ ID NO: 3 or 280), ITGB2 (SEQ ID NO: 4), DOCK2 (SEQ ID NO: 5 or 223), EVL (SEQ ID NO: 6), SACS (SEQ ID NO: 7), PTPRCAP (SEQ ID NO: 8), and EBI2 (SEQ ID NO: 9);
(ix) COL5A2 (SEQ ID NO 73 or 211), ITGA4 (SEQ ID NO: 1), MSN (SEQ ID NO: 2), FAM46A (SEQ ID NO: 3 or 280), ITGB2 (SEQ ID NO: 4), DOCK2 (SEQ ID NO: 5 or 223), EVL (SEQ ID NO: 6), SACS (SEQ ID NO: 7), PTPRCAP (SEQ ID NO: 8), EBI2 (SEQ ID NO: 9), and PTPRC (SEQ ID NO: 10, 18, 25, or 243);
(x) COL5A2 (SEQ ID NO 73 or 211), ITGA4 (SEQ ID NO: 1), MSN (SEQ ID NO: 2), FAM46A (SEQ ID NO: 3 or 280), ITGB2 (SEQ ID NO: 4), DOCK2 (SEQ ID NO: 5 or 223), EVL (SEQ ID NO: 6), SACS (SEQ ID NO: 7), PTPRCAP (SEQ ID NO: 8), EBI2 (SEQ ID NO: 9), PTPRC (SEQ ID NO: 10, 18, 25, or 243), and ANP32E (SEQ ID NO: 11);
(xi) COL5A2 (SEQ ID NO 73 or 211), ITGA4 (SEQ ID NO: 1), MSN (SEQ ID NO: 2), FAM46A (SEQ ID NO: 3 or 280), ITGB2 (SEQ ID NO: 4), DOCK2 (SEQ ID NO: 5 or 223), EVL (SEQ ID NO: 6), SACS (SEQ ID NO: 7), PTPRCAP (SEQ ID NO: 8), EBI2 (SEQ ID NO: 9), PTPRC (SEQ ID NO: 10, 18, 25, or 243), ANP32E (SEQ ID NO: 11), and SFPQ (SEQ ID NO: 12, 38 or 272);
(xii) COL5A2 (SEQ ID NO 73 or 211), ITGA4 (SEQ ID NO: 1), MSN (SEQ ID NO: 2), FAM46A (SEQ ID NO: 3 or 280), ITGB2 (SEQ ID NO: 4), DOCK2 (SEQ ID NO: 5 or 223), EVL (SEQ ID NO: 6), SACS (SEQ ID NO: 7), PTPRCAP (SEQ ID NO: 8), EBI2 (SEQ ID NO: 9), PTPRC (SEQ ID NO: 10, 18, 25, or 243), ANP32E (SEQ ID NO: 11), SFPQ (SEQ ID NO: 12, 38 or 272), and C1QR1 (SEQ ID NO: 13);
(xiii) COL5A2 (SEQ ID NO 73 or 211), ITGA4 (SEQ ID NO: 1), MSN (SEQ ID NO: 2), FAM46A (SEQ ID NO: 3 or 280), ITGB2 (SEQ ID NO: 4), DOCK2 (SEQ ID NO: 5 or 223), EVL (SEQ ID NO: 6), SACS (SEQ ID NO: 7), PTPRCAP (SEQ ID NO: 8), EBI2 (SEQ ID NO: 9), PTPRC (SEQ ID NO: 10, 18, 25, or 243), ANP32E (SEQ ID NO: 11), SFPQ (SEQ ID NO: 12, 38 or 272), C1QR1 (SEQ ID NO: 13), and FNBP1 (SEQ ID NO: 14 or 28);
(xiv) COL5A2 (SEQ ID NO 73 or 211), ITGA4 (SEQ ID NO: 1), MSN (SEQ ID NO: 2), FAM46A (SEQ ID NO: 3 or 280), ITGB2 (SEQ ID NO: 4), DOCK2 (SEQ ID NO: 5 or 223), EVL (SEQ ID NO: 6), SACS (SEQ ID NO: 7), PTPRCAP (SEQ ID NO: 8), EBI2 (SEQ ID NO: 9), PTPRC (SEQ ID NO: 10, 18, 25, or 243), ANP32E (SEQ ID NO: 11), SFPQ (SEQ ID NO: 12, 38 or 272), C1QR1 (SEQ ID NO: 13), FNBP1 (SEQ ID NO: 14 or 28), and CBFB (SEQ ID NO: 15);
(xv) COL5A2 (SEQ ID NO: 73 or 211), ITGA4 (SEQ ID NO: 1), MSN (SEQ ID NO: 2), FAM46A (SEQ ID NO: 3 or 280), ITGB2 (SEQ ID NO: 4), DOCK2 (SEQ ID NO: 5 or 223), EVL (SEQ ID NO: 6), SACS (SEQ ID NO: 7), EBI2 (SEQ ID NO: 9), ANP32E (SEQ ID NO: 11), SFPQ (SEQ ID NO: 12, 38 or 272), FNBP1 (SEQ ID NO: 14 or 28), CBFB (SEQ ID NO: 15), and SFRS7 (SEQ ID NO: 19 or 54); or
(xvi) COL5A2 (SEQ ID NO: 73 or 211), ITGA4 (SEQ ID NO: 1), MSN (SEQ ID NO: 2), FAM46A (SEQ ID NO: 3 or 280), ITGB2 (SEQ ID NO: 4), DOCK2 (SEQ ID NO: 5 or 223), EVL (SEQ ID NO: 6), SACS (SEQ ID NO: 7), EBI2 (SEQ ID NO: 9), ANP32E (SEQ ID NO: 11), SFPQ (SEQ ID NO: 12, 38 or 272), FNBP1 (SEQ ID NO: 14 or 28), CBFB (SEQ ID NO: 15), SFRS7 (SEQ ID NO: 19 or 54), and CAP350 (SEQ ID NO: 20 or 61); and/or
(b) the biomarker of sensitivity is selected from one or more of CALD1 (SEQ ID NO: 206), COL6A2 (SEQ ID NO: 207), FERMT2 (SEQ ID NO: 208), BNIP3 (SEQ ID NO: 209 or 263), RAB31 (SEQ ID NO: 210), COL5A2 (SEQ ID NO: 73 or 211), MPO (SEQ ID NO: 212), SRPX (SEQ ID NO: 213), ARHGDIB (SEQ ID NO: 214), TMEM47 (SEQ ID NO: 215), CSRP2 (SEQ ID NO: 216), DPYSL3 (SEQ ID NO: 217), HTRA1 (SEQ ID NO: 218), SLC39A6 (SEQ ID NO: 219), and LAT2 (SEQ ID NO: 220), wherein optionally the biomarkers of sensitivity comprise:
(i) CALD1 (SEQ ID NO: 206) and COL6A2 (SEQ ID NO: 207);
(ii) CALD1 (SEQ ID NO: 206), COL6A2 (SEQ ID NO: 207), and FERMT2 (SEQ ID NO: 208);
(iii) CALD1 (SEQ ID NO: 206), COL6A2 (SEQ ID NO: 207), FERMT2 (SEQ ID NO: 208), and BNIP3 (SEQ ID NO: 209 or 263);
(iv) CALD1 (SEQ ID NO: 206), COL6A2 (SEQ ID NO: 207), FERMT2 (SEQ ID NO: 208), BNIP3 (SEQ ID NO: 209 or 263), and RAB31 (SEQ ID NO: 210);
(v) CALD1 (SEQ ID NO: 206), COL6A2 (SEQ ID NO: 207), FERMT2 (SEQ ID NO: 208), BNIP3 (SEQ ID NO: 209 or 263), RAB31 (SEQ ID NO: 210), and COL5A2 (SEQ ID NO: 73 or 211);
(vi) CALD1 (SEQ ID NO: 206), COL6A2 (SEQ ID NO: 207), FERMT2 (SEQ ID NO: 208), BNIP3 (SEQ ID NO: 209 or 263), RAB31 (SEQ ID NO: 210), COL5A2 (SEQ ID NO: 73 or 211), and MPO (SEQ ID NO: 212);
(vii) CALD1 (SEQ ID NO: 206), COL6A2 (SEQ ID NO: 207), FERMT2 (SEQ ID NO: 208), BNIP3 (SEQ ID NO: 209 or 263), RAB31 (SEQ ID NO: 210), COL5A2 (SEQ ID NO: 73 or 211), MPO (SEQ ID NO: 212), and SRPX (SEQ ID NO: 213);
(viii) CALD1 (SEQ ID NO: 206), COL6A2 (SEQ ID NO: 207), FERMT2 (SEQ ID NO: 208), BNIP3 (SEQ ID NO: 209 or 263), RAB31 (SEQ ID NO: 210), COL5A2 (SEQ ID NO: 73 or 211), MPO (SEQ ID NO: 212), SRPX (SEQ ID NO: 213), and ARHGDIB (SEQ ID NO: 214);
(ix) CALD1 (SEQ ID NO: 206), COL6A2 (SEQ ID NO: 207), FERMT2 (SEQ ID NO: 208), BNIP3 (SEQ ID NO: 209 or 263), RAB31 (SEQ ID NO: 210), COL5A2 (SEQ ID NO: 73 or 211), MPO (SEQ ID NO: 212), SRPX (SEQ ID NO: 213), ARHGDIB (SEQ ID NO: 214), and TMEM47 (SEQ ID NO: 215);
(x) CALD1 (SEQ ID NO: 206), COL6A2 (SEQ ID NO: 207), FERMT2 (SEQ ID NO: 208), BNIP3 (SEQ ID NO: 209 or 263), RAB31 (SEQ ID NO: 210), COL5A2 (SEQ ID NO: 73 or 211), MPO (SEQ ID NO: 212), SRPX (SEQ ID NO: 213), ARHGDIB (SEQ ID NO: 214), TMEM47 (SEQ ID NO: 215), and CSRP2 (SEQ ID NO: 216);
(xi) CALD1 (SEQ ID NO: 206), COL6A2 (SEQ ID NO: 207), FERMT2 (SEQ ID NO: 208), BNIP3 (SEQ ID NO: 209 or 263), RAB31 (SEQ ID NO: 210), COL5A2 (SEQ ID NO: 73 or 211), MPO (SEQ ID NO: 212), SRPX (SEQ ID NO: 213), ARHGDIB (SEQ ID NO: 214), TMEM47 (SEQ ID NO: 215), CSRP2 (SEQ ID NO: 216), and DPYSL3 (SEQ ID NO: 217);
(xii) CALD1 (SEQ ID NO: 206), COL6A2 (SEQ ID NO: 207), FERMT2 (SEQ ID NO: 208), BNIP3 (SEQ ID NO: 209 or 263), RAB31 (SEQ ID NO: 210), COL5A2 (SEQ ID NO: 73 or 211), MPO (SEQ ID NO: 212), SRPX (SEQ ID NO: 213), ARHGDIB (SEQ ID NO: 214), TMEM47 (SEQ ID NO: 215), CSRP2 (SEQ ID NO: 216), DPYSL3 (SEQ ID NO: 217), and HTRA1 (SEQ ID NO: 218);
(xiii) CALD1 (SEQ ID NO: 206), COL6A2 (SEQ ID NO: 207), FERMT2 (SEQ ID NO: 208), BNIP3 (SEQ ID NO: 209 or 263), RAB31 (SEQ ID NO: 210), COL5A2 (SEQ ID NO: 73 or 211), MPO (SEQ ID NO: 212), SRPX (SEQ ID NO: 213), ARHGDIB (SEQ ID NO: 214), TMEM47 (SEQ ID NO: 215), CSRP2 (SEQ ID NO: 216), DPYSL3 (SEQ ID NO: 217), HTRA1 (SEQ ID NO: 218), and SLC39A6 (SEQ ID NO: 219); or
(xiv) CALD1 (SEQ ID NO: 206), COL6A2 (SEQ ID NO: 207), FERMT2 (SEQ ID NO: 208), BNIP3 (SEQ ID NO: 209 or 263), RAB31 (SEQ ID NO: 210), COL5A2 (SEQ ID NO: 73 or 211), MPO (SEQ ID NO: 212), SRPX (SEQ ID NO: 213), ARHGDIB (SEQ ID NO: 214), TMEM47 (SEQ ID NO: 215), CSRP2 (SEQ ID NO: 216), DPYSL3 (SEQ ID NO: 217), HTRA1 (SEQ ID NO: 218), SLC39A6 (SEQ ID NO: 219), and LAT2 (SEQ ID NO: 220).

46.-61. (canceled)

62. The method of claim 17, wherein:
(a) the biomarker of resistance is selected from one or more of SFN (SEQ ID NO: 96 or 324), LISCH7 (SEQ ID NO: 97), EPB41L4B (SEQ ID NO: 98), MST1R (SEQ ID NO: 99), ITGB4 (SEQ ID NO: 100), DBNDD2 (SEQ ID NO: 102 or 365), TACSTD1 (SEQ ID NO: 104), MISP (SEQ ID NO: 105), KRT8 (SEQ ID NO: 106), JUP (SEQ ID NO: 107 or 400), KRT18 (SEQ ID NO: 108 or 306), FA2H (SEQ ID NO: 109), MGAT4B (SEQ ID NO: 110), DSG2 (SEQ ID NO:111 or 312), and LRP5 (SEQ ID NO: 112), wherein optionally the biomarkers of resistance comprise:
(i) SFN (SEQ ID NO: 96 or 324) and LISCH7 (SEQ ID NO: 97);
(ii) SFN (SEQ ID NO: 96 or 324), LISCH7 (SEQ ID NO: 97), and EPB41L4B (SEQ ID NO: 98);
(iii) SFN (SEQ ID NO: 96 or 324), LISCH7 (SEQ ID NO: 97), EPB41L4B (SEQ ID NO: 98), and MST1R (SEQ ID NO: 99);
(iv) SFN (SEQ ID NO: 96 or 324), LISCH7 (SEQ ID NO: 97), EPB41L4B (SEQ ID NO: 98), MST1R (SEQ ID NO: 99), and ITGB4 (SEQ ID NO: 100);
(v) SFN (SEQ ID NO: 96 or 324), LISCH7 (SEQ ID NO: 97), EPB41L4B (SEQ ID NO: 98), MST1R (SEQ ID NO: 99), ITGB4 (SEQ ID NO: 100), and DBNDD2 (SEQ ID NO: 102 or 365);
(vi) SFN (SEQ ID NO: 96 or 324), LISCH7 (SEQ ID NO: 97), EPB41L4B (SEQ ID NO: 98), MST1R (SEQ ID NO: 99), ITGB4 (SEQ ID NO: 100), DBNDD2 (SEQ ID NO: 102 or 365), and TACSTD1 (SEQ ID NO: 104);
(vii) SFN (SEQ ID NO: 96 or 324), LISCH7 (SEQ ID NO: 97), EPB41L4B (SEQ ID NO: 98), MST1R (SEQ ID NO: 99), ITGB4 (SEQ ID NO: 100), DBNDD2 (SEQ ID NO: 102 or 365), TACSTD1 (SEQ ID NO: 104), and MISP (SEQ ID NO: 105);
(viii) SFN (SEQ ID NO: 96 or 324), LISCH7 (SEQ ID NO: 97), EPB41L4B (SEQ ID NO: 98), MST1R (SEQ ID NO: 99), ITGB4 (SEQ ID NO: 100), DBNDD2 (SEQ ID NO: 102 or 365), TACSTD1 (SEQ ID NO: 104), MISP (SEQ ID NO: 105), and KRT8 (SEQ ID NO: 106);
(ix) SFN (SEQ ID NO: 96 or 324), LISCH7 (SEQ ID NO: 97), EPB41L4B (SEQ ID NO: 98), MST1R (SEQ ID NO: 99), ITGB4 (SEQ ID NO: 100), DBNDD2 (SEQ ID NO: 102 or 365), TACSTD1 (SEQ ID NO: 104), MISP (SEQ ID NO: 105), KRT8 (SEQ ID NO: 106), and JUP (SEQ ID NO: 107 or 400);
(x) SFN (SEQ ID NO: 96 or 324), LISCH7 (SEQ ID NO: 97), EPB41L4B (SEQ ID NO: 98), MST1R (SEQ ID NO: 99), ITGB4 (SEQ ID NO: 100), DBNDD2 (SEQ ID NO: 102 or 365), TACSTD1 (SEQ ID NO: 104), MISP (SEQ ID NO: 105), KRT8 (SEQ ID NO: 106), JUP (SEQ ID NO: 107 or 400), and KRT18 (SEQ ID NO: 108 or 306);
(xi) SFN (SEQ ID NO: 96 or 324), LISCH7 (SEQ ID NO: 97), EPB41L4B (SEQ ID NO: 98), MST1R (SEQ ID NO: 99), ITGB4 (SEQ ID NO: 100), DBNDD2 (SEQ ID NO: 102 or 365), TACSTD1 (SEQ ID NO: 104), MISP (SEQ ID NO: 105), KRT8 (SEQ ID NO: 106), JUP (SEQ ID NO: 107 or 400), KRT18 (SEQ ID NO: 108 or 306), and FA2H (SEQ ID NO: 109);
(xii) SFN (SEQ ID NO: 96 or 324), LISCH7 (SEQ ID NO: 97), EPB41L4B (SEQ ID NO: 98), MST1R (SEQ ID NO: 99), ITGB4 (SEQ ID NO: 100), DBNDD2 (SEQ ID NO: 102 or 365), TACSTD1 (SEQ ID NO: 104), MISP (SEQ ID NO: 105), KRT8 (SEQ ID NO: 106), JUP (SEQ ID NO: 107 or 400), KRT18 (SEQ ID NO: 108 or 306), FA2H (SEQ ID NO: 109), and MGAT4B (SEQ ID NO: 110);
(xiii) SFN (SEQ ID NO: 96 or 324), LISCH7 (SEQ ID NO: 97), EPB41L4B (SEQ ID NO: 98), MST1R (SEQ ID NO: 99), ITGB4 (SEQ ID NO: 100), DBNDD2 (SEQ ID NO: 102 or 365), TACSTD1 (SEQ ID NO: 104), MISP (SEQ ID NO: 105), KRT8 (SEQ ID NO: 106), JUP (SEQ ID NO: 107 or 400), KRT18 (SEQ ID NO: 108 or 306), FA2H (SEQ ID NO: 109), MGAT4B (SEQ ID NO: 110), and DSG2 (SEQ ID NO:111 or 312); or
(xiv) SFN (SEQ ID NO: 96 or 324), LISCH7 (SEQ ID NO: 97), EPB41L4B (SEQ ID NO: 98), MST1R (SEQ ID NO: 99), ITGB4 (SEQ ID NO: 100), DBNDD2 (SEQ ID NO: 102 or 365), TACSTD1 (SEQ ID NO: 104), MISP (SEQ ID NO: 105), KRT8 (SEQ ID NO: 106), JUP (SEQ ID NO: 107 or 400), KRT18 (SEQ ID NO: 108 or 306), FA2H (SEQ ID NO: 109), MGAT4B (SEQ ID NO: 110), DSG2 (SEQ ID NO:111 or 312), and LRP5 (SEQ ID NO: 112); and/or
(b) the biomarker of resistance is selected from one or more of KRT18 (SEQ ID NO: 108 or 306), LGALS3 (SEQ ID NO: 307), DSP (SEQ ID NO: 308), IGFBP4 (SEQ ID NO: 309), SPINT2 (SEQ ID NO: 310), CDH1 (SEQ ID NO: 311), DSG2 (SEQ ID NO: 111 or 312), RAB25 (SEQ ID NO: 313), PTPRF (SEQ ID NO: 314, 371, or 387), SOX9 (SEQ ID NO: 121, 315, or 319), LYZ (SEQ ID NO: 316), IER3 (SEQ ID NO: 127 or 317), PERP (SEQ ID NO: 318), ATP1B1 (SEQ ID NO: 320), and IFI27 (SEQ ID NO: 321), wherein optionally the biomarkers of resistance comprise:
(i) KRT18 (SEQ ID NO: 108 or 306) and LGALS3 (SEQ ID NO: 307);
(ii) KRT18 (SEQ ID NO: 108 or 306), LGALS3 (SEQ ID NO: 307), and DSP (SEQ ID NO: 308);
(iii) KRT18 (SEQ ID NO: 108 or 306), LGALS3 (SEQ ID NO: 307), DSP (SEQ ID NO: 308), and IGFBP4 (SEQ ID NO: 309);
(iv) KRT18 (SEQ ID NO: 108 or 306), LGALS3 (SEQ ID NO: 307), DSP (SEQ ID NO: 308), IGFBP4 (SEQ ID NO: 309), and SPINT2 (SEQ ID NO: 310);
(v) KRT18 (SEQ ID NO: 108 or 306), LGALS3 (SEQ ID NO: 307), DSP (SEQ ID NO: 308), IGFBP4 (SEQ ID NO: 309), SPINT2 (SEQ ID NO: 310), and CDH1 (SEQ ID NO: 311);
(vi) KRT18 (SEQ ID NO: 108 or 306), LGALS3 (SEQ ID NO: 307), DSP (SEQ ID NO: 308), IGFBP4 (SEQ ID NO: 309), SPINT2 (SEQ ID NO: 310), CDH1 (SEQ ID NO: 311), and DSG2 (SEQ ID NO: 111 or 312);
(vii) KRT18 (SEQ ID NO: 108 or 306), LGALS3 (SEQ ID NO: 307), DSP (SEQ ID NO: 308), IGFBP4 (SEQ ID NO: 309), SPINT2 (SEQ ID NO: 310), CDH1 (SEQ ID NO: 311), DSG2 (SEQ ID NO: 111 or 312), and RAB25 (SEQ ID NO: 313);
(viii) KRT18 (SEQ ID NO: 108 or 306), LGALS3 (SEQ ID NO: 307), DSP (SEQ ID NO: 308), IGFBP4 (SEQ ID NO: 309), SPINT2 (SEQ ID NO: 310), CDH1 (SEQ ID NO: 311), DSG2 (SEQ ID NO: 111 or 312), RAB25 (SEQ ID NO: 313), and PTPRF (SEQ ID NO: 314, 371, or 387);
(ix) KRT18 (SEQ ID NO: 108 or 306), LGALS3 (SEQ ID NO: 307), DSP (SEQ ID NO: 308), IGFBP4 (SEQ ID NO: 309), SPINT2 (SEQ ID NO: 310), CDH1 (SEQ ID NO: 311), DSG2 (SEQ ID NO: 111 or 312), RAB25 (SEQ ID NO: 313), PTPRF (SEQ ID NO: 314, 371, or 387), and SOX9 (SEQ ID NO: 121, 315, or 319);
(x) KRT18 (SEQ ID NO: 108 or 306), LGALS3 (SEQ ID NO: 307), DSP (SEQ ID NO: 308), IGFBP4 (SEQ ID NO: 309), SPINT2 (SEQ ID NO: 310), CDH1 (SEQ ID NO: 311), DSG2 (SEQ ID NO: 111 or 312), RAB25 (SEQ ID NO: 313), PTPRF (SEQ ID NO: 314, 371, or 387), SOX9 (SEQ ID NO: 121, 315, or 319), and LYZ (SEQ ID NO: 316);
(xi) KRT18 (SEQ ID NO: 108 or 306), LGALS3 (SEQ ID NO: 307), DSP (SEQ ID NO: 308), IGFBP4 (SEQ ID NO: 309), SPINT2 (SEQ ID NO: 310), CDH1 (SEQ ID NO: 311), DSG2 (SEQ ID NO: 111 or 312), RAB25 (SEQ ID NO: 313), PTPRF (SEQ ID NO: 314, 371, or 387), SOX9 (SEQ ID NO: 121, 315, or 319), LYZ (SEQ ID NO: 316), and IER3 (SEQ ID NO: 127 or 317);
(xii) KRT18 (SEQ ID NO: 108 or 306), LGALS3 (SEQ ID NO: 307), DSP (SEQ ID NO: 308), IGFBP4 (SEQ ID NO: 309), SPINT2 (SEQ ID NO: 310), CDH1 (SEQ ID NO: 311), DSG2 (SEQ ID NO: 111 or 312), RAB25 (SEQ ID NO: 313), PTPRF (SEQ ID NO: 314, 371, or 387), SOX9 (SEQ ID NO: 121, 315, or 319), LYZ (SEQ ID NO: 316), IER3 (SEQ ID NO: 127 or 317), and PERP (SEQ ID NO: 318);
(xiii) KRT18 (SEQ ID NO: 108 or 306), LGALS3 (SEQ ID NO: 307), DSP (SEQ ID NO: 308), IGFBP4 (SEQ ID NO: 309), SPINT2 (SEQ ID NO: 310), CDH1 (SEQ ID NO: 311), DSG2 (SEQ ID NO: 111 or 312), RAB25 (SEQ ID NO: 313), PTPRF (SEQ ID NO: 314, 371, or 387), SOX9 (SEQ ID NO: 121, 315, or 319), LYZ (SEQ ID NO: 316), IER3 (SEQ ID NO: 127 or 317), PERP (SEQ ID NO: 318), and ATP1B1 (SEQ ID NO: 320); or
(xiv) KRT18 (SEQ ID NO: 108 or 306), LGALS3 (SEQ ID NO: 307), DSP (SEQ ID NO: 308), IGFBP4 (SEQ ID NO: 309), SPINT2 (SEQ ID NO: 310), CDH1 (SEQ ID NO: 311), DSG2 (SEQ ID NO: 111 or 312), RAB25 (SEQ ID NO: 313), PTPRF (SEQ ID NO: 314, 371, or 387), SOX9 (SEQ ID NO: 121, 315, or 319), LYZ (SEQ ID NO: 316), IER3 (SEQ ID NO: 127 or 317), PERP (SEQ ID NO: 318), ATP1B1 (SEQ ID NO: 320), and IFI27 (SEQ ID NO: 321).

63.-108. (canceled)

109. A kit comprising:
i) a composition comprising sPLA₂ hydrolysable, cisplatin-containing liposome for use in treating cancer, wherein the composition is formulated for administration of at least two doses of cisplatin, wherein each of the doses comprises cisplatin in an amount of about 75 mg to about 90 mg or cisplatin in an amount of about 40 mg/m² body surface area to about 55 mg/m² body surface area, wherein the formulation is characterized to be administered on day 1 and day 8, respectively, of at least one three week treatment cycle; and, optionally,
ii) instructions for administering the composition to a subject in need thereof.


SEQUENCE LISTING (405 sequences)

SEQ ID NO: 1 - DNA, 25 bp, Homo sapiens
caggcctctcagatacaaggggaac

SEQ ID NO: 2 - DNA, 25 bp, Homo sapiens
atagctgccttaaagtcagtaactt

SEQ ID NO: 3 - DNA, 25 bp, Homo sapiens
caccatgctggctatccgggtgtta

SEQ ID NO: 4 - DNA, 25 bp, Homo sapiens
ctccactctgactggcacagtcttt

SEQ ID NO: 5 - DNA, 25 bp, Homo sapiens
gattcctgaactcaaggtaccagca

SEQ ID NO: 6 - DNA, 25 bp, Homo sapiens
gatcatcgacgccatcaggcaggag

SEQ ID NO: 7 - DNA, 25 bp, Homo sapiens
gtgtggttgaacaggatgcaatctt

SEQ ID NO: 8 - DNA, 25 bp, Homo sapiens
gcttcccaagatgccatggctggac

SEQ ID NO: 9 - DNA, 25 bp, Homo sapiens
gcaggacttcccttataaagcaaaa

SEQ ID NO: 10 - DNA, 25 bp, Homo sapiens
gattataaccgtgttgaactctctg

SEQ ID NO: 11 - DNA, 25 bp, Homo sapiens
gttttcggtcctattttaatgctct

SEQ ID NO: 12 - DNA, 25 bp, Homo sapiens
aaagaccaacaaatctcaagcccta

SEQ ID NO: 13 - DNA, 25 bp, Homo sapiens
ggtctgttcttgtagataatgccct

SEQ ID NO: 14 - DNA, 25 bp, Homo sapiens
tgctggccacggattttgacgacga

SEQ ID NO: 15 - DNA, 25 bp, Homo sapiens
ggtgttgtacagctcacatgtttac

SEQ ID NO: 16 - DNA, 25 bp, Homo sapiens
ggtttgcctcattgtgctatttgcc

SEQ ID NO: 17 - DNA, 25 bp, Homo sapiens
ataagcattgattcctgcatttctg

SEQ ID NO: 18 - DNA, 25 bp, Homo sapiens
gcatttagtccaatgtctttttaag

SEQ ID NO: 19 - DNA, 25 bp, Homo sapiens
atcatgctgaggcgccttgcaaatc

SEQ ID NO: 20 - DNA, 25 bp, Homo sapiens
atgactggtatgatagctcttgaca

SEQ ID NO: 21 - DNA, 25 bp, Homo sapiens
caatccaagcataactcagtgacgc

SEQ ID NO: 22 - DNA, 25 bp, Homo sapiens
gaatgtgtagctcaaatgcaaacca

SEQ ID NO: 23 - DNA, 25 bp, Homo sapiens
ttccctccttatagtcaaggaccgt

SEQ ID NO: 24 - DNA, 25 bp, Homo sapiens
tgacctcggtcacaaaagcagtttt

SEQ ID NO: 25 - DNA, 25 bp, Homo sapiens
gaacagtttgtacagacgtatgctt

SEQ ID NO: 26 - DNA, 25 bp, Homo sapiens
tacaacactatacatacacaccacc

SEQ ID NO: 27 - DNA, 25 bp, Homo sapiens
ttatgccagcttatattgtgagaac

SEQ ID NO: 28 - DNA, 25 bp, Homo sapiens
gagttgcctgtttgtctctggagat

SEQ ID NO: 29 - DNA, 25 bp, Homo sapiens
gctgcagtgtagatggctcttgttt

SEQ ID NO: 30 - DNA, 25 bp, Homo sapiens
acgttgtcaccggagcactgaagat

SEQ ID NO: 31 - DNA, 25 bp, Homo sapiens
atagcagcacacattttcacgtttc

SEQ ID NO: 32 - DNA, 25 bp, Homo sapiens
aagaccggcagatggtggtgctgga

SEQ ID NO: 33 - DNA, 25 bp, Homo sapiens
atttggctcaagtccatttggctgt

SEQ ID NO: 34 - DNA, 25 bp, Homo sapiens
gcatgaagttgcccttaaccactaa

SEQ ID NO: 35 - DNA, 25 bp, Homo sapiens
taaccatgcttacacactaaactat

SEQ ID NO: 36 - DNA, 25 bp, Homo sapiens
tgcccggtcggagtaaacacagaat

SEQ ID NO: 37 - DNA, 25 bp, Homo sapiens
gatgtcaactgggtcaatgggggcc

SEQ ID NO: 38 - DNA, 25 bp, Homo sapiens
gttggctgatattggagtgctcatt

SEQ ID NO: 39 - DNA, 25 bp, Homo sapiens
ccttttgtacttcactcagatacta

SEQ ID NO: 40 - DNA, 25 bp, Homo sapiens
taagcatccttagggttctgcctct

SEQ ID NO: 41 - DNA, 25 bp, Homo sapiens
ctcatctccctcaaggatggctacg

SEQ ID NO: 42 - DNA, 25 bp, Homo sapiens
tgtgaatcatcctgcctttcaaatt

SEQ ID NO: 43 - DNA, 25 bp, Homo sapiens
tacaaaccacattacttctgtcact

SEQ ID NO: 44 - DNA, 25 bp, Homo sapiens
gtcccggatccaggacctggtagca

SEQ ID NO: 45 - DNA, 25 bp, Homo sapiens
agtgaagaggtcgtcctctccatct

SEQ ID NO: 46 - DNA, 25 bp, Homo sapiens
gctgaaggcacctactcagtatctt

SEQ ID NO: 47 - DNA, 25 bp, Homo sapiens
taagcattccgtccatctaagctca

SEQ ID NO: 48 - DNA, 25 bp, Homo sapiens
tgatggagcataacgggtcccagcc

SEQ ID NO: 49 - DNA, 25 bp, Homo sapiens
atgatttcttagggtctgtgtactt

SEQ ID NO: 50 - DNA, 25 bp, Homo sapiens
gttccatttctctcattcacaagat

SEQ ID NO: 51 - DNA, 25 bp, Homo sapiens
gaggccaagaaattccatgttgttt

SEQ ID NO: 52 - DNA, 25 bp, Homo sapiens
aaagctgtgaatctgttccctgctg

SEQ ID NO: 53 - DNA, 25 bp, Homo sapiens
atgagctcccagattcgtcagaatt

SEQ ID NO: 54 - DNA, 25 bp, Homo sapiens
tccccatcaggaagtcctcgcagaa

SEQ ID NO: 55 - DNA, 25 bp, Homo sapiens
tgagtccctggaacgccagatgcgt

SEQ ID NO: 56 - DNA, 25 bp, Homo sapiens
ttagagccagtcttcacactcggaa

SEQ ID NO: 57 - DNA, 25 bp, Homo sapiens
aaaacttccccggtatgatgattgt

SEQ ID NO: 58 - DNA, 25 bp, Homo sapiens
tcagtgggcacagttcttcagctac

SEQ ID NO: 59 - DNA, 25 bp, Homo sapiens
actagctcattatttccatctttgg

SEQ ID NO: 60 - DNA, 25 bp, Homo sapiens
aattatgagtttctatctgtgtcca

SEQ ID NO: 61 - DNA, 25 bp, Homo sapiens
gggaagtccacatagcgtcattaat

SEQ ID NO: 62 - DNA, 25 bp, Homo sapiens
ttcagctaccctgactttctcagga

SEQ ID NO: 63 - DNA, 25 bp, Homo sapiens
ggcatgatgtccggtgatttctgta

SEQ ID NO: 64 - DNA, 25 bp, Homo sapiens
actcttgagggttgattatgctgca

SEQ ID NO: 65 - DNA, 25 bp, Homo sapiens
ggacctcatctctgagctgaaacgg

SEQ ID NO: 66 - DNA, 25 bp, Homo sapiens
gttggtgtttaaagatctgaagtgt

SEQ ID NO: 67 - DNA, 25 bp, Homo sapiens
gaagcaccataactttgtttagccc

SEQ ID NO: 68 - DNA, 25 bp, Homo sapiens
acagcatgaggcggccggggagctg

SEQ ID NO: 69 - DNA, 25 bp, Homo sapiens
gctatggttatattagcaccaaact

SEQ ID NO: 70 - DNA, 25 bp, Homo sapiens
ggccgacaacagctgcatctatgtc

SEQ ID NO: 71 - DNA, 25 bp, Homo sapiens
tgaaaaagggtttctattctctctg

SEQ ID NO: 72 - DNA, 25 bp, Homo sapiens
agtatcagtcggtgcaacagttggc

SEQ ID NO: 73 - DNA, 25 bp, Homo sapiens
tgaagttgatcctgagactcttgaa

SEQ ID NO: 74 - DNA, 25 bp, Homo sapiens
tactcagaggtgtgaccctcgccag

SEQ ID NO: 75 - DNA, 25 bp, Homo sapiens
gtcgtactatcttactgagccacag

SEQ ID NO: 76 - DNA, 25 bp, Homo sapiens
aaggcgtaacgagttcatctttctt

SEQ ID NO: 77 - DNA, 25 bp, Homo sapiens
ccttgataccagctctctgtggaaa

SEQ ID NO: 78 - DNA, 25 bp, Homo sapiens
aaatcactaaacctcgttttctcag

SEQ ID NO: 79 - DNA, 25 bp, Homo sapiens
agcgtccttatctttcagagctaca

SEQ ID NO: 80 - DNA, 25 bp, Homo sapiens
aaaccccagacgccatgaaagctgc

SEQ ID NO: 81 - DNA, 25 bp, Homo sapiens
gggaacactgctctcagacattaca

SEQ ID NO: 82 - DNA, 25 bp, Homo sapiens
ctttttcctgggtcatgctgcaaca

SEQ ID NO: 83 - DNA, 25 bp, Homo sapiens
gatggggtgcatgtagtctttggac

SEQ ID NO: 84 - DNA, 25 bp, Homo sapiens
gaaggtgtgatctgtgggactgtct

SEQ ID NO: 85 - DNA, 25 bp, Homo sapiens
gtacgtttttactcagttcatgcgt

SEQ ID NO: 86 - DNA, 25 bp, Homo sapiens
gcttctcgtgctgcacatatttcct

SEQ ID NO: 87 - DNA, 25 bp, Homo sapiens
gtgaacagacttgaaactccagagc

SEQ ID NO: 88 - DNA, 25 bp, Homo sapiens
atcattttcaggcttctgcagctgt

SEQ ID NO: 89 - DNA, 25 bp, Homo sapiens
gcatgttgtttgccaggacactgtg

SEQ ID NO: 90 - DNA, 25 bp, Homo sapiens
ttgtgcttgctcttcagatggatgg

SEQ ID NO: 91 - DNA, 25 bp, Homo sapiens
tagccaggatccttggtgtcctagg

SEQ ID NO: 92 - DNA, 25 bp, Homo sapiens
gcggcgtgtataccaatgcatggcc

SEQ ID NO: 93 - DNA, 25 bp, Homo sapiens
gaataacttttggctgttgtgctaa

SEQ ID NO: 94 - DNA, 25 bp, Homo sapiens
tggcccgcgtgattgtggcatttaa

SEQ ID NO: 95 - DNA, 25 bp, Homo sapiens
cataactgttagacttcccgtttct

SEQ ID NO: 96 - DNA, 25 bp, Homo sapiens
tcaataaagttcccctgtgacactc

SEQ ID NO: 97 - DNA, 25 bp, Homo sapiens
ctcccctatgatgggcggctactgg

SEQ ID NO: 98 - DNA, 25 bp, Homo sapiens
atcagttgattcttgtgccattttt

SEQ ID NO: 99 - DNA, 25 bp, Homo sapiens
tgagccagtgagggcagtcctgcaa

SEQ ID NO: 100 - DNA, 25 bp, Homo sapiens
gcatcatcaccatagagtcccagga

SEQ ID NO: 101 - DNA, 25 bp, Homo sapiens
tcttgctccaaagggctccgtggag

SEQ ID NO: 102 - DNA, 25 bp, Homo sapiens
atacgcccttggcacagtcggatga

SEQ ID NO: 103 - DNA, 25 bp, Homo sapiens
gtctgctgggtgtgaccatgtttcc

SEQ ID NO: 104 - DNA, 25 bp, Homo sapiens
gtgcgtgggacgaagacatctttga

SEQ ID NO: 105 - DNA, 25 bp, Homo sapiens
tggtccccttcacctgggagaaaag

SEQ ID NO: 106 - DNA, 25 bp, Homo sapiens
gggccaagcaggacatggcgcggca

SEQ ID NO: 107 - DNA, 25 bp, Homo sapiens
agcttcagactcaagtacccattct

SEQ ID NO: 108 - DNA, 25 bp, Homo sapiens
gagctgctgagacgacgctcacaga

SEQ ID NO: 109 - DNA, 25 bp, Homo sapiens
gagaagcagtttgacggaccttgtg

SEQ ID NO: 110 - DNA, 25 bp, Homo sapiens
ggtgattctgagcgagatcttcctg

SEQ ID NO: 111 - DNA, 25 bp, Homo sapiens
gcagccttggaaacctaacctgcct

SEQ ID NO: 112 - DNA, 25 bp, Homo sapiens
cctgcagcaccgacgtgtgtgacag

SEQ ID NO: 113 - DNA, 25 bp, Homo sapiens
acttggctcagtggaagccctcttt

SEQ ID NO: 114 - DNA, 25 bp, Homo sapiens
acattgcccggaaactcagtctatt

SEQ ID NO: 115 - DNA, 25 bp, Homo sapiens
gctgtggatctgtttggccagggtc

SEQ ID NO: 116 - DNA, 25 bp, Homo sapiens
gttagagccgatatcactggaagat

SEQ ID NO: 117 - DNA, 25 bp, Homo sapiens
agatttgtcagccctatctcaaact

SEQ ID NO: 118 - DNA, 25 bp, Homo sapiens
aggtcttcccagaggctggatacca

SEQ ID NO: 119 - DNA, 25 bp, Homo sapiens
gtcttccctacctcaggcaggaagg

SEQ ID NO: 120 - DNA, 25 bp, Homo sapiens
ctacccttatgatgacccattttcc

SEQ ID NO: 121 - DNA, 25 bp, Homo sapiens
aaatgctcttatttttccaacagct

SEQ ID NO: 122 - DNA, 25 bp, Homo sapiens
gtgtatagtgttttaccctcttctt

SEQ ID NO: 123 - DNA, 25 bp, Homo sapiens
tcatcaagggctatgtgcctcccac

SEQ ID NO: 124 - DNA, 25 bp, Homo sapiens
ccccgcaccagatcaagtagtttgg

SEQ ID NO: 125 - DNA, 25 bp, Homo sapiens
ccctcctaccagatgacacagacaa

SEQ ID NO: 126 - DNA, 25 bp, Homo sapiens
tgtatggttttcacctggacaccgt

SEQ ID NO: 127 - DNA, 25 bp, Homo sapiens
aactccgtctgtctactgtgtgaga

SEQ ID NO: 128 - DNA, 25 bp, Homo sapiens
cccactggcctgaatctacactgga

SEQ ID NO: 129 - DNA, 25 bp, Homo sapiens
acattacatccgtggattctcctgc

SEQ ID NO: 130 - DNA, 25 bp, Homo sapiens
ggccctgggccagggtgattggact

SEQ ID NO: 131 - DNA, 25 bp, Homo sapiens
tttagccctcatgactgtattttct

SEQ ID NO: 132 - DNA, 25 bp, Homo sapiens
acacgctgcagatcgaggactttct

SEQ ID NO: 133 - DNA, 25 bp, Homo sapiens
accggcagccctggaaggggcactt

SEQ ID NO: 134 - DNA, 25 bp, Homo sapiens
taccagcaggaggttctgggcctct

SEQ ID NO: 135 - DNA, 25 bp, Homo sapiens
catctttcagggctgccagtttcgc

SEQ ID NO: 136 - DNA, 25 bp, Homo sapiens
gggctacgccaaggactttgaccct

SEQ ID NO: 137 - DNA, 25 bp, Homo sapiens
aatctagtcacctaaccttgtggtt

SEQ ID NO: 138 - DNA, 25 bp, Homo sapiens
atttcaaaatttctgcattcacgga

SEQ ID NO: 139 - DNA, 25 bp, Homo sapiens
aacacctgtccattgaagatttcac

SEQ ID NO: 140 - DNA, 25 bp, Homo sapiens
gacattcttatgcttcttttacaac

SEQ ID NO: 141 - DNA, 25 bp, Homo sapiens
aatgtttcctaacagttgtgatgtt

SEQ ID NO: 142 - DNA, 25 bp, Homo sapiens
gggtgaagagagactcggtgcgggc

SEQ ID NO: 143 - DNA, 25 bp, Homo sapiens
cgaccgcctgtatgtttgtgtaatt

SEQ ID NO: 144 - DNA, 25 bp, Homo sapiens
aaggcctatcagcttctatcagccc

SEQ ID NO: 145 - DNA, 25 bp, Homo sapiens
gtcactggtctgtttgcatttgata

SEQ ID NO: 146 - DNA, 25 bp, Homo sapiens
agctcaaaacttgctaggcatcaga

SEQ ID NO: 147 - DNA, 25 bp, Homo sapiens
gcagcaaagctggctccaacatgct

SEQ ID NO: 148 - DNA, 25 bp, Homo sapiens
aaacactatctacttcctttgtcat

SEQ ID NO: 149 - DNA, 25 bp, Homo sapiens
gaggatcatgcccttagcaagtact

SEQ ID NO: 150 - DNA, 25 bp, Homo sapiens
aacatcattttagcaaaggccagga

SEQ ID NO: 151 - DNA, 25 bp, Homo sapiens
tgtccttgttacattgaggttaaga

SEQ ID NO: 152 - DNA, 25 bp, Homo sapiens
tccctactgcatggggacttccacg

SEQ ID NO: 153 - DNA, 25 bp, Homo sapiens
ccaccttcacctcggagggacggag

SEQ ID NO: 154 - DNA, 25 bp, Homo sapiens
taattacacgttcaccgtccaagca

SEQ ID NO: 155 - DNA, 25 bp, Homo sapiens
ttccctttctaccattgatttaaat

SEQ ID NO: 156 - DNA, 25 bp, Homo sapiens
agtactggttgaacctgaccacttc

SEQ ID NO: 157 - DNA, 25 bp, Homo sapiens
aaatacataagctagtttctgttct

SEQ ID NO: 158 - DNA, 25 bp, Homo sapiens
gtaacgtgattgattcagtatctta

SEQ ID NO: 159 - DNA, 25 bp, Homo sapiens
aatacatccgatgcgtagattcttg

SEQ ID NO: 160 - DNA, 25 bp, Homo sapiens
agaatgctcgggtgctagactggat

SEQ ID NO: 161 - DNA, 25 bp, Homo sapiens
ggacggactctatgaggggctcaca

SEQ ID NO: 162 - DNA, 25 bp, Homo sapiens
ggaatgactcaaatgcccaaaacca

SEQ ID NO: 163 - DNA, 25 bp, Homo sapiens
tcctctcagctcctaaagcacaact

SEQ ID NO: 164 - DNA, 25 bp, Homo sapiens
acatgttcgcacccaagtgtggcgg

SEQ ID NO: 165 - DNA, 25 bp, Homo sapiens
tttcagatggagtaccagcaccgaa

SEQ ID NO: 166 - DNA, 25 bp, Homo sapiens
tggcactcgcggttggagtcagcga

SEQ ID NO: 167 - DNA, 25 bp, Homo sapiens
ggccatctatgggactgaactttga

SEQ ID NO: 168 - DNA, 25 bp, Homo sapiens
gaagaaactggacaccaacagtgat

SEQ ID NO: 169 - DNA, 25 bp, Homo sapiens
gctgtagccagtgcagacctcactg

SEQ ID NO: 170 - DNA, 25 bp, Homo sapiens
aggtgtccagtacctaatcacgctc

SEQ ID NO: 171 - DNA, 25 bp, Homo sapiens
ttgccgtgatgaccgtggctatcct

SEQ ID NO: 172 - DNA, 25 bp, Homo sapiens
gaatacgttaggagccagtacccca

SEQ ID NO: 173 - DNA, 25 bp, Homo sapiens
gaagctgctcaacaaagtgtggaag

SEQ ID NO: 174 - DNA, 25 bp, Homo sapiens
ggaatgtcccagacagacctgtctc

SEQ ID NO: 175 - DNA, 25 bp, Homo sapiens
ccttcatggacgtcaacagcacctg

SEQ ID NO: 176 - DNA, 25 bp, Homo sapiens
ggcccggatatggctcgtggacagc

SEQ ID NO: 177 - DNA, 25 bp, Homo sapiens
aggagtctccaccagagggaggctc

SEQ ID NO: 178 - DNA, 25 bp, Homo sapiens
gagcaccactactgctgcattgtgc

SEQ ID NO: 179 - DNA, 25 bp, Homo sapiens
actctgacatttcttgttctcaagc

SEQ ID NO: 180 - DNA, 25 bp, Homo sapiens
ccagcatttccagagcagagcctac

SEQ ID NO: 181 - DNA, 25 bp, Homo sapiens
gcgtgtcttgagttccatgcaaatt

SEQ ID NO: 182 - DNA, 25 bp, Homo sapiens
aatggtgacagtccaaaccactcca

SEQ ID NO: 183 - DNA, 25 bp, Homo sapiens
ggccctgcatgtcagatggcgtggt

SEQ ID NO: 184 - DNA, 25 bp, Homo sapiens
tgatcataaattctccccaactata

SEQ ID NO: 185 - DNA, 25 bp, Homo sapiens
aaagttgccaagatgctccttgttg

SEQ ID NO: 186 - DNA, 25 bp, Homo sapiens
gtctcacccaactgcagtttactat

SEQ ID NO: 187 - DNA, 25 bp, Homo sapiens
gacggagttcctaagcttcatgaat

SEQ ID NO: 188 - DNA, 25 bp, Homo sapiens
tcagcctgggcagtcttaccaaaat

SEQ ID NO: 189 - DNA, 25 bp, Homo sapiens
tgctgtggcttcaccaactatacgg

SEQ ID NO: 190 - DNA, 25 bp, Homo sapiens
ccttgactcctttggtatttcactg

SEQ ID NO: 191 - DNA, 25 bp, Homo sapiens
acagcaggcatcgcaactttctgca

SEQ ID NO: 192 - DNA, 25 bp, Homo sapiens
caagagctacaatgtcacctccgtc

SEQ ID NO: 193 - DNA, 25 bp, Homo sapiens
gtttgtctcttgttgttctgaagga

SEQ ID NO: 194 - DNA, 25 bp, Homo sapiens
ctctctagttagatatctgacttgg

SEQ ID NO: 195 - DNA, 25 bp, Homo sapiens
tccagcctcggggtgtaggtttctg

SEQ ID NO: 196 - DNA, 25 bp, Homo sapiens
actcgtctcacgccgtgtttgagat

SEQ ID NO: 197 - DNA, 25 bp, Homo sapiens
gccagagttcaaatgtgactccacc

SEQ ID NO: 198 - DNA, 25 bp, Homo sapiens
caagcccctgtattttgctgatcgg

SEQ ID NO: 199 - DNA, 25 bp, Homo sapiens
agaccacagatatcttctagacata

SEQ ID NO: 200 - DNA, 25 bp, Homo sapiens
gtcactgtaaatcattcttaagccc

SEQ ID NO: 201 - DNA, 25 bp, Homo sapiens
aaggctgtcagggcttctgtttgtt

SEQ ID NO: 202 - DNA, 25 bp, Homo sapiens
gtagacacctgcacgcataggattg

SEQ ID NO: 203 - DNA, 25 bp, Homo sapiens
ttgccactttcttgcgatatgctgt

SEQ ID NO: 204 - DNA, 25 bp, Homo sapiens
gccattccagaagtagcttatccta

SEQ ID NO: 205 - DNA, 25 bp, Homo sapiens
ccaataccccaccgtgatgacttga

SEQ ID NO: 206 - DNA, 25 bp, Homo sapiens
aattctctgttatctttacgaggta

SEQ ID NO: 207 - DNA, 25 bp, Homo sapiens
cacgagaaggactatgacagcctgg

SEQ ID NO: 208 - DNA, 25 bp, Homo sapiens
tgatttgccacaatgtccttaactc

SEQ ID NO: 209 - DNA, 25 bp, Homo sapiens
gctgaaggcacctactcagtatctt

SEQ ID NO: 210 - DNA, 25 bp, Homo sapiens
agacctggcacttcagtaactcagc

SEQ ID NO: 211 - DNA, 25 bp, Homo sapiens
gactcttgaagtaatggctgatcct

SEQ ID NO: 212 - DNA, 25 bp, Homo sapiens
gggactttgtcaactgcagtacact

SEQ ID NO: 213 - DNA, 25 bp, Homo sapiens
cctttctttactccatcatggctgg

SEQ ID NO: 214 - DNA, 25 bp, Homo sapiens
atcactaacaggtctttgactcagg

SEQ ID NO: 215 - DNA, 25 bp, Homo sapiens
gaattcatggtatcctggttatttt

SEQ ID NO: 216 - DNA, 25 bp, Homo sapiens
aactactgtgaaattctaccagcat

SEQ ID NO: 217 - DNA, 25 bp, Homo sapiens
gacacctgagcctggattttcactc

SEQ ID NO: 218 - DNA, 25 bp, Homo sapiens
tcaaacggccgaagttgcctctttt

SEQ ID NO: 219 - DNA, 25 bp, Homo sapiens
atactaggcctgtctgtggcattct

SEQ ID NO: 220 - DNA, 25 bp, Homo sapiens
ggatttaggataagctgtcacccag

SEQ ID NO: 221 - DNA, 25 bp, Homo sapiens
ggtcagcaacctcttttgattttgt

SEQ ID NO: 222 - DNA, 25 bp, Homo sapiens
gacaggtgaacatttccgcctggtc

SEQ ID NO: 223 - DNA, 25 bp, Homo sapiens
gattcctgaactcaaggtaccagca

SEQ ID NO: 224 - DNA, 25 bp, Homo sapiens
tgttcctttttgttcaaagtctatt

SEQ ID NO: 225 - DNA, 25 bp, Homo sapiens
aggtgttcgaaatccgcaccactga

SEQ ID NO: 226 - DNA, 25 bp, Homo sapiens
gtggagttgatgactttctgttttc

SEQ ID NO: 227 - DNA, 25 bp, Homo sapiens
tgcaggtcatgcacacagttttgat

SEQ ID NO: 228 - DNA, 25 bp, Homo sapiens
gctccagaagcgcttggacaggctg

SEQ ID NO: 229 - DNA, 25 bp, Homo sapiens
ggcataatggcaaatccttcaagca

SEQ ID NO: 230 - DNA, 25 bp, Homo sapiens
ccccatagagacccaagttctgcgg

SEQ ID NO: 231 - DNA, 25 bp, Homo sapiens
gtaaaccacatcttttttgcacttt

SEQ ID NO: 232 - DNA, 25 bp, Homo sapiens
cactttttgtatttatcgtttcata

SEQ ID NO: 233 - DNA, 25 bp, Homo sapiens
gacgcaggacgagctcagttgtaga

SEQ ID NO: 234 - DNA, 25 bp, Homo sapiens
tgttgtgcagtgtgcctgtcactac

SEQ ID NO: 235 - DNA, 25 bp, Homo sapiens
cacttactgactttgcattttcgtt

SEQ ID NO: 236 - DNA, 25 bp, Homo sapiens
ggtggtttctcttgagactcgttac

SEQ ID NO: 237 - DNA, 25 bp, Homo sapiens
ttggcagttgcattagtaactttga

SEQ ID NO: 238 - DNA, 25 bp, Homo sapiens
tcatgtgcacatgccgttgcagcac

SEQ ID NO: 239 - DNA, 25 bp, Homo sapiens
cgttggagaactgcagctgctgtgc

SEQ ID NO: 240 - DNA, 25 bp, Homo sapiens
agcagctgaggtctcttgagggagc

SEQ ID NO: 241 - DNA, 25 bp, Homo sapiens
cattggcctgagtttcttgtgcatt

SEQ ID NO: 242 - DNA, 25 bp, Homo sapiens
gagcctacctggagggcgagtgcgt

SEQ ID NO: 243 - DNA, 25 bp, Homo sapiens
gttttcaattttgcatgctcgatta

SEQ ID NO: 244 - DNA, 25 bp, Homo sapiens
ttatgtgtacattattgttgctatt

SEQ ID NO: 245 - DNA, 25 bp, Homo sapiens
cttcccggtcactggtaacaatagc

SEQ ID NO: 246 - DNA, 25 bp, Homo sapiens
gtacctgtgggttagcatcaagttc

SEQ ID NO: 247 - DNA, 25 bp, Homo sapiens
ctgagagcctacctggagggcctgt

SEQ ID NO: 248 - DNA, 25 bp, Homo sapiens
ttgggccttttatctgtctatccat

SEQ ID NO: 249 - DNA, 25 bp, Homo sapiens
cagtgtgacaagtgccgggagatct

SEQ ID NO: 250 - DNA, 25 bp, Homo sapiens
agaatcttttctatgcctctattcc

SEQ ID NO: 251 - DNA, 25 bp, Homo sapiens
gccatatagcaggcacgtccgggtc

SEQ ID NO: 252 - DNA, 25 bp, Homo sapiens
ggcaagggataactcttctaacaca

SEQ ID NO: 253 - DNA, 25 bp, Homo sapiens
ttccctccttatagtcaaggaccgt

SEQ ID NO: 254 - DNA, 25 bp, Homo sapiens
aataacgcaaatggcttcctctttc

SEQ ID NO: 255 - DNA, 25 bp, Homo sapiens
ggcctagcacggacatggtctgtcc

SEQ ID NO: 256 - DNA, 25 bp, Homo sapiens
tgatggacgaatgccttttccggtg

SEQ ID NO: 257 - DNA, 25 bp, Homo sapiens
gatcctctgcaatgtgcttgaaaac

SEQ ID NO: 258 - DNA, 25 bp, Homo sapiens
tcacaagctattccctcaaatctga

SEQ ID NO: 259 - DNA, 25 bp, Homo sapiens
tgatgagctttcctttgatccggac

SEQ ID NO: 260 - DNA, 25 bp, Homo sapiens
gagacagctgtcttgtgagggactg

SEQ ID NO: 261 - DNA, 25 bp, Homo sapiens
tacacaccaccatatatactagctg

SEQ ID NO: 262 - DNA, 25 bp, Homo sapiens
gtgtaggtgaatgcaaactccatcg

SEQ ID NO: 263 - DNA, 25 bp, Homo sapiens
ttcctctttaaacacccgaagcgca

SEQ ID NO: 264 - DNA, 25 bp, Homo sapiens
aactgttcctgactttatactattt

SEQ ID NO: 265 - DNA, 25 bp, Homo sapiens
gcataatgatattcacatcccctca

SEQ ID NO: 266 - DNA, 25 bp, Homo sapiens
tgttgtttctgcactttataataaa

SEQ ID NO: 267 - DNA, 25 bp, Homo sapiens
agaggtggggctggatgtctccatc

SEQ ID NO: 268 - DNA, 25 bp, Homo sapiens
aagtgcaaagttattccccatcttc

SEQ ID NO: 269 - DNA, 25 bp, Homo sapiens
tacttacacccaaacagatcctgaa

SEQ ID NO: 270 - DNA, 25 bp, Homo sapiens
ttgcgtgtggagctgtattcccgag

SEQ ID NO: 271 - DNA, 25 bp, Homo sapiens
ccctttcactgttctcacaggacat

SEQ ID NO: 272 - DNA, 25 bp, Homo sapiens
gttggctgatattggagtgctcatt

SEQ ID NO: 273 - DNA, 25 bp, Homo sapiens
cagcagggtttcaggttccaatcag

SEQ ID NO: 274 - DNA, 25 bp, Homo sapiens
ctgtatttgtggtctctgtatttat

SEQ ID NO: 275 - DNA, 25 bp, Homo sapiens
acatccaaaatgacggctgctatat

SEQ ID NO: 276 - DNA, 25 bp, Homo sapiens
gtggaccctactattcatgttttga

SEQ ID NO: 277 - DNA, 25 bp, Homo sapiens
gcttaaacttacgtgccttacaggt

SEQ ID NO: 278 - DNA, 25 bp, Homo sapiens
catgcagactgttagcttttacctt

SEQ ID NO: 279 - DNA, 25 bp, Homo sapiens
gtcagtgcataaagacatactccaa

SEQ ID NO: 280 - DNA, 25 bp, Homo sapiens
ggagtcctatttgcagaaccacttt

SEQ ID NO: 281 - DNA, 25 bp, Homo sapiens
ataaccaacctattgcctatgagaa

SEQ ID NO: 282 - DNA, 25 bp, Homo sapiens
cgaattagtctccagcctctaaata

SEQ ID NO: 283 - DNA, 25 bp, Homo sapiens
tcgggtctctccataattcagccca

SEQ ID NO: 284 - DNA, 25 bp, Homo sapiens
acgattgccttcagtttgtgttgtg

SEQ ID NO: 285 - DNA, 25 bp, Homo sapiens
aattctttttattggtgcctatatt

SEQ ID NO: 286 - DNA, 25 bp, Homo sapiens
aagctcactggcatggccttccgtg

SEQ ID NO: 287 - DNA, 25 bp, Homo sapiens
gagatatttctgaattactgttgta

SEQ ID NO: 288 - DNA, 25 bp, Homo sapiens
tttgacgctggggctggcattgccc

SEQ ID NO: 289 - DNA, 25 bp, Homo sapiens
ggacagcagactgccggtaacgcgc

SEQ ID NO: 290 - DNA, 25 bp, Homo sapiens
gctctgaagtcaccaccaaaatgct

SEQ ID NO: 291 - DNA, 25 bp, Homo sapiens
ggtgccaatttcaagttccaagttg

SEQ ID NO: 292 - DNA, 25 bp, Homo sapiens
tagggagccgcaccttgtcatgtac

SEQ ID NO: 293 - DNA, 25 bp, Homo sapiens
acgtatatttacctgtgacttgtat

SEQ ID NO: 294 - DNA, 25 bp, Homo sapiens
taaactgctgcccgtagaggccttt

SEQ ID NO: 295 - DNA, 25 bp, Homo sapiens
tggatgttagcggtactcttccact

SEQ ID NO: 296 - DNA, 25 bp, Homo sapiens
ttttcctggatatctgtgtattttc

SEQ ID NO: 297 - DNA, 25 bp, Homo sapiens
actgtgcgttgtacatagttctaat

SEQ ID NO: 298 - DNA, 25 bp, Homo sapiens
gaacactggccataggaaatgctgt

SEQ ID NO: 299 - DNA, 25 bp, Homo sapiens
gatcctttctgtaggctaattcctc

SEQ ID NO: 300 - DNA, 25 bp, Homo sapiens
aacgcagcagaacttgccacatcag

SEQ ID NO: 301 - DNA, 25 bp, Homo sapiens
gaagcttggctttagtggtagaatg

SEQ ID NO: 302 - DNA, 25 bp, Homo sapiens
agcctggctcaatatctaatcaatg

SEQ ID NO: 303 - DNA, 25 bp, Homo sapiens
gaacttcaaacatttgggaccacct

SEQ ID NO: 304 - DNA, 25 bp, Homo sapiens
ccaccctagtgtctcatgtttgtat

SEQ ID NO: 305 - DNA, 25 bp, Homo sapiens
tggtgatgtctgctactatgccagc

SEQ ID NO: 306 - DNA, 25 bp, Homo sapiens
aagctggaggctgagatcgccacct

SEQ ID NO: 307 - DNA, 25 bp, Homo sapiens
cactttaacccacgcttcaatgaga

SEQ ID NO: 308 - DNA, 25 bp, Homo sapiens
tggaatgagtctcctttagtttcag

SEQ ID NO: 309 - DNA, 25 bp, Homo sapiens
agagacatgtaccttgaccatcgtc

SEQ ID NO: 310 - DNA, 25 bp, Homo sapiens
tggaaatcctctaggaggctcctcc

SEQ ID NO: 311 - DNA, 25 bp, Homo sapiens
tgtgtgggtgctgataattgtgtat

SEQ ID NO: 312 - DNA, 25 bp, Homo sapiens
tactcttccatcatctagaattgtt

SEQ ID NO: 313 - DNA, 25 bp, Homo sapiens
gcaccctcagggtcttaaggtcttc

SEQ ID NO: 314 - DNA, 25 bp, Homo sapiens
gtacacagtctgttttctatttgtt

SEQ ID NO: 315 - DNA, 25 bp, Homo sapiens
tgggctgccttatattgtgtgtgtg

SEQ ID NO: 316 - DNA, 25 bp, Homo sapiens
taacccagacttaatcttgaatgat

SEQ ID NO: 317 - DNA, 25 bp, Homo sapiens
gagacttcggcggaccattaggaat

SEQ ID NO: 318 - DNA, 25 bp, Homo sapiens
atgcacgtgaaacttaacactttat

SEQ ID NO: 319 - DNA, 25 bp, Homo sapiens
agttgaacagtgtgccctagctttt

SEQ ID NO: 320 - DNA, 25 bp, Homo sapiens
gatcttgtattcagtcaggttaaaa

SEQ ID NO: 321 - DNA, 25 bp, Homo sapiens
ccaaagtggtcagggtggcctctgg

SEQ ID NO: 322 - DNA, 25 bp, Homo sapiens
ggacgagtcggaccgaggctaggac

SEQ ID NO: 323 - DNA, 25 bp, Homo sapiens
atttgtggccactcactttgtagga

SEQ ID NO: 324 - DNA, 25 bp, Homo sapiens
tcttgctccaaagggctccgtggag

SEQ ID NO: 325 - DNA, 25 bp, Homo sapiens
ctcctccatgagtctgacatctcgg

SEQ ID NO: 326 - DNA, 25 bp, Homo sapiens
ggttgaggagaggctccagacccgc

SEQ ID NO: 327 - DNA, 25 bp, Homo sapiens
acagcaacttgtcaaacctaagcat

SEQ ID NO: 328 - DNA, 25 bp, Homo sapiens
acgcagaggacgtctctatgccggt

SEQ ID NO: 329 - DNA, 25 bp, Homo sapiens
gtagtcattcatttctagctgtaca

SEQ ID NO: 330 - DNA, 25 bp, Homo sapiens
gtagctgatgaagtatgtcgcattt

SEQ ID NO: 331 - DNA, 25 bp, Homo sapiens
gatgatcttctgtggtgcttaagga

SEQ ID NO: 332 - DNA, 25 bp, Homo sapiens
ctgtgattgctgccaggcactgttc

SEQ ID NO: 333 - DNA, 25 bp, Homo sapiens
ttcacgcggaaatacacgctgcccc

SEQ ID NO: 334 - DNA, 25 bp, Homo sapiens
tcatctgctggtccgtgggacggtg

SEQ ID NO: 335 - DNA, 25 bp, Homo sapiens
gaaggagagccatgcgtactttcta

SEQ ID NO: 336 - DNA, 25 bp, Homo sapiens
gtgtatagtgttttaccctcttctt

SEQ ID NO: 337 - DNA, 25 bp, Homo sapiens
caggacttttgttccaggttgccag

SEQ ID NO: 338 - DNA, 25 bp, Homo sapiens
gtgcagtttctgacacttgttgttg

SEQ ID NO: 339 - DNA, 25 bp, Homo sapiens
gggagcaccgtgatggagaggacag

SEQ ID NO: 340 - DNA, 25 bp, Homo sapiens
aaggccaagatcaccatcgtggcag

SEQ ID NO: 341 - DNA, 25 bp, Homo sapiens
tattcaaatggcccctccagaaagt

SEQ ID NO: 342 - DNA, 25 bp, Homo sapiens
aagccacatctgagcacttcaagac

SEQ ID NO: 343 - DNA, 25 bp, Homo sapiens
gttccaagaactctggtgtctgacc

SEQ ID NO: 344 - DNA, 25 bp, Homo sapiens
gaggatcatgcccttagcaagtact

SEQ ID NO: 345 - DNA, 25 bp, Homo sapiens
ggtctacgcctattacaacctggag

SEQ ID NO: 346 - DNA, 25 bp, Homo sapiens
aatcgttctccttacaatcaagttc

SEQ ID NO: 347 - DNA, 25 bp, Homo sapiens
ggaactaggctcttatgtgtgcctt

SEQ ID NO: 348 - DNA, 25 bp, Homo sapiens
tctgtcctcctaagaaatctgccca

SEQ ID NO: 349 - DNA, 25 bp, Homo sapiens
gaggctcagttagcaacctgtgttg

SEQ ID NO: 350 - DNA, 25 bp, Homo sapiens
agactgtccctgaaacctagtactg

SEQ ID NO: 351 - DNA, 25 bp, Homo sapiens
actttctgcatgttgatgcccgaag

SEQ ID NO: 352 - DNA, 25 bp, Homo sapiens
gttcttgtattgtattgcccagggg

SEQ ID NO: 353 - DNA, 25 bp, Homo sapiens
aaaaaccgcagcccaggttctgatc

SEQ ID NO: 354 - DNA, 25 bp, Homo sapiens
gacatgaacggctgttactcacgcc

SEQ ID NO: 355 - DNA, 25 bp, Homo sapiens
tgttcttggcgctgtatgtgcaggc

SEQ ID NO: 356 - DNA, 25 bp, Homo sapiens
cttctacaggcttttgggaagtagg

SEQ ID NO: 357 - DNA, 25 bp, Homo sapiens
tggaggacacagatgactctttggt

SEQ ID NO: 358 - DNA, 25 bp, Homo sapiens
gagtgggtggggaattttctcctct

SEQ ID NO: 359 - DNA, 25 bp, Homo sapiens
ctgcaccttcatcagcggcatgatg

SEQ ID NO: 360 - DNA, 25 bp, Homo sapiens
tataaaccactcttcaacagctggc

SEQ ID NO: 361 - DNA, 25 bp, Homo sapiens
aatacatccgatgcgtagattcttg

SEQ ID NO: 362 - DNA, 25 bp, Homo sapiens
tggcttagcctgggcaggtcgtgtc

SEQ ID NO: 363 - DNA, 25 bp, Homo sapiens
gagcagggtctctctaaaggggact

SEQ ID NO: 364 - DNA, 25 bp, Homo sapiens
gtctctgagttacaaaagtgctaat

SEQ ID NO: 365 - DNA, 25 bp, Homo sapiens
accagtttttggcttactcctgaga

SEQ ID NO: 366 - DNA, 25 bp, Homo sapiens
ttgtctacactcaggctgcagtatt

SEQ ID NO: 367 - DNA, 25 bp, Homo sapiens
tcctgagagatacctcacattccaa

SEQ ID NO: 368 - DNA, 25 bp, Homo sapiens
ggttagccttagtttctcagacttg

SEQ ID NO: 369 - DNA, 25 bp, Homo sapiens
tgtcctcatctctgcaaagttcagc

SEQ ID NO: 370 - DNA, 25 bp, Homo sapiens
ttcccaaggacaggcttcagcatca

SEQ ID NO: 371 - DNA, 25 bp, Homo sapiens
ctcctacgcagatgctgtcactggc

SEQ ID NO: 372 - DNA, 25 bp, Homo sapiens
tgagcaagggggcccgaatcgacca

SEQ ID NO: 373 - DNA, 25 bp, Homo sapiens
aagcctgctctcttgcaaagacagc

SEQ ID NO: 374 - DNA, 25 bp, Homo sapiens
taactataaggtgcctcagttttcc

SEQ ID NO: 375 - DNA, 25 bp, Homo sapiens
catatggcatctctcaacttttctt

SEQ ID NO: 376 - DNA, 25 bp, Homo sapiens
gaaagttcagccagaatcttcgtcc

SEQ ID NO: 377 - DNA, 25 bp, Homo sapiens
gggcagcttcctaatatgctttaca

SEQ ID NO: 378 - DNA, 25 bp, Homo sapiens
gcctgtcttgagtagacttggaccc

SEQ ID NO: 379 - DNA, 25 bp, Homo sapiens
gatccacctcatatgtgagtccgtc

SEQ ID NO: 380 - DNA, 25 bp, Homo sapiens
cgctgctgtgtcactcatgactgtt

SEQ ID NO: 381 - DNA, 25 bp, Homo sapiens
gcctgttgaaggtacatcgtttgca

SEQ ID NO: 382 - DNA, 25 bp, Homo sapiens
tctcccgattcccaactgagtgtta

SEQ ID NO: 383 - DNA, 25 bp, Homo sapiens
aggcaattttaatcccctgcactag

SEQ ID NO: 384 - DNA, 25 bp, Homo sapiens
ggacaaactgccagttttgtttcct

SEQ ID NO: 385 - DNA, 25 bp, Homo sapiens
gttcttcaatcagctggcacacact

SEQ ID NO: 386 - DNA, 25 bp, Homo sapiens
accacccaggaaaccatcgacaaga

SEQ ID NO: 387 - DNA, 25 bp, Homo sapiens
aaggacagaacattgccttcctcgt

SEQ ID NO: 388 - DNA, 25 bp, Homo sapiens
ggatatacaagttctgtggtttcag

SEQ ID NO: 389 - DNA, 25 bp, Homo sapiens
gtggttggcactagactggtggcag

SEQ ID NO: 390 - DNA, 25 bp, Homo sapiens
atgtacttgttctacctggattgtc

SEQ ID NO: 391 - DNA, 25 bp, Homo sapiens
cgcggctgcatgacactggaggtca

SEQ ID NO: 392 - DNA, 25 bp, Homo sapiens
ggtgcccggatccagagtgtgaaga

SEQ ID NO: 393 - DNA, 25 bp, Homo sapiens
ctccatcctatgctgctacaattgc

SEQ ID NO: 394 - DNA, 25 bp, Homo sapiens
tgaatgccagtccagatgtgcctag

SEQ ID NO: 395 - DNA, 25 bp, Homo sapiens
ttacattcatttaacctgccgatta

SEQ ID NO: 396 - DNA, 25 bp, Homo sapiens
cacccagtggtactttggtgcctac

SEQ ID NO: 397 - DNA, 25 bp, Homo sapiens
agtgctggtcactaaccaacaaggt

SEQ ID NO: 398 - DNA, 25 bp, Homo sapiens
ctctctgaaacgcttcctataactc

SEQ ID NO: 399 - DNA, 25 bp, Homo sapiens
ttgtcagttctcaatttcctgtgtt

SEQ ID NO: 400 - DNA, 25 bp, Homo sapiens
attatcgctttatgtttttggttat

SEQ ID NO: 401 - DNA, 25 bp, Homo sapiens
ggggctcaagggcaagatcagcgag

SEQ ID NO: 402 - DNA, 25 bp, Homo sapiens
gaataggtatcttgagcttggttct

SEQ ID NO: 403 - DNA, 25 bp, Homo sapiens
acactcgcttctggaacgtctgagg

SEQ ID NO: 404 - DNA, 25 bp, Homo sapiens
aatctatatttgtcttccgatcaac

SEQ ID NO: 405 - DNA, 25 bp, Homo sapiens
acaaggcccaggctggggccagggc